(19) United States
(12) Patent Application Publication
(10) Pub. No.: US 2012/0070411 A1
(43) Pub. Date: Mar. 22, 2012

(54) SUBSTITUTED NUCLEOTIDE ANALOGS

(75) Inventors: Leonid Beigelman, San Mateo, CA (US); Lawrence Blatt, San Francisco, CA (US); Guangyi Wang, Carlsbad, CA (US); Vivek Kumar Rajwanshi, Cupertino, CA (US); Natalia Dyatkina, Mountain View, CA (US); David Bernard Smith, San Mateo, CA (US)

(73) Assignee: Alios BioPharma, Inc., South San Francisco, CA (US)

(21) Appl. No.: 13/236,450

(22) Filed: Sep. 19, 2011

Related U.S. Application Data
(60) Provisional application No. 61/385,425, filed on Sep. 22, 2010.
(60) Provisional application No. 61/426,467, filed on Dec. 22, 2010.

(51) Int. Cl. A61K 38/21; C07H 19/10; A61K 31/7076; A61K 31/708; A61K 31/7068; A61P 31/22; C12N 9/99; C12N 5/071; A61P 31/14; A61P 31/12; A61P 31/20; C07H 19/207; A61K 31/7072

(52) U.S. Cl. 424/85.4; 536/26.7; 536/26.8; 514/47; 514/48; 514/49; 514/51; 435/184; 435/375

(57) ABSTRACT

Disclosed herein are phosphoroamidate nucleotide analogs, methods of synthesizing phosphoroamidate nucleotide analogs and methods of treating diseases and/or conditions such as viral infections, cancer, and/or parasitic diseases with the phosphoroamidate nucleotide analogs.

### CROSS-REFERENCE TO RELATED APPLICATIONS

This application claims the benefit of U.S. Provisional Application Nos. 61/385,425, filed Sep. 22, 2010; and 61/426,467, filed Dec. 22, 2010; both of which are incorporated herein by reference in their entirety; including any drawings.

### BACKGROUND

### Field

The present application relates to the fields of chemistry, biochemistry and medicine. More particularly, disclosed herein are phosphoroamidate nucleotide analogs, pharmaceutical compositions that include one or more nucleotide analogs and methods of synthesizing the same. Also disclosed herein are methods of treating diseases and/or conditions with a phosphoroamidate nucleotide analog, alone or in combination therapy with other agents.

### DESCRIPTION

Nucleoside analogs are a class of compounds that have been shown to exert antiviral and anticancer activity both in vitro and in vivo, and thus, have been the subject of widespread research for the treatment of viral infections and cancer. Nucleoside analogs are usually therapeutically inactive compounds that are converted by host or viral enzymes to their respective active anti-metabolites, which, in turn, may inhibit polymerases involved in viral or cell proliferation. The activation occurs by a variety of mechanisms, such as the addition of one or more phosphate groups and, or in combination with, other metabolic processes.

### SUMMARY

Some embodiments disclosed herein relate to a compound of Formula (I) or a pharmaceutically acceptable salt thereof.

Some embodiments disclosed herein relate to methods of ameliorating and/or treating a neoplastic disease that can include administering to a subject suffering from the neoplastic disease a therapeutically effective amount of one or more compounds of Formula (I), or a pharmaceutically acceptable salt thereof, or a pharmaceutical composition that includes one or more compounds of Formula (I), or a pharmaceutically acceptable salt thereof. Other embodiments described herein relate to using one or more compounds of Formula (I), or a pharmaceutically acceptable salt thereof, in the manufacture of a medicament for ameliorating and/or treating a neoplastic disease. Still other embodiments described herein relate to one or more compounds of Formula (I), or a pharmaceutically acceptable salt thereof, that can be used for ameliorating and/or treating a neoplastic disease.

Some embodiments disclosed herein relate to methods of inhibiting the growth of a tumor that can include administering to a subject having a tumor a therapeutically effective amount of one or more compounds of Formula (I), or a pharmaceutically acceptable salt thereof, or a pharmaceutical composition that includes one or more compounds of Formula (I), or a pharmaceutically acceptable salt thereof. Other embodiments described herein relate to using one or more compounds of Formula (I), or a pharmaceutically acceptable salt thereof, in the manufacture of a medicament for inhibiting the growth of a tumor. Still other embodiments described herein relate to one or more compounds of Formula (I), or a pharmaceutically acceptable salt of thereof, that can be used for inhibiting the growth of a tumor.

Some embodiments disclosed herein relate to methods of ameliorating and/or treating a viral infection that can include administering to a subject suffering from the viral infection a therapeutically effective amount of one or more compounds of Formula (I), or a pharmaceutically acceptable salt thereof, or a pharmaceutical composition that includes one or more compounds of Formula (I), or a pharmaceutically acceptable salt thereof. Other embodiments described herein relate to using one or more compounds of Formula (I), or a pharmaceutically acceptable salt thereof, in the manufacture of a medicament for ameliorating and/or treating a viral infection. Still other embodiments described herein relate to one or more compounds of Formula (I), or a pharmaceutically acceptable salt thereof, that can be used for ameliorating and/or treating a viral infection.

Some embodiments disclosed herein relate to methods of ameliorating and/or treating a viral infection that can include contacting a cell infected with the virus with an effective amount of one or more compounds described herein, or a pharmaceutically acceptable salt of one or more compounds described herein, or a pharmaceutical composition that includes one or more compounds described herein, or a pharmaceutically acceptable salt thereof. Other embodiments described herein relate to using one or more compounds described herein, or a pharmaceutically acceptable salt of one or more compounds described herein, in the manufacture of a medicament for ameliorating and/or treating a viral infection that can include contacting a cell infected with the virus with an effective amount of said compound(s). Still other embodiments described herein relate to one or more compounds described herein, or a pharmaceutically acceptable salt of one or more compounds described herein, that can be used for ameliorating and/or treating a viral infection by contacting a cell infected with the virus with an effective amount of said compound(s).

Some embodiments disclosed herein relate to methods of inhibiting replication of a virus that can include contacting a cell infected with the virus with an effective amount of one or more compounds described herein, or a pharmaceutically acceptable salt of one or more compounds described herein, or a pharmaceutical composition that includes one or more compounds described herein, or a pharmaceutically acceptable salt thereof. Other embodiments described herein relate to using one or more compounds described herein, or a pharmaceutically acceptable salt of one or more compounds described herein, in the manufacture of a medicament for inhibiting replication of a virus that can include contacting a cell infected with the virus with an effective amount of said compound(s). Still other embodiments described herein relate to one or more compounds described herein, or a pharmaceutically acceptable salt of one or more compound described herein, that can be used for inhibiting replication of a virus by contacting a cell infected with the virus with an effective amount of said compound(s).

Some embodiments disclosed herein relate to methods of ameliorating and/or treating a parasitic disease that can include administering to a subject suffering from the parasitic disease a therapeutically effective amount of one or more compounds of Formula (I), or a pharmaceutically acceptable salt thereof, or a pharmaceutical composition that includes one or more compounds of Formula (I), or a pharmaceutically acceptable salt thereof. Other embodiments described herein relate to using one or more compounds of Formula (I), or a pharmaceutically acceptable salt thereof, in the manufacture of a medicament for ameliorating and/or treating a parasitic disease. Still other embodiments described herein relate to one or more compounds of Formula (I), or a pharmaceutically acceptable salt thereof, that can be used for ameliorating and/or treating a parasitic disease.

Some embodiments disclosed herein relate to methods of ameliorating and/or treating a viral infection that can include administering to a subject suffering from the viral infection a therapeutically effective amount of a compound described herein or a pharmaceutically acceptable salt thereof (for example, a compound of Formula (I), its mono-, di-, and/or tri-phosphate, or a pharmaceutically acceptable salt of the foregoing), or a pharmaceutical composition that includes a compound described herein, or a pharmaceutically acceptable salt thereof, in combination with an agent selected from an interferon, ribavirin, a HCV protease inhibitor, a HCV polymerase inhibitor, a NS5A inhibitor, an other antiviral compound, a compound of Formula (BB), or a pharmaceutically acceptable salt thereof, a compound of Formula (CC), or a pharmaceutically acceptable salt thereof and a compound of Formula (DD), or a pharmaceutically acceptable salt thereof. Some embodiments disclosed herein relate to methods of ameliorating and/or treating a viral infection that can include contacting a cell infected with the viral infection with a therapeutically effective amount of a compound described herein or a pharmaceutically acceptable salt thereof (for example, a compound of Formula (I), its mono-, di-, and/or tri-phosphate, or a pharmaceutically acceptable salt of the foregoing), or a pharmaceutical composition that includes a compound described herein, or a pharmaceutically acceptable salt thereof, in combination with an agent selected from an interferon, ribavirin, a HCV protease inhibitor, a HCV polymerase inhibitor, a NS5A inhibitor, an other antiviral compound, a compound of Formula (BB), or a pharmaceutically acceptable salt thereof, a compound of Formula (CC), or a pharmaceutically acceptable salt thereof and a compound of Formula (DD), or a pharmaceutically acceptable salt thereof. Some embodiments disclosed herein relate to methods of inhibiting replication of a virus that can include administering to a subject a therapeutically effective amount of a compound described herein or a pharmaceutically acceptable salt thereof (for example, a compound of Formula (I), its mono-, di-, and/or tri-phosphate, or a pharmaceutically acceptable salt of the foregoing), or a pharmaceutical composition that includes a compound described herein, or a pharmaceutically acceptable salt thereof, in combination with an agent selected from an interferon, ribavirin, a HCV protease inhibitor, a HCV polymerase inhibitor, a NS5A inhibitor, an other antiviral compound, a compound of Formula (BB), or a pharmaceutically acceptable salt thereof, a compound of Formula (CC), or a pharmaceutically acceptable salt thereof and a compound of Formula (DD), or a pharmaceutically acceptable salt thereof. In some embodiments, the agent can be a compound, or a pharmaceutically acceptable salt thereof, selected from Compound 1001-1014, 2001-2010, 3001-3008, 4001-4005, 5001-5002, 6000-6078, 8000-8012 or 9000, or a pharmaceutical composition that includes one or more of the aforementioned compounds, or pharmaceutically acceptable salt thereof. In some embodiments, the method can further include administering a second agent selected from an interferon, ribavirin, a HCV protease inhibitor, a HCV polymerase inhibitor, a NS5A inhibitor, an other antiviral compound, a compound of Formula (BB), or a pharmaceutically acceptable salt thereof, a compound of Formula (CC), or a pharmaceutically acceptable salt thereof and a compound of Formula (DD), or a pharmaceutically acceptable salt thereof. In some embodiments, the viral infection is HCV.

### BRIEF DESCRIPTION OF THE DRAWINGS

FIG. 1 shows example HCV protease inhibitors.

FIG. 2 shows example nucleoside HCV polymerase inhibitors.

FIG. 3 shows example non-nucleoside HCV polymerase inhibitors.

FIG. 4 shows example NS5A inhibitors.

FIG. 5 shows example other antivirals.

FIGS. 6A-6I show example compounds of Formula (CC).

FIGS. 7A-7I show example compounds of Formula (I) and triphosphates thereof.

FIGS. 8A-8B show example compounds of Formula (BB).

FIG. 9 shows Formula (DD).

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R¹, R², R^{3a}, R^{3b}, R⁴, R⁵, R⁶, R⁷, R⁸, R⁹, R¹⁰, R¹¹, R¹², R¹³, R¹⁴, R¹⁵, R¹⁶, R¹⁷, R^{1A}, R^{2A}, R^{3A}, R^{3B}, R^{4A}, R^{5A}, R^{6A}, R^{7A}, R^{8A} and R″ represent substituents that can be attached to the indicated atom. An R group may be substituted or unsubstituted. If two “R” groups are described as being “taken together” the R groups and the atoms they are attached to can form a cycloalkyl, aryl, heteroaryl or heterocycle. For example, without limitation, if R^{1a} and R^{1b} of an NR^{1a}R^{1b} group are indicated to be “taken together,” it means that they are covalently bonded to one another to form a ring:

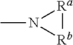

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amino and a di-substituted amino group, and protected derivatives thereof.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

As used herein, “cycloalkynyl” refers to a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl, and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs.

A “(heteroalicyclyl)alkyl” and “(heterocyclyl)alkyl” refer to a heterocyclic or a heteroalicyclylic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl or a cycloalkynyl is defined as above. A non-limiting list of alkoxys are methoxy, ethoxy, n-propoxy, 1-methylethoxy(isopropoxy), n-butoxy, iso-butoxy, sec-butoxy and tert-butoxy. An alkoxy may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

As used herein, “aryloxy” and “arylthio” refers to RO— and RS—, in which R is an aryl, such as but not limited to phenyl. Both an aryloxy and arylthio may be substituted or unsubstituted.

A “sulfenyl” group refers to an “—SR” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein X is a halogen.

A “trihalomethanesulfonamido” group refers to an “X₃CS(O)₂N(R_{A})—” group wherein X is a halogen and R_{A} hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl.

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An S-sulfonamido may be substituted or unsubstituted.

An “N-sulfonamido” group refers to a “RSO₂N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-sulfonamido may be substituted or unsubstituted.

An “O-carbamyl” group refers to a “—OC(═O)N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-carbamyl may be substituted or unsubstituted.

An “N-carbamyl” group refers to an “ROC(═O)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-carbamyl may be substituted or unsubstituted.

An “O-thiocarbamyl” group refers to a “—OC(═S)—N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-thiocarbamyl may be substituted or unsubstituted.

An “N-thiocarbamyl” group refers to an “ROC(═S)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-thiocarbamyl may be substituted or unsubstituted.

A “C-amido” group refers to a “—C(═O)N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A C-amido may be substituted or unsubstituted.

An “N-amido” group refers to a “RC(═O)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-amido may be substituted or unsubstituted.

The term “halogen atom” or “halogen” as used herein, means any one of the radio-stable atoms of column 7 of the Periodic Table of the Elements, such as, fluorine, chlorine, bromine and iodine.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “nucleoside” is used herein in its ordinary sense as understood by those skilled in the art, and refers to a compound composed of an optionally substituted pentose moiety or modified pentose moiety attached to a heterocyclic base or tautomer thereof via a N-glycosidic bond, such as attached via the 9-position of a purine-base or the 1-position of a pyrimidine-base. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers. In some instances, the nucleoside can be a nucleoside analog drug.

As used herein, the term “heterocyclic base” refers to an optionally substituted nitrogen-containing heterocyclyl that can be attached to an optionally substituted pentose moiety or modified pentose moiety. In some embodiments, the heterocyclic base can be selected from an optionally substituted purine-base, an optionally substituted pyrimidine-base and an optionally substituted triazole-base (for example, a 1,2,4-triazole). The term “purine-base” is used herein in its ordinary sense as understood by those skilled in the art, and includes its tautomers. Similarly, the term “pyrimidine-base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g., 7-methylguanine), theobromine, caffeine, uric acid and isoguanine. Examples of pyrimidine-bases include, but are not limited to, cytosine, thymine, uracil, 5,6-dihydrouracil and 5-alkylcytosine (e.g., 5-methylcytosine). An example of an optionally substituted triazole-base is 1,2,4-triazole-3-carboxamide. Other non-limiting examples of heterocyclic bases include diaminopurine, 8-oxo-N⁶-alkyladenine (e.g., 8-oxo-N⁶-methyladenine), 7-deazaxanthine, 7-deazaguanine, 7-deazaadenine, N⁴,N⁴-ethanocytosin, N⁶,N⁶-ethano-2,6-diaminopurine, 5-halouracil (e.g., 5-fluorouracil and 5-bromouracil), pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases. In some embodiments, a heterocyclic base can be optionally substituted with an amine or an enol protecting group(s).

The term “—N-linked amino acid” refers to an amino acid that is attached to the indicated moiety via a main-chain amino or mono-substituted amino group. When the amino acid is attached in an —N-linked amino acid, one of the hydrogens that is part of the main-chain amino or mono-substituted amino group is not present and the amino acid is attached via the nitrogen. As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine. N-linked amino acids can be substituted or unsubstituted.

The term “—N-linked amino acid ester derivative” refers to an amino acid in which a main-chain carboxylic acid group has been converted to an ester group. In some embodiments, the ester group has a formula selected from alkyl-O—C(═O)—, cycloalkyl-O—C(═O)—, aryl-O—C(═O)— and aryl(alkyl)-O—C(═O)—. A non-limiting list of ester groups include, methyl-O—C(═O)—, ethyl-O—C(═O)—, n-propyl-O—C(═O)—, isopropyl-O—C(═O)—, n-butyl-β—C(═O)—, isobutyl-O—C(═O)—, tert-butyl-O—C(═O)—, neopentyl-O—C(═O)—, cyclopropyl-β—C(═O)—, cyclobutyl-O—C(═O)—, cyclopentyl-O—C(═O)—, cyclohexyl-O—C(═O)—, phenyl-O—C(═O)—, and benzyl-O—C(═O)—. N-linked amino acid ester derivatives can be substituted or unsubstituted.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

“Leaving group” as used herein refers to any atom or moiety that is capable of being displaced by another atom or moiety in a chemical reaction. More specifically, in some embodiments, “leaving group” refers to the atom or moiety that is displaced in a nucleophilic substitution reaction. In some embodiments, “leaving groups” are any atoms or moieties that are conjugate bases of strong acids. Examples of suitable leaving groups include, but are not limited to, tosylates and halogens. Non-limiting characteristics and examples of leaving groups can be found, for example in Organic Chemistry, 2d ed., Francis Carey (1992), pages 328-331; Introduction to Organic Chemistry, 2d ed., Andrew John McMurry (2000), pages 398 and 408; all of which are incorporated herein by reference for the limited purpose of disclosing characteristics and examples of leaving groups.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of phosphate groups are intended to be included. Furthermore, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Some embodiments disclosed herein relate to a compound of Formula (I) or a pharmaceutically acceptable salt thereof:

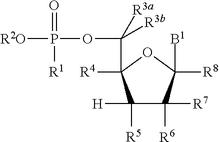

wherein: B¹ can be an optionally substituted heterocyclic base or an optionally substituted heterocyclic base with a protected amino group; R¹ can be an optionally substituted N-linked amino acid or an optionally substituted N-linked amino acid ester derivative; R² can be selected from an optionally substituted aryl, an optionally substituted heteroaryl and an optionally substituted heterocyclyl; R^{3a} and R^{3b} can be independently selected from hydrogen, an optionally substituted C₁₋₆ alkyl, an optionally substituted C₂₋₆ alkenyl, an optionally substituted C₂₋₆ alkynyl, an optionally substituted C₁₋₆ haloalkyl and aryl(C₁₋₆ alkyl), provided that at least one of R^{3a} and R^{3b} cannot be hydrogen; or R^{3a} and R^{3b} can be taken together to form a group selected from an optionally substituted C₃₋₆ cycloalkyl, an optionally substituted C₃₋₆ cycloalkenyl, an optionally substituted C₃₋₆ aryl, and an optionally substituted C₃₋₆ heteroaryl; R⁴ can be hydrogen; R⁵ can be selected from hydrogen, —OR⁹ and —OC(═O)R¹⁰; R⁶ can be selected from hydrogen, halogen, —OR¹¹ and —OC(═O)R¹²; or R⁵ and R⁶ can be both oxygen atoms and linked together by a carbonyl group; R⁷ can be selected from hydrogen, halogen, an optionally substituted C₁₋₆ alkyl, —OR¹³ and —OC(═O)R¹⁴; R⁸ can be hydrogen or an optionally substituted C₁₋₆ alkyl; R⁹, R¹¹ and R¹³ can be independently selected from hydrogen and an optionally substituted C₁₋₆ alkyl; and R¹⁰, R¹² and R¹⁴ can be independently selected from an optionally substituted C₁₋₆ alkyl and an optionally substituted C₃₋₆ cycloalkyl.

In some embodiments, a compound of Formula (I) can have a structure selected from:

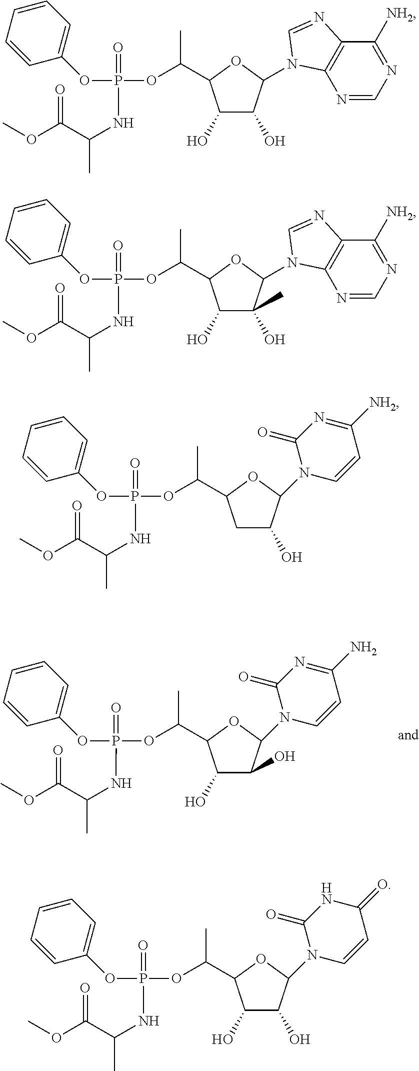

With respect to R², in some embodiments, R² can be an optionally substituted heteroaryl. In other embodiments, R² can be an optionally substituted heterocyclyl. In still other embodiments, R² can be an optionally substituted aryl. For example, R² can be an optionally substituted phenyl or an optionally substituted naphthyl. If R² is a substituted phenyl or a substituted naphthyl, the phenyl ring and the naphthyl ring(s) can be substituted one or more times. Suitable substituents that can be present on optionally substituted phenyl and an optionally substituted naphthyl include electron-donating groups and electron-withdrawing groups. In some embodiments, R² can be a para-substituted phenyl. In other embodiment, R² can be an unsubstituted phenyl or an unsubstituted naphthyl.

Various amino acids and amino acid ester derivatives can be used, including those described herein. In some embodiment, R¹ can be an optionally substituted N-linked α-amino acid. Suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional suitable amino acids include, but are not limited to, alpha-ethyl-glycine, alpha-propyl-glycine and beta-alanine. In other embodiments, R¹ can be an optionally substituted N-linked α-amino acid ester derivative. For example, R¹ can be an ester derivative of any of the following amino acids: alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of N-linked amino acid ester derivatives include, but are not limited to, an ester derivative of any of the following amino acids: alpha-ethyl-glycine, alpha-propyl-glycine and beta-alanine.

In an embodiment, R¹ can be an ester derivative of alanine. In an embodiment, R¹ can be selected from alanine methyl ester, alanine ethyl ester, alanine isopropyl ester, alanine cyclohexyl ester, alanine neopentyl ester, valine isopropyl ester and leucine isopropyl ester. In some embodiments, when R¹ is an optionally substituted N-linked α-amino acid ester derivative, then R² can be an optionally substituted aryl. In some embodiments, the optionally substituted N-linked amino acid or the optionally substituted N-linked amino acid ester derivative can be in the L-configuration. In other embodiments, the optionally substituted N-linked amino acid or the optionally substituted N-linked amino acid ester derivative can be in the D-configuration.

In some embodiments, when R¹ is an optionally substituted N-linked α-amino acid or an optionally substituted N-linked α-amino acid ester derivative, then R² can be selected from optionally substituted aryl, an optionally substituted heteroaryl and an optionally substituted heterocyclyl. In some embodiments, when R¹ is an optionally substituted N-linked α-amino acid ester derivative, then R² can be an optionally substituted aryl. In other embodiments, when R¹ is an optionally substituted N-linked α-amino acid ester derivative, then R² can be an optionally substituted heteroaryl. In still other embodiments, when R¹ is an optionally substituted N-linked α-amino acid ester derivative, then R² can be an optionally substituted heterocyclyl.

In some embodiments, R¹ can have the structure

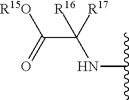

wherein R¹⁵ can be selected from hydrogen, an optionally substituted C₁₋₆-alkyl, an optionally substituted C₃₋₆ cycloalkyl, an optionally substituted aryl, an optionally substituted aryl(C₁₋₆ alkyl) and an optionally substituted C₁₋₆ haloalkyl; and R¹⁶ can be selected from hydrogen, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl(C₁₋₆ alkyl); and R¹⁷ can be hydrogen or an optionally substituted C₁₋₄-alkyl; or R¹⁶ and R¹⁷ can be taken together to form an optionally substituted C₃₋₆ cycloalkyl.

When R¹ has the structure shown above, R¹⁶ can be an optionally substituted C₁₋₆-alkyl. Examples of suitable optionally substituted C₁₋₆-alkyls include optionally substituted variants of the following: methyl, ethyl, n-propyl, isopropyl, n-butyl, isobutyl, tert-butyl, pentyl (branched and straight-chained), and hexyl (branched and straight-chained). When R¹⁶ is substituted, R¹⁶ can be substituted with one or more substituents selected from N-amido, mercapto, alkylthio, an optionally substituted aryl, hydroxy, an optionally substituted heteroaryl, O-carboxy, and amino. In some embodiment, R¹⁶ can be an unsubstituted C₁₋₆-alkyl, such as methyl, ethyl, n-propyl, isopropyl, n-butyl, isobutyl, tert-butyl, pentyl (branched and straight-chained), and hexyl (branched and straight-chained). In an embodiment, R¹⁶ can be methyl.

As to R¹⁵, in some embodiments, R¹⁵ can be an optionally substituted C₁₋₆ alkyl. Examples of optionally substituted C₁₋₆-alkyls include optionally substituted variants of the following: methyl, ethyl, n-propyl, isopropyl, n-butyl, isobutyl, tert-butyl, pentyl (branched and straight-chained), and hexyl (branched and straight-chained). In some embodiments, R¹⁵ can be methyl or isopropyl. In some embodiments, R¹⁵ can be ethyl or neopentyl. In other embodiments, R¹⁵ can be an optionally substituted C₃₋₆ cycloalkyl. Examples of optionally substituted C₃₋₆ cycloalkyl include optionally substituted variants of the following: cyclopropyl, cyclobutyl, cyclopentyl, and cyclohexyl. In an embodiment, R¹⁵ can be an optionally substituted cyclohexyl. In still other embodiments, R¹⁵ can be an optionally substituted aryl, such as phenyl and naphthyl. In yet still other embodiments, R¹⁵ can be an optionally substituted aryl(C₁₋₆ alkyl). In some embodiments, R¹⁵ can be an optionally substituted benzyl. In some embodiments, R¹⁵ can be an optionally substituted C₁₋₆ haloalkyl, for example, CF₃.

In some embodiments, R¹⁷ can be hydrogen. In other embodiments, R¹⁷ can be an optionally substituted C₁₋₄-alkyl, such as methyl, ethyl, n-propyl, isopropyl, n-butyl, isobutyl and tert-butyl. In an embodiment, R¹⁷ can be methyl. In some embodiments, R¹⁶ and R¹⁷ can be taken together to form an optionally substituted C₃₋₆ cycloalkyl. Examples of optionally substituted C₃₋₆ cycloalkyl include optionally substituted variants of the following: cyclopropyl, cyclobutyl, cyclopentyl, and cyclohexyl. Depending on the groups that are selected for R¹⁶ and R¹⁷, the carbon to which R¹⁶ and R¹⁷ are attached may be a chiral center. In some embodiment, the carbon to which R¹⁶ and R¹⁷ are attached may be a (R)-chiral center. In other embodiments, the carbon to which R¹⁶ and R¹⁷ are attached may be a (S)-chiral center.

Examples of a suitable

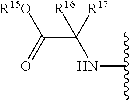

groups include the following:

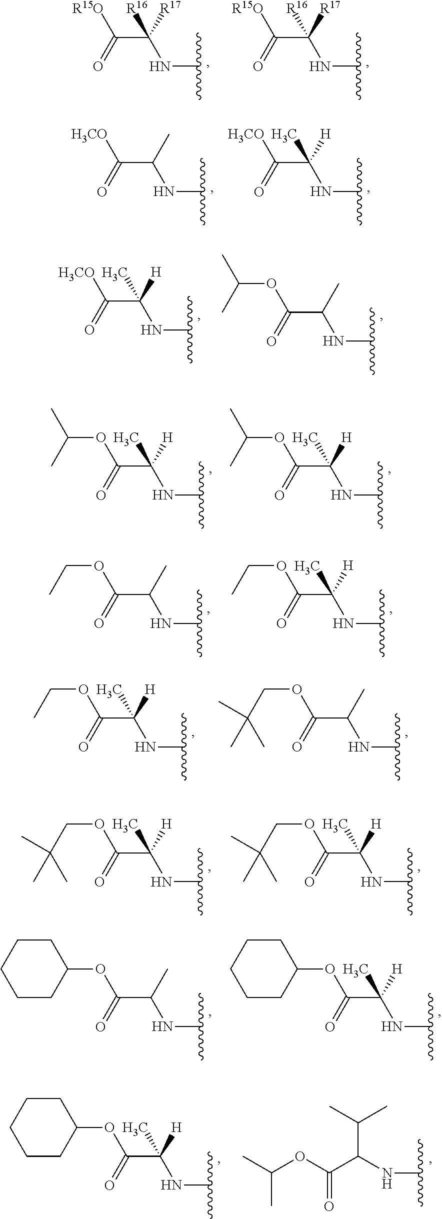

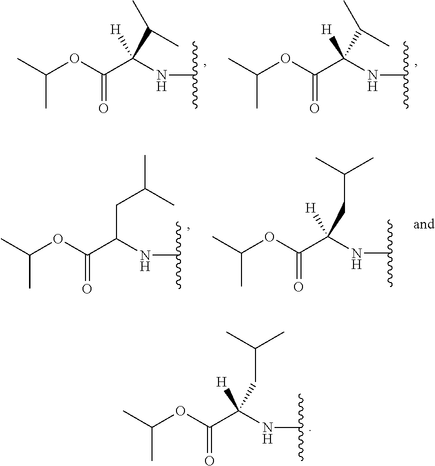

Depending upon the substituents attached to the phosphorus atom, the phosphorus atom can be a chiral center. In some embodiments, the phosphorus can be a (R)-stereocenter. In other embodiments, the phosphorus can be a (S)-stereocenter.

The substituents attached to the 5′-position of a compound of Formula (I) can vary. In some embodiments, R^{3a} and R^{3b} can be the same. In other embodiments, R^{3a} and R^{3b} can be different. In some embodiments, at least one of R^{3a} and R^{3b} cannot be hydrogen. In some embodiments, R^{3a} can be hydrogen. In some embodiments, R^{3a} can be an optionally substituted C₁₋₆ alkyl. Examples of suitable optionally substituted C₁₋₆ alkyls include optionally substituted variants of the following: methyl, ethyl, n-propyl, isopropyl, n-butyl, isobutyl, tert-butyl, pentyl (branched and straight-chained), and hexyl (branched and straight-chained). In some embodiments, R^{3a} can be an optionally substituted C₂₋₆ alkyl. In some embodiments, R^{3a} can be an optionally substituted C₂₋₆ alkenyl. In some embodiments, R^{3a} can be an optionally substituted C₂₋₆ alkynyl. In some embodiments, R^{3a} can be an optionally substituted C₁₋₆ haloalkyl. One example of a suitable optionally substituted C₁₋₆-haloalkyl is CF₃. In some embodiments, R^{3a} can be aryl(C₁₋₆ alkyl). One example of a suitable optionally substituted aryl(C₁₋₆ alkyl) is benzyl. In some embodiments, R^{3b} can be hydrogen. In some embodiments, R^{3b} can be an optionally substituted C₁₋₆ alkyl. In some embodiments, R^{3b} can be an optionally substituted C₂₋₆ alkyl. In some embodiments, R^{3b} can be an optionally substituted C₂₋₆ alkenyl. In some embodiments, R^{3b} can be an optionally substituted C₂₋₆ alkynyl. In some embodiments, R^{3b} can be an optionally substituted C₁₋₆ haloalkyl. In some embodiments, R^{3b} can be aryl(C₁₋₆ alkyl). In some embodiments, R^{3a} and R^{3b} can be taken together to form a group selected from an optionally substituted C₃₋₆ cycloalkyl, an optionally substituted C₃₋₆ cycloalkenyl, an optionally substituted C₃₋₆ aryl, and an optionally substituted C₃₋₆ heteroaryl. In some embodiments, R^{3a} and R^{3b} can be taken together to form an optionally substituted C₃₋₆ cycloalkyl.

In some embodiments, at least one of R^{3a} and R^{3b} can be an optionally substituted C₁₋₆-alkyl; and the other of R^{3a} and R^{3b} can be hydrogen. Examples of suitable optionally substituted C₁₋₆ alkyls include optionally substituted variants of the following: methyl, ethyl, n-propyl, isopropyl, n-butyl, isobutyl, tert-butyl, pentyl (branched and straight-chained), and hexyl (branched and straight-chained). In an embodiment, at least one of R^{3a} and R^{3b} can be methyl, and the other of R^{3a} and R^{3b} can be hydrogen. In some embodiments, at least one of R^{3a} and R^{3b} can be an optionally substituted C₂₋₆-alkyl; and the other of R^{3a} and R^{3b} can be hydrogen. In other embodiments, at least one of R^{3a} and R^{3b} can be an optionally substituted C₁₋₆-haloalkyl, and the other of R^{3a} and R^{3b} can be hydrogen. One example of a suitable optionally substituted C₁₋₆-haloalkyl is CF₃. When the substituents attached to the 5′-carbon make the 5′-carbon chiral, in some embodiments, the 5′-carbon can be a (R)-stereocenter. In other embodiments, the 5′-carbon can be an (S)-stereocenter.

The substituents attached to the 2′-carbon and the 3′-carbon can also vary. In some embodiments, R⁷ can be hydrogen. In other embodiments, R⁷ can be halogen. In still other embodiments, R⁷ can be —OR¹³. When R¹³ is hydrogen, R⁷ can be hydroxy. Alternatively, when R¹³ is an optionally substituted C₁₋₆ alkyl, R⁷ can be an optionally substituted C₁₋₆ alkoxy. Suitable alkoxy groups include methoxy, ethoxy, n-propoxy, isopropoxy, n-butoxy, isobutoxy, tert-butoxy, pentoxy (branched and straight-chained), and hexoxy (branched and straight-chained). In yet still other embodiments, R⁷ can be an optionally substituted C₁₋₆ alkyl. Examples of optionally substituted C₁₋₆ alkyl groups include, but are not limited to, methyl, ethyl, n-propyl, isopropyl, n-butyl, isobutyl, tert-butyl, pentyl (branched and straight-chained), and hexyl (branched and straight-chained). In some embodiments, R⁷ can be —OC(═O)R¹⁴ in which R¹⁴ is an optionally substituted C₁₋₆ alkyl or an optionally substituted C₃₋₆ cycloalkyl. Examples of suitable C₁₋₆ alkyl and C₃₋₆ cycloalkyl groups are described herein.

In some embodiments, R⁴ can be hydrogen. In some embodiments, R⁵ can be hydrogen. In other embodiments, R⁵ can be —OR⁹, wherein R⁹ can be hydrogen. In yet still other embodiments, R⁵ can be —OR⁹, wherein R⁹ can be an optionally substituted C₁₋₆ alkyl. A non-limiting list of examples of R⁵ being —OR⁹, wherein R⁹ can be an optionally substituted C₁₋₆ alkyl are methoxy, ethoxy, n-propoxy, isopropoxy, n-butoxy, isobutoxy, tert-butoxy, pentoxy (straight-chained or branched) and hexoxy (straight-chained or branched). In some embodiments, R⁵ can be —OC(═O)R¹⁰, wherein R¹⁰ can be selected from an optionally substituted C₁₋₆ alkyl and an optionally substituted C₃₋₆ cycloalkyl. Examples of optionally substituted C₁₋₆ alkyls and optionally substituted C₃₋₆ cycloalkyls are described herein.

In some embodiments, R⁶ can be hydrogen. In some embodiments, R⁶ can be halogen. In still other embodiments, R⁶ can be —OR¹¹. In an embodiment, when R¹¹ is hydrogen, R⁶ can be a hydroxy group. In yet still other embodiments, when R¹¹ is an optionally substituted C₁₋₆ alkyl, R⁶ can be an optionally substituted C₁₋₆ alkoxy. Suitable optionally substituted C₁₋₆ alkoxy groups are described herein. In some embodiments, R⁶ can be —OC(═O)R¹², wherein R¹² can be an optionally substituted C₁₋₆ alkyl, such as optionally substituted variants of the following: methyl, ethyl, n-propyl, isopropyl, n-butyl, isobutyl, tert-butyl, pentyl (branched and straight-chained), and hexyl (branched and straight-chained). In other embodiments, R⁶ can be —OC(═O)R¹², wherein R¹² can be an optionally substituted C₃₋₆ cycloalkyl. Examples of optionally substituted C₁₋₆ alkyls and optionally substituted C₃₋₆ cycloalkyls are described herein.

In some embodiments, R⁵ and R⁶ can both be hydroxy. In still other embodiments, R⁵ and R⁶ can both be both oxygen atoms and linked together by a carbonyl group, for example, —O—C(═O)—O—. In some embodiments, at least one of R⁶ and R⁷ can be a halogen. In some embodiments, R⁶ and R⁷ can both be a halogen. In other embodiments, R⁶ can be a halogen and R⁷ can be an optionally substituted C₁₋₆ alkyl, such as those described herein. In other embodiments, R⁶ can be hydrogen and R⁷ can be a halogen. In some embodiments, R⁵ can be —OC(═O)R¹⁰ and R⁶ can be —OC(═O)R¹². In some embodiments, R⁶ can be hydrogen and R⁷ can be hydroxy. Those skilled in the art understand that when a hydrogen atom is removed from an oxygen atom, the oxygen atoms can have a negative charge. For example, when R⁵ and/or R⁶ is a hydroxy group and the hydrogen is removed, the oxygen atom to which to hydrogen atom was associated with can be O⁻. In some embodiments, R^{3a}, R^{3b}, R⁴ and R⁸ can be hydrogen in any of the embodiments described in this paragraph. In some embodiments, B¹ can be an optionally substituted adenine, an optionally substituted guanine, and optionally substituted thymine, optionally substituted cytosine, or an optionally substituted uracil in any of the embodiments described in this paragraph.

In some embodiments, R⁸ can be hydrogen. In other embodiments, R⁸ can be an optionally substituted C₁₋₆ alkyl. For example, R⁸ can be selected from methyl, ethyl, n-propyl, isopropyl, n-butyl, isobutyl, tert-butyl, pentyl (branched and straight-chained), and hexyl (branched and straight-chained).

Various optionally substituted heterocyclic bases can be attached to the pentose ring. In some embodiments, one or more of the amine and/or amino groups may be protected with a suitable protecting group. For example, an amino group may be protected by transforming the amine and/or amino group to an amide or a carbamate. In some embodiments, an optionally substituted heterocyclic base or an optionally substituted heterocyclic base with one or more protected amino groups can have one of the following structures:

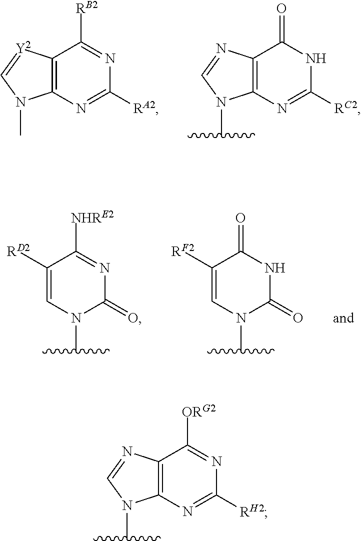

wherein: R^{A2} can be selected from hydrogen, halogen and NHR^{J2}, wherein R^{J2} can be selected from hydrogen, —C(═O)R^{K2} and —C(═O)OR^{L2}; R^{B2} can be halogen or NHR^{W2}, wherein R^{W2} is selected from hydrogen, an optionally substituted C₁₋₆ alkyl, an optionally substituted C₂₋₆ alkenyl, an optionally substituted C₃₋₈ cycloalkyl, —C(═O)R^{M2} and —C(═O)OR^{N2}; R^{C2} can be hydrogen or NHR^{O2}, wherein R^{O2} can be selected from hydrogen, —C(═O)R^{P2} and —C(═O)OR^{Q2}; R^{D2} can be selected from hydrogen, halogen, an optionally substituted C₁₋₆ alkyl, an optionally substituted C₂₋₆ alkenyl and an optionally substituted C₂₋₆ alkynyl; R^{E2} can be selected from hydrogen, an optionally substituted C₁₋₆ alkyl, an optionally substituted C₃₋₈ cycloalkyl, —C(═O)R^{R2} and —C(═O)OR^{S2}; R^{F2} can be selected from hydrogen, halogen, an optionally substituted C₁₋₆ alkyl, an optionally substituted C₂₋₆ alkenyl and an optionally substituted C₂₋₆ alkynyl; Y² can be N (nitrogen) or CR^{I2}, wherein R^{I2} can be selected from hydrogen, halogen, an optionally substituted C₁₋₆-alkyl, an optionally substituted C₂₋₆-alkenyl and an optionally substituted C₂₋₆-alkynyl; R^{G2} can be an optionally substituted C₁₋₆ alkyl; R^{H2} can be hydrogen or NHR^{T2}, wherein R^{T2} can be independently selected from hydrogen, —C(═O)R^{U2} and —C(═O)OR^{V2}, and R^{K2}, R^{L2}, R^{M2}, R^{N2}, R^{P2}, R^{Q2} R^{R2}, R^{S2}, R^{U2} and R^{V2} can be independently selected from C₁₋₆ alkyl, C₂₋₆ alkenyl, C₂₋₆ alkynyl, C₃₋₆ cycloalkyl, C₃₋₆ cycloalkenyl, C₃₋₆ cycloalkynyl, C₆₋₁₀ aryl, heteroaryl, heteroalicyclyl, aryl(C₁₋₆ alkyl), heteroaryl(C₁₋₆ alkyl) and heteroalicyclyl(C₁₋₆ alkyl). In some embodiments, the structures shown above can be modified by replacing one or more hydrogens with substituents selected from the list of substituents provided for the definition of “substituted.”

In some embodiments, can be selected from adenine, guanine, thymine, cytosine and uracil. In some embodiments, B¹ can be

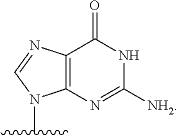

In other embodiments, B¹ can be

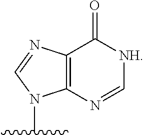

In still other embodiments, B¹ can be

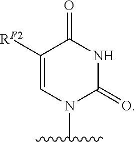

In yet still other embodiments, B¹ can be

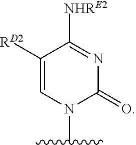

In some embodiments, B¹ can be

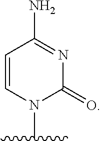

In other embodiments, B¹ can be

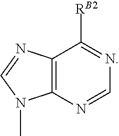

In some embodiments, R^{B2} can be NH₂. In some embodiments, R^{B2} can be NHR^{W2}, R^{W2} can be —C(═O)R^{M2}, and R^{M2} can be C₁₋₆ alkyl. In yet still other embodiments, B¹ can be

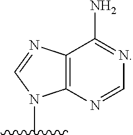

In some embodiments, B¹ can be

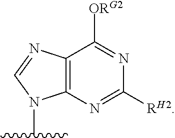

In some embodiments, a compound of Formula (I) cannot have a structure selected from:

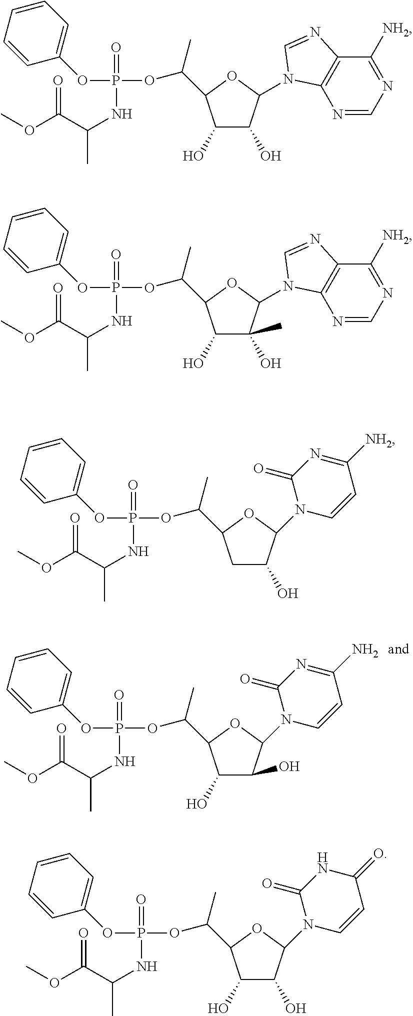

In some embodiments, when R² is a substituted or unsubstituted phenyl, then R¹ cannot be

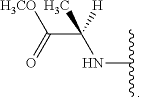

In other embodiments, when R² is a substituted or unsubstituted phenyl, then R¹ cannot be

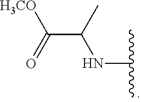

In still other embodiments, when R² is a substituted or unsubstituted phenyl and R¹ is

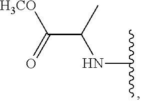

then at least one of R⁵ and R⁶ cannot be hydroxy. In some embodiments, when R¹ is

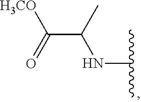

R² is phenyl, one of R^{3a} and R^{3b} are methyl and the other of R^{3a} and R^{3b} is hydrogen, then B¹ cannot be adenosine, cytosine, or uracil. In some embodiments, when R¹ is

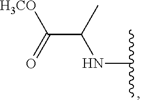

R² is phenyl, one of R^{3a} and R^{3b} are methyl and the other of R^{3a} and R^{3b} is hydrogen, then R⁶ cannot be OH. In some embodiments, when R¹ is

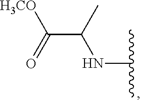

R² is phenyl, one of R^{3a} and R^{3b} are methyl and the other of R^{3a} and R^{3b} is hydrogen, then at least one of R⁵, R⁶ and R⁷ is halogen. In some embodiments, when R¹ is

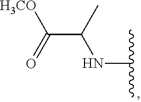

R² is phenyl, one of R^{3a} and R^{3b} are methyl and the other of R^{3a} and R^{3b} is hydrogen, and B¹ is cytosine, then R⁷ cannot be hydroxy.

In some embodiments, R^{3a} cannot be hydrogen. In some embodiments, R^{3b} cannot be hydrogen. In some embodiments, R^{3a} cannot be an optionally substituted C₁₋₆ alkyl. In some embodiments, R^{3b} cannot be an optionally substituted C₁₋₆ alkyl. In some embodiments, R^{3a} cannot be methyl. In some embodiments, R^{3b} cannot be methyl. In other embodiments, R^{3a} cannot be an optionally substituted C₁₋₆-haloalkyl. In other embodiments, R^{3b} cannot be an optionally substituted C₁₋₆-haloalkyl.

In other embodiments, at least one of R⁵ and R⁶ cannot be hydroxy. For example, R⁵ cannot be hydroxy, R⁶ cannot be hydroxy, or both of R⁵ and R⁶ cannot be hydroxy. In some embodiments, R⁵ cannot be hydrogen. In some embodiments, R⁵ cannot be halogen. In still other embodiments, R⁵ cannot be —OR⁹. In some embodiments, R⁶ cannot be hydrogen. In some embodiments, R⁶ cannot be halogen. In still other embodiments, R⁶ cannot be —OR¹¹. In some embodiments, R⁷ cannot be hydrogen. In other embodiments, R⁷ cannot be halogen. In still other embodiments, R⁷ cannot be —OR¹³. In some embodiments, R⁷ cannot be hydroxy.

In some embodiments, B¹ cannot be

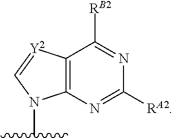

In some embodiments, B¹ cannot be

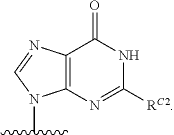

In some embodiments, B¹ cannot be

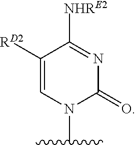

In some embodiments, B¹ cannot be

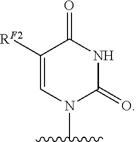

In some embodiments, B¹ cannot be

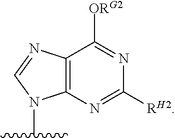

In some embodiments, B¹ cannot be adenine. In still other embodiments, B¹ cannot be thymine. In yet still other embodiments, B¹ cannot be uracil. In some embodiments, B¹ cannot be cytosine. In other embodiments, B¹ cannot be guanine. In other embodiments, B¹ cannot be hypoxanthine.

Some embodiments disclosed herein relate to a compound of Formula (I) or a pharmaceutically acceptable salt thereof, wherein: B¹ can be an optionally substituted heterocyclic base as described in paragraph [0106]; R¹ can be an optionally substituted N-linked amino acid or an optionally substituted N-linked amino acid ester derivative; R² can be an optionally substituted aryl; R^{3a} and R^{3b}) can be independently hydrogen or an optionally substituted C₁₋₆ alkyl, provided that at least one of R^{3a} and R^{3b}) cannot be hydrogen; R⁴ can be hydrogen; R⁵ can be selected from hydrogen, —OR⁹ and —OC(═O)R¹⁰; R⁶ can be selected from hydrogen, halogen, —OR¹¹ and —OC(═O)R¹²; or R⁵ and R⁶ can be both oxygen atoms and linked together by a carbonyl group; R⁷ can be selected from hydrogen, halogen, an optionally substituted C₁₋₆ alkyl, and —OR¹³; R⁸ can be hydrogen; R⁹, R¹¹ and R¹³ can be independently hydrogen or an optionally substituted C₁₋₆ alkyl; and R¹⁰ and R¹² can be independently an optionally substituted C₁₋₆ alkyl.

Some embodiments disclosed herein relate to a compound of Formula (I) or a pharmaceutically acceptable salt thereof, wherein: B¹ can be an optionally substituted heterocyclic base or an optionally substituted heterocyclic base with a protected amino group selected from

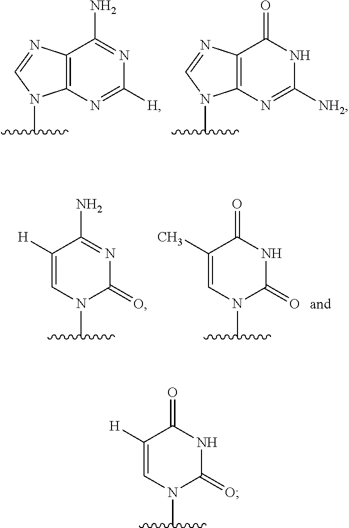

R¹ can be an optionally substituted N-linked amino acid or an optionally substituted N-linked amino acid ester derivative; R² can be an optionally substituted aryl; R^{3a} and R^{3b}) can be independently hydrogen or an optionally substituted C₁₋₆ alkyl, provided that at least one of R^{3a} and R^{3b}) cannot be hydrogen; R⁴ can be hydrogen; R⁵ can be selected from hydrogen, —OR⁹ and —OC(═O)R¹⁰; R⁶ can be selected from hydrogen, halogen, —OR¹¹ and —OC(═O)R¹²; or R⁵ and R⁶ can be both oxygen atoms and linked together by a carbonyl group; R⁷ can be selected from hydrogen, halogen, an optionally substituted C₁₋₆ alkyl, and —OR¹³; R⁸ can be hydrogen; R⁹, R¹¹ and R¹³ can be independently hydrogen or an optionally substituted C₁₋₆ alkyl; and R¹⁹ and R¹² can be independently an optionally substituted C₁₋₆ alkyl.

In some embodiments, Formula (I) can be a compound of Formula (Iα), wherein: B¹ can be an optionally substituted heterocyclic base or an optionally substituted heterocyclic base with a protected amino group selected from uridine, thymidine, guanine, adenine and

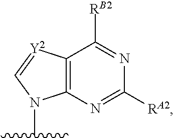

wherein R^{A2} can be hydrogen, R^{B2} can be NHR^{W2}, R^{W2} can be —C(═O)OR^{N2}, R^{N2} can be C₁₋₆ alkyl, and Y² can be N; R¹ can be an optionally substituted N-linked amino acid ester derivative selected from alanine methyl ester, alanine ethyl ester, alanine isopropyl ester, alanine cyclohexyl ester, alanine neopentyl ester and alanine benzyl ester; R² can be selected from an optionally substituted phenyl, an optionally substituted naphthyl, an optionally substituted pyridyl, an optionally substituted quinolyl; R^{3a} and R^{3b} can be selected from hydrogen and an optionally substituted C₁₋₆ alkyl, provided that at least one of R^{3a} and R^{3b} cannot be hydrogen; R⁴ can be hydrogen; R⁵ can be selected from hydrogen, —OR⁹ and —OC(═O)R¹⁹; R⁶ can be selected from hydrogen, halogen, —OR¹¹ and —OC(═O)R¹²; or R⁵ and R⁶ can be both oxygen atoms and linked together by a carbonyl group; R⁷ can be selected from hydrogen, halogen, an optionally substituted C₁₋₆ alkyl and —OR¹³; R⁸ can be hydrogen; R⁹, R¹¹ and R¹³ can be independently selected from hydrogen and an optionally substituted C₁₋₆ alkyl; and R¹⁹ and R¹² can be an optionally substituted C₁₋₆ alkyl.

In some embodiments, a compound of Formula (I) can be a single diastereomer. In other embodiments, a compound of Formula (I) can be a mixture of diastereomers. In some embodiments, a compound of Formula (I) can be a 1:1 mixture of two diastereomers. In some embodiments, a compound of Formula (I) can be diasteriometrically enriched (for example, one diastereomer can be present at a concentration of >55%, ≧75%, ≧80%, ≧90%, ≧95%, ≧98%, or ≧99% as compared to the total concentration of the other diastereomers).

Some embodiments of R¹ and R² of a compound of Formula (I), or a pharmaceutically acceptable salt thereof, are provided in Table 1. Tables 2-4 provide the structures of the variables bb01-bb12, aa01-aa11 and es01-es14, respectively. For example, the first entry in Table 1 is “bb01,aa01,es01,” which corresponds to a compound of Formula (I), wherein R²

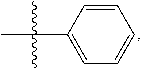

### and R¹ is

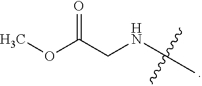

TABLE 1

| R², R¹, R_{α} | R², R¹, R_{α} | R², R¹, R_{α} | R², R¹, R_{α} | R², R¹, R_{α} |
|---|---|---|---|---|
| bb01, aa01, es01 | bb03, aa01, es01 | bb05, aa01, es01 | bb07, aa01, es01 | bb09, aa01, es01 |
| bb01, aa01, es02 | bb03, aa01, es02 | bb05, aa01, es02 | bb07, aa01, es02 | bb09, aa01, es02 |
| bb01, aa01, es03 | bb03, aa01, es03 | bb05, aa01, es03 | bb07, aa01, es03 | bb09, aa01, es03 |
| bb01, aa01, es04 | bb03, aa01, es04 | bb05, aa01, es04 | bb07, aa01, es04 | bb09, aa01, es04 |
| bb01, aa01, es05 | bb03, aa01, es05 | bb05, aa01, es05 | bb07, aa01, es05 | bb09, aa01, es05 |
| bb01, aa01, es06 | bb03, aa01, es06 | bb05, aa01, es06 | bb07, aa01, es06 | bb09, aa01, es06 |
| bb01, aa01, es07 | bb03, aa01, es07 | bb05, aa01, es07 | bb07, aa01, es07 | bb09, aa01, es07 |
| bb01, aa01, es08 | bb03, aa01, es08 | bb05, aa01, es08 | bb07, aa01, es08 | bb09, aa01, es08 |
| bb01, aa01, es09 | bb03, aa01, es09 | bb05, aa01, es09 | bb07, aa01, es09 | bb09, aa01, es09 |
| bb01, aa01, es10 | bb03, aa01, es10 | bb05, aa01, es10 | bb07, aa01, es10 | bb09, aa01, es10 |
| bb01, aa01, es11 | bb03, aa01, es11 | bb05, aa01, es11 | bb07, aa01, es11 | bb09, aa01, es11 |
| bb01, aa01, es12 | bb03, aa01, es12 | bb05, aa01, es12 | bb07, aa01, es12 | bb09, aa01, es12 |
| bb01, aa02, es01 | bb03, aa02, es01 | bb05, aa02, es01 | bb07, aa02, es01 | bb09, aa02, es01 |
| bb01, aa02, es02 | bb03, aa02, es02 | bb05, aa02, es02 | bb07, aa02, es02 | bb09, aa02, es02 |
| bb01, aa02, es03 | bb03, aa02, es03 | bb05, aa02, es03 | bb07, aa02, es03 | bb09, aa02, es03 |
| bb01, aa02, es04 | bb03, aa02, es04 | bb05, aa02, es04 | bb07, aa02, es04 | bb09, aa02, es04 |
| bb01, aa02, es05 | bb03, aa02, es05 | bb05, aa02, es05 | bb07, aa02, es05 | bb09, aa02, es05 |
| bb01, aa02, es06 | bb03, aa02, es06 | bb05, aa02, es06 | bb07, aa02, es06 | bb09, aa02, es06 |
| bb01, aa02, es07 | bb03, aa02, es07 | bb05, aa02, es07 | bb07, aa02, es07 | bb09, aa02, es07 |
| bb01, aa02, es08 | bb03, aa02, es08 | bb05, aa02, es08 | bb07, aa02, es08 | bb09, aa02, es08 |
| bb01, aa02, es09 | bb03, aa02, es09 | bb05, aa02, es09 | bb07, aa02, es09 | bb09, aa02, es09 |
| bb01, aa02, es10 | bb03, aa02, es10 | bb05, aa02, es10 | bb07, aa02, es10 | bb09, aa02, es10 |
| bb01, aa02, es11 | bb03, aa02, es11 | bb05, aa02, es11 | bb07, aa02, es11 | bb09, aa02, es11 |
| bb01, aa02, es12 | bb03, aa02, es12 | bb05, aa02, es12 | bb07, aa02, es12 | bb09, aa02, es12 |
| bb01, aa03, es01 | bb03, aa03, es01 | bb05, aa03, es01 | bb07, aa03, es01 | bb09, aa03, es01 |
| bb01, aa03, es02 | bb03, aa03, es02 | bb05, aa03, es02 | bb07, aa03, es02 | bb09, aa03, es02 |
| bb01, aa03, es03 | bb03, aa03, es03 | bb05, aa03, es03 | bb07, aa03, es03 | bb09, aa03, es03 |
| bb01, aa03, es04 | bb03, aa03, es04 | bb05, aa03, es04 | bb07, aa03, es04 | bb09, aa03, es04 |
| bb01, aa03, es05 | bb03, aa03, es05 | bb05, aa03, es05 | bb07, aa03, es05 | bb09, aa03, es05 |
| bb01, aa03, es06 | bb03, aa03, es06 | bb05, aa03, es06 | bb07, aa03, es06 | bb09, aa03, es06 |
| bb01, aa03, es07 | bb03, aa03, es07 | bb05, aa03, es07 | bb07, aa03, es07 | bb09, aa03, es07 |
| bb01, aa03, es08 | bb03, aa03, es08 | bb05, aa03, es08 | bb07, aa03, es08 | bb09, aa03, es08 |
| bb01, aa03, es09 | bb03, aa03, es09 | bb05, aa03, es09 | bb07, aa03, es09 | bb09, aa03, es09 |
| bb01, aa03, es10 | bb03, aa03, es10 | bb05, aa03, es10 | bb07, aa03, es10 | bb09, aa03, es10 |
| bb01, aa03, es11 | bb03, aa03, es11 | bb05, aa03, es11 | bb07, aa03, es11 | bb09, aa03, es11 |
| bb01, aa03, es12 | bb03, aa03, es12 | bb05, aa03, es12 | bb07, aa03, es12 | bb09, aa03, es12 |
| bb01, aa04, es01 | bb03, aa04, es01 | bb05, aa04, es01 | bb07, aa04, es01 | bb09, aa04, es01 |
| bb01, aa04, es02 | bb03, aa04, es02 | bb05, aa04, es02 | bb07, aa04, es02 | bb09, aa04, es02 |
| bb01, aa04, es03 | bb03, aa04, es03 | bb05, aa04, es03 | bb07, aa04, es03 | bb09, aa04, es03 |
| bb01, aa04, es04 | bb03, aa04, es04 | bb05, aa04, es04 | bb07, aa04, es04 | bb09, aa04, es04 |
| bb01, aa04, es05 | bb03, aa04, es05 | bb05, aa04, es05 | bb07, aa04, es05 | bb09, aa04, es05 |
| bb01, aa04, es06 | bb03, aa04, es06 | bb05, aa04, es06 | bb07, aa04, es06 | bb09, aa04, es06 |
| bb01, aa04, es07 | bb03, aa04, es07 | bb05, aa04, es07 | bb07, aa04, es07 | bb09, aa04, es07 |
| bb01, aa04, es08 | bb03, aa04, es08 | bb05, aa04, es08 | bb07, aa04, es08 | bb09, aa04, es08 |
| bb01, aa04, es09 | bb03, aa04, es09 | bb05, aa04, es09 | bb07, aa04, es09 | bb09, aa04, es09 |
| bb01, aa04, es10 | bb03, aa04, es10 | bb05, aa04, es10 | bb07, aa04, es10 | bb09, aa04, es10 |
| bb01, aa04, es11 | bb03, aa04, es11 | bb05, aa04, es11 | bb07, aa04, es11 | bb09, aa04, es11 |
| bb01, aa04, es12 | bb03, aa04, es12 | bb05, aa04, es12 | bb07, aa04, es12 | bb09, aa04, es12 |
| bb01, aa05, es01 | bb03, aa05, es01 | bb05, aa05, es01 | bb07, aa05, es01 | bb09, aa05, es01 |
| bb01, aa05, es02 | bb03, aa05, es02 | bb05, aa05, es02 | bb07, aa05, es02 | bb09, aa05, es02 |
| bb01, aa05, es03 | bb03, aa05, es03 | bb05, aa05, es03 | bb07, aa05, es03 | bb09, aa05, es03 |
| bb01, aa05, es04 | bb03, aa05, es04 | bb05, aa05, es04 | bb07, aa05, es04 | bb09, aa05, es04 |
| bb01, aa05, es05 | bb03, aa05, es05 | bb05, aa05, es05 | bb07, aa05, es05 | bb09, aa05, es05 |
| bb01, aa05, es06 | bb03, aa05, es06 | bb05, aa05, es06 | bb07, aa05, es06 | bb09, aa05, es06 |
| bb01, aa05, es07 | bb03, aa05, es07 | bb05, aa05, es07 | bb07, aa05, es07 | bb09, aa05, es07 |
| bb01, aa05, es08 | bb03, aa05, es08 | bb05, aa05, es08 | bb07, aa05, es08 | bb09, aa05, es08 |
| bb01, aa05, es09 | bb03, aa05, es09 | bb05, aa05, es09 | bb07, aa05, es09 | bb09, aa05, es09 |
| bb01, aa05, es10 | bb03, aa05, es10 | bb05, aa05, es10 | bb07, aa05, es10 | bb09, aa05, es10 |
| bb01, aa05, es11 | bb03, aa05, es11 | bb05, aa05, es11 | bb07, aa05, es11 | bb09, aa05, es11 |
| bb01, aa05, es12 | bb03, aa05, es12 | bb05, aa05, es12 | bb07, aa05, es12 | bb09, aa05, es12 |
| bb01, aa06, es01 | bb03, aa06, es01 | bb05, aa06, es01 | bb07, aa06, es01 | bb09, aa06, es01 |
| bb01, aa06, es02 | bb03, aa06, es02 | bb05, aa06, es02 | bb07, aa06, es02 | bb09, aa06, es02 |
| bb01, aa06, es03 | bb03, aa06, es03 | bb05, aa06, es03 | bb07, aa06, es03 | bb09, aa06, es03 |
| bb01, aa06, es04 | bb03, aa06, es04 | bb05, aa06, es04 | bb07, aa06, es04 | bb09, aa06, es04 |
| bb01, aa06, es05 | bb03, aa06, es05 | bb05, aa06, es05 | bb07, aa06, es05 | bb09, aa06, es05 |
| bb01, aa06, es06 | bb03, aa06, es06 | bb05, aa06, es06 | bb07, aa06, es06 | bb09, aa06, es06 |
| bb01, aa06, es07 | bb03, aa06, es07 | bb05, aa06, es07 | bb07, aa06, es07 | bb09, aa06, es07 |
| bb01, aa06, es08 | bb03, aa06, es08 | bb05, aa06, es08 | bb07, aa06, es08 | bb09, aa06, es08 |
| bb01, aa06, es09 | bb03, aa06, es09 | bb05, aa06, es09 | bb07, aa06, es09 | bb09, aa06, es09 |
| bb01, aa06, es10 | bb03, aa06, es10 | bb05, aa06, es10 | bb07, aa06, es10 | bb09, aa06, es10 |
| bb01, aa06, es11 | bb03, aa06, es11 | bb05, aa06, es11 | bb07, aa06, es11 | bb09, aa06, es11 |
| bb01, aa06, es12 | bb03, aa06, es12 | bb05, aa06, es12 | bb07, aa06, es12 | bb09, aa06, es12 |
| bb01, aa07, es01 | bb03, aa07, es01 | bb05, aa07, es01 | bb07, aa07, es01 | bb09, aa07, es01 |
| bb01, aa07, es02 | bb03, aa07, es02 | bb05, aa07, es02 | bb07, aa07, es02 | bb09, aa07, es02 |
| bb01, aa07, es03 | bb03, aa07, es03 | bb05, aa07, es03 | bb07, aa07, es03 | bb09, aa07, es03 |
| bb01, aa07, es04 | bb03, aa07, es04 | bb05, aa07, es04 | bb07, aa07, es04 | bb09, aa07, es04 |
| bb01, aa07, es05 | bb03, aa07, es05 | bb05, aa07, es05 | bb07, aa07, es05 | bb09, aa07, es05 |
| bb01, aa07, es06 | bb03, aa07, es06 | bb05, aa07, es06 | bb07, aa07, es06 | bb09, aa07, es06 |
| bb01, aa07, es07 | bb03, aa07, es07 | bb05, aa07, es07 | bb07, aa07, es07 | bb09, aa07, es07 |
| bb01, aa07, es08 | bb03, aa07, es08 | bb05, aa07, es08 | bb07, aa07, es08 | bb09, aa07, es08 |
| bb01, aa07, es09 | bb03, aa07, es09 | bb05, aa07, es09 | bb07, aa07, es09 | bb09, aa07, es09 |
| bb01, aa07, es10 | bb03, aa07, es10 | bb05, aa07, es10 | bb07, aa07, es10 | bb09, aa07, es10 |
| bb01, aa07, es11 | bb03, aa07, es11 | bb05, aa07, es11 | bb07, aa07, es11 | bb09, aa07, es11 |
| bb01, aa07, es12 | bb03, aa07, es12 | bb05, aa07, es12 | bb07, aa07, es12 | bb09, aa07, es12 |
| bb01, aa08, es01 | bb03, aa08, es01 | bb05, aa08, es01 | bb07, aa08, es01 | bb09, aa08, es01 |
| bb01, aa08, es02 | bb03, aa08, es02 | bb05, aa08, es02 | bb07, aa08, es02 | bb09, aa08, es02 |
| bb01, aa08, es03 | bb03, aa08, es03 | bb05, aa08, es03 | bb07, aa08, es03 | bb09, aa08, es03 |
| bb01, aa08, es04 | bb03, aa08, es04 | bb05, aa08, es04 | bb07, aa08, es04 | bb09, aa08, es04 |
| bb01, aa08, es05 | bb03, aa08, es05 | bb05, aa08, es05 | bb07, aa08, es05 | bb09, aa08, es05 |
| bb01, aa08, es06 | bb03, aa08, es06 | bb05, aa08, es06 | bb07, aa08, es06 | bb09, aa08, es06 |
| bb01, aa08, es07 | bb03, aa08, es07 | bb05, aa08, es07 | bb07, aa08, es07 | bb09, aa08, es07 |
| bb01, aa08, es08 | bb03, aa08, es08 | bb05, aa08, es08 | bb07, aa08, es08 | bb09, aa08, es08 |
| bb01, aa08, es09 | bb03, aa08, es09 | bb05, aa08, es09 | bb07, aa08, es09 | bb09, aa08, es09 |
| bb01, aa08, es10 | bb03, aa08, es10 | bb05, aa08, es10 | bb07, aa08, es10 | bb09, aa08, es10 |
| bb01, aa08, es11 | bb03, aa08, es11 | bb05, aa08, es11 | bb07, aa08, es11 | bb09, aa08, es11 |
| bb01, aa08, es12 | bb03, aa08, es12 | bb05, aa08, es12 | bb07, aa08, es12 | bb09, aa08, es12 |
| bb01, aa09, es01 | bb03, aa09, es01 | bb05, aa09, es01 | bb07, aa09, es01 | bb09, aa09, es01 |
| bb01, aa09, es02 | bb03, aa09, es02 | bb05, aa09, es02 | bb07, aa09, es02 | bb09, aa09, es02 |
| bb01, aa09, es03 | bb03, aa09, es03 | bb05, aa09, es03 | bb07, aa09, es03 | bb09, aa09, es03 |
| bb01, aa09, es04 | bb03, aa09, es04 | bb05, aa09, es04 | bb07, aa09, es04 | bb09, aa09, es04 |
| bb01, aa09, es05 | bb03, aa09, es05 | bb05, aa09, es05 | bb07, aa09, es05 | bb09, aa09, es05 |
| bb01, aa09, es06 | bb03, aa09, es06 | bb05, aa09, es06 | bb07, aa09, es06 | bb09, aa09, es06 |
| bb01, aa09, es07 | bb03, aa09, es07 | bb05, aa09, es07 | bb07, aa09, es07 | bb09, aa09, es07 |
| bb01, aa09, es08 | bb03, aa09, es08 | bb05, aa09, es08 | bb07, aa09, es08 | bb09, aa09, es08 |
| bb01, aa09, es09 | bb03, aa09, es09 | bb05, aa09, es09 | bb07, aa09, es09 | bb09, aa09, es09 |
| bb01, aa09, es10 | bb03, aa09, es10 | bb05, aa09, es10 | bb07, aa09, es10 | bb09, aa09, es10 |
| bb01, aa09, es11 | bb03, aa09, es11 | bb05, aa09, es11 | bb07, aa09, es11 | bb09, aa09, es11 |
| bb01, aa09, es12 | bb03, aa09, es12 | bb05, aa09, es12 | bb07, aa09, es12 | bb09, aa09, es12 |
| bb01, aa10, es01 | bb03, aa10, es01 | bb05, aa10, es01 | bb07, aa10, es01 | bb09, aa10, es01 |
| bb01, aa10, es02 | bb03, aa10, es02 | bb05, aa10, es02 | bb07, aa10, es02 | bb09, aa10, es02 |
| bb01, aa10, es03 | bb03, aa10, es03 | bb05, aa10, es03 | bb07, aa10, es03 | bb09, aa10, es03 |
| bb01, aa10, es04 | bb03, aa10, es04 | bb05, aa10, es04 | bb07, aa10, es04 | bb09, aa10, es04 |
| bb01, aa10, es05 | bb03, aa10, es05 | bb05, aa10, es05 | bb07, aa10, es05 | bb09, aa10, es05 |
| bb01, aa10, es06 | bb03, aa10, es06 | bb05, aa10, es06 | bb07, aa10, es06 | bb09, aa10, es06 |
| bb01, aa10, es07 | bb03, aa10, es07 | bb05, aa10, es07 | bb07, aa10, es07 | bb09, aa10, es07 |
| bb01, aa10, es08 | bb03, aa10, es08 | bb05, aa10, es08 | bb07, aa10, es08 | bb09, aa10, es08 |
| bb01, aa10, es09 | bb03, aa10, es09 | bb05, aa10, es09 | bb07, aa10, es09 | bb09, aa10, es09 |
| bb01, aa10, es10 | bb03, aa10, es10 | bb05, aa10, es10 | bb07, aa10, es10 | bb09, aa10, es10 |
| bb01, aa10, es11 | bb03, aa10, es11 | bb05, aa10, es11 | bb07, aa10, es11 | bb09, aa10, es11 |
| bb01, aa10, es12 | bb03, aa10, es12 | bb05, aa10, es12 | bb07, aa10, es12 | bb09, aa10, es12 |
| bb02, aa01, es01 | bb04, aa01, es01 | bb06, aa01, es01 | bb08, aa01, es01 | bb10, aa01, es01 |
| bb02, aa01, es02 | bb04, aa01, es02 | bb06, aa01, es02 | bb08, aa01, es02 | bb10, aa01, es02 |
| bb02, aa01, es03 | bb04, aa01, es03 | bb06, aa01, es03 | bb08, aa01, es03 | bb10, aa01, es03 |
| bb02, aa01, es04 | bb04, aa01, es04 | bb06, aa01, es04 | bb08, aa01, es04 | bb10, aa01, es04 |
| bb02, aa01, es05 | bb04, aa01, es05 | bb06, aa01, es05 | bb08, aa01, es05 | bb10, aa01, es05 |
| bb02, aa01, es06 | bb04, aa01, es06 | bb06, aa01, es06 | bb08, aa01, es06 | bb10, aa01, es06 |
| bb02, aa01, es07 | bb04, aa01, es07 | bb06, aa01, es07 | bb08, aa01, es07 | bb10, aa01, es07 |
| bb02, aa01, es08 | bb04, aa01, es08 | bb06, aa01, es08 | bb08, aa01, es08 | bb10, aa01, es08 |
| bb02, aa01, es09 | bb04, aa01, es09 | bb06, aa01, es09 | bb08, aa01, es09 | bb10, aa01, es09 |
| bb02, aa01, es10 | bb04, aa01, es10 | bb06, aa01, es10 | bb08, aa01, es10 | bb10, aa01, es10 |
| bb02, aa01, es11 | bb04, aa01, es11 | bb06, aa01, es11 | bb08, aa01, es11 | bb10, aa01, es11 |
| bb02, aa01, es12 | bb04, aa01, es12 | bb06, aa01, es12 | bb08, aa01, es12 | bb10, aa01, es12 |
| bb02, aa02, es01 | bb04, aa02, es01 | bb06, aa02, es01 | bb08, aa02, es01 | bb10, aa02, es01 |
| bb02, aa02, es02 | bb04, aa02, es02 | bb06, aa02, es02 | bb08, aa02, es02 | bb10, aa02, es02 |
| bb02, aa02, es03 | bb04, aa02, es03 | bb06, aa02, es03 | bb08, aa02, es03 | bb10, aa02, es03 |
| bb02, aa02, es04 | bb04, aa02, es04 | bb06, aa02, es04 | bb08, aa02, es04 | bb10, aa02, es04 |
| bb02, aa02, es05 | bb04, aa02, es05 | bb06, aa02, es05 | bb08, aa02, es05 | bb10, aa02, es05 |
| bb02, aa02, es06 | bb04, aa02, es06 | bb06, aa02, es06 | bb08, aa02, es06 | bb10, aa02, es06 |
| bb02, aa02, es07 | bb04, aa02, es07 | bb06, aa02, es07 | bb08, aa02, es07 | bb10, aa02, es07 |
| bb02, aa02, es08 | bb04, aa02, es08 | bb06, aa02, es08 | bb08, aa02, es08 | bb10, aa02, es08 |
| bb02, aa02, es09 | bb04, aa02, es09 | bb06, aa02, es09 | bb08, aa02, es09 | bb10, aa02, es09 |
| bb02, aa02, es10 | bb04, aa02, es10 | bb06, aa02, es10 | bb08, aa02, es10 | bb10, aa02, es10 |
| bb02, aa02, es11 | bb04, aa02, es11 | bb06, aa02, es11 | bb08, aa02, es11 | bb10, aa02, es11 |
| bb02, aa02, es12 | bb04, aa02, es12 | bb06, aa02, es12 | bb08, aa02, es12 | bb10, aa02, es12 |
| bb02, aa03, es01 | bb04, aa03, es01 | bb06, aa03, es01 | bb08, aa03, es01 | bb10, aa03, es01 |
| bb02, aa03, es02 | bb04, aa03, es02 | bb06, aa03, es02 | bb08, aa03, es02 | bb10, aa03, es02 |
| bb02, aa03, es03 | bb04, aa03, es03 | bb06, aa03, es03 | bb08, aa03, es03 | bb10, aa03, es03 |
| bb02, aa03, es04 | bb04, aa03, es04 | bb06, aa03, es04 | bb08, aa03, es04 | bb10, aa03, es04 |
| bb02, aa03, es05 | bb04, aa03, es05 | bb06, aa03, es05 | bb08, aa03, es05 | bb10, aa03, es05 |
| bb02, aa03, es06 | bb04, aa03, es06 | bb06, aa03, es06 | bb08, aa03, es06 | bb10, aa03, es06 |
| bb02, aa03, es07 | bb04, aa03, es07 | bb06, aa03, es07 | bb08, aa03, es07 | bb10, aa03, es07 |
| bb02, aa03, es08 | bb04, aa03, es08 | bb06, aa03, es08 | bb08, aa03, es08 | bb10, aa03, es08 |
| bb02, aa03, es09 | bb04, aa03, es09 | bb06, aa03, es09 | bb08, aa03, es09 | bb10, aa03, es09 |
| bb02, aa03, es10 | bb04, aa03, es10 | bb06, aa03, es10 | bb08, aa03, es10 | bb10, aa03, es10 |
| bb02, aa03, es11 | bb04, aa03, es11 | bb06, aa03, es11 | bb08, aa03, es11 | bb10, aa03, es11 |
| bb02, aa03, es12 | bb04, aa03, es12 | bb06, aa03, es12 | bb08, aa03, es12 | bb10, aa03, es12 |
| bb02, aa04, es01 | bb04, aa04, es01 | bb06, aa04, es01 | bb08, aa04, es01 | bb10, aa04, es01 |
| bb02, aa04, es02 | bb04, aa04, es02 | bb06, aa04, es02 | bb08, aa04, es02 | bb10, aa04, es02 |
| bb02, aa04, es03 | bb04, aa04, es03 | bb06, aa04, es03 | bb08, aa04, es03 | bb10, aa04, es03 |
| bb02, aa04, es04 | bb04, aa04, es04 | bb06, aa04, es04 | bb08, aa04, es04 | bb10, aa04, es04 |
| bb02, aa04, es05 | bb04, aa04, es05 | bb06, aa04, es05 | bb08, aa04, es05 | bb10, aa04, es05 |
| bb02, aa04, es06 | bb04, aa04, es06 | bb06, aa04, es06 | bb08, aa04, es06 | bb10, aa04, es06 |
| bb02, aa04, es07 | bb04, aa04, es07 | bb06, aa04, es07 | bb08, aa04, es07 | bb10, aa04, es07 |
| bb02, aa04, es08 | bb04, aa04, es08 | bb06, aa04, es08 | bb08, aa04, es08 | bb10, aa04, es08 |
| bb02, aa04, es09 | bb04, aa04, es09 | bb06, aa04, es09 | bb08, aa04, es09 | bb10, aa04, es09 |
| bb02, aa04, es10 | bb04, aa04, es10 | bb06, aa04, es10 | bb08, aa04, es10 | bb10, aa04, es10 |
| bb02, aa04, es11 | bb04, aa04, es11 | bb06, aa04, es11 | bb08, aa04, es11 | bb10, aa04, es11 |
| bb02, aa04, es12 | bb04, aa04, es12 | bb06, aa04, es12 | bb08, aa04, es12 | bb10, aa04, es12 |
| bb02, aa05, es01 | bb04, aa05, es01 | bb06, aa05, es01 | bb08, aa05, es01 | bb10, aa05, es01 |
| bb02, aa05, es02 | bb04, aa05, es02 | bb06, aa05, es02 | bb08, aa05, es02 | bb10, aa05, es02 |
| bb02, aa05, es03 | bb04, aa05, es03 | bb06, aa05, es03 | bb08, aa05, es03 | bb10, aa05, es03 |
| bb02, aa05, es04 | bb04, aa05, es04 | bb06, aa05, es04 | bb08, aa05, es04 | bb10, aa05, es04 |
| bb02, aa05, es05 | bb04, aa05, es05 | bb06, aa05, es05 | bb08, aa05, es05 | bb10, aa05, es05 |
| bb02, aa05, es06 | bb04, aa05, es06 | bb06, aa05, es06 | bb08, aa05, es06 | bb10, aa05, es06 |
| bb02, aa05, es07 | bb04, aa05, es07 | bb06, aa05, es07 | bb08, aa05, es07 | bb10, aa05, es07 |
| bb02, aa05, es08 | bb04, aa05, es08 | bb06, aa05, es08 | bb08, aa05, es08 | bb10, aa05, es08 |
| bb02, aa05, es09 | bb04, aa05, es09 | bb06, aa05, es09 | bb08, aa05, es09 | bb10, aa05, es09 |
| bb02, aa05, es10 | bb04, aa05, es10 | bb06, aa05, es10 | bb08, aa05, es10 | bb10, aa05, es10 |
| bb02, aa05, es11 | bb04, aa05, es11 | bb06, aa05, es11 | bb08, aa05, es11 | bb10, aa05, es11 |
| bb02, aa05, es12 | bb04, aa05, es12 | bb06, aa05, es12 | bb08, aa05, es12 | bb10, aa05, es12 |
| bb02, aa06, es01 | bb04, aa06, es01 | bb06, aa06, es01 | bb08, aa06, es01 | bb10, aa06, es01 |
| bb02, aa06, es02 | bb04, aa06, es02 | bb06, aa06, es02 | bb08, aa06, es02 | bb10, aa06, es02 |
| bb02, aa06, es03 | bb04, aa06, es03 | bb06, aa06, es03 | bb08, aa06, es03 | bb10, aa06, es03 |
| bb02, aa06, es04 | bb04, aa06, es04 | bb06, aa06, es04 | bb08, aa06, es04 | bb10, aa06, es04 |
| bb02, aa06, es05 | bb04, aa06, es05 | bb06, aa06, es05 | bb08, aa06, es05 | bb10, aa06, es05 |
| bb02, aa06, es06 | bb04, aa06, es06 | bb06, aa06, es06 | bb08, aa06, es06 | bb10, aa06, es06 |
| bb02, aa06, es07 | bb04, aa06, es07 | bb06, aa06, es07 | bb08, aa06, es07 | bb10, aa06, es07 |
| bb02, aa06, es08 | bb04, aa06, es08 | bb06, aa06, es08 | bb08, aa06, es08 | bb10, aa06, es08 |
| bb02, aa06, es09 | bb04, aa06, es09 | bb06, aa06, es09 | bb08, aa06, es09 | bb10, aa06, es09 |
| bb02, aa06, es10 | bb04, aa06, es10 | bb06, aa06, es10 | bb08, aa06, es10 | bb10, aa06, es10 |
| bb02, aa06, es11 | bb04, aa06, es11 | bb06, aa06, es11 | bb08, aa06, es11 | bb10, aa06, es11 |
| bb02, aa06, es12 | bb04, aa06, es12 | bb06, aa06, es12 | bb08, aa06, es12 | bb10, aa06, es12 |
| bb02, aa07, es01 | bb04, aa07, es01 | bb06, aa07, es01 | bb08, aa07, es01 | bb10, aa07, es01 |
| bb02, aa07, es02 | bb04, aa07, es02 | bb06, aa07, es02 | bb08, aa07, es02 | bb10, aa07, es02 |
| bb02, aa07, es03 | bb04, aa07, es03 | bb06, aa07, es03 | bb08, aa07, es03 | bb10, aa07, es03 |
| bb02, aa07, es04 | bb04, aa07, es04 | bb06, aa07, es04 | bb08, aa07, es04 | bb10, aa07, es04 |
| bb02, aa07, es05 | bb04, aa07, es05 | bb06, aa07, es05 | bb08, aa07, es05 | bb10, aa07, es05 |
| bb02, aa07, es06 | bb04, aa07, es06 | bb06, aa07, es06 | bb08, aa07, es06 | bb10, aa07, es06 |
| bb02, aa07, es07 | bb04, aa07, es07 | bb06, aa07, es07 | bb08, aa07, es07 | bb10, aa07, es07 |
| bb02, aa07, es08 | bb04, aa07, es08 | bb06, aa07, es08 | bb08, aa07, es08 | bb10, aa07, es08 |
| bb02, aa07, es09 | bb04, aa07, es09 | bb06, aa07, es09 | bb08, aa07, es09 | bb10, aa07, es09 |
| bb02, aa07, es10 | bb04, aa07, es10 | bb06, aa07, es10 | bb08, aa07, es10 | bb10, aa07, es10 |
| bb02, aa07, es11 | bb04, aa07, es11 | bb06, aa07, es11 | bb08, aa07, es11 | bb10, aa07, es11 |
| bb02, aa07, es12 | bb04, aa07, es12 | bb06, aa07, es12 | bb08, aa07, es12 | bb10, aa07, es12 |
| bb02, aa08, es01 | bb04, aa08, es01 | bb06, aa08, es01 | bb08, aa08, es01 | bb10, aa08, es01 |
| bb02, aa08, es02 | bb04, aa08, es02 | bb06, aa08, es02 | bb08, aa08, es02 | bb10, aa08, es02 |
| bb02, aa08, es03 | bb04, aa08, es03 | bb06, aa08, es03 | bb08, aa08, es03 | bb10, aa08, es03 |
| bb02, aa08, es04 | bb04, aa08, es04 | bb06, aa08, es04 | bb08, aa08, es04 | bb10, aa08, es04 |
| bb02, aa08, es05 | bb04, aa08, es05 | bb06, aa08, es05 | bb08, aa08, es05 | bb10, aa08, es05 |
| bb02, aa08, es06 | bb04, aa08, es06 | bb06, aa08, es06 | bb08, aa08, es06 | bb10, aa08, es06 |
| bb02, aa08, es07 | bb04, aa08, es07 | bb06, aa08, es07 | bb08, aa08, es07 | bb10, aa08, es07 |
| bb02, aa08, es08 | bb04, aa08, es08 | bb06, aa08, es08 | bb08, aa08, es08 | bb10, aa08, es08 |
| bb02, aa08, es09 | bb04, aa08, es09 | bb06, aa08, es09 | bb08, aa08, es09 | bb10, aa08, es09 |
| bb02, aa08, es10 | bb04, aa08, es10 | bb06, aa08, es10 | bb08, aa08, es10 | bb10, aa08, es10 |
| bb02, aa08, es11 | bb04, aa08, es11 | bb06, aa08, es11 | bb08, aa08, es11 | bb10, aa08, es11 |
| bb02, aa08, es12 | bb04, aa08, es12 | bb06, aa08, es12 | bb08, aa08, es12 | bb10, aa08, es12 |
| bb02, aa09, es01 | bb04, aa09, es01 | bb06, aa09, es01 | bb08, aa09, es01 | bb10, aa09, es01 |
| bb02, aa09, es02 | bb04, aa09, es02 | bb06, aa09, es02 | bb08, aa09, es02 | bb10, aa09, es02 |
| bb02, aa09, es03 | bb04, aa09, es03 | bb06, aa09, es03 | bb08, aa09, es03 | bb10, aa09, es03 |
| bb02, aa09, es04 | bb04, aa09, es04 | bb06, aa09, es04 | bb08, aa09, es04 | bb10, aa09, es04 |
| bb02, aa09, es05 | bb04, aa09, es05 | bb06, aa09, es05 | bb08, aa09, es05 | bb10, aa09, es05 |
| bb02, aa09, es06 | bb04, aa09, es06 | bb06, aa09, es06 | bb08, aa09, es06 | bb10, aa09, es06 |
| bb02, aa09, es07 | bb04, aa09, es07 | bb06, aa09, es07 | bb08, aa09, es07 | bb10, aa09, es07 |
| bb02, aa09, es08 | bb04, aa09, es08 | bb06, aa09, es08 | bb08, aa09, es08 | bb10, aa09, es08 |
| bb02, aa09, es09 | bb04, aa09, es09 | bb06, aa09, es09 | bb08, aa09, es09 | bb10, aa09, es09 |
| bb02, aa09, es10 | bb04, aa09, es10 | bb06, aa09, es10 | bb08, aa09, es10 | bb10, aa09, es10 |
| bb02, aa09, es11 | bb04, aa09, es11 | bb06, aa09, es11 | bb08, aa09, es11 | bb10, aa09, es11 |
| bb02, aa09, es12 | bb04, aa09, es12 | bb06, aa09, es12 | bb08, aa09, es12 | bb10, aa09, es12 |
| bb02, aa10, es01 | bb04, aa10, es01 | bb06, aa10, es01 | bb08, aa10, es01 | bb10, aa10, es01 |
| bb02, aa10, es02 | bb04, aa10, es02 | bb06, aa10, es02 | bb08, aa10, es02 | bb10, aa10, es02 |
| bb02, aa10, es03 | bb04, aa10, es03 | bb06, aa10, es03 | bb08, aa10, es03 | bb10, aa10, es03 |
| bb02, aa10, es04 | bb04, aa10, es04 | bb06, aa10, es04 | bb08, aa10, es04 | bb10, aa10, es04 |
| bb02, aa10, es05 | bb04, aa10, es05 | bb06, aa10, es05 | bb08, aa10, es05 | bb10, aa10, es05 |
| bb02, aa10, es06 | bb04, aa10, es06 | bb06, aa10, es06 | bb08, aa10, es06 | bb10, aa10, es06 |
| bb02, aa10, es07 | bb04, aa10, es07 | bb06, aa10, es07 | bb08, aa10, es07 | bb10, aa10, es07 |
| bb02, aa10, es08 | bb04, aa10, es08 | bb06, aa10, es08 | bb08, aa10, es08 | bb10, aa10, es08 |
| bb02, aa10, es09 | bb04, aa10, es09 | bb06, aa10, es09 | bb08, aa10, es09 | bb10, aa10, es09 |
| bb02, aa10, es10 | bb04, aa10, es10 | bb06, aa10, es10 | bb08, aa10, es10 | bb10, aa10, es10 |
| bb02, aa10, es11 | bb04, aa10, es11 | bb06, aa10, es11 | bb08, aa10, es11 | bb10, aa10, es11 |
| bb02, aa10, es12 | bb04, aa10, es12 | bb06, aa10, es12 | bb08, aa10, es12 | bb10, aa10, es12 |

TABLE 2

| | |
|---|---|
| 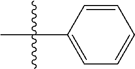 | bb01 |
| 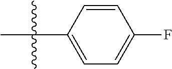 | bb02 |
| 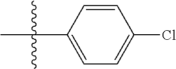 | bb03 |
| 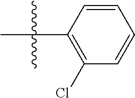 | bb04 |
| 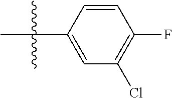 | bb05 |
| 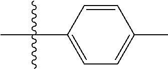 | bb06 |
| 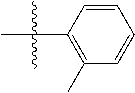 | bb07 |
| 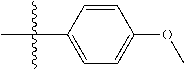 | bb08 |
| 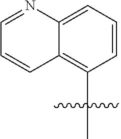 | bb09 |
| 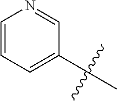 | bb10 |

TABLE 3

| | |
|---|---|
| 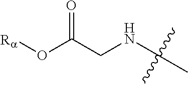 | aa01 |
| 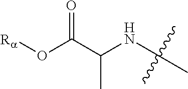 | aa02 |
| 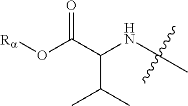 | aa03 |
| 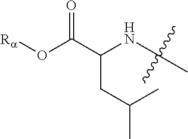 | aa04 |
| 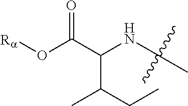 | aa05 |
| 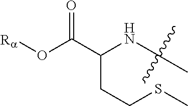 | aa06 |
| 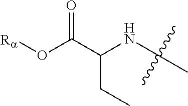 | aa07 |
| 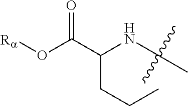 | aa08 |
| 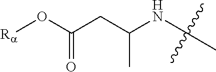 | aa09 |
| 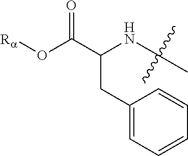 | aa10 |

TABLE 4

| | | |
|---|---|---|
| es01 R_{α} = methyl | es02 R_{α} = ethyl | es03 R_{α} = isopropyl |
| es04 R_{α} = propyl | es05 R_{α} = cyclohexyl | es06 R_{α} = cyclopentyl |
| es07 R_{α} = cyclobutyl | es08 R_{α} = cyclopropyl | es09 R_{α} = benzyl |
| es11 R_{α} = neopentyl | es10 R_{α} = t-butyl | es12 R_{α} = hydrogen |

In some embodiments, one of R^{3a} and R^{3b} is methyl and the other of R^{1a} and R^{3b} is hydrogen, and R⁴ and R⁸ can be both hydrogens in any of the embodiments described in Table 1. In some embodiments, at least one of R⁵ and R⁶ can be OH in any of the embodiments described in Table 1. In some embodiments, R⁷ can be hydrogen, halogen or C₁₋₆ alkyl in any of the embodiments described in Table 1. In some embodiments, B¹ can be adenine, guanine, uracil, thymine or cystine in any of the embodiments described in Table 1. In some embodiments, R^{3a}, R^{3b}, R⁴, R⁵, R⁶, R⁷, R⁸ and B¹ can be the groups provided with respect to Formula (Iα) in any of the embodiments described in Table 1.

Examples of compounds of Formula (I) include, but are not limited to the following:

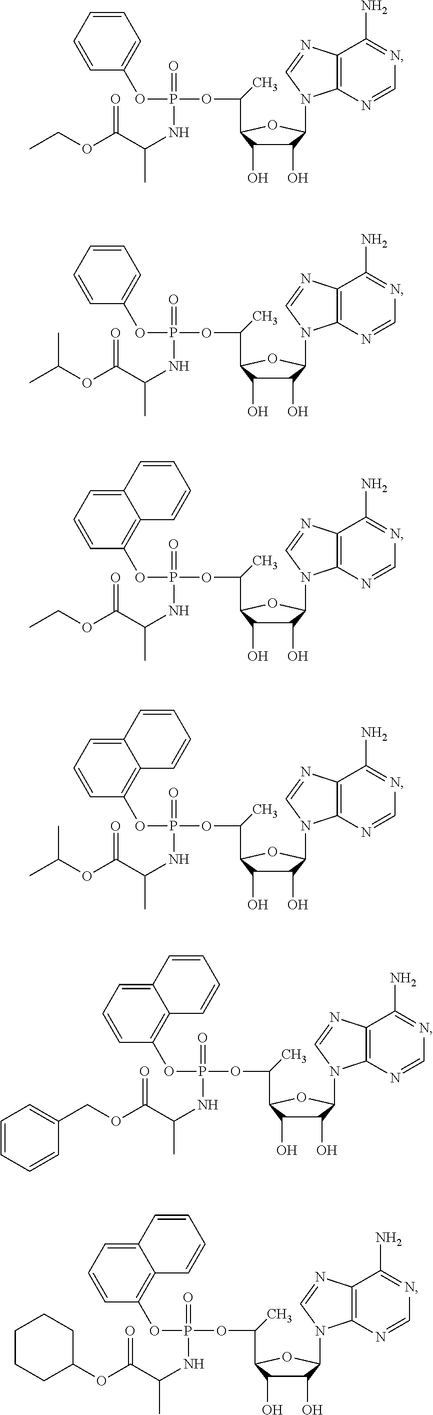

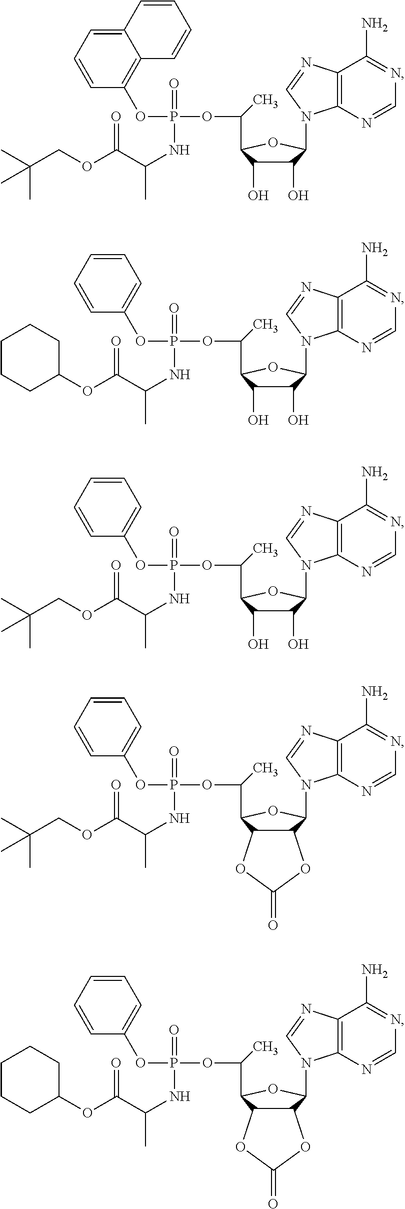

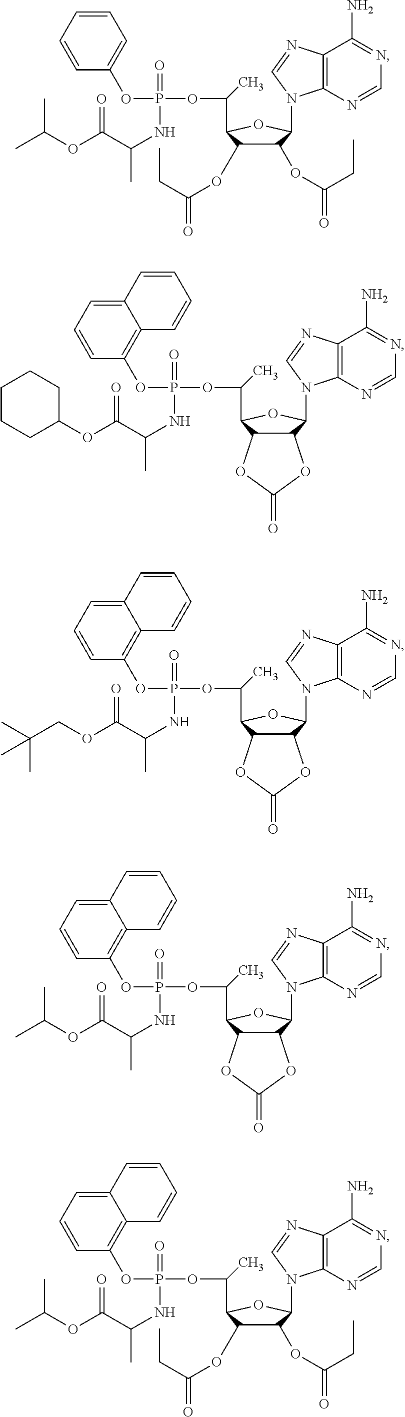

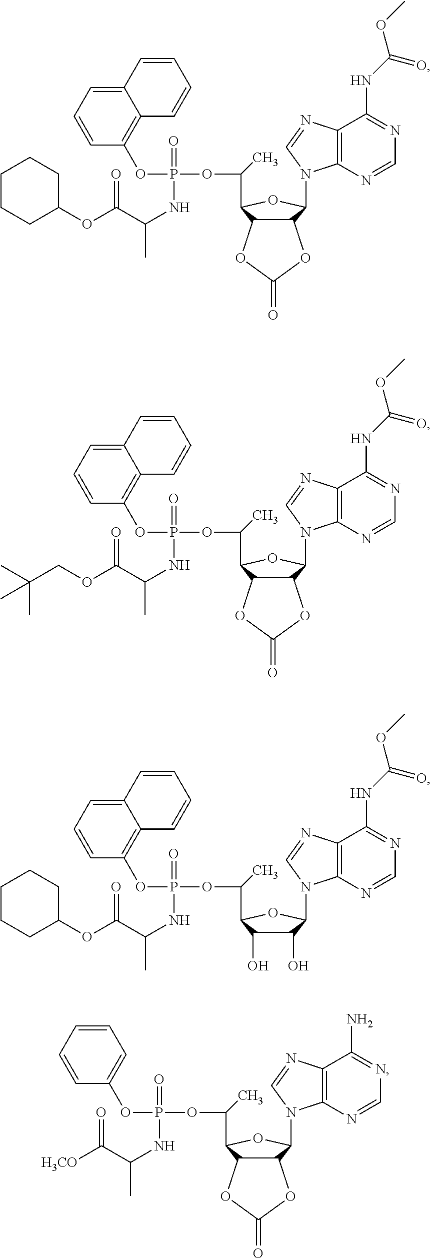

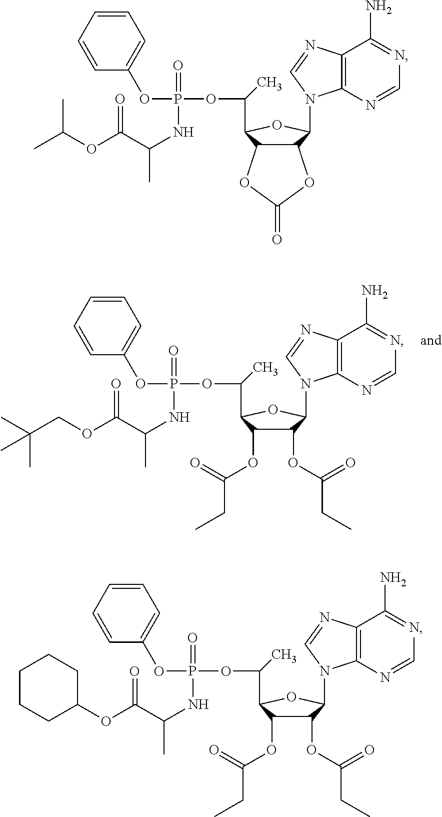

Additional examples of compounds of Formula (I) are shown below.

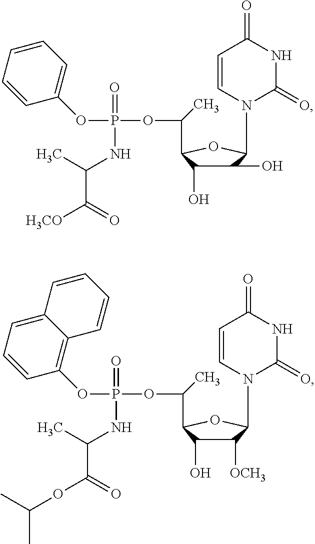

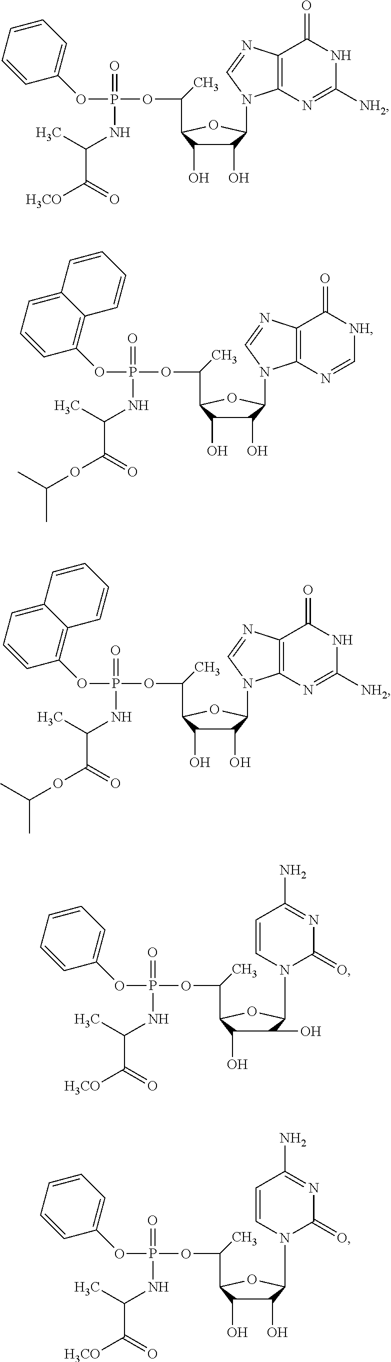

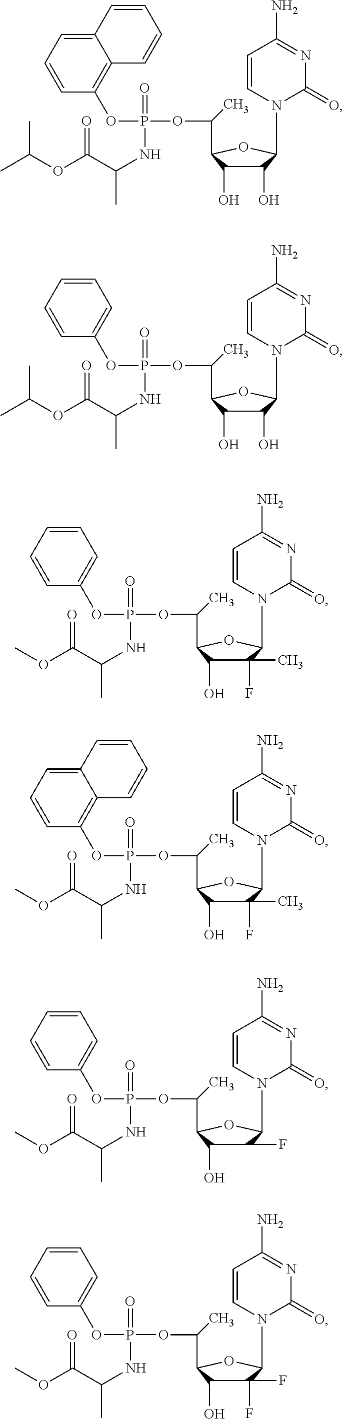

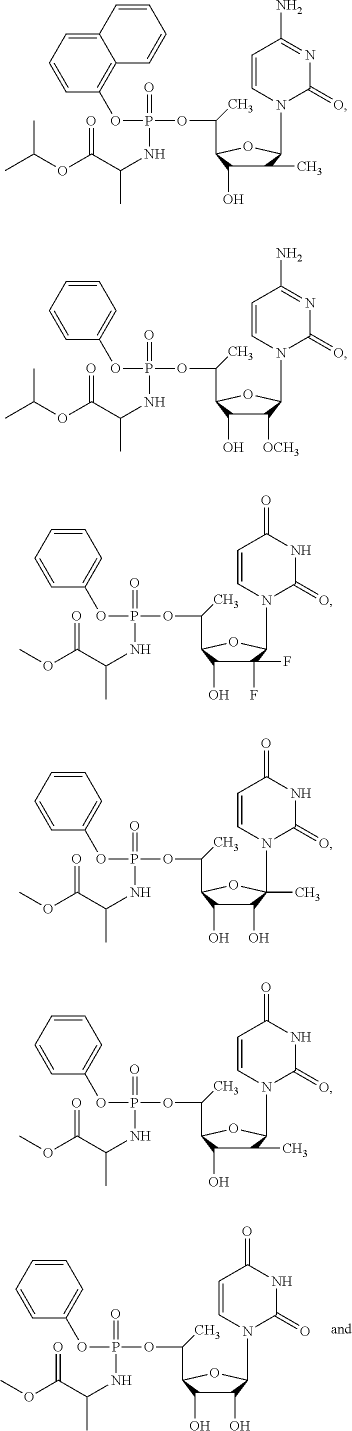

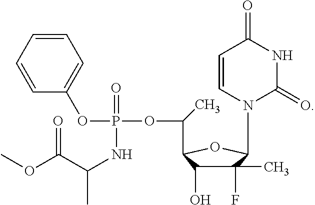

Additional examples of compounds of Formula (I) include, but are not limited to the following:

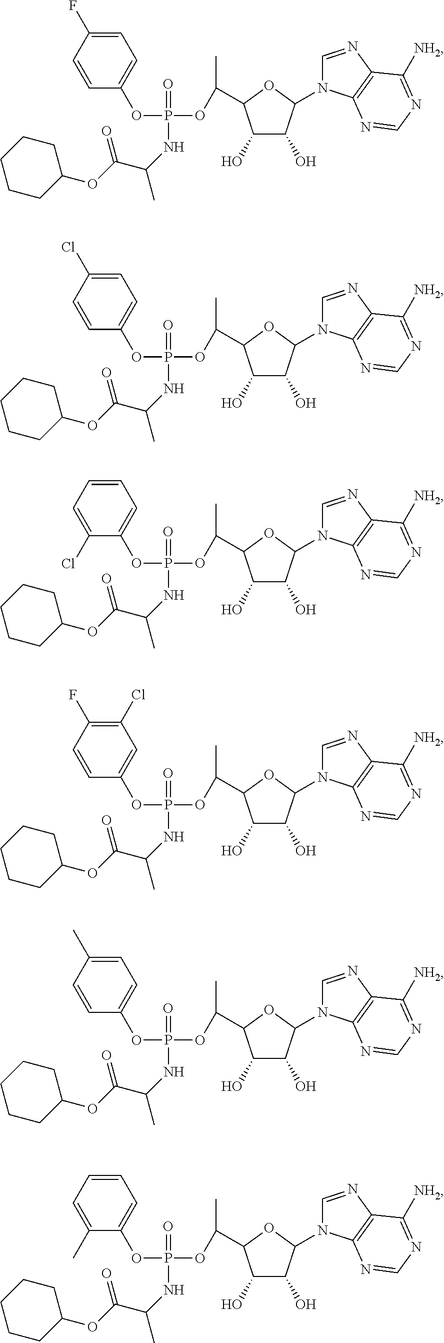

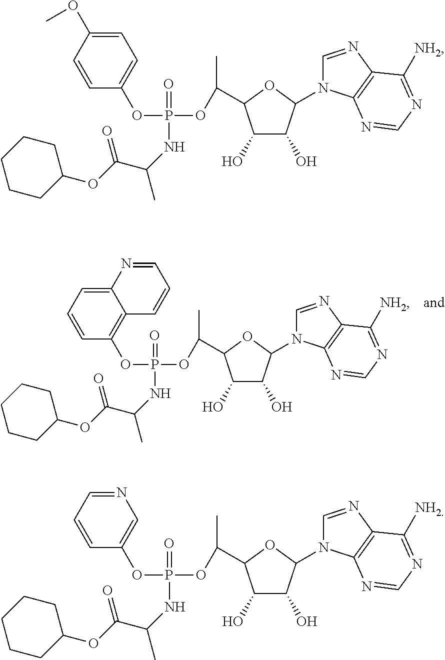

Additional examples of compounds of Formula (I) include, but are not limited to the following:

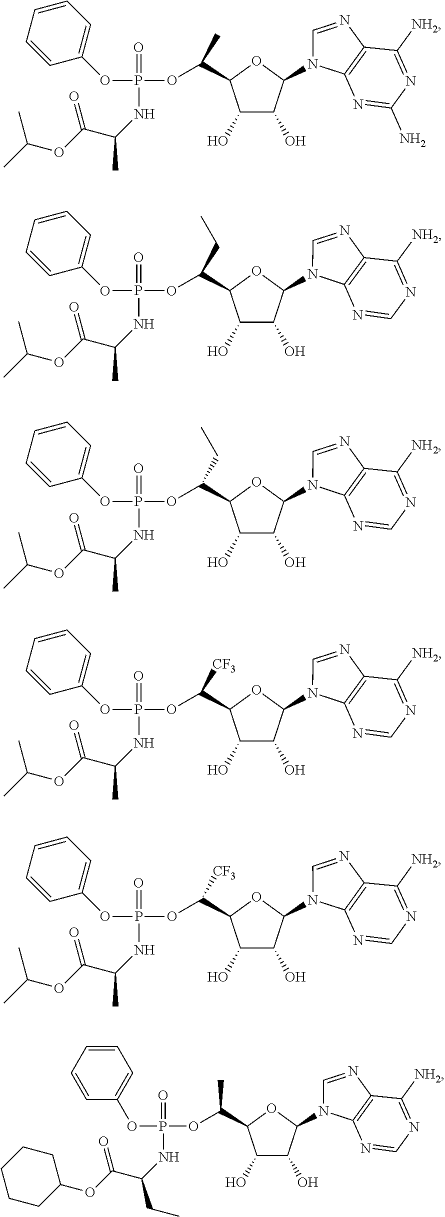

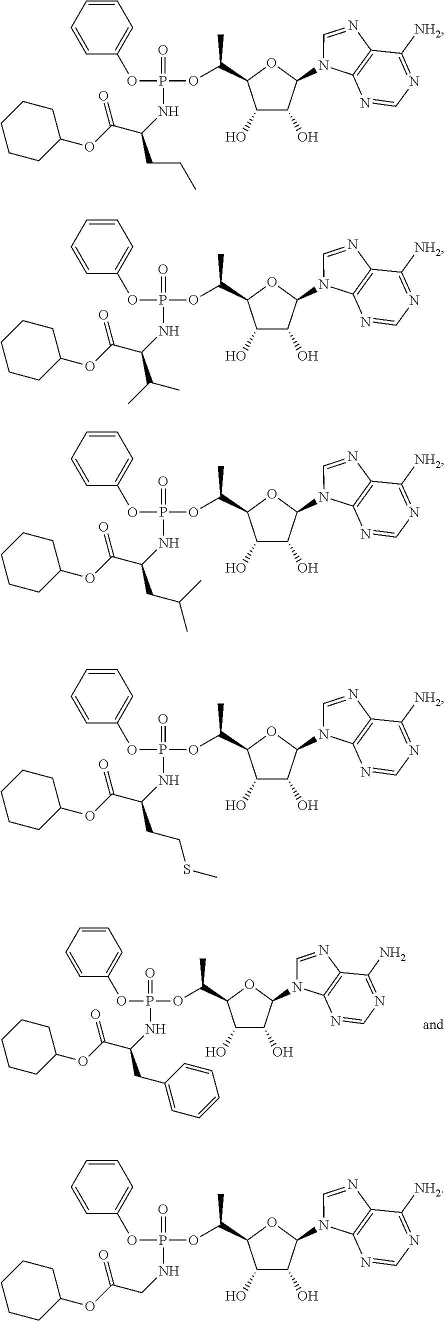

In some embodiments, neutralizing the charge on the phosphate group of a nucleoside monophosphate, or nucleotide, may facilitate the penetration of the cell membrane by oral administration of a compound of Formula (I) (including a compound of Formula (Iα)) by making the compound more lipophilic compared to a nucleotide having a comparable structure with one or more charges present on the phosphate. Once absorbed and taken inside the cell, the groups attached to the phosphate can be easily removed by esterases, proteases or other enzymes. In some embodiments, the groups attached to the phosphate can be removed by simple hydrolysis. Inside the cell, the monophosphate thus released may then be metabolized by cellular enzymes to the diphosphate or the active triphosphate.

In some embodiments, a compound of Formula (I) (including a compound of Formula (Iα)), or a pharmaceutically acceptable salt thereof, can act as a chain terminator of HCV replication. For example, incorporation of a compound of Formula (I) containing a moiety at the 2′-carbon position can terminate further elongation of the RNA chain of HCV. For example, a compound of Formula (I) can contain a 2′-carbon modification when R⁷ is a non-hydrogen group selected from halogen or an optionally substituted C₁₋₆ alkyl.

In some embodiments, a compound of Formula (I) (including a compound of Formula (Iα)), or a pharmaceutically acceptable salt thereof, can have increased metabolic and/or plasma stability. In some embodiments, a compound of Formula (I) (including a compound of Formula (Iα)), or a pharmaceutically acceptable salt thereof, can have improved properties. A non-limiting list of example properties include, but are not limited to, increased biological half life, increased bioavailability, increase potency, a sustained in vivo response, increased dosing intervals, decreased dosing amounts, decreased cytotoxicity, reduction in required amounts for treating disease conditions, reduction in viral load, reduction in time to seroconversion (i.e., the virus becomes undetectable in patient serum), increased sustained viral response, a reduction of morbidity or mortality in clinical outcomes, increased subject compliance, decreased liver conditions (such as liver fibrosis, liver cirrohis and/or liver cancer), and compatibility with other medications. In some embodiments, a compound of Formula (I), or a pharmaceutically acceptable salt thereof, can have a biological half life of greater than 24 hours. In some embodiments, a compound of Formula (I), or a pharmaceutically acceptable salt thereof, can have a biological half life in the range of about 28 hours to about 36 hours. In some embodiments, a compound of Formula (I), or a pharmaceutically acceptable salt thereof, can have more potent antiviral activity (for example, a lower IC₅₀ in an HCV replicon assay) as compared to the current standard of care.

### Synthesis

Compounds of Formula (I) (including compounds of Formula (Iα)), and those described herein may be prepared in various ways. General synthetic routes to the compound of Formula (I), and some examples of starting materials used to synthesize the compounds of Formula (I) are shown in Scheme 1, and described herein. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

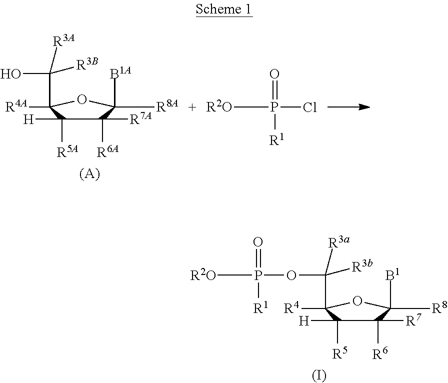

One method for forming a compound of Formula (I) is shown in Scheme 1. In Scheme 1, R^{3A}, R^{3B}, R^{5A}, R^{6A}, R^{7A}, R^{8A} and B^{1A} can be the same as R^{3a}, R^{3b}, R⁴, R⁵, R⁶, R⁷, R⁸ and B¹ as described herein for Formula (I); and R¹ and R² can be the same as described herein for Formula (I). As shown in Scheme 1, a compound of Formula (A) can be reacted with a phosphorochloridate of formula R²O—P(═O)(R¹)—Cl to form a compound of Formula (I).

To reduce the formation of side products, one or more the groups attached to the pentose ring can be protected with one or more suitable protecting groups. As an example, if R^{5A} and/or R^{6A} is/are hydroxy group(s), the hydroxy group(s) can be protected with suitable protecting groups, such as triarylmethyl and/or silyl groups. Examples of triarylmethyl groups include but are not limited to, trityl, monomethoxytrityl (MMTr), 4,4′-dimethoxytrityl (DMTr), 4,4′,4″-trimethoxytrityl (TMTr), 4,4′,4″-tris-(benzoyloxy)trityl (TBTr), 4,4′,4″-tris(4,5-dichlorophthalimido) trityl (CPTr), 4,4′,4″-tris(levulinyloxy)trityl (TLTr), p-anisyl-1-naphthylphenylmethyl, di-o-anisyl-1-naphthylmethyl, p-tolyldipheylmethyl, 3-(imidazolylmethyl)-4,4′-dimethoxytrityl, 9-phenylxanthen-9-yl (Pixyl), 9-(p-methoxyphenyl)xanthen-9-yl (Mox), 4-decyloxytrityl, 4-hexadecyloxytrityl, 4,4′-dioctadecyltrityl, 9-(4-octadecyloxyphenyl)xanthen-9-yl, 1,1′-bis-(4-methoxyphenyl)-1′-pyrenylmethyl, 4,4′,4″-tris-(tert-butylphenyl)methyl (TTTr) and 4,4′-di-3,5-hexadienoxytrityl. Examples of suitable silyl groups are described herein. Alternatively, R^{5A} and/or R^{6A} can be protected by a single achiral or chiral protecting group, for example, by forming an orthoester, a cyclic acetal or a cyclic ketal. Suitable orthoesters include methoxymethylene acetal, ethoxymethylene acetal, 2-oxacyclopentylidene orthoester, dimethoxymethylene orthoester, 1-methoxyethylidene orthoester, 1-ethoxyethylidene orthoester, methylidene orthoester, phthalide orthoester 1,2-dimethoxyethylidene orthoester, and alpha-methoxybenzylidene orthoester; suitable cyclic acetals include methylene acetal, ethylidene acetal, t-butylmethylidene acetal, 3-(benzyloxy)propyl acetal, benzylidene acetal, 3,4-dimethoxybenzylidene acetal and p-acetoxybenzylidene acetal; and suitable cyclic ketals include 1-t-butylethylidene ketal, 1-phenylethylidene ketal, isopropylidene ketal, cyclopentylidene ketal, cyclohexylidene ketal, cycloheptylidene ketal and 1-(4-methoxyphenyl)ethylidene ketal.

If desired, any —NH and/or NH₂ groups present on the B^{1A} can also be protected with one or more suitable protecting groups. Examples of suitable protecting groups include triarylmethyl groups and silyl groups. Examples of silyl groups include, but are not limited to, trimethylsilyl (TMS), tert-butyldimethylsilyl (TBDMS), triisopropylsilyl (TIPS), tert-butyldiphenylsilyl (TBDPS), tri-iso-propylsilyloxymethyl and [2-(trimethylsilyl)ethoxy]methyl.

Suitable phosphorochloridates can be commercially obtained or prepared by a synthetic method described herein. An example of a general structure of a phosphorochloridate is shown in Scheme 1. In some embodiments, the phosphorochloridate can be coupled to a compound of Formula (A). In some embodiments, to facilitate the coupling, a Grignard reagent can be used. Suitable Grignard reagents are known to those skilled in the art and include, but are not limited to, alkylmagnesium chlorides and alkylmagnesium bromides. In other embodiments, the phosphorochloridate can be added to a compound of Formula (A) using a base. Suitable bases are known to those skilled in the art. Examples of bases include, but are not limited to, an amine base, such as an alkylamine (including mono-, di- and tri-alkylamines (e.g., triethylamine)), optionally substituted pyridines (e.g. collidine) and optionally substituted imidzoles (e.g., N-methylimidazole)).

When at least one of R^{3a} and R^{3b} is an optionally substituted C₁₋₆ alkyl or an optionally substituted C₁₋₆ haloalkyl, the optionally substituted C₁₋₆ alkyl or the optionally substituted C₁₋₆ haloalkyl can be added to the 5′-position using methods known to those skilled in the art. In some embodiments, the hydroxy attached to the 5′-carbon can be oxidized to an aldehyde. Suitable oxidation conditions include, but are not limited to, DMSO in combination with an activating agent (usually an acylating agent or an acid) and an amine base, Moffatt oxidation, Swern oxidation and Corey-Kim oxidation, and suitable oxidizing agents include, but are not limited to, Dess-Martin periodinane, TPAP/NMO (tetrapropylammonium perruthenate/N-methylmorpholine N-oxide), Swern oxidation reagent, PCC (pyridinium chlorochromate), and/or PDC (pyridinium dichromate), sodium periodate, Collin's reagent, ceric ammonium nitrate CAN, Na₂Cr₂O₇ in water, Ag₂CO₃ on celite, hot HNO₃ in aqueous glyme, O₂-pyridine CuCl, Pb(OAc)₄-pyridine and benzoyl peroxide-NiBr₂. The resulting aldehyde compound can be reacted with a Grignard reagent, an organolithium reagent or trialkylaluminum (e.g. trimethylaluminum) to form a compound of Formula (A) where at least one of R^{3A} and R^{3B} is an optionally substituted C₁₋₆ alkyl or an optionally substituted C₁₋₆ haloalkyl. Optionally, the alkylating reagents can be in the presence of a Lewis acid. Suitable Lewis acids are known to those skilled in the art.

The chirality of the 5′-carbon of compounds of Formulae (A) and/or (I) can be inverted using methods known to the skilled in the art. For example, the oxygen attached to the 5′-carbon can be oxidized, for example to an aldehyde, for a compound of Formula (A), or ketone, for a compound of Formula (I), using a suitable oxidizing agent. The aldehyde and/or ketone can then be reduced using a suitable reducing agent. Examples of suitable reducing agents include, but are not limited to, NaH, LiH, NaBH₄, LiAlH₄ and CaH₂. Suitable oxidizing and reducing agents are known to those skilled in the art. Examples of suitable oxidizing agents and conditions are described herein.

As described herein, in some embodiments, R⁵ and R⁶ can be both oxygen atoms linked together by a carbonyl groups. The —O—C(═O)—O— group can be formed using methods known to those skilled in the art. For example, a compound of Formula (I), wherein R⁵ and R⁶ are both hydroxy groups, can be treated with 1,1′-carbonyldiimidazole (CDI).

In some embodiments, R⁵ and/or R⁶ can be —OC(═O)R¹⁰ and —OC(═O)R¹², respectively. The —OC(═O)R¹⁰ and —OC(═O)R¹² groups can be formed at the 2′- and 3′-positions using various methods known to those skilled in the art. As an example, a compound of Formula (I), wherein R⁵ and R⁶ are both hydroxy groups, can be treated with an alkyl anhydride (e.g., acetic anhydride and propionic anhydride) or an alkyl acid chloride (e.g., acetylchloride). If desired, a catalyst can be used to facilitate the reaction. An example of suitable catalyst is 4-dimethylaminopyridine (DMAP). Alternatively, the —OC(═O)R¹⁰ and —OC(═O)R¹² groups can be formed at the 2′- and 3′-positions by reacting an alkyl acid (e.g. acetic acid and propionic acid) in the presences of a carbodiimide or a coupling reagent. Examples of carbodiimides include, but are not limited to, N,N′-dicyclohexylcarbodiimide (DCC), N,N′-diisopropylcarbodiimide (DIC) and 1-ethyl-3-(3-dimethylaminopropyl) carbodiimide (EDC).

As described herein, B^{1A} can include a carbamate and/or an amide. Those skilled in the art know methods for forming a carbamate and/or an amide on B^{1A}. In some embodiments, the carbamate can be formed using 1,1′-carbonyldiimidazole and an alcohol.

B^{1A} can be added to the pentose ring using various methods known to those skilled in the art. In some embodiments, a compound of Formula (B) can be reacted with a nitrogenous base. In some embodiments, R^{3A}, R^{3B}, R^{4A}, R^{5A}, R^{6A}, R^{7A}, R^{8A} and B^{1A} of a compound of Formula (B) can be the same as disclosed herein, with respect to R^{3a}, R^{3b}, R⁴, R⁵, R⁶, R⁷, R⁸ and B¹; and PG¹ can be an appropriate protecting group. In some embodiments, PG¹ can be p-nitrobenzyl group. In some embodiments, any hydroxy groups attached to the pentose ring can be protected with one or more suitable protecting groups. In some embodiments, any hydroxy groups attached to the pentose ring can be protected with benzoyl groups. Examples of nitrogenous bases include an optionally substituted heterocyclic bases described herein, wherein the nitrogen atom (—N) connected to the pentose ring is —NH. If desired, any —NH and/or NH₂ groups present on the nitrogenous base can be protected with one or more suitable protecting groups. Suitable protecting groups are described herein. In some embodiments, the nitrogenous base can be added via a coupling reaction in the presence of a Lewis acid or TMSOTf (trimethylsilyl trifluoromethanesulfonate). Suitable Lewis acids are known to those skilled in the art.

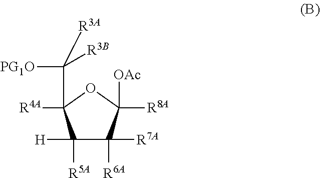

During the synthesis of any of the compounds described herein, if desired, any hydroxy groups attached to the pentose ring, and any —NH and/or NH₂ groups present on the B^{1A} can be protected with one or more suitable protecting groups. Suitable protecting groups are described herein. Those skilled in the art will appreciate that groups attached to the pentose ring and any —NH and/or NH₂ groups present on the B^{1A} can be protected with various protecting groups, and any protecting groups present can be exchanged for other protecting groups. The selection and exchange of the protecting groups is within the skill of those of ordinary skill in the art. Any protecting group(s) can be removed by methods known in the art, for example, with an acid (e.g., a mineral or an organic acid), a base or a fluoride source.

### Pharmaceutical Compositions

Some embodiments described herein relates to a pharmaceutical composition, that can include a therapeutically effective amount of one or more compounds described herein (e.g., a compound of Formulae (I) or (Iα)), or a pharmaceutically acceptable salt thereof) and a pharmaceutically acceptable carrier, diluent, excipient or combination thereof. In some embodiments, the pharmaceutical composition can include a single diastereomer of a compound of Formula (I), or a pharmaceutically acceptable salt thereof, (for example, a single diastereomer is present in the pharmaceutical composition at a concentration of greater than 99% compared to the total concentration of the other diastereomers). In other embodiments, the pharmaceutical composition can include a mixture of diastereomers of a compound of Formula (I), or a pharmaceutically acceptable salt thereof. For example, the pharmaceutical composition can include a concentration of one diastereomer of >50%, ≧60%, ≧70%, ≧80%, ≧90%, ≧95%, or ≧98%, as compared to the total concentration of the other diastereomers. In some embodiments, the pharmaceutical composition includes a 1:1 mixture of two diastereomers of a compound of Formula (I), or a pharmaceutically acceptable salt thereof.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes will be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Use

One embodiment disclosed herein relates to a method of treating and/or ameliorating a disease or condition that can include administering to a subject a therapeutically effective amount of one or more compounds described herein, such as a compound of Formula (I) (including compounds of Formula (Iα)), or a pharmaceutically acceptable salt thereof, or a pharmaceutical composition that includes a compound described herein.

Some embodiments disclosed herein relate to a method of ameliorating or treating a neoplastic disease that can include administering to a subject suffering from a neoplastic disease a therapeutically effective amount of one or more compounds described herein (e.g., a compound of Formulae (I) and/or (Iα), or a pharmaceutically acceptable salt thereof), or a pharmaceutical composition that includes a compound described herein. In an embodiment, the neoplastic disease can be cancer. In some embodiments, the neoplastic disease can be a tumor such as a solid tumor. In an embodiment, the neoplastic disease can be leukemia. Exemplary leukemias include, but are not limited to, acute lymphoblastic leukemia (ALL), acute myeloid leukemia (AML) and juvenile myelomonocytic leukemia (JMML).

Some embodiments disclosed herein relate to a method of inhibiting the growth of a tumor that can include administering to a subject having a tumor a therapeutically effective amount of one or more compounds described herein (for example, a compound of Formulae (I) and/or (Iα)), or a pharmaceutical composition that includes one or more compounds described herein.

Other embodiments disclosed herein relates to a method of ameliorating or treating a viral infection that can include administering to a subject suffering from a viral infection a therapeutically effective amount of one or more compounds described herein (for example, a compound of Formulae (I) and/or (Iα)), or a pharmaceutical composition that includes one or more compounds described herein. In an embodiment, the viral infection can be caused by a virus selected from an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae, a Togaviridae an Arenaviridae and/or a Bornaviridae. In some embodiments, the viral infection can be a hepatitis C viral (HCV) infection. In other embodiments, the viral infection can be influenza. In still other embodiments, the viral infection can be HIV.

Some embodiments disclosed herein relate to methods of ameliorating and/or treating a viral infection that can include contacting a cell infected with the virus with an effective amount of one or more compounds described herein, or a pharmaceutically acceptable salt of a compound described herein, or a pharmaceutical composition that includes one or more compounds described herein, or a pharmaceutically acceptable salt thereof. Other embodiments described herein relate to using one or more compounds described herein, or a pharmaceutically acceptable salt of a compound described herein, in the manufacture of a medicament for ameliorating and/or treating a viral infection that can include contacting a cell infected with the virus with an effective amount of said compound(s). Still other embodiments described herein relate to one or more compounds described herein, or a pharmaceutically acceptable salt of a compound described herein, that can be used for ameliorating and/or treating a viral infection by contacting a cell infected with the virus with an effective amount of said compound(s). In some embodiments, the compound can be a compound of Formulae (I) and/or (Iα), or a pharmaceutical acceptable salt thereof. In other embodiments, the compound can be a mono-, di- and/or tri-phosphate of a compound of Formulae (I) and/or (Iα), or a pharmaceutically acceptable salt of the foregoing. In some embodiments, the virus can be a HCV virus.

Some embodiments disclosed herein relate to methods of inhibiting replication of a virus that can include contacting a cell infected with the virus with an effective amount of one or more compounds described herein, or a pharmaceutically acceptable salt of a compound described herein, or a pharmaceutical composition that includes one or more compounds described herein, or a pharmaceutically acceptable salt thereof. Other embodiments described herein relate to using one or more compounds described herein, or a pharmaceutically acceptable salt of a compound described herein, in the manufacture of a medicament for inhibiting replication of a virus that can include contacting a cell infected with the virus with an effective amount of said compound(s). Still other embodiments described herein relate to a compound described herein, or a pharmaceutically acceptable salt of a compound described herein, that can be used for inhibiting replication of a virus by contacting a cell infected with the virus with an effective amount of said compound(s). In some embodiments, the compound can be a compound of Formulae (I) and/or (Iα), or a pharmaceutical acceptable salt thereof. In other embodiments, the compound can be a mono-, di- and/or tri-phosphate of a compound of Formulae (I) and/or (Iα), or a pharmaceutically acceptable salt of the foregoing. In some embodiments, the virus can be a HCV virus.

HCV is an enveloped positive strand RNA virus in the Flaviviridae family. There are various nonstructural proteins of HCV, such as NS2, NS3, NS4, NS4A, NS4B, NS5A, and NS5B. NS5B is believed to be an RNA-dependent RNA polymerase involved in the replication of HCV RNA.

Some embodiments described herein relate to a method of inhibiting NS5B polymerase activity can include contacting a cell (for example, a cell infected with HCV) with an effective amount of a compound of Formulae (I) and/or (Iα), or a pharmaceutical acceptable salt thereof. Some embodiments described herein relate to a method of inhibiting NS5B polymerase activity can include administering a cell (for example, a cell infected with HCV) with an effective amount of a compound of Formulae (I) and/or (Iα), or a pharmaceutical acceptable salt thereof. In some embodiments, a compound of Formula (I) (including a compound of Formula (Iα)), or a pharmaceutically acceptable salt thereof, can inhibit an RNA dependent RNA polymerase. In some embodiments, a compound of Formula (I) (including a compound of Formula (Iα)), or a pharmaceutically acceptable salt thereof, can inhibit a HCV polymerase (for example, NS5B polymerase).

Some embodiments described herein relate to a method of treating HCV infection in a subject suffering from a HCV infection that can include administering to the subject an effective amount of a compound of Formulae (I) and/or (Iα), or a pharmaceutical acceptable salt thereof, or a pharmaceutical composition that includes an effective amount of a compound of Formulae (I) and/or (Iα), or a pharmaceutical acceptable salt thereof. Some embodiments described herein relate to a method of treating a condition selected from liver fibrosis, liver cirrohis, and liver cancer in a subject suffering from one or more of the aforementioned liver conditions that can include administering to the subject an effective amount of a compound or a pharmaceutical composition described herein (for example, a compound of Formulae (I) and/or (Iα), or a pharmaceutical acceptable salt thereof). One cause of the liver fibrosis, liver cirrohis, and/or liver cancer can be a HCV infection. Some embodiments described herein relate to a method of increasing liver function in a subject having a HCV infection that can include administering to the subject an effective amount of a compound or a pharmaceutical composition described herein (for example, a compound of Formulae (I) and/or (Iα), or a pharmaceutical acceptable salt thereof). Also contemplated is a method for reducing or eliminating further virus-caused liver damage in a subject having an HCV infection by administering to the subject an effective amount of a compound or a pharmaceutical composition described herein (for example, a compound of Formulae (I) and/or (Iα), or a pharmaceutical acceptable salt thereof). In one embodiment, this method comprises slowing or halting the progression of liver disease. In another embodiment, the course of the disease is reversed, and stasis or improvement in liver function is contemplated.

There are a variety of genotypes of HCV, and a variety of subtypes within each genotype. For example, at present it is known that there are eleven (numbered 1 through 11) main genotypes of HCV, although others have classified the genotypes as 6 main genotypes. Each of these genotypes is further subdivided into subtypes (1a-1c; 2a-2c; 3a-3b; 4a-4e; 5a; 6a; 7a-7b; 8a-8b; 9a; 10a; and 11a). In some embodiments, an effective amount of a compound of Formulae (I) and/or (Iα), or a pharmaceutical acceptable salt thereof, or a pharmaceutical composition that includes an effective amount of a compound of Formulae (I) and/or (Iα), or a pharmaceutical acceptable salt thereof, can be effective to treat at least one genotype of HCV. In some embodiments, a compound described herein (for example, a compound of Formulae (I) and/or (Iα), or a pharmaceutical acceptable salt thereof) can be effective to treat all 11 genotypes of HCV. In some embodiments, a compound described herein (for example, a compound of Formulae (I) and/or (Iα), or a pharmaceutical acceptable salt thereof) can be effective to treat 3 or more, 5 or more, 7 or more of 9 more genotypes of HCV. In some embodiments, a compound of Formula (I) and/or (Iα), or a pharmaceutical acceptable salt thereof is more effective against a larger number of HCV genotypes than the standard of care. In some embodiments, a compound of Formula (I) and/or (Iα), or a pharmaceutical acceptable salt thereof, is more effective against a particular HCV genotype than the standard of care (such as genotype 1, 2, 3, 4, 5 and/or 6).

Various indicators for determining the effectiveness of a method for treating a HCV infection are known to those skilled in the art. Example of suitable indicators include, but are not limited to, a reduction in viral load, a reduction in viral replication, a reduction in time to seroconversion (virus undetectable in patient serum), an increase in the rate of sustained viral response to therapy, a reduction of morbidity or mortality in clinical outcomes, a reduction in the rate of liver function decrease; stasis in liver function; improvement in liver function; reduction in one or more markers of liver dysfunction, including alanine transaminase, aspartate transaminase, total bilirubin, conjugated bilirubin, gamma glutamyl transpeptidase, and/or other indicator of disease response. Similarly, successful therapy with an effective amount of a compound or a pharmaceutical composition described herein (for example, a compound of Formulae (I) and/or (Iα), or a pharmaceutical acceptable salt thereof) can reduce the incidence of liver cancer in HCV patients.

In some embodiments, an effective amount of a compound of Formulae (I) and/or (Iα), or a pharmaceutically acceptable salt thereof, is an amount that is effective to reduce viral titers to undetectable levels, for example, to about 1000 to about 5000, to about 500 to about 1000, or to about 100 to about 500 genome copies/mL serum. In some embodiments, an effective amount of a compound of Formula (I) and/or (Iα), or a pharmaceutically acceptable salt thereof, is an amount that is effective to reduce viral load compared to the viral load before administration of the compound of Formula (I) and/or (Iα), or a pharmaceutically acceptable salt thereof. For example, wherein the viral load is measure before administration of the compound of Formula (I) and/or (Iα), or a pharmaceutically acceptable salt thereof, and again after completion of the treatment regime with the compound of Formula (I) and/or (Iα), or a pharmaceutically acceptable salt thereof (for example, 1 month after completion). In some embodiments, an effective amount of a compound of Formula (I) and/or (Iα), or a pharmaceutically acceptable salt thereof, can be an amount that is effective to reduce viral load to lower than about 100 genome copies/mL serum. In some embodiments, an effective amount of a compound of Formula (I) and/or (Iα), or a pharmaceutically acceptable salt thereof, is an amount that is effective to achieve a reduction in viral titer in the serum of the subject in the range of about 1.5-log to about a 2.5-log reduction, about a 3-log to about a 4-log reduction, or a greater than about 5-log reduction compared to the viral load before administration of the compound of Formula (I) and/or (Iα), or a pharmaceutically acceptable salt thereof. For example, the viral load can be measured before administration of the compound of Formula (I) and/or (Iα), or a pharmaceutically acceptable salt thereof, and again after completion of the treatment regime with the compound of Formula (I) and/or (Iα), or a pharmaceutically acceptable salt thereof (for example, 1 month after completion).

In some embodiments, a compound of Formula (I) and/or (Iα), or a pharmaceutically acceptable salt thereof, can result in at least a 1, 2, 3, 4, 5, 10, 15, 20, 25, 50, 75, 100-fold or more reduction in the replication of HCV relative to pre-treatment levels in a subject, as determined after completion of the treatment regime (for example 1 month after completion). In some embodiments, a compound of Formula (I) and/or (Iα), or a pharmaceutically acceptable salt thereof, can result in a reduction of the replication of HCV relative to pre-treatment levels in the range of about 2 to about 5 fold, about 10 to about 20 fold, about 15 to about 40 fold, or about 50 to about 100 fold. In some embodiments, a compound of Formula (I) and/or (Iα), or a pharmaceutically acceptable salt thereof, can result in a reduction of HCV replication in the range of 1 to 1.5 log, 1.5 log to 2 log, 2 log to 2.5 log, 2.5 to 3 log, 3 log to 3.5 log or 3.5 to 4 log more reduction of HCV replication compared to the reduction of HCV reduction achieved by pegylated interferon in combination with ribavirin, administered according to the standard of care, or may achieve the same reduction as that standard of care therapy in a shorter period of time, for example, in one month, two months, or three months, as compared to the reduction achieved after six months of standard of care therapy with ribavirin and pegylated interferon.

In some embodiments, an effective amount of a compound of Formula (I) and/or (Iα), or a pharmaceutically acceptable salt thereof, is an amount that is effective to achieve a sustained viral response, for example, non-detectable or substantially non-detectable HCV RNA (e.g., less than about 500, less than about 400, less than about 200, or less than about 100 genome copies per milliliter serum) is found in the subject's serum for a period of at least about one month, at least about two months, at least about three months, at least about four months, at least about five months, or at least about six months following cessation of therapy.

In some embodiments, a therapeutically effective amount of a compound of Formula (I) and/or (Iα), or a pharmaceutically acceptable salt thereof, can reduce a level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated subject, or to a placebo-treated subject. Methods of measuring serum markers are known to those skilled in the art and include immunological-based methods, e.g., enzyme-linked immunosorbent assays (ELISA), radioimmunoassays, and the like, using antibody specific for a given serum marker. A non-limiting list of examples of a markers includes measuring the levels of serum alanine aminotransferase (ALT), asparatate aminotransferacse (AST), alkaline phosphatase (ALP), gamma-glutamyl transpeptidase (GGT) and total bilirubin (TBIL) using known methods. In general, an ALT level of less than about 45 IU/L (international units/liter), an AST in the range of 10-34 IU/L, ALP in the range of 44-147 IU/L, GGT in the range of 0-51 IU/L, TBIL in the range of 0.3-1.9 mg/dL is considered normal. In some embodiments, an effective amount of a compound of Formula (I) and/or (Iα) is an amount effective to reduce ALT, AST, ALP, GGT and/or TBIL levels to with what is considered a normal level.

Subjects who are clinically diagnosed with HCV infection include “naïve” subjects (e.g., subjects not previously treated for HCV, particularly those who have not previously received IFN-alpha-based and/or ribavirin-based therapy) and individuals who have failed prior treatment for HCV (“treatment failure” subjects). Treatment failure subjects include “non-responders” (i.e., subjects in whom the HCV titer was not significantly or sufficiently reduced by a previous treatment for HCV (≦0.5 log IU/mL), for example, a previous IFN-alpha monotherapy, a previous IFN-alpha and ribavirin combination therapy, or a previous pegylated IFN-alpha and ribavirin combination therapy); and “relapsers” (i.e., subjects who were previously treated for HCV, for example, who received a previous IFN-alpha monotherapy, a previous IFN-alpha and ribavirin combination therapy, or a previous pegylated IFN-alpha and ribavirin combination therapy, whose HCV titer decreased, and subsequently increased).

In some embodiments, a compound of Formula (I), or a pharmaceutically acceptable salt thereof, can be administered to a treatment failure subject suffering from HCV. In some embodiments, a compound of Formula (I), or a pharmaceutically acceptable salt thereof, can be administered to a non-responder subject suffering from HCV. In some embodiments, a compound of Formula (I), or a pharmaceutically acceptable salt thereof, can be administered to a relapsed subject suffering from HCV.

After a period of time, infectious agents can develop resistance to one or more therapeutic agents. The term “resistance” as used herein refers to a viral strain displaying a delayed, lessened and/or null response to a therapeutic agent(s). For example, after treatment with an antiviral agent, the viral load of a subject infected with a resistant virus may be reduced to a lesser degree compared to the amount in viral load reduction exhibited by a subject infected with a non-resistant strain. In some embodiments, a compound of Formula (I), or a pharmaceutically acceptable salt thereof, can be administered to a subject infected with an HCV strain that is resistant to one or more different anti-HCV agents. In some embodiments, development of resistant HCV strains is delayed when patients are treated with a compound of Formula (I), or a pharmaceutically acceptable salt thereof, compared to the development of HCV strains resistant to other HCV drugs.

In some embodiments, an effective amount of a compound of Formula (I), or a pharmaceutically acceptable salt thereof, can be administered to a subject for whom other anti-HCV medications are contraindicated. For example, administration of pegylated interferon alpha in combination with ribavirin is contraindicated in subjects with hemoglobinopathies (e.g., thalassemia major, sickle-cell anemia) and other subjects at risk from the hematologic side effects of current therapy. In some embodiments, a compound of Formula (I), or a pharmaceutically acceptable salt thereof, can be provided to a subject that is hypersensitive to interferon or ribavirin.

Some subjects being treated for HCV experience a viral load rebound. The term “viral load rebound” as used herein refers to a sustained ≧0.5 log IU/mL increase of viral load above nadir before the end of treatment, where nadir is a ≧0.5 log IU/mL decrease from baseline. In some embodiments, a compound of Formula (I), or a pharmaceutically acceptable salt thereof, can be administered to a subject experiencing viral load rebound, or can prevent such viral load rebound when used to treat the subject.

The standard of care for treating HCV has been associated with several side effects (adverse events). In some embodiments, a compound of Formula (I) (including a compound of Formula (Iα)), or a pharmaceutically acceptable salt thereof, can decrease the number and/or severity of side effects that can be observed in HCV patients being treated with ribavirin and pegylated interferon according to the standard of care. Examples of side effects include, but are not limited to fever, malaise, tachycardia, chills, headache, arthralgias, myalgias, fatigue, apathy, loss of apetite, nausea, vomiting, cognitive changes, asthenia, drowsiness, lack of initiative, irritability, confusion, depression, severe depression, suicidal ideation, anemia, low white blood cell counts, and thinning of hair. In some embodiments, a compound of Formula (I), or a pharmaceutically acceptable salt thereof, can be provided to a subject that discontinued a HCV therapy because of one or more adverse effects or side effects associated with one or more other HCV agents.

Table 5 provides some embodiments of a compound of Formula (I), or a pharmaceutically acceptable salt thereof, compared to the standard of care. Examples include the following: in some embodiments, a compound of Formula (I), or a pharmaceutically acceptable salt thereof, results in a percentage of non-responders that is 10% less than the percentage of non-responders receiving the standard of care; in some embodiments, a compound of Formula (I), or a pharmaceutically acceptable salt thereof, results number of side effects that is in the range of about 10% to about 30% less than compared to the number of side effects experienced by a subject receiving the standard of care; and in some embodiments, a compound of Formula (I), or a pharmaceutically acceptable salt thereof, results a severity of a side effect (such as one of those described herein) that is 25% less than compared to the severity of the same side effect experienced by a subject receiving the standard of care. Methods of quantifying the severity of a side effect are known to those skilled in the art.

TABLE 5

| Percent- | | | Percent- | | |
|---|---|---|---|---|---|
| age of | Percent- | Percent- | age of | Number | Severity |
| non- | age of | age of | viral load | of side | of side |
| responders | relapsers | resistance | rebound | effects | effects |
| 10% less | 10% less | 10% less | 10% less | 10% less | 10% less |
| 25% less | 25% less | 25% less | 25% less | 25% less | 25% less |
| 40% less | 40% less | 40% less | 40% less | 40% less | 40% less |
| 50% less | 50% less | 50% less | 50% less | 50% less | 50% less |
| 60% less | 60% less | 60% less | 60% less | 60% less | 60% less |
| 70% less | 70% less | 70% less | 70% less | 70% less | 70% less |
| 80% less | 80% less | 80% less | 80% less | 80% less | 80% less |
| 90% less | 90% less | 90% less | 90% less | 90% less | 90% less |
| about 10% | about 10% | about 10% | about 10% | about 10% | about 10% |
| to about | to about | to about | to about | to about | to about |
| 30% less | 30% less | 30% less | 30% less | 30% less | 30% less |
| about 20% | about 20% | about 20% | about 20% | about 20% | about 20% |
| to about | to about | to about | to about | to about | to about |
| 50% less | 50% less | 50% less | 50% less | 50% less | 50% less |
| about 30% | about 30% | about 30% | about 30% | about 30% | about 30% |
| to about | to about | to about | to about | to about | to about |
| 70% less | 70% less | 70% less | 70% less | 70% less | 70% less |
| about 20% | about 20% | about 20% | about 20% | about 20% | about 20% |
| to about | to about | to about | to about | to about | to about |
| 80% less | 80% less | 80% less | 80% less | 80% less | 80% less |

Yet still other embodiments disclosed herein relates to a method of ameliorating or treating a parasitic disease that can include administering to a subject suffering from a parasitic disease a therapeutically effective amount of one or more compounds described herein (for example, a compound of Formula (I) and/or (Iα)), or a pharmaceutical composition that includes one or more compounds described herein. In an embodiment, the parasite disease can be Chagas' disease.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. For example, a therapeutically effective amount of compound can be the amount needed to prevent, alleviate or ameliorate symptoms of disease or prolong the survival of the subject being treated This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years. In some embodiments, a compound of Formula (I) (including a compound of Formula (Iα)), or a pharmaceutically acceptable salt thereof, can be administered less frequently compared to the frequency of administration of an agent within the standard of care. In some embodiments, a compound of Formula (I) (including a compound of Formula (Iα)), or a pharmaceutically acceptable salt thereof, can be administered one time per day. For example, a compound of Formula (I), or a pharmaceutically acceptable salt thereof, can be administered one time per day to a subject suffering from a HCV infection. In some embodiments, the total time of the treatment regime with a compound of Formula (I) (including a compound of Formula (Iα)), or a pharmaceutically acceptable salt thereof, can less compared to the total time of the treatment regime with the standard of care.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Combination Therapies

In some embodiments, the compounds disclosed herein, such as a compound of Formula (I) (including compounds of Formula (Iα)), or a pharmaceutically acceptable salt thereof, or a pharmaceutical composition that includes a compound described herein, can be used in combination with one or more additional agent(s). Examples of additional agents that can be used in combination with a compound of Formula (I), or a pharmaceutically acceptable salt thereof, or a pharmaceutical composition that includes a compound of Formula (I), or a pharmaceutically acceptable salt thereof, include, but are not limited to, agents currently used in a conventional standard of care for treating HCV, HCV protease inhibitors, HCV polymerase inhibitors, NS5A inhibitors, other antiviral compounds, compounds of Formula (BB) (including pharmaceutically acceptable salts and pharmaceutical compositions that can include a compound of Formula (BB), or a pharmaceutically acceptable salt thereof), compounds of Formula (CC) (including pharmaceutically acceptable salts and pharmaceutical compositions that can include a compound of Formula (CC), or a pharmaceutically acceptable salt thereof), compounds of Formula (DD) (including pharmaceutically acceptable salts and pharmaceutical compositions that can include a compound of Formula (DD), or a pharmaceutically acceptable salt thereof), and/or combinations thereof. In some embodiments, a compound of Formula (I), or a pharmaceutically acceptable salt thereof, or a pharmaceutical composition that includes a compound of Formula (I), or a pharmaceutically acceptable salt thereof, can be used with one, two, three or more additional agents described herein. A non-limiting list of examples of combinations of a compound of Formula (I), or a pharmaceutically acceptable salt thereof, or a pharmaceutical composition that includes a compound of Formula (I), or a pharmaceutically acceptable salt thereof, is provided in Tables A, B, C and D.

In some embodiments, a compound of Formula (I), or a pharmaceutically acceptable salt thereof, or a pharmaceutical composition that includes a compound of Formula (I), or a pharmaceutically acceptable salt thereof, can be used in combination with an agent(s) currently used in a conventional standard of care therapy. For example, for the treatment of HCV, a compound disclosed herein can be used in combination with Pegylated interferon-alpha-2a (brand name PEGASYS®) and ribavirin, or Pegylated interferon-alpha-2b (brand name PEG-INTRON®) and ribavirin. As another example, a compound disclosed herein can be used in combination with oseltamivir (TAMIFLU®) or zanamivin (RELENZA®) for treating an influenza infection.

In some embodiments, a compound of Formula (I), or a pharmaceutically acceptable salt thereof, or a pharmaceutical composition that includes a compound of Formula (I), or a pharmaceutically acceptable salt thereof, can be substituted for an agent currently used in a conventional standard of care therapy. For example, for the treatment of HCV, a compound of Formula (I), or a pharmaceutically acceptable salt thereof, or a pharmaceutical composition that includes a compound of Formula (I), or a pharmaceutically acceptable salt thereof, can be used in place of ribavirin.

In some embodiments, a compound of Formula (I), or a pharmaceutically acceptable salt thereof, or a pharmaceutical composition that includes a compound of Formula (I), or a pharmaceutically acceptable salt thereof, can be used in combination with an interferon, such as a pegylated interferon. Examples of suitable interferons include, but are not limited to, Pegylated interferon-alpha-2a (brand name PEGASYS®), Pegylated interferon-alpha-2b (brand name PEG-INTRON®), interferon alfacon-1 (brand name INFERGEN®), pegylated interferon lambda and/or a combination thereof.

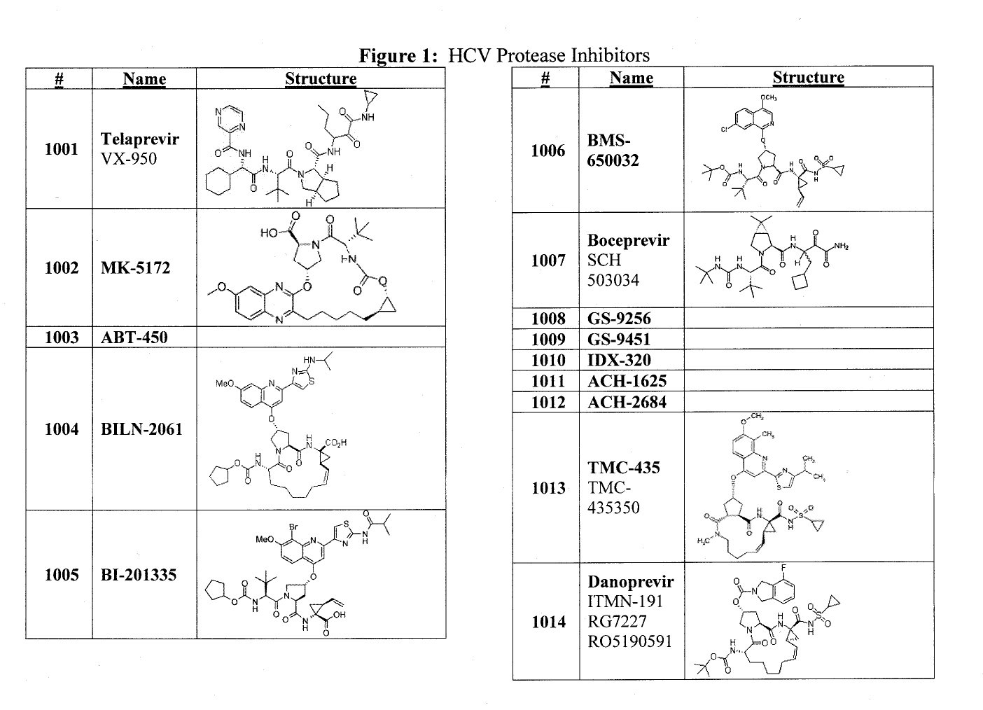

In some embodiments, a compound of Formula (I), or a pharmaceutically acceptable salt thereof, or a pharmaceutical composition that includes a compound of Formula (I), or a pharmaceutically acceptable salt thereof, can be used in combination with a HCV protease inhibitor. A non-limiting list of example HCV protease inhibitors include the following: VX-950 (TELAPREVIR®), MK-5172, ABT-450, BILN-2061, BI-201335, BMS-650032, SCH 503034 (BOCEPREVIR®), GS-9256, GS-9451, IDX-320, ACH-1625, ACH-2684, TMC-435, ITMN-191 (DANOPREVIR®) and/or a combination thereof. A non-limiting list of example HCV protease inhibitors includes the compounds numbered 1001-1014 in FIG. 1.

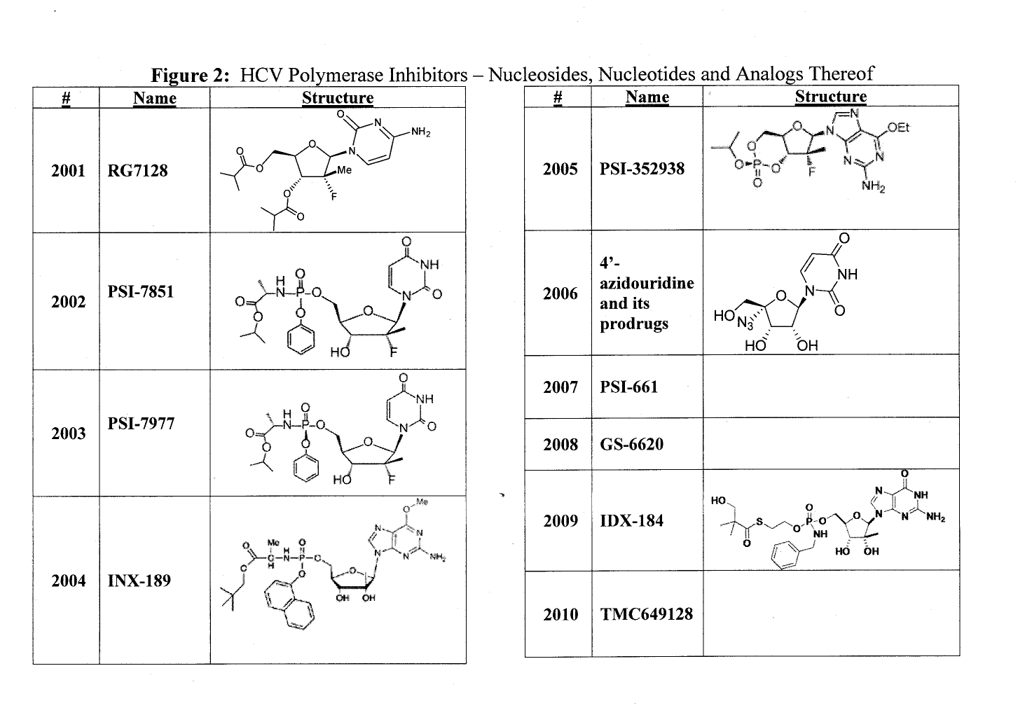

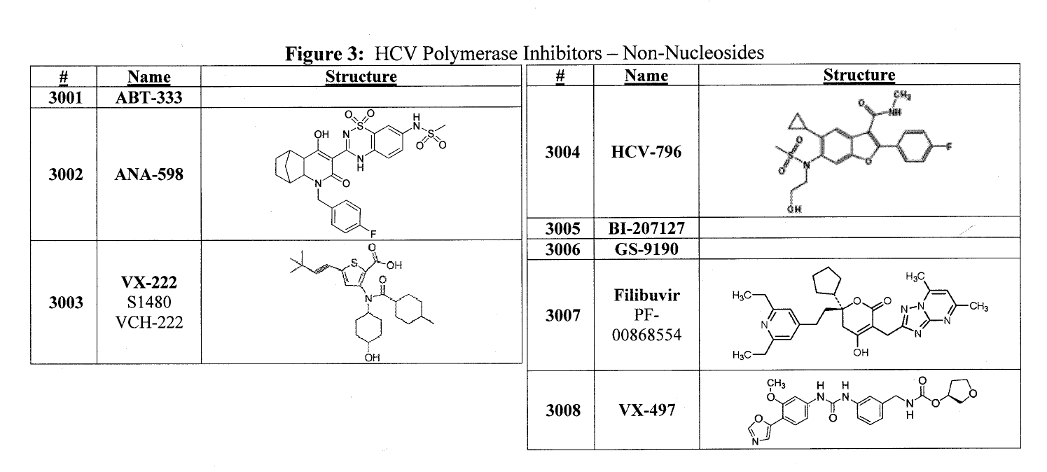

In some embodiments, a compound of Formula (I), or a pharmaceutically acceptable salt thereof, or a pharmaceutical composition that includes a compound of Formula (I), or a pharmaceutically acceptable salt thereof, can be used in combination with a HCV polymerase inhibitor. In some embodiments, the HCV polymerase inhibitor can be a nucleoside inhibitor. In other embodiments, the HCV polymerase inhibitor can be a non-nucleoside inhibitor. Examples of suitable nucleoside inhibitors include, but are not limited to, RG7128, PSI-7851, PSI-7977, INX-189, PSI-352938, PSI-661, 4′-azidouridine (including known prodrugs of 4′-azidouridine), GS-6620, IDX-184, and TMC649128, and/or combinations thereof. A non-limiting list of example nucleoside inhibitors includes compounds numbered 2001-2010 in FIG. 2. Examples of suitable non-nucleoside inhibitors include, but are not limited to, ABT-333, ANA-598, VX-222, HCV-796, BI-207127, GS-9190, PF-00868554 (FILIBUVIR®), VX-497 and/or combinations thereof. A non-limiting list of example non-nucleoside inhibitors includes the compounds numbered 3001-3008 in FIG. 3.

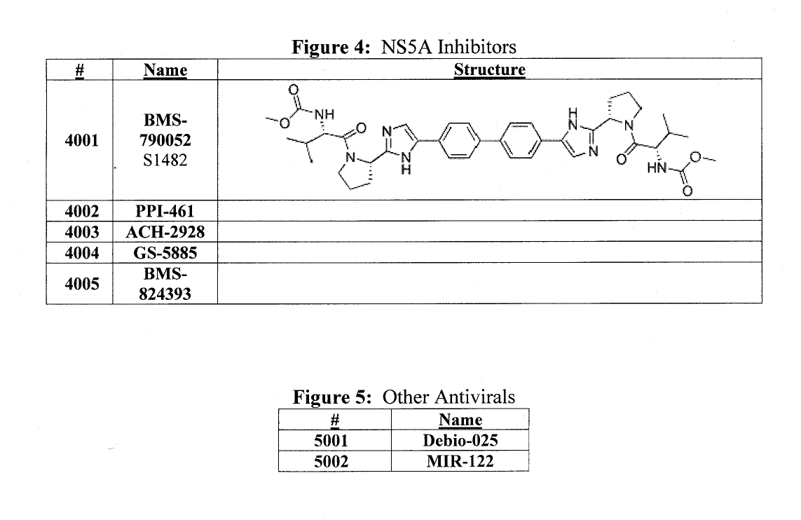

In some embodiments, a compound of Formula (I), or a pharmaceutically acceptable salt thereof, or a pharmaceutical composition that includes a compound of Formula (I), or a pharmaceutically acceptable salt thereof, can be used in combination with a NS5A inhibitor. A non-limiting list of example NS5A inhibitors include BMS-790052, PPI-461, ACH-2928, GS-5885, BMS-824393 and/or combinations thereof. A non-limiting list of example NS5A inhibitors includes the compounds numbered 4001-4005 in FIG. 4.

In some embodiments, a compound of Formula (I), or a pharmaceutically acceptable salt thereof, or a pharmaceutical composition that includes a compound of Formula (I), or a pharmaceutically acceptable salt thereof, can be used in combination with other antiviral compounds. Examples of other antiviral compounds include, but are not limited to, Debio-025, MIR-122 and/or combinations thereof. A non-limiting list of example other antiviral compounds includes the compounds numbered 5001-5002 in FIG. 5.

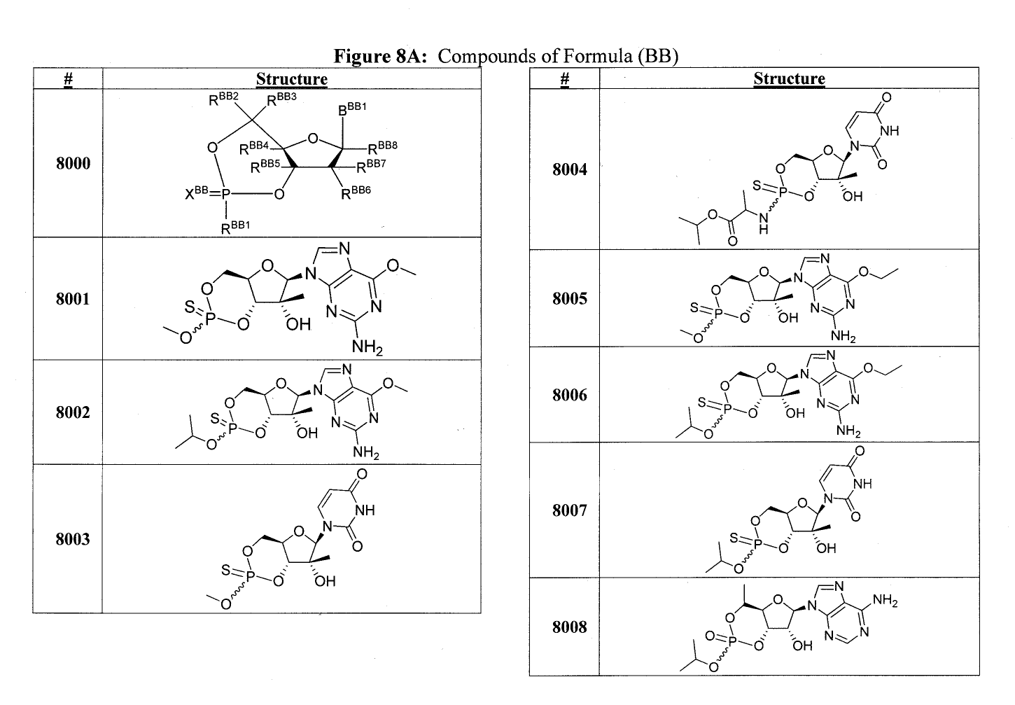

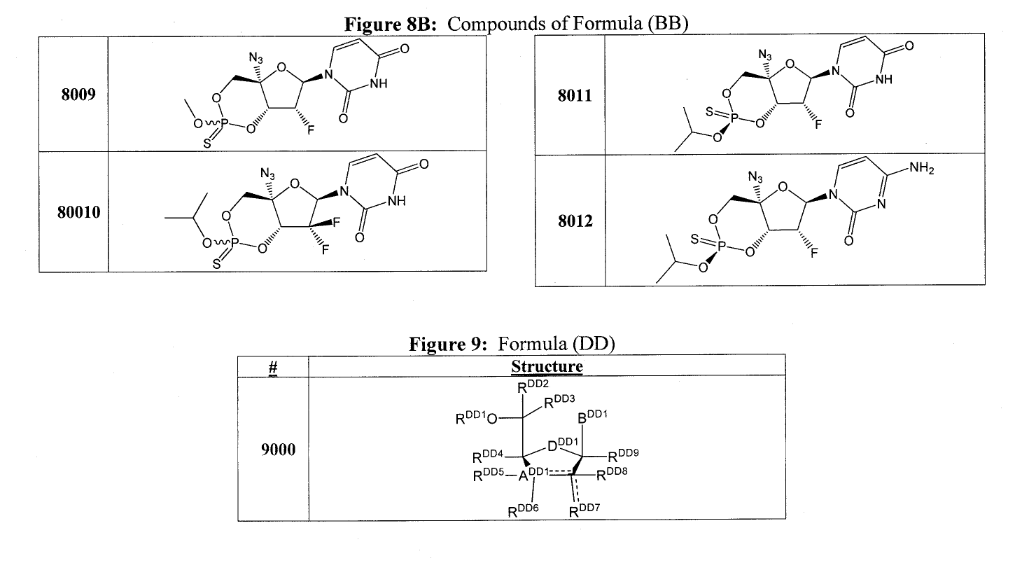

In some embodiments, a compound of Formula (I), or a pharmaceutically acceptable salt thereof, or a pharmaceutical composition that includes a compound of Formula (I), or a pharmaceutically acceptable salt thereof, can be used in combination with a compound of Formula (BB), or a pharmaceutically acceptable salt thereof, or a pharmaceutical composition that includes a compound of Formula (BB), or a pharmaceutically acceptable salt thereof (see, U.S. Provisional Application No. 61/426,471, filed Dec. 22, 2010, the contents of which are incorporated by reference in its entirety):

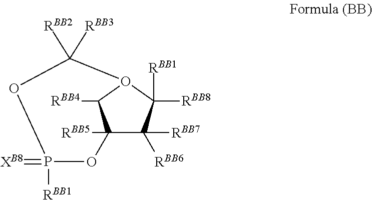

wherein B^{BB1} can be an optionally substituted heterocyclic base or an optionally substituted heterocyclic base with a protected amino group; X^{BB} can be O (oxygen) or S (sulfur); R^{BB1} can be selected from —Z^{BB}—R^{BB9} an optionally substituted N-linked amino acid and an optionally substituted N-linked amino acid ester derivative; Z^{BB} can be selected from O (oxygen), S (sulfur) and N(R^{BB10}); R^{BB2} and R^{BB3} can be independently selected from hydrogen, an optionally substituted C₁₋₆ alkyl, an optionally substituted C₂₋₆ alkenyl, an optionally substituted C₂₋₆ alkynyl, an optionally substituted C₁₋₆ haloalkyl and an optionally substituted aryl(C₁₋₆ alkyl); or R^{BB2} and R^{BB3} can be taken together to form a group selected from an optionally substituted C₃₋₆ cycloalkyl, an optionally substituted C₃₋₆ cycloalkenyl, an optionally substituted C₃₋₆ aryl and an optionally substituted C₃₋₆ heteroaryl; R^{BB4} can be selected from hydrogen, halogen, azido, cyano, an optionally substituted C₁₋₆ alkyl, an optionally substituted C₂₋₆ alkenyl, an optionally substituted C₂₋₆ alkynyl and an optionally substituted allenyl; R^{BB5} can be hydrogen or an optionally substituted C₁₋₆ alkyl; R^{BB6} can be selected from hydrogen, halogen, azido, amino, cyano, an optionally substituted C₁₋₆ alkyl, —OR^{BB11} and —OC(═O)R^{BB12}; R^{BB7} can be selected from hydrogen, halogen, azido, cyano, an optionally substituted C₁₋₆ alkyl, —OR^{BB13} and —OC(═O)RBB¹⁴; R^{BB8} can be selected from hydrogen, halogen, azido, cyano, an optionally substituted C₁₋₆ alkyl, —OR^{BB15} and —OC(═O)R^{BB16}; R^{BB9} can be selected from an optionally substituted alkyl, an optionally substituted alkenyl, an optionally substituted alkynyl, an optionally substituted cycloalkyl, an optionally substituted cycloalkenyl, an optionally substituted aryl, an optionally substituted heteroaryl, an optionally substituted heterocyclyl, an optionally substituted aryl(C₁₋₆alkyl), an optionally substituted heteroaryl(C₁₋₆alkyl) and an optionally substituted heterocyclyl(C₁₋₆alkyl); R^{BB10} can be selected from hydrogen, an optionally substituted alkyl, an optionally substituted alkenyl, an optionally substituted alkynyl, an optionally substituted cycloalkyl, an optionally substituted cycloalkenyl, an optionally substituted aryl, an optionally substituted heteroaryl, an optionally substituted heterocyclyl, an optionally substituted aryl(C₁₋₆alkyl), an optionally substituted heteroaryl(C₁₋₆alkyl) and an optionally substituted heterocyclyl(C₁₋₆alkyl); R^{BB11}, R^{BB13} and R^{BB15} can be independently hydrogen or an optionally substituted C₁₋₆ alkyl; and R^{BB12}, R^{BB14} and R^{BB16} can be independently an optionally substituted C₁₋₆ alkyl or an optionally substituted C₃₋₆ cycloalkyl. In some embodiments, at least one of R^{BB2} and R^{BB3} is not hydrogen. A non-limiting list of example compounds of Formula (BB) includes the compound numbered 8000-8012 in FIGS. 8A-8B.

In some embodiments, a compound of Formula (I), or a pharmaceutically acceptable salt thereof, or a pharmaceutical composition that includes a compound of Formula (I), or a pharmaceutically acceptable salt thereof, can be used in combination with a compound of Formula (CC), or a pharmaceutically acceptable salt thereof, or a pharmaceutical composition that includes a compound of Formula (CC), or a pharmaceutically acceptable salt thereof (see, U.S. Provisional Application Nos. 61/385,363, filed Sep. 22, 2010, and 61/426,461, filed Dec. 22, 2010, the contents of which are incorporated by reference in its entirety):

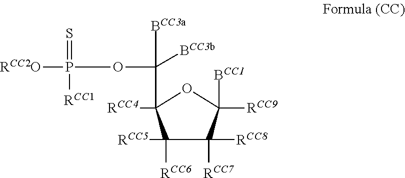

wherein B^{CC1} can be an optionally substituted heterocyclic base or an optionally substituted heterocyclic base with a protected amino group; R^{CC1} can be selected from O⁻, OH, an optionally substituted N-linked amino acid and an optionally substituted N-linked amino acid ester derivative; R^{CC2} can be selected from an optionally substituted aryl, an optionally substituted heteroaryl, an optionally substituted heterocyclyl and

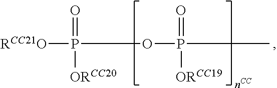

wherein R^{CC19}, R^{CC20} and R^{CC21} can be independently absent or hydrogen, and n^{cc} can be 0 or 1; provided that when R^{CC1} is O⁻ or OH, then R^{CC2} is

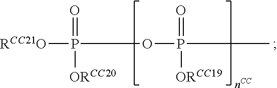

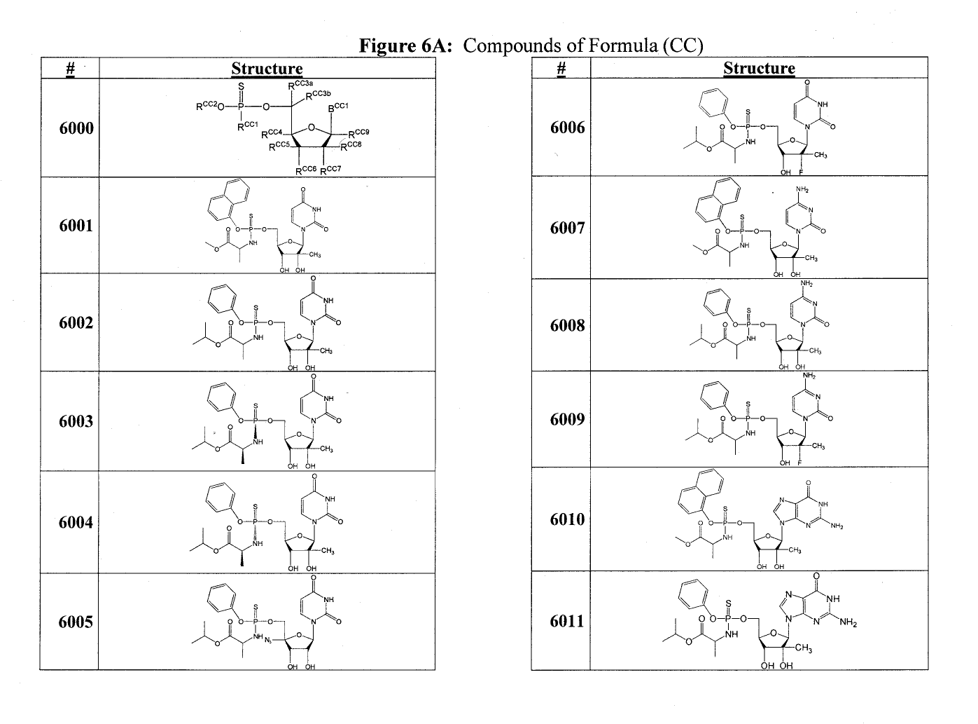

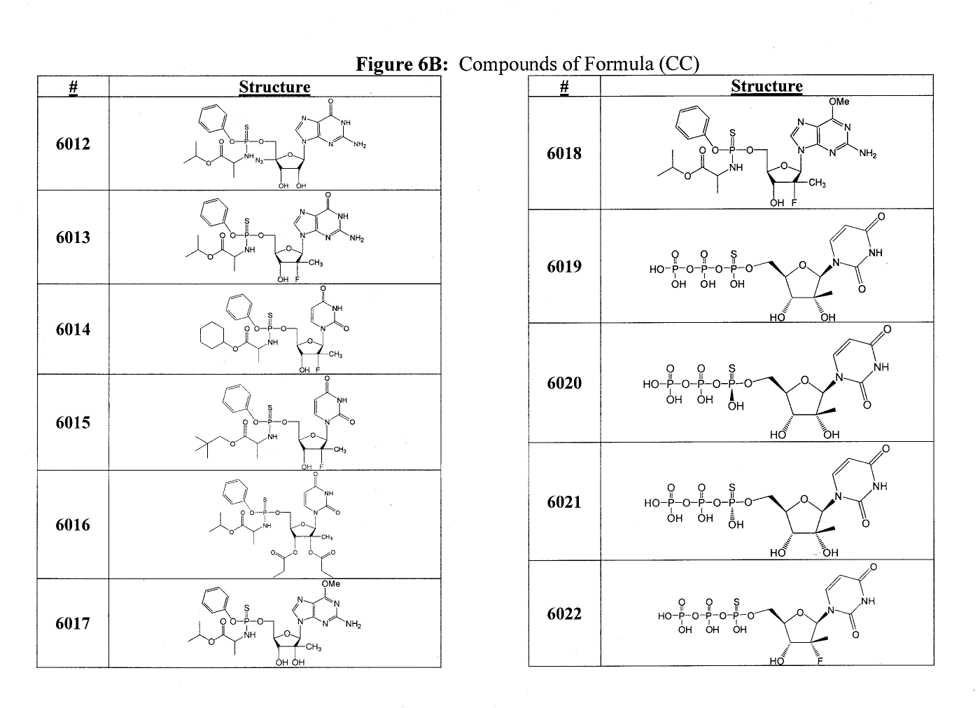

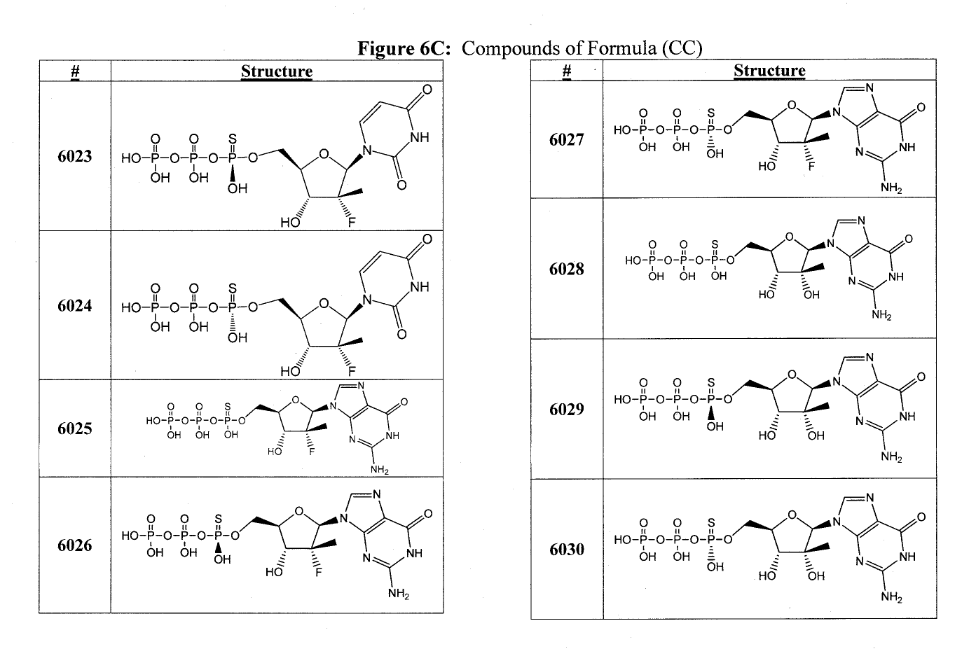

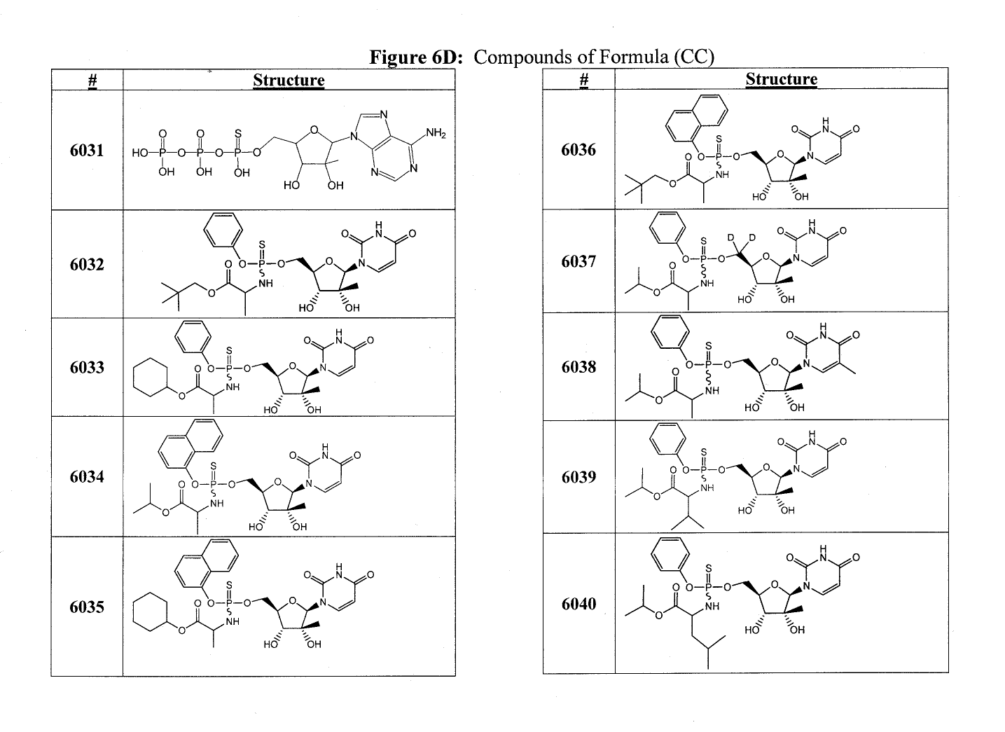

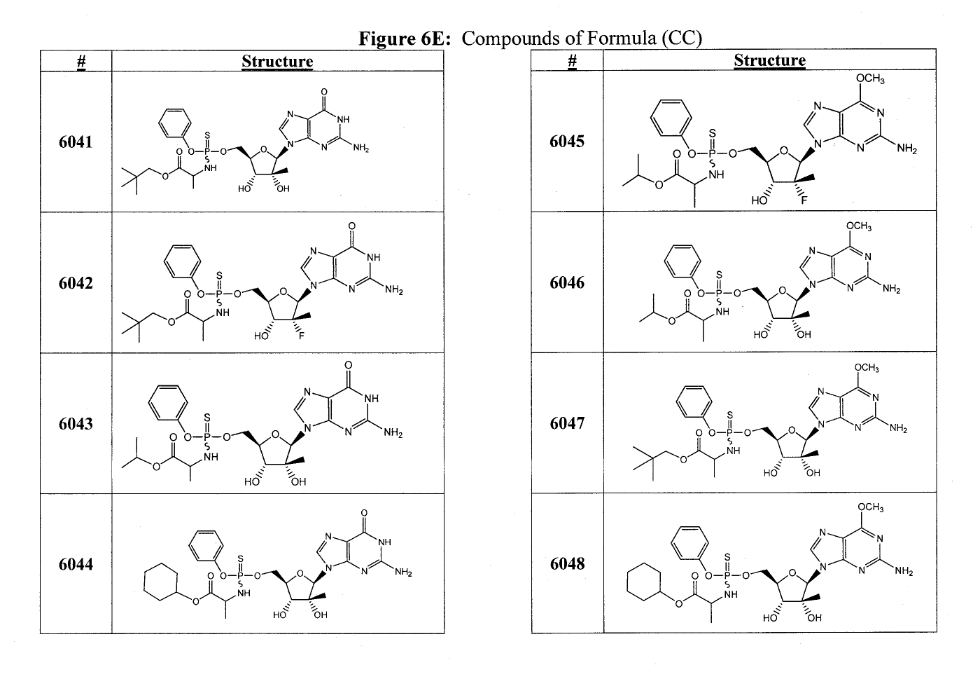

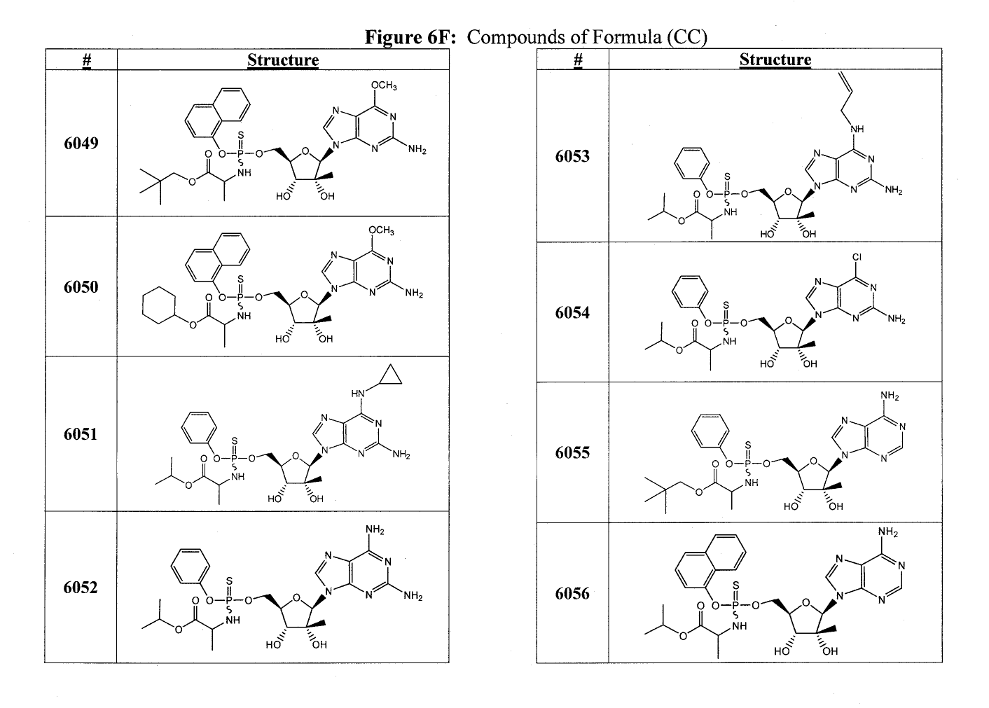

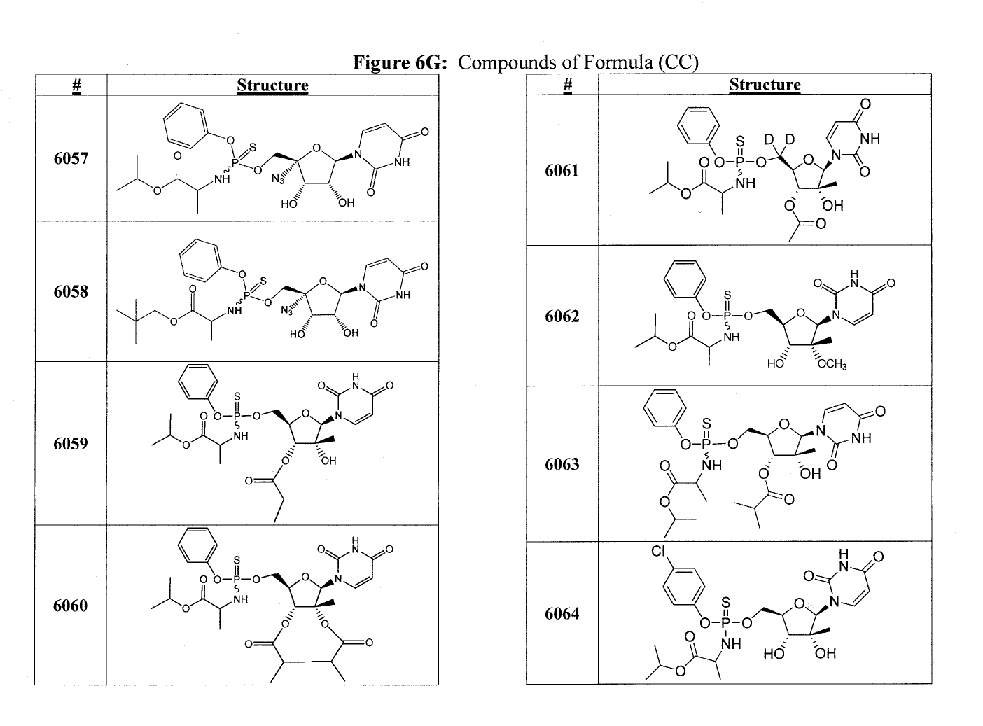

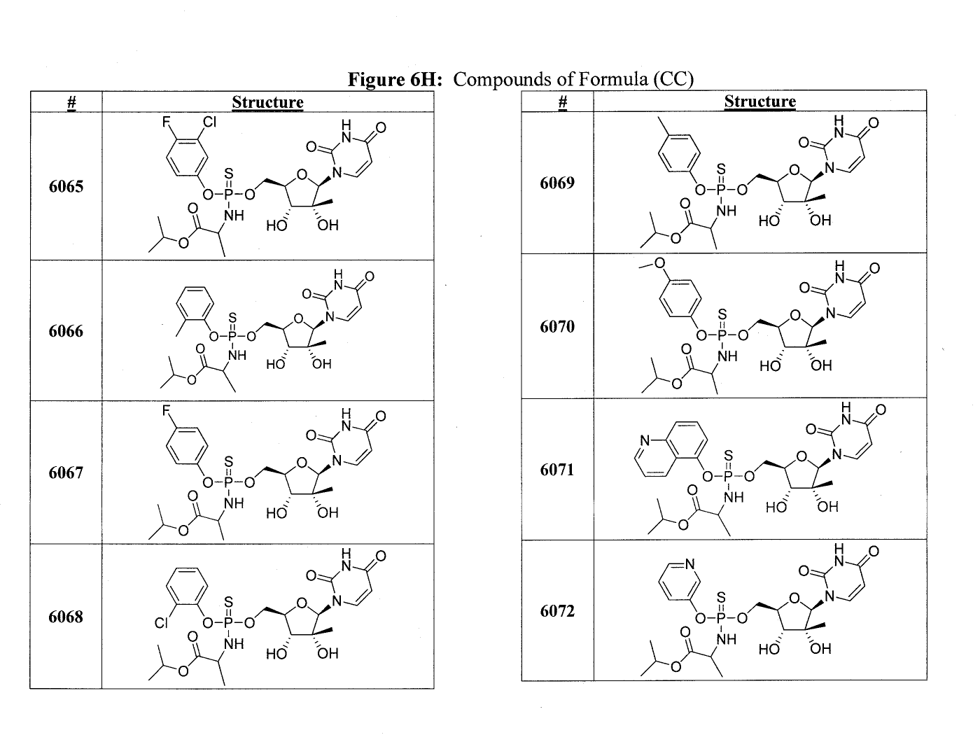

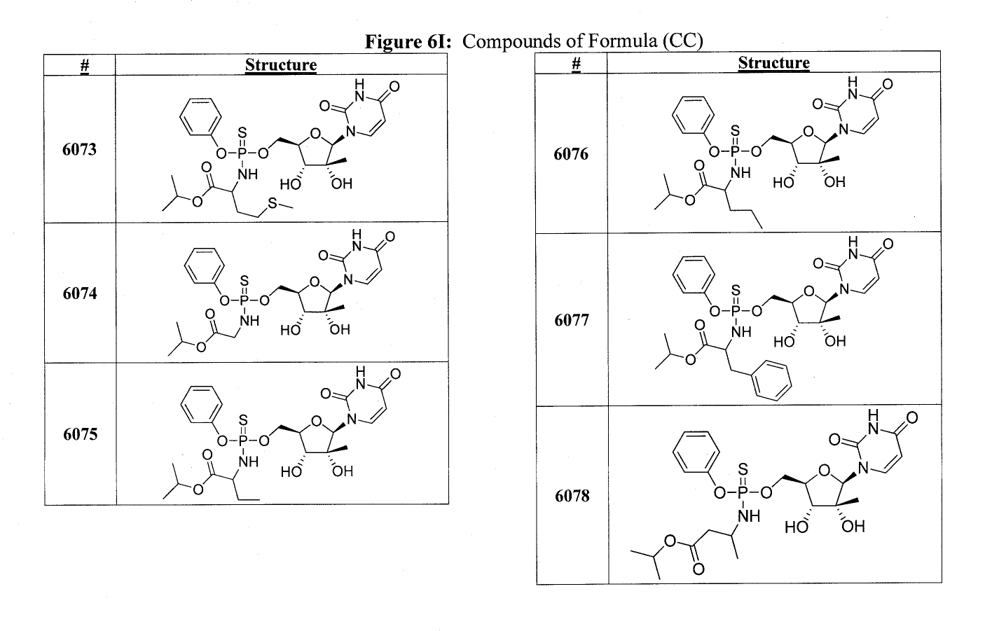

R^{CC3a} and R^{CC3b} can be independently selected from hydrogen, deuterium, an optionally substituted C₁₋₆ alkyl, an optionally substituted C₂₋₆ alkenyl, an optionally substituted C₂₋₆ alkynyl, an optionally substituted C₁₋₆ haloalkyl and aryl(C₁₋₆ alkyl); or R^{CC3a} and R^{CC3b} can be taken together to form an optionally substituted C₃₋₆ cycloalkyl; R^{CC4} can be selected from hydrogen, azido, an optionally substituted C₁₋₆ alkyl, an optionally substituted C₂₋₆ alkenyl and an optionally substituted C₂₋₆ alkynyl; R^{CC5} can be selected from hydrogen, halogen, azido, cyano, an optionally substituted C₁₋₆ alkyl, —OR^{CC10} and —OC(═O)R^{CC11}; R^{CC6} can be selected from hydrogen, halogen, azido, cyano, an optionally substituted C₁₋₆ alkyl, —OR^{CC12} and —OC(═O)R^{CC13}; R^{CC7} can be selected from hydrogen, halogen, azido, cyano, an optionally substituted C₁₋₆ alkyl, —OR^{CC14} and —OC(═O)R^{CC15}; or R^{CC6} and R^{CC7} can be both oxygen atoms and linked together by a carbonyl group; R^{CC8} can be selected from hydrogen, halogen, azido, cyano, an optionally substituted C₁₋₆ alkyl, —OR^{CC16} and —OC(═O)R^{CC17}; R^{CC9} can be selected from hydrogen, azido, cyano, an optionally substituted C₁₋₆ alkyl and —OR^{CC18}; R^{CC10}; R^{CC12}; R^{CC14}; R^{CC16} and R^{CC18} can be independently selected from hydrogen and an optionally substituted C₁₋₆ alkyl; and R^{CC11}, R^{CC13}; R^{CC15} and R^{CC17} can be independently selected from an optionally substituted C₁₋₆ alkyl and an optionally substituted C₃₋₆ cycloalkyl. In some embodiments, when R^{CC3a}; R^{CC3b}; R^{CC4}; R^{CC5}; R^{CC7}; R^{CC8} and R^{CC9} are all hydrogen, then R^{CC6} is not azido. A non-limiting list of examples of compounds of Formula (CC) includes the compounds numbered 6000-6078 in FIGS. 6A-6I.

In some embodiments, a compound of Formula (I), or a pharmaceutically acceptable salt thereof, or a pharmaceutical composition that includes a compound of Formula (I), or a pharmaceutically acceptable salt thereof, can be used in combination with a compound of Formula (DD), or a pharmaceutically acceptable salt thereof, or a pharmaceutical composition that includes a compound of Formula (DD), or a pharmaceutically acceptable salt thereof (see, U.S. Publication No. 2010-0249068, filed Mar. 19, 2010, the contents of which are incorporated by reference in its entirety):

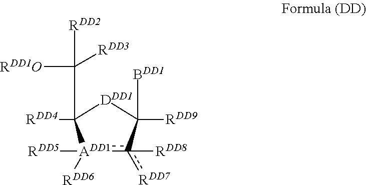

wherein each [symbol] can be independently a double or single bond; A^{DD1} can be selected from C (carbon), O (oxygen) and S (sulfur); B^{DD1} can be an optionally substituted heterocyclic base or a derivative thereof; D^{DD1} can be selected from C═CH₂, CH₂, O (oxygen), S (sulfur), CHF, and CF₂; R^{DD1} can be hydrogen, an optionally substituted alkyl, an optionally substituted cycloalkyl, an optionally substituted aralkyl, dialkylaminoalkylene, alkyl-C(═O)—, aryl-C(═O)—, alkoxyalkyl-C(═O)—, aryloxyalkyl-C(═O)—, alkylsulfonyl, arylsulfonyl, aralkylsulfonyl,

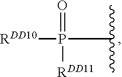

an —O-linked amino acid, diphosphate, triphosphate or derivatives thereof; R^{DD2} and R^{DD3} can be each independently selected from hydrogen, an optionally substituted C₁₋₆ alkyl, an optionally substituted C₂₋₆ alkenyl, an optionally substituted C₂₋₆ alkynyl and an optionally substituted C₁₋₆ haloalkyl, provided that at least one of R^{DD2} and R^{DD3} cannot be hydrogen; or R^{DD2} and R^{DD3} are taken together to form a group selected from among C₃₋₆ cycloalkyl, C₃₋₆ cycloalkenyl, C₃₋₆ aryl, and a C₃₋₆ heteroaryl; R^{DD4} and R^{DD9} can be independently selected from hydrogen, halogen, —NH₂, —NHR^{DDa1}, NR^{DDa1}R^{DDb1}, —OR^{DDa1}, —SR^{DDa1}, —CN, —NC, —N₃, —NO₂, —N(R^{DDc1})—NR^{DDa1}R^{DDb1}, —N(R^{DDc1})—OR^{DDa1}, —S—SR^{DDa1}, —(C═O)R^{DDa1}, —C(═O)OR^{DDa1}, —C(═O)R^{DDa1}R^{DDb1}, —O—(C═O)OR^{DDa1}, —O—C(═O)OR^{DDa1}, —O—C(═O)NR^{DDa1}R^{DDb1}, —N(R^{DDc1})—C(═O)NR^{DDa1}R^{DDb1}, —S(═O)R^{DDa1}, S(═O)₂R^{DDa1}, —O—S(═O)₂NR^{DDa1}R^{DDb1}, —N(R^{DDc1})—S(═O)₂NR^{DDa1}R^{DDb1}, an optionally substituted C₁₋₆ alkyl, an optionally substituted C₂₋₆ alkenyl, an optionally substituted C₂₋₆ alkynyl, an optionally substituted aralkyl and an —O-linked amino acid; R^{DD5}, R^{DD6} and R^{DD7} can be independently absent or selected from hydrogen, halogen, —NH₂, —NHR^{DDa1}, NR^{DDa1}R^{DDb1}, —OR^{DDa1}, —SR^{DDa1}, —CN, —NC, —N₃, —NO₂, —N(R^{DDc1})—NR^{DDa1}R^{DDb1}, —N(R^{DDc1})—OR^{DDa1}, —S—SR^{DDa1}, —C(═O)R^{DDa1}, —C(═O)OR^{DDa1}, —C(═O)NR^{DDa1}R^{DDb1}, —O—(C═O)R^{DDa1}, —O—C(═O)OR^{DDa1}, —O—C(═O)NR^{DDa1}R^{DDb1}, —N(R^{DDc1})—C(═O)NR^{DDa1}R^{DDb1}, —S(═O)R^{DDa1}, S(═O)₂R^{DDa1}, —O—S(═O)₂NR^{DDa1}R^{DDb1}, —N(R^{DDc1})—S(═O)₂NR^{DDa1}R^{DDb1}, an optionally substituted C₁₋₆ alkyl, an optionally substituted C₂₋₆ alkenyl, an optionally substituted C₂₋₆ alkynyl, an optionally substituted aralkyl and an —O-linked amino acid; or R^{DD6} and R^{DD7} taken together form —O—C(═O)—O—; R^{DD8} can be absent or selected from the group consisting of hydrogen, halogen, —NH₂, —NHR^{DDa1}, NR^{DDa1}R^{DDb1}, —OR^{DDa1}, —SR^{DDa1}, —CN, —NC, —N₃, —NO₂, —N(R^{DDc1})—NR^{DDa1}R^{DDb1}, —N(R^{DDc1})—OR^{DDa1}, —S—SR^{DDa1}, —C(═O)R^{DDa1}, —C(═O)OR^{DDa1}, —C(═O)NR^{DDa1}R^{DDb1}, —O—C(═O)OR^{DDa1}, —O—C(═O)NR^{DDa1}R^{DDb1}, —N(R^{DDc1})—C(═O)NR^{DDa1}R^{DDb1}, —S(O)R^{DDa1}, S(═O)₂R^{DDa1}, —O—S(═O)₂NR^{DDa1}R^{DDb1}, —N(R^{DDc1})—S(═O)₂NR^{DDa1}R^{DDb1}, an optionally substituted C₁₋₆ alkyl, an optionally substituted C₂₋₆ alkenyl, an optionally substituted C₂₋₆ alkynyl, an optionally substituted haloalkyl, an optionally substituted hydroxyalkyl and an —O-linked amino acid, or when the bond to R^{DD7} indicated by [symbol] is a double bond, then R^{DD7} is a C₂₋₆ alkylidene and R^{DD8} is absent; R^{DDa1}, R^{DDb1} and R^{DDc1} can be each independently selected from hydrogen, an optionally substituted alkyl, an optionally substituted alkenyl, an optionally substituted alkynyl, an optionally substituted aryl, an optionally substituted heteroaryl, an optionally substituted aralkyl and an optionally substituted heteroaryl(C₁₋₆ alkyl); R^{DD10} can be selected from O⁻, —OH, an optionally substituted aryloxy or aryl-O—,

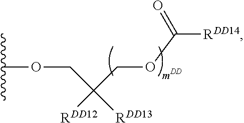

alkyl-C(═O)—O—CH₂—O—, alkyl-C(═O)—S—CH₂CH₂—O— and an —N-linked amino acid; R^{DD11} can be selected from O⁻, —OH, an optionally substituted aryloxy or aryl-O—,

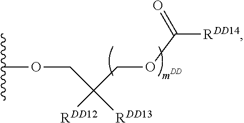

alkyl-C(═O)—O—CH₂—O—, alkyl-C(═O)—S—CH₂CH₂—O— and an —N-linked amino acid; each R^{DD12} and each R^{DD13} can be independently or an optionally substituted substituent selected from C₁₋₈ organylcarbonyl, C₁₋₈ alkoxycarbonyl and C₁₋₈ organylaminocarbonyl; each R^{DD14} can be hydrogen or an optionally substituted C₁₋₆-alkyl; each m^{DD} can be independently 1 or 2, and if both R^{DD10} and R^{DD11} are

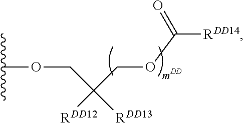

each R^{DD12}, each R^{DD13}, each R^{DD14} and each m^{DD} can be the same or different. In some embodiments, R^{DD8} can be halogen, —OR^{DDa1}, an optionally substituted C₁₋₆ alkyl, an optionally substituted C₂₋₆ alkenyl, an optionally substituted C₂₋₆ alkynyl and an optionally substituted C₁₋₆ haloalkyl.

Some embodiments described herein relate to a method of ameliorating or treating a viral infection that can include contacting a cell infected with the viral infection with a therapeutically effective amount of a compound selected from a compound of Formula (I) (including a compound of Formula (Iα)), compound 7072, compound 7073, compound 7074, compound 7075, compound 7076 and compound 7077, a monophosphate of any of the foregoing, and a diphosphate of any of the foregoing, or a pharmaceutically acceptable salt the foregoing, in combination with one or more agents selected from an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

Some embodiments described herein relate to a method of ameliorating or treating a viral infection that can include administering to a subject suffering from the viral infection a therapeutically effective amount of a compound selected from a compound of Formula (I) (including a compound of Formula (Iα)), compound 7072, compound 7073, compound 7074, compound 7075, compound 7076 and compound 7077, a monophosphate of any of the foregoing, and a diphosphate of any of the foregoing, or a pharmaceutically acceptable salt the foregoing, in combination with one or more agents selected from an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

Some embodiments described herein relate to a method of inhibiting viral replication of a virus that can include contacting a cell infected with the virus with an effective amount of a compound selected from a compound of Formula (I) (including a compound of Formula (Iα)), compound 7072, compound 7073, compound 7074, compound 7075, compound 7076 and compound 7077, a monophosphate of any of the foregoing, and a diphosphate of any of the foregoing, or a pharmaceutically acceptable salt the foregoing, in combination with one or more agents selected from an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

Some embodiments described herein relate to a method of ameliorating or treating a viral infection that can include contacting a cell infected with the viral infection with a therapeutically effective amount of a compound selected from a compound of Formula (I) (including a compound of Formula (Iα)), or a pharmaceutically acceptable salt the foregoing, in combination with one or more agents selected from an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

Some embodiments described herein relate to a method of ameliorating or treating a viral infection that can include administering to a subject suffering from the viral infection a therapeutically effective amount of a compound selected from a compound of Formula (I) (including a compound of Formula (Iα)), or a pharmaceutically acceptable salt the foregoing, in combination with one or more agents selected from an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

Some embodiments described herein relate to a method of inhibiting viral replication of a virus that can include contacting a cell infected with the virus with an effective amount of Formula (I) (including a compound of Formula (Iα)), or a pharmaceutically acceptable salt the foregoing, in combination with one or more agents selected from an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

In some embodiments, a compound of Formula (I) (including a compound of Formula (Iα)), or a pharmaceutically acceptable salt thereof, can be administered with one or more additional agent(s) together in a single pharmaceutical composition. In some embodiments, a compound of Formula (I) (including a compound of Formula (Iα)), or a pharmaceutically acceptable salt the thereof, can be administered with one or more additional agent(s) as two or more separate pharmaceutical compositions. For example, a compound of Formula (I) (including a compound of Formula (Iα)), or a pharmaceutically acceptable salt thereof, can be administered in one pharmaceutical composition, and at least one of the additional agents can be administered in a second pharmaceutical composition. If there are at least two additional agents, one or more of the additional agents can be in a first pharmaceutical composition that includes a compound of Formula (I) (including a compound of Formula (Iα)), or a pharmaceutically acceptable salt thereof, and at least one of the other additional agent(s) can be in a second pharmaceutical composition.

The dosing amount(s) and dosing schedule(s) when using a compound of Formula (I), or a pharmaceutically acceptable salt thereof, or a pharmaceutical composition that includes a compound of Formula (I), or a pharmaceutically acceptable salt thereof, and one or more additional agents are within the knowledge of those skilled in the art. For example, when performing a conventional standard of care therapy using art-recognized dosing amounts and dosing schedules, a compound of Formula (I), or a pharmaceutically acceptable salt thereof, or a pharmaceutical composition that includes a compound of Formula (I), or a pharmaceutically acceptable salt thereof, can be administered in addition to that therapy, or in place of one of the agents of a combination therapy, using effective amounts and dosing protocols as described herein.

The order of administration of a compound of Formula (I), or a pharmaceutically acceptable salt thereof, with one or more additional agent(s) can vary. In some embodiments, a compound of Formula (I) (including a compound of Formula (Iα)), or a pharmaceutically acceptable salt thereof, can be administered prior to all additional agents. In other embodiments, a compound of Formula (I) (including a compound of Formula (Iα)), or a pharmaceutically acceptable salt thereof, can be administered prior to at least one additional agent. In still other embodiments, a compound of Formula (I) (including a compound of Formula (Iα)), or a pharmaceutically acceptable salt thereof, can be administered concomitantly with one or more additional agent(s). In yet still other embodiments, a compound of Formula (I) (including a compound of Formula (Iα)), or a pharmaceutically acceptable salt thereof, can be administered subsequent to the administration of at least one additional agent. In some embodiments, a compound of Formula (I) (including a compound of Formula (Iα)), or a pharmaceutically acceptable salt thereof, can be administered subsequent to the administration of all additional agents.

In some embodiments, the combination of a compound of Formula (I), or a pharmaceutically acceptable salt thereof, in combination with one or more additional agent(s) in FIGS. 1-6 and 8-9 (including pharmaceutically acceptable salts and prodrugs thereof) can result in an additive effect. In some embodiments, the combination of a compound of Formula (I), or a pharmaceutically acceptable salt thereof, in combination with one or more additional agent(s) in FIGS. 1-6 and 8-9 (including pharmaceutically acceptable salts and prodrugs thereof) can result in a synergistic effect. In some embodiments, the combination of a compound of Formula (I), or a pharmaceutically acceptable salt thereof, in combination with one or more additional agent(s) in FIGS. 1-6 and 8-9 (including pharmaceutically acceptable salts and prodrugs thereof) can result in a strongly synergistic effect. In some embodiments, the combination of a compound of Formula (I), or a pharmaceutically acceptable salt thereof, in combination with one or more additional agent(s) in FIGS. 1-6 and 8-9 (including pharmaceutically acceptable salts and prodrugs thereof) is not antagonistic.

As used herein, the term “antagonistic” means that the activity of the combination of compounds is less compared to the sum of the activities of the compounds in combination when the activity of each compound is determined individually (i.e. as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compound in the combination when the activity of each compound is determined individually.

A potential advantage of utilizing a compound of Formula (I), or a pharmaceutically acceptable salt thereof, in combination with one or more additional agent(s) in FIGS. 1-6 and 8-9 (including pharmaceutically acceptable salts and prodrugs thereof) may be a reduction in the required amount(s) of one or more compounds of FIGS. 1-6 and 8-9 (including pharmaceutically acceptable salts and prodrugs thereof) that is effective in treating a disease condition disclosed herein (for example, HCV), as compared to the amount required to achieve same therapeutic result when one or more compounds of FIGS. 1-6 and 8-9 (including pharmaceutically acceptable salts and prodrugs thereof) are administered without a compound of Formula (I), or a pharmaceutically acceptable salt thereof. For example, the amount of a compound in FIGS. 1-6 and 8-9 (including a pharmaceutically acceptable salt and prodrug thereof), can be less compared to the amount of the compound in FIGS. 1-6 and 8-9 (including a pharmaceutically acceptable salt and prodrug thereof), needed to achieve the same viral load reduction when administered as a monotherapy. Another potential advantage of utilizing a compound of Formula (I), or a pharmaceutically acceptable salt thereof, in combination with one or more additional agent(s) in FIGS. 1-6 and 8-9 (including pharmaceutically acceptable salts and prodrugs thereof) is that the use of two or more compounds having different mechanism of actions can create a higher barrier to the development of resistant viral strains compared to the barrier when a compound is administered as monotherapy.

Additional advantages of utilizing a compound of Formula (I), or a pharmaceutically acceptable salt thereof, in combination with one or more additional agent(s) in FIGS. 1-6 and 8-9 (including pharmaceutically acceptable salts and prodrugs thereof) may include little to no cross resistance between a compound of Formula (I), or a pharmaceutically acceptable salt thereof, and one or more additional agent(s) in FIGS. 1-6 and 8-9 (including pharmaceutically acceptable salts and prodrugs thereof) thereof; different routes for elimination of a compound of Formula (I), or a pharmaceutically acceptable salt thereof, and one or more additional agent(s) in FIGS. 1-6 and 8-9 (including pharmaceutically acceptable salts and prodrugs thereof); little to no overlapping toxicities between a compound of Formula (I), or a pharmaceutically acceptable salt thereof, and one or more additional agent(s) in FIGS. 1-6 and 8-9 (including pharmaceutically acceptable salts and prodrugs thereof); little to no significant effects on cytochrome P450; and/or little to no pharmacokinetic interactions between a compound of Formula (I), or a pharmaceutically acceptable salt thereof, and one or more additional agent(s) in FIGS. 1-6 and 8-9 (including pharmaceutically acceptable salts and prodrugs thereof).

A non-limiting list of example combination of compounds of Formula (I), or a pharmaceutically acceptable salt thereof, or a pharmaceutical composition that includes a compound described herein, with one or more additional agent(s) are provided in Tables A, B, C and D. In addition, a compound selected from Compounds 7072-7077, or a pharmaceutically acceptable salt or a pharmaceutical composition thereof, can be used in combination with one or more additional agent(s), as provided in Tables A, B, C and D. Each numbered X and Y compound in Tables A, B, C and D has a corresponding name and/or structure provided in FIGS. 1 to 9. The numbered compounds in Tables A, B, C and D includes pharmaceutically acceptable salts of the compounds and pharmaceutical compositions containing the compounds or a pharmaceutically acceptable salt thereof. For example, 1001 includes the compound corresponding to 1001, pharmaceutically acceptable salts thereof, and pharmaceutical compositions that include compound 1001 and/or a pharmaceutically acceptable salt thereof. The combinations exemplified in Tables A, B, C and D are designated by the formula X:Y, which represents a combination of a compound X with a compound Y. For example, the combination designated as 1001:7001 in Table A represents a combination of compound 1001 with compound 7001, including pharmaceutically acceptable salts of compound 1001 and/or 7001, and pharmaceutical compositions including compound 1001 and 7001 (including pharmaceutical compositions that include pharmaceutically acceptable salts of compound 1001 and/or compound 7001). Thus, the combination designated as 1001:7001 in Table A represents the combination of Telaprevir (compound 1001, as shown in FIG. 1) and

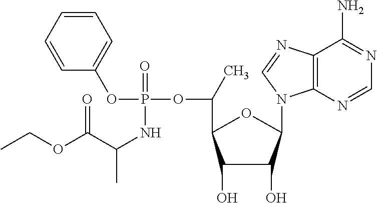

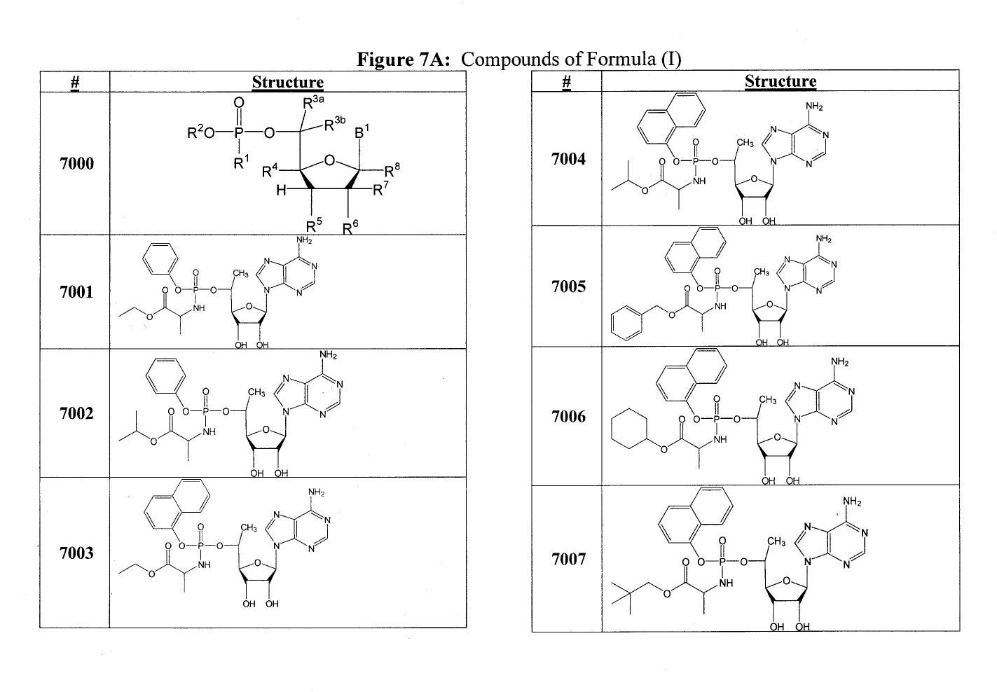

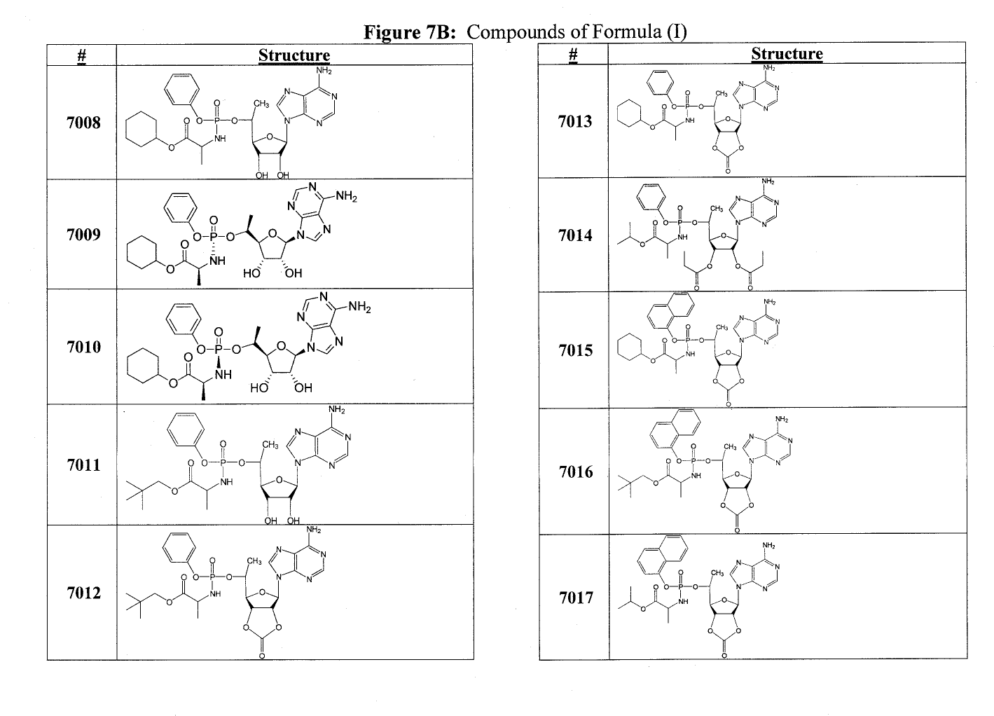

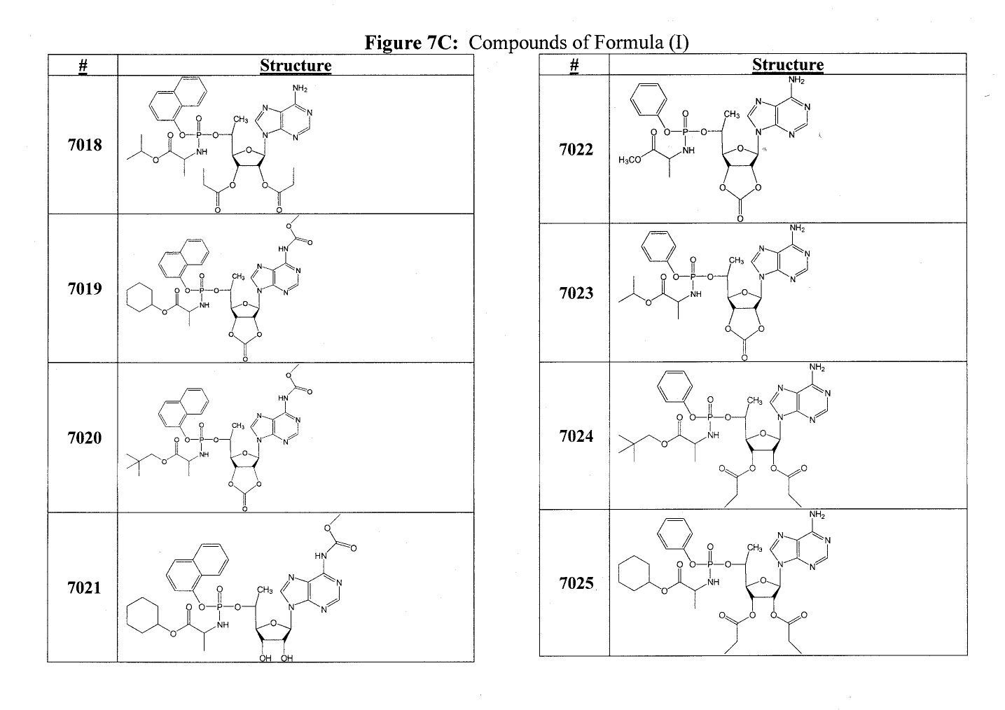

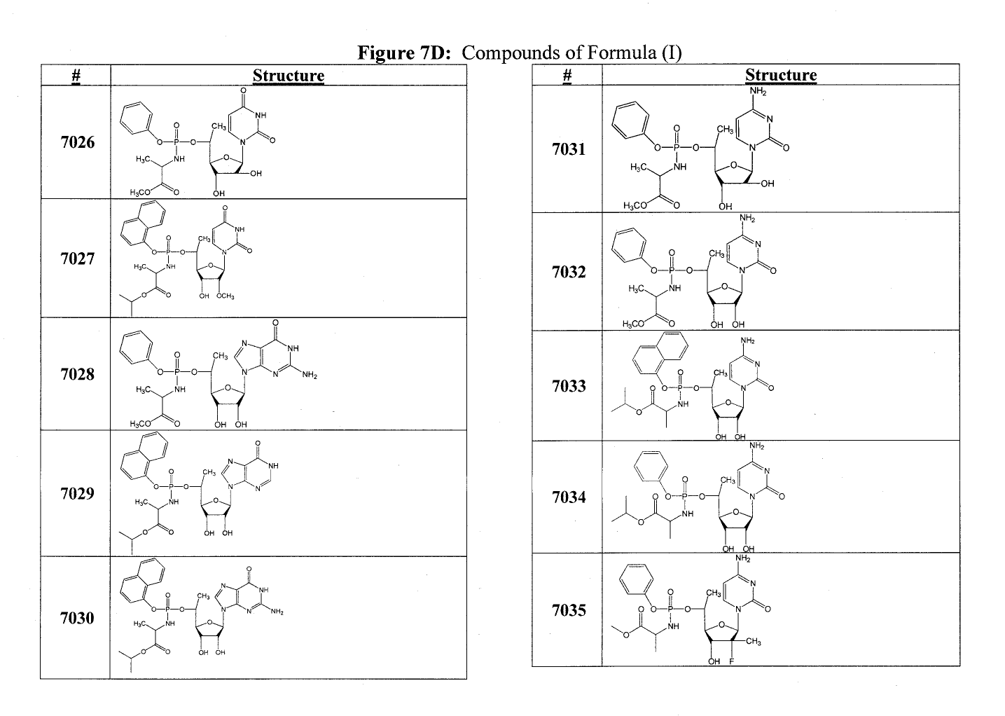

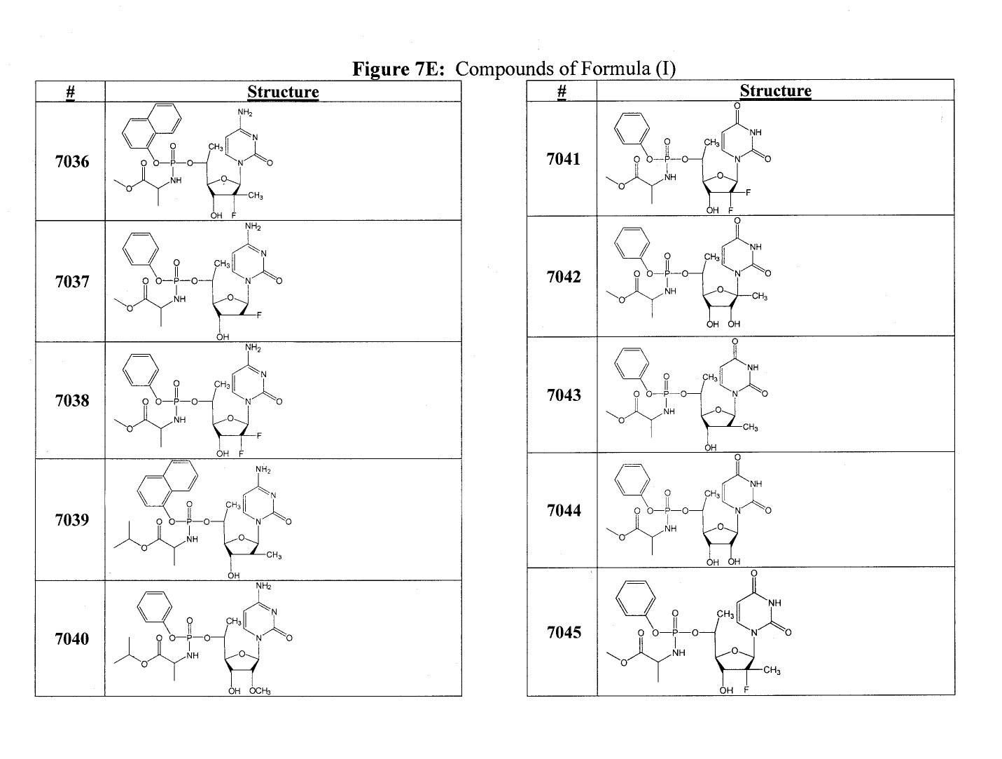

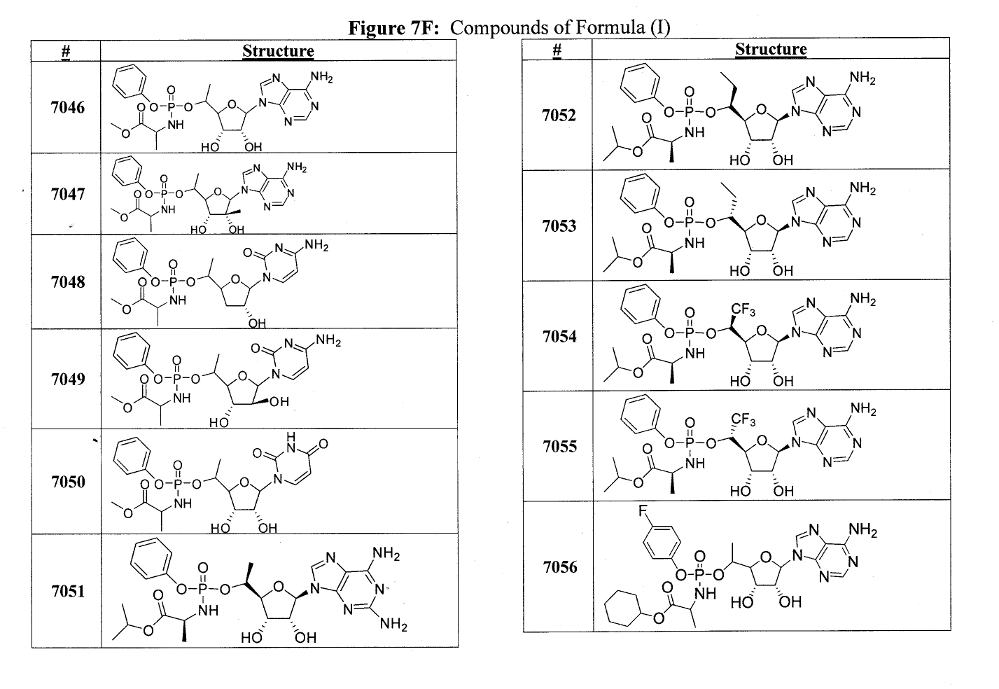

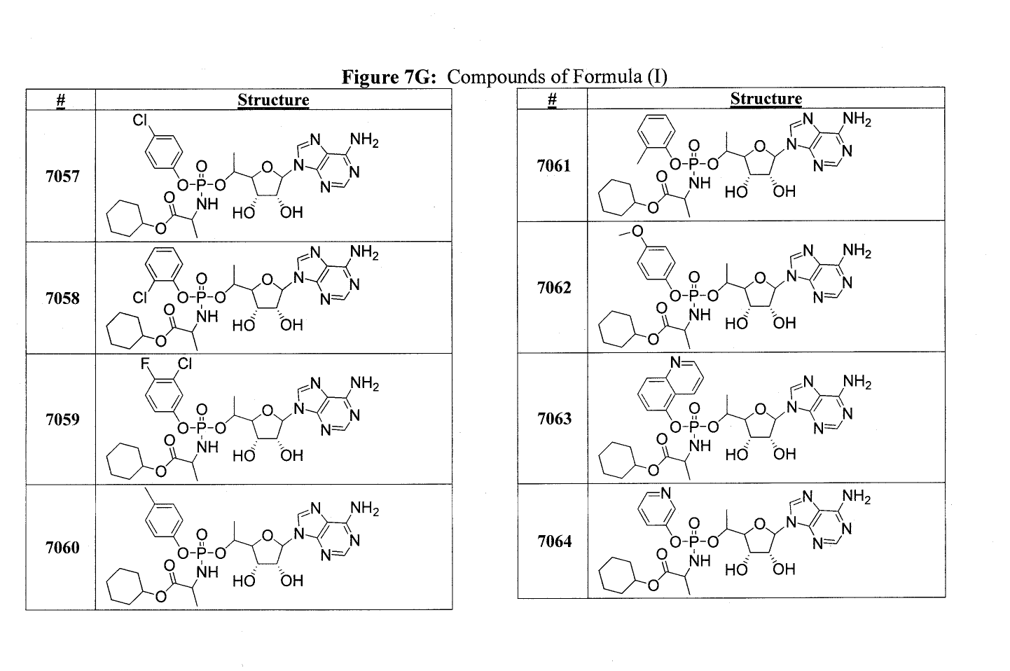

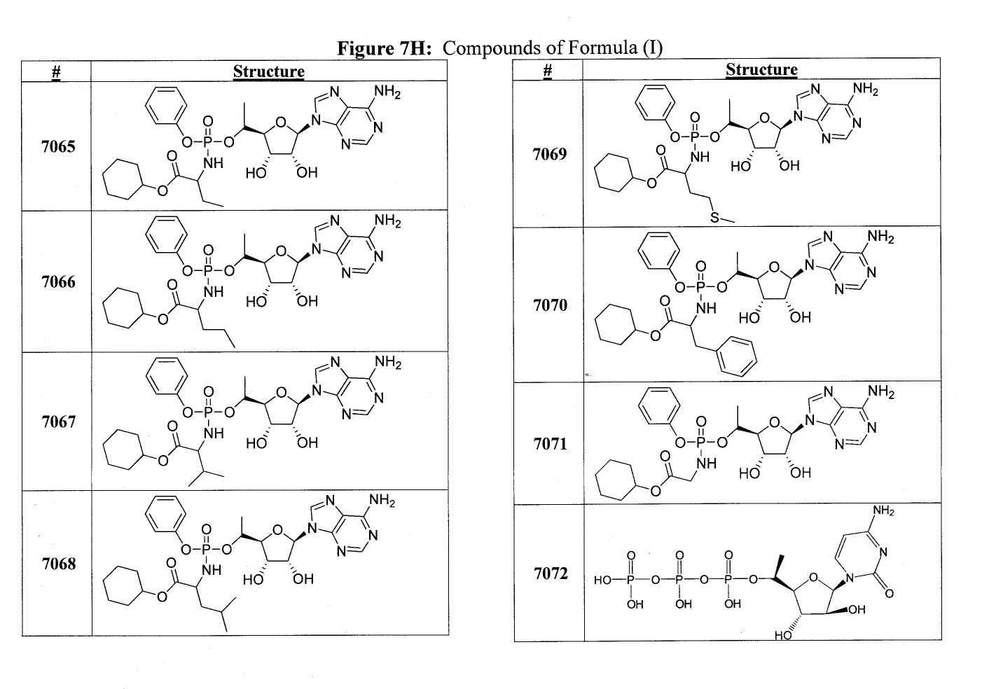

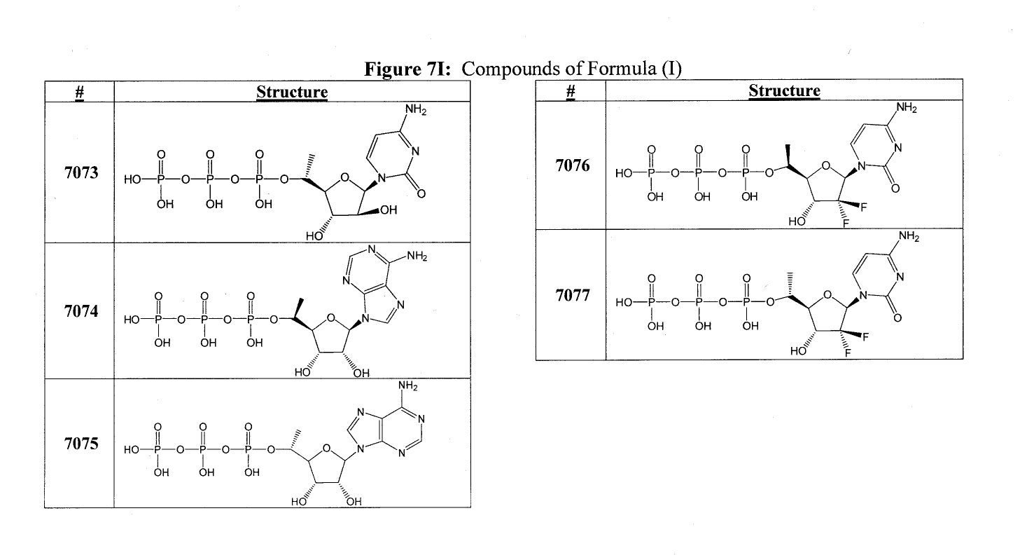

(compound 7001, as shown in FIG. 7A), including pharmaceutically acceptable salts of compound 1001 and/or 7001, and pharmaceutical compositions including compound 1001 and 7001 (including pharmaceutical compositions that include pharmaceutically acceptable salts of compound 1001 and/or compound 7001). Each of the combinations provided in Tables A, B, C and D can be used with one, two, three or more additional agents described herein. In some embodiments, embodiments described herein, the combination of agents can be used to treat, amerliorate and/or inhibit a virus and/or a viral infection, wherein the virus can be HCV and the viral infection can be an HCV viral infection.

TABLE A
Example combinations of a compound X with a compound Y.

| X:Y | X:Y | X:Y | X:Y | X:Y | X:Y | X:Y |
|---|---|---|---|---|---|---|
| 1001:7000 | 1001:7001 | 1001:7002 | 1001:7003 | 1001:7004 | 1001:7005 | 1001:7006 |
| 1002:7000 | 1002:7001 | 1002:7002 | 1002:7003 | 1002:7004 | 1002:7005 | 1002:7006 |
| 1003:7000 | 1003:7001 | 1003:7002 | 1003:7003 | 1003:7004 | 1003:7005 | 1003:7006 |
| 1004:7000 | 1004:7001 | 1004:7002 | 1004:7003 | 1004:7004 | 1004:7005 | 1004:7006 |
| 1005:7000 | 1005:7001 | 1005:7002 | 1005:7003 | 1005:7004 | 1005:7005 | 1005:7006 |
| 1006:7000 | 1006:7001 | 1006:7002 | 1006:7003 | 1006:7004 | 1006:7005 | 1006:7006 |
| 1007:7000 | 1007:7001 | 1007:7002 | 1007:7003 | 1007:7004 | 1007:7005 | 1007:7006 |
| 1008:7000 | 1008:7001 | 1008:7002 | 1008:7003 | 1008:7004 | 1008:7005 | 1008:7006 |
| 1009:7000 | 1009:7001 | 1009:7002 | 1009:7003 | 1009:7004 | 1009:7005 | 1009:7006 |
| 1010:7000 | 1010:7001 | 1010:7002 | 1010:7003 | 1010:7004 | 1010:7005 | 1010:7006 |
| 1011:7000 | 1011:7001 | 1011:7002 | 1011:7003 | 1011:7004 | 1011:7005 | 1011:7006 |
| 1012:7000 | 1012:7001 | 1012:7002 | 1012:7003 | 1012:7004 | 1012:7005 | 1012:7006 |
| 1013:7000 | 1013:7001 | 1013:7002 | 1013:7003 | 1013:7004 | 1013:7005 | 1013:7006 |
| 1014:7000 | 1014:7001 | 1014:7002 | 1014:7003 | 1014:7004 | 1014:7005 | 1014:7006 |
| 2001:7000 | 2001:7001 | 2001:7002 | 2001:7003 | 2001:7004 | 2001:7005 | 2001:7006 |
| 2002:7000 | 2002:7001 | 2002:7002 | 2002:7003 | 2002:7004 | 2002:7005 | 2002:7006 |
| 2003:7000 | 2003:7001 | 2003:7002 | 2003:7003 | 2003:7004 | 2003:7005 | 2003:7006 |
| 2004:7000 | 2004:7001 | 2004:7002 | 2004:7003 | 2004:7004 | 2004:7005 | 2004:7006 |
| 2005:7000 | 2005:7001 | 2005:7002 | 2005:7003 | 2005:7004 | 2005:7005 | 2005:7006 |
| 2006:7000 | 2006:7001 | 2006:7002 | 2006:7003 | 2006:7004 | 2006:7005 | 2006:7006 |
| 2007:7000 | 2007:7001 | 2007:7002 | 2007:7003 | 2007:7004 | 2007:7005 | 2007:7006 |
| 2008:7000 | 2008:7001 | 2008:7002 | 2008:7003 | 2008:7004 | 2008:7005 | 2008:7006 |
| 2009:7000 | 2009:7001 | 2009:7002 | 2009:7003 | 2009:7004 | 2009:7005 | 2009:7006 |
| 2010:7000 | 2010:7001 | 2010:7002 | 2010:7003 | 2010:7004 | 2010:7005 | 2010:7006 |
| 3001:7000 | 3001:7001 | 3001:7002 | 3001:7003 | 3001:7004 | 3001:7005 | 3001:7006 |
| 3002:7000 | 3002:7001 | 3002:7002 | 3002:7003 | 3002:7004 | 3002:7005 | 3002:7006 |
| 3003:7000 | 3003:7001 | 3003:7002 | 3003:7003 | 3003:7004 | 3003:7005 | 3003:7006 |
| 3004:7000 | 3004:7001 | 3004:7002 | 3004:7003 | 3004:7004 | 3004:7005 | 3004:7006 |
| 3005:7000 | 3005:7001 | 3005:7002 | 3005:7003 | 3005:7004 | 3005:7005 | 3005:7006 |
| 3006:7000 | 3006:7001 | 3006:7002 | 3006:7003 | 3006:7004 | 3006:7005 | 3006:7006 |
| 3007:7000 | 3007:7001 | 3007:7002 | 3007:7003 | 3007:7004 | 3007:7005 | 3007:7006 |
| 3008:7000 | 3008:7001 | 3008:7002 | 3008:7003 | 3008:7004 | 3008:7005 | 3008:7006 |
| 4001:7000 | 4001:7001 | 4001:7002 | 4001:7003 | 4001:7004 | 4001:7005 | 4001:7006 |
| 4002:7000 | 4002:7001 | 4002:7002 | 4002:7003 | 4002:7004 | 4002:7005 | 4002:7006 |
| 4003:7000 | 4003:7001 | 4003:7002 | 4003:7003 | 4003:7004 | 4003:7005 | 4003:7006 |
| 4004:7000 | 4004:7001 | 4004:7002 | 4004:7003 | 4004:7004 | 4004:7005 | 4004:7006 |
| 4005:7000 | 4005:7001 | 4005:7002 | 4005:7003 | 4005:7004 | 4005:7005 | 4005:7006 |
| 5001:7000 | 5001:7001 | 5001:7002 | 5001:7003 | 5001:7004 | 5001:7005 | 5001:7006 |
| 5002:7000 | 5002:7001 | 5002:7002 | 5002:7003 | 5002:7004 | 5002:7005 | 5002:7006 |
| X:Y | X:Y | X:Y | X:Y | X:Y | X:Y | X:Y |
| 1001:7007 | 1001:7008 | 1001:7009 | 1001:7010 | 1001:7011 | 1001:7012 | 1001:7013 |
| 1002:7007 | 1002:7008 | 1002:7009 | 1002:7010 | 1002:7011 | 1002:7012 | 1002:7013 |
| 1003:7007 | 1003:7008 | 1003:7009 | 1003:7010 | 1003:7011 | 1003:7012 | 1003:7013 |
| 1004:7007 | 1004:7008 | 1004:7009 | 1004:7010 | 1004:7011 | 1004:7012 | 1004:7013 |
| 1005:7007 | 1005:7008 | 1005:7009 | 1005:7010 | 1005:7011 | 1005:7012 | 1005:7013 |
| 1006:7007 | 1006:7008 | 1006:7009 | 1006:7010 | 1006:7011 | 1006:7012 | 1006:7013 |
| 1007:7007 | 1007:7008 | 1007:7009 | 1007:7010 | 1007:7011 | 1007:7012 | 1007:7013 |
| 1008:7007 | 1008:7008 | 1008:7009 | 1008:7010 | 1008:7011 | 1008:7012 | 1008:7013 |
| 1009:7007 | 1009:7008 | 1009:7009 | 1009:7010 | 1009:7011 | 1009:7012 | 1009:7013 |
| 1010:7007 | 1010:7008 | 1010:7009 | 1010:7010 | 1010:7011 | 1010:7012 | 1010:7013 |
| 1011:7007 | 1011:7008 | 1011:7009 | 1011:7010 | 1011:7011 | 1011:7012 | 1011:7013 |
| 1012:7007 | 1012:7008 | 1012:7009 | 1012:7010 | 1012:7011 | 1012:7012 | 1012:7013 |
| 1013:7007 | 1013:7008 | 1013:7009 | 1013:7010 | 1013:7011 | 1013:7012 | 1013:7013 |
| 1014:7007 | 1014:7008 | 1014:7009 | 1014:7010 | 1014:7011 | 1014:7012 | 1014:7013 |
| 2001:7007 | 2001:7008 | 2001:7009 | 2001:7010 | 2001:7011 | 2001:7012 | 2001:7013 |
| 2002:7007 | 2002:7008 | 2002:7009 | 2002:7010 | 2002:7011 | 2002:7012 | 2002:7013 |
| 2003:7007 | 2003:7008 | 2003:7009 | 2003:7010 | 2003:7011 | 2003:7012 | 2003:7013 |
| 2004:7007 | 2004:7008 | 2004:7009 | 2004:7010 | 2004:7011 | 2004:7012 | 2004:7013 |
| 2005:7007 | 2005:7008 | 2005:7009 | 2005:7010 | 2005:7011 | 2005:7012 | 2005:7013 |
| 2006:7007 | 2006:7008 | 2006:7009 | 2006:7010 | 2006:7011 | 2006:7012 | 2006:7013 |
| 2007:7007 | 2007:7008 | 2007:7009 | 2007:7010 | 2007:7011 | 2007:7012 | 2007:7013 |
| 2008:7007 | 2008:7008 | 2008:7009 | 2008:7010 | 2008:7011 | 2008:7012 | 2008:7013 |
| 2009:7007 | 2009:7008 | 2009:7009 | 2009:7010 | 2009:7011 | 2009:7012 | 2009:7013 |
| 2010:7007 | 2010:7008 | 2010:7009 | 2010:7010 | 2010:7011 | 2010:7012 | 2010:7013 |
| 3001:7007 | 3001:7008 | 3001:7009 | 3001:7010 | 3001:7011 | 3001:7012 | 3001:7013 |
| 3002:7007 | 3002:7008 | 3002:7009 | 3002:7010 | 3002:7011 | 3002:7012 | 3002:7013 |
| 3003:7007 | 3003:7008 | 3003:7009 | 3003:7010 | 3003:7011 | 3003:7012 | 3003:7013 |
| 3004:7007 | 3004:7008 | 3004:7009 | 3004:7010 | 3004:7011 | 3004:7012 | 3004:7013 |
| 3005:7007 | 3005:7008 | 3005:7009 | 3005:7010 | 3005:7011 | 3005:7012 | 3005:7013 |
| 3006:7007 | 3006:7008 | 3006:7009 | 3006:7010 | 3006:7011 | 3006:7012 | 3006:7013 |
| 3007:7007 | 3007:7008 | 3007:7009 | 3007:7010 | 3007:7011 | 3007:7012 | 3007:7013 |
| 3008:7007 | 3008:7008 | 3008:7009 | 3008:7010 | 3008:7011 | 3008:7012 | 3008:7013 |
| 4001:7007 | 4001:7008 | 4001:7009 | 4001:7010 | 4001:7011 | 4001:7012 | 4001:7013 |
| 4002:7007 | 4002:7008 | 4002:7009 | 4002:7010 | 4002:7011 | 4002:7012 | 4002:7013 |
| 4003:7007 | 4003:7008 | 4003:7009 | 4003:7010 | 4003:7011 | 4003:7012 | 4003:7013 |
| 4004:7007 | 4004:7008 | 4004:7009 | 4004:7010 | 4004:7011 | 4004:7012 | 4004:7013 |
| 4005:7007 | 4005:7008 | 4005:7009 | 4005:7010 | 4005:7011 | 4005:7012 | 4005:7013 |
| 5001:7007 | 5001:7008 | 5001:7009 | 5001:7010 | 5001:7011 | 5001:7012 | 5001:7013 |
| 5002:7007 | 5002:7008 | 5002:7009 | 5002:7010 | 5002:7011 | 5002:7012 | 5002:7013 |
| X:Y | X:Y | X:Y | X:Y | X:Y | X:Y | X:Y |
| 1001:7014 | 1001:7015 | 1001:7016 | 1001:7017 | 1001:7018 | 1001:7019 | 1001:7020 |
| 1002:7014 | 1002:7015 | 1002:7016 | 1002:7017 | 1002:7018 | 1002:7019 | 1002:7020 |
| 1003:7014 | 1003:7015 | 1003:7016 | 1003:7017 | 1003:7018 | 1003:7019 | 1003:7020 |
| 1004:7014 | 1004:7015 | 1004:7016 | 1004:7017 | 1004:7018 | 1004:7019 | 1004:7020 |
| 1005:7014 | 1005:7015 | 1005:7016 | 1005:7017 | 1005:7018 | 1005:7019 | 1005:7020 |
| 1006:7014 | 1006:7015 | 1006:7016 | 1006:7017 | 1006:7018 | 1006:7019 | 1006:7020 |
| 1007:7014 | 1007:7015 | 1007:7016 | 1007:7017 | 1007:7018 | 1007:7019 | 1007:7020 |
| 1008:7014 | 1008:7015 | 1008:7016 | 1008:7017 | 1008:7018 | 1008:7019 | 1008:7020 |
| 1009:7014 | 1009:7015 | 1009:7016 | 1009:7017 | 1009:7018 | 1009:7019 | 1009:7020 |
| 1010:7014 | 1010:7015 | 1010:7016 | 1010:7017 | 1010:7018 | 1010:7019 | 1010:7020 |
| 1011:7014 | 1011:7015 | 1011:7016 | 1011:7017 | 1011:7018 | 1011:7019 | 1011:7020 |
| 1012:7014 | 1012:7015 | 1012:7016 | 1012:7017 | 1012:7018 | 1012:7019 | 1012:7020 |
| 1013:7014 | 1013:7015 | 1013:7016 | 1013:7017 | 1013:7018 | 1013:7019 | 1013:7020 |
| 1014:7014 | 1014:7015 | 1014:7016 | 1014:7017 | 1014:7018 | 1014:7019 | 1014:7020 |
| 2001:7014 | 2001:7015 | 2001:7016 | 2001:7017 | 2001:7018 | 2001:7019 | 2001:7020 |
| 2002:7014 | 2002:7015 | 2002:7016 | 2002:7017 | 2002:7018 | 2002:7019 | 2002:7020 |
| 2003:7014 | 2003:7015 | 2003:7016 | 2003:7017 | 2003:7018 | 2003:7019 | 2003:7020 |
| 2004:7014 | 2004:7015 | 2004:7016 | 2004:7017 | 2004:7018 | 2004:7019 | 2004:7020 |
| 2005:7014 | 2005:7015 | 2005:7016 | 2005:7017 | 2005:7018 | 2005:7019 | 2005:7020 |
| 2006:7014 | 2006:7015 | 2006:7016 | 2006:7017 | 2006:7018 | 2006:7019 | 2006:7020 |
| 2007:7014 | 2007:7015 | 2007:7016 | 2007:7017 | 2007:7018 | 2007:7019 | 2007:7020 |
| 2008:7014 | 2008:7015 | 2008:7016 | 2008:7017 | 2008:7018 | 2008:7019 | 2008:7020 |
| 2009:7014 | 2009:7015 | 2009:7016 | 2009:7017 | 2009:7018 | 2009:7019 | 2009:7020 |
| 2010:7014 | 2010:7015 | 2010:7016 | 2010:7017 | 2010:7018 | 2010:7019 | 2010:7020 |
| 3001:7014 | 3001:7015 | 3001:7016 | 3001:7017 | 3001:7018 | 3001:7019 | 3001:7020 |
| 3002:7014 | 3002:7015 | 3002:7016 | 3002:7017 | 3002:7018 | 3002:7019 | 3002:7020 |
| 3003:7014 | 3003:7015 | 3003:7016 | 3003:7017 | 3003:7018 | 3003:7019 | 3003:7020 |
| 3004:7014 | 3004:7015 | 3004:7016 | 3004:7017 | 3004:7018 | 3004:7019 | 3004:7020 |
| 3005:7014 | 3005:7015 | 3005:7016 | 3005:7017 | 3005:7018 | 3005:7019 | 3005:7020 |
| 3006:7014 | 3006:7015 | 3006:7016 | 3006:7017 | 3006:7018 | 3006:7019 | 3006:7020 |
| 3007:7014 | 3007:7015 | 3007:7016 | 3007:7017 | 3007:7018 | 3007:7019 | 3007:7020 |
| 3008:7014 | 3008:7015 | 3008:7016 | 3008:7017 | 3008:7018 | 3008:7019 | 3008:7020 |
| 4001:7014 | 4001:7015 | 4001:7016 | 4001:7017 | 4001:7018 | 4001:7019 | 4001:7020 |
| 4002:7014 | 4002:7015 | 4002:7016 | 4002:7017 | 4002:7018 | 4002:7019 | 4002:7020 |
| 4003:7014 | 4003:7015 | 4003:7016 | 4003:7017 | 4003:7018 | 4003:7019 | 4003:7020 |
| 4004:7014 | 4004:7015 | 4004:7016 | 4004:7017 | 4004:7018 | 4004:7019 | 4004:7020 |
| 4005:7014 | 4005:7015 | 4005:7016 | 4005:7017 | 4005:7018 | 4005:7019 | 4005:7020 |
| 5001:7014 | 5001:7015 | 5001:7016 | 5001:7017 | 5001:7018 | 5001:7019 | 5001:7020 |
| 5002:7014 | 5002:7015 | 5002:7016 | 5002:7017 | 5002:7018 | 5002:7019 | 5002:7020 |
| X:Y | X:Y | X:Y | X:Y | X:Y | X:Y | X:Y |
| 1001:7021 | 1001:7022 | 1001:7023 | 1001:7024 | 1001:7025 | 1001:7026 | 1001:7027 |
| 1002:7021 | 1002:7022 | 1002:7023 | 1002:7024 | 1002:7025 | 1002:7026 | 1002:7027 |
| 1003:7021 | 1003:7022 | 1003:7023 | 1003:7024 | 1003:7025 | 1003:7026 | 1003:7027 |
| 1004:7021 | 1004:7022 | 1004:7023 | 1004:7024 | 1004:7025 | 1004:7026 | 1004:7027 |
| 1005:7021 | 1005:7022 | 1005:7023 | 1005:7024 | 1005:7025 | 1005:7026 | 1005:7027 |
| 1006:7021 | 1006:7022 | 1006:7023 | 1006:7024 | 1006:7025 | 1006:7026 | 1006:7027 |
| 1007:7021 | 1007:7022 | 1007:7023 | 1007:7024 | 1007:7025 | 1007:7026 | 1007:7027 |
| 1008:7021 | 1008:7022 | 1008:7023 | 1008:7024 | 1008:7025 | 1008:7026 | 1008:7027 |
| 1009:7021 | 1009:7022 | 1009:7023 | 1009:7024 | 1009:7025 | 1009:7026 | 1009:7027 |
| 1010:7021 | 1010:7022 | 1010:7023 | 1010:7024 | 1010:7025 | 1010:7026 | 1010:7027 |
| 1011:7021 | 1011:7022 | 1011:7023 | 1011:7024 | 1011:7025 | 1011:7026 | 1011:7027 |
| 1012:7021 | 1012:7022 | 1012:7023 | 1012:7024 | 1012:7025 | 1012:7026 | 1012:7027 |
| 1013:7021 | 1013:7022 | 1013:7023 | 1013:7024 | 1013:7025 | 1013:7026 | 1013:7027 |
| 1014:7021 | 1014:7022 | 1014:7023 | 1014:7024 | 1014:7025 | 1014:7026 | 1014:7027 |
| 2001:7021 | 2001:7022 | 2001:7023 | 2001:7024 | 2001:7025 | 2001:7026 | 2001:7027 |
| 2002:7021 | 2002:7022 | 2002:7023 | 2002:7024 | 2002:7025 | 2002:7026 | 2002:7027 |
| 2003:7021 | 2003:7022 | 2003:7023 | 2003:7024 | 2003:7025 | 2003:7026 | 2003:7027 |
| 2004:7021 | 2004:7022 | 2004:7023 | 2004:7024 | 2004:7025 | 2004:7026 | 2004:7027 |
| 2005:7021 | 2005:7022 | 2005:7023 | 2005:7024 | 2005:7025 | 2005:7026 | 2005:7027 |
| 2006:7021 | 2006:7022 | 2006:7023 | 2006:7024 | 2006:7025 | 2006:7026 | 2006:7027 |
| 2007:7021 | 2007:7022 | 2007:7023 | 2007:7024 | 2007:7025 | 2007:7026 | 2007:7027 |
| 2008:7021 | 2008:7022 | 2008:7023 | 2008:7024 | 2008:7025 | 2008:7026 | 2008:7027 |
| 2009:7021 | 2009:7022 | 2009:7023 | 2009:7024 | 2009:7025 | 2009:7026 | 2009:7027 |
| 2010:7021 | 2010:7022 | 2010:7023 | 2010:7024 | 2010:7025 | 2010:7026 | 2010:7027 |
| 3001:7021 | 3001:7022 | 3001:7023 | 3001:7024 | 3001:7025 | 3001:7026 | 3001:7027 |
| 3002:7021 | 3002:7022 | 3002:7023 | 3002:7024 | 3002:7025 | 3002:7026 | 3002:7027 |
| 3003:7021 | 3003:7022 | 3003:7023 | 3003:7024 | 3003:7025 | 3003:7026 | 3003:7027 |
| 3004:7021 | 3004:7022 | 3004:7023 | 3004:7024 | 3004:7025 | 3004:7026 | 3004:7027 |
| 3005:7021 | 3005:7022 | 3005:7023 | 3005:7024 | 3005:7025 | 3005:7026 | 3005:7027 |
| 3006:7021 | 3006:7022 | 3006:7023 | 3006:7024 | 3006:7025 | 3006:7026 | 3006:7027 |
| 3007:7021 | 3007:7022 | 3007:7023 | 3007:7024 | 3007:7025 | 3007:7026 | 3007:7027 |
| 3008:7021 | 3008:7022 | 3008:7023 | 3008:7024 | 3008:7025 | 3008:7026 | 3008:7027 |
| 4001:7021 | 4001:7022 | 4001:7023 | 4001:7024 | 4001:7025 | 4001:7026 | 4001:7027 |
| 4002:7021 | 4002:7022 | 4002:7023 | 4002:7024 | 4002:7025 | 4002:7026 | 4002:7027 |
| 4003:7021 | 4003:7022 | 4003:7023 | 4003:7024 | 4003:7025 | 4003:7026 | 4003:7027 |
| 4004:7021 | 4004:7022 | 4004:7023 | 4004:7024 | 4004:7025 | 4004:7026 | 4004:7027 |
| 4005:7021 | 4005:7022 | 4005:7023 | 4005:7024 | 4005:7025 | 4005:7026 | 4005:7027 |
| 5001:7021 | 5001:7022 | 5001:7023 | 5001:7024 | 5001:7025 | 5001:7026 | 5001:7027 |
| 5002:7021 | 5002:7022 | 5002:7023 | 5002:7024 | 5002:7025 | 5002:7026 | 5002:7027 |
| X:Y | X:Y | X:Y | X:Y | X:Y | X:Y | X:Y |
| 1001:7028 | 1001:7029 | 1001:7030 | 1001:7031 | 1001:7032 | 1001:7033 | 1001:7034 |
| 1002:7028 | 1002:7029 | 1002:7030 | 1002:7031 | 1002:7032 | 1002:7033 | 1002:7034 |
| 1003:7028 | 1003:7029 | 1003:7030 | 1003:7031 | 1003:7032 | 1003:7033 | 1003:7034 |
| 1004:7028 | 1004:7029 | 1004:7030 | 1004:7031 | 1004:7032 | 1004:7033 | 1004:7034 |
| 1005:7028 | 1005:7029 | 1005:7030 | 1005:7031 | 1005:7032 | 1005:7033 | 1005:7034 |
| 1006:7028 | 1006:7029 | 1006:7030 | 1006:7031 | 1006:7032 | 1006:7033 | 1006:7034 |
| 1007:7028 | 1007:7029 | 1007:7030 | 1007:7031 | 1007:7032 | 1007:7033 | 1007:7034 |
| 1008:7028 | 1008:7029 | 1008:7030 | 1008:7031 | 1008:7032 | 1008:7033 | 1008:7034 |
| 1009:7028 | 1009:7029 | 1009:7030 | 1009:7031 | 1009:7032 | 1009:7033 | 1009:7034 |
| 1010:7028 | 1010:7029 | 1010:7030 | 1010:7031 | 1010:7032 | 1010:7033 | 1010:7034 |
| 1011:7028 | 1011:7029 | 1011:7030 | 1011:7031 | 1011:7032 | 1011:7033 | 1011:7034 |
| 1012:7028 | 1012:7029 | 1012:7030 | 1012:7031 | 1012:7032 | 1012:7033 | 1012:7034 |
| 1013:7028 | 1013:7029 | 1013:7030 | 1013:7031 | 1013:7032 | 1013:7033 | 1013:7034 |
| 1014:7028 | 1014:7029 | 1014:7030 | 1014:7031 | 1014:7032 | 1014:7033 | 1014:7034 |
| 2001:7028 | 2001:7029 | 2001:7030 | 2001:7031 | 2001:7032 | 2001:7033 | 2001:7034 |
| 2002:7028 | 2002:7029 | 2002:7030 | 2002:7031 | 2002:7032 | 2002:7033 | 2002:7034 |
| 2003:7028 | 2003:7029 | 2003:7030 | 2003:7031 | 2003:7032 | 2003:7033 | 2003:7034 |
| 2004:7028 | 2004:7029 | 2004:7030 | 2004:7031 | 2004:7032 | 2004:7033 | 2004:7034 |
| 2005:7028 | 2005:7029 | 2005:7030 | 2005:7031 | 2005:7032 | 2005:7033 | 2005:7034 |
| 2006:7028 | 2006:7029 | 2006:7030 | 2006:7031 | 2006:7032 | 2006:7033 | 2006:7034 |
| 2007:7028 | 2007:7029 | 2007:7030 | 2007:7031 | 2007:7032 | 2007:7033 | 2007:7034 |
| 2008:7028 | 2008:7029 | 2008:7030 | 2008:7031 | 2008:7032 | 2008:7033 | 2008:7034 |
| 2009:7028 | 2009:7029 | 2009:7030 | 2009:7031 | 2009:7032 | 2009:7033 | 2009:7034 |
| 2010:7028 | 2010:7029 | 2010:7030 | 2010:7031 | 2010:7032 | 2010:7033 | 2010:7034 |
| 3001:7028 | 3001:7029 | 3001:7030 | 3001:7031 | 3001:7032 | 3001:7033 | 3001:7034 |
| 3002:7028 | 3002:7029 | 3002:7030 | 3002:7031 | 3002:7032 | 3002:7033 | 3002:7034 |
| 3003:7028 | 3003:7029 | 3003:7030 | 3003:7031 | 3003:7032 | 3003:7033 | 3003:7034 |
| 3004:7028 | 3004:7029 | 3004:7030 | 3004:7031 | 3004:7032 | 3004:7033 | 3004:7034 |
| 3005:7028 | 3005:7029 | 3005:7030 | 3005:7031 | 3005:7032 | 3005:7033 | 3005:7034 |
| 3006:7028 | 3006:7029 | 3006:7030 | 3006:7031 | 3006:7032 | 3006:7033 | 3006:7034 |
| 3007:7028 | 3007:7029 | 3007:7030 | 3007:7031 | 3007:7032 | 3007:7033 | 3007:7034 |
| 3008:7028 | 3008:7029 | 3008:7030 | 3008:7031 | 3008:7032 | 3008:7033 | 3008:7034 |
| 4001:7028 | 4001:7029 | 4001:7030 | 4001:7031 | 4001:7032 | 4001:7033 | 4001:7034 |
| 4002:7028 | 4002:7029 | 4002:7030 | 4002:7031 | 4002:7032 | 4002:7033 | 4002:7034 |
| 4003:7028 | 4003:7029 | 4003:7030 | 4003:7031 | 4003:7032 | 4003:7033 | 4003:7034 |
| 4004:7028 | 4004:7029 | 4004:7030 | 4004:7031 | 4004:7032 | 4004:7033 | 4004:7034 |
| 4005:7028 | 4005:7029 | 4005:7030 | 4005:7031 | 4005:7032 | 4005:7033 | 4005:7034 |
| 5001:7028 | 5001:7029 | 5001:7030 | 5001:7031 | 5001:7032 | 5001:7033 | 5001:7034 |
| 5002:7028 | 5002:7029 | 5002:7030 | 5002:7031 | 5002:7032 | 5002:7033 | 5002:7034 |
| X:Y | X:Y | X:Y | X:Y | X:Y | X:Y | X:Y |
| 1001:7035 | 1001:7036 | 1001:7037 | 1001:7038 | 1001:7039 | 1001:7040 | 1001:7041 |
| 1002:7035 | 1002:7036 | 1002:7037 | 1002:7038 | 1002:7039 | 1002:7040 | 1002:7041 |
| 1003:7035 | 1003:7036 | 1003:7037 | 1003:7038 | 1003:7039 | 1003:7040 | 1003:7041 |
| 1004:7035 | 1004:7036 | 1004:7037 | 1004:7038 | 1004:7039 | 1004:7040 | 1004:7041 |
| 1005:7035 | 1005:7036 | 1005:7037 | 1005:7038 | 1005:7039 | 1005:7040 | 1005:7041 |
| 1006:7035 | 1006:7036 | 1006:7037 | 1006:7038 | 1006:7039 | 1006:7040 | 1006:7041 |
| 1007:7035 | 1007:7036 | 1007:7037 | 1007:7038 | 1007:7039 | 1007:7040 | 1007:7041 |
| 1008:7035 | 1008:7036 | 1008:7037 | 1008:7038 | 1008:7039 | 1008:7040 | 1008:7041 |
| 1009:7035 | 1009:7036 | 1009:7037 | 1009:7038 | 1009:7039 | 1009:7040 | 1009:7041 |
| 1010:7035 | 1010:7036 | 1010:7037 | 1010:7038 | 1010:7039 | 1010:7040 | 1010:7041 |
| 1011:7035 | 1011:7036 | 1011:7037 | 1011:7038 | 1011:7039 | 1011:7040 | 1011:7041 |
| 1012:7035 | 1012:7036 | 1012:7037 | 1012:7038 | 1012:7039 | 1012:7040 | 1012:7041 |
| 1013:7035 | 1013:7036 | 1013:7037 | 1013:7038 | 1013:7039 | 1013:7040 | 1013:7041 |
| 1014:7035 | 1014:7036 | 1014:7037 | 1014:7038 | 1014:7039 | 1014:7040 | 1014:7041 |
| 2001:7035 | 2001:7036 | 2001:7037 | 2001:7038 | 2001:7039 | 2001:7040 | 2001:7041 |
| 2002:7035 | 2002:7036 | 2002:7037 | 2002:7038 | 2002:7039 | 2002:7040 | 2002:7041 |
| 2003:7035 | 2003:7036 | 2003:7037 | 2003:7038 | 2003:7039 | 2003:7040 | 2003:7041 |
| 2004:7035 | 2004:7036 | 2004:7037 | 2004:7038 | 2004:7039 | 2004:7040 | 2004:7041 |
| 2005:7035 | 2005:7036 | 2005:7037 | 2005:7038 | 2005:7039 | 2005:7040 | 2005:7041 |
| 2006:7035 | 2006:7036 | 2006:7037 | 2006:7038 | 2006:7039 | 2006:7040 | 2006:7041 |
| 2007:7035 | 2007:7036 | 2007:7037 | 2007:7038 | 2007:7039 | 2007:7040 | 2007:7041 |
| 2008:7035 | 2008:7036 | 2008:7037 | 2008:7038 | 2008:7039 | 2008:7040 | 2008:7041 |
| 2009:7035 | 2009:7036 | 2009:7037 | 2009:7038 | 2009:7039 | 2009:7040 | 2009:7041 |
| 2010:7035 | 2010:7036 | 2010:7037 | 2010:7038 | 2010:7039 | 2010:7040 | 2010:7041 |
| 3001:7035 | 3001:7036 | 3001:7037 | 3001:7038 | 3001:7039 | 3001:7040 | 3001:7041 |
| 3002:7035 | 3002:7036 | 3002:7037 | 3002:7038 | 3002:7039 | 3002:7040 | 3002:7041 |
| 3003:7035 | 3003:7036 | 3003:7037 | 3003:7038 | 3003:7039 | 3003:7040 | 3003:7041 |
| 3004:7035 | 3004:7036 | 3004:7037 | 3004:7038 | 3004:7039 | 3004:7040 | 3004:7041 |
| 3005:7035 | 3005:7036 | 3005:7037 | 3005:7038 | 3005:7039 | 3005:7040 | 3005:7041 |
| 3006:7035 | 3006:7036 | 3006:7037 | 3006:7038 | 3006:7039 | 3006:7040 | 3006:7041 |
| 3007:7035 | 3007:7036 | 3007:7037 | 3007:7038 | 3007:7039 | 3007:7040 | 3007:7041 |
| 3008:7035 | 3008:7036 | 3008:7037 | 3008:7038 | 3008:7039 | 3008:7040 | 3008:7041 |
| 4001:7035 | 4001:7036 | 4001:7037 | 4001:7038 | 4001:7039 | 4001:7040 | 4001:7041 |
| 4002:7035 | 4002:7036 | 4002:7037 | 4002:7038 | 4002:7039 | 4002:7040 | 4002:7041 |
| 4003:7035 | 4003:7036 | 4003:7037 | 4003:7038 | 4003:7039 | 4003:7040 | 4003:7041 |
| 4004:7035 | 4004:7036 | 4004:7037 | 4004:7038 | 4004:7039 | 4004:7040 | 4004:7041 |
| 4005:7035 | 4005:7036 | 4005:7037 | 4005:7038 | 4005:7039 | 4005:7040 | 4005:7041 |
| 5001:7035 | 5001:7036 | 5001:7037 | 5001:7038 | 5001:7039 | 5001:7040 | 5001:7041 |
| 5002:7035 | 5002:7036 | 5002:7037 | 5002:7038 | 5002:7039 | 5002:7040 | 5002:7041 |
| X:Y | X:Y | X:Y | X:Y | X:Y | X:Y | X:Y |
| 1001:7042 | 1001:7043 | 1001:7044 | 1001:7045 | 1001:7046 | 1001:7047 | 1001:7048 |
| 1002:7042 | 1002:7043 | 1002:7044 | 1002:7045 | 1002:7046 | 1002:7047 | 1002:7048 |
| 1003:7042 | 1003:7043 | 1003:7044 | 1003:7045 | 1003:7046 | 1003:7047 | 1003:7048 |
| 1004:7042 | 1004:7043 | 1004:7044 | 1004:7045 | 1004:7046 | 1004:7047 | 1004:7048 |
| 1005:7042 | 1005:7043 | 1005:7044 | 1005:7045 | 1005:7046 | 1005:7047 | 1005:7048 |
| 1006:7042 | 1006:7043 | 1006:7044 | 1006:7045 | 1006:7046 | 1006:7047 | 1006:7048 |
| 1007:7042 | 1007:7043 | 1007:7044 | 1007:7045 | 1007:7046 | 1007:7047 | 1007:7048 |
| 1008:7042 | 1008:7043 | 1008:7044 | 1008:7045 | 1008:7046 | 1008:7047 | 1008:7048 |
| 1009:7042 | 1009:7043 | 1009:7044 | 1009:7045 | 1009:7046 | 1009:7047 | 1009:7048 |
| 1010:7042 | 1010:7043 | 1010:7044 | 1010:7045 | 1010:7046 | 1010:7047 | 1010:7048 |
| 1011:7042 | 1011:7043 | 1011:7044 | 1011:7045 | 1011:7046 | 1011:7047 | 1011:7048 |
| 1012:7042 | 1012:7043 | 1012:7044 | 1012:7045 | 1012:7046 | 1012:7047 | 1012:7048 |
| 1013:7042 | 1013:7043 | 1013:7044 | 1013:7045 | 1013:7046 | 1013:7047 | 1013:7048 |
| 1014:7042 | 1014:7043 | 1014:7044 | 1014:7045 | 1014:7046 | 1014:7047 | 1014:7048 |
| 2001:7042 | 2001:7043 | 2001:7044 | 2001:7045 | 2001:7046 | 2001:7047 | 2001:7048 |
| 2002:7042 | 2002:7043 | 2002:7044 | 2002:7045 | 2002:7046 | 2002:7047 | 2002:7048 |
| 2003:7042 | 2003:7043 | 2003:7044 | 2003:7045 | 2003:7046 | 2003:7047 | 2003:7048 |
| 2004:7042 | 2004:7043 | 2004:7044 | 2004:7045 | 2004:7046 | 2004:7047 | 2004:7048 |
| 2005:7042 | 2005:7043 | 2005:7044 | 2005:7045 | 2005:7046 | 2005:7047 | 2005:7048 |
| 2006:7042 | 2006:7043 | 2006:7044 | 2006:7045 | 2006:7046 | 2006:7047 | 2006:7048 |
| 2007:7042 | 2007:7043 | 2007:7044 | 2007:7045 | 2007:7046 | 2007:7047 | 2007:7048 |
| 2008:7042 | 2008:7043 | 2008:7044 | 2008:7045 | 2008:7046 | 2008:7047 | 2008:7048 |
| 2009:7042 | 2009:7043 | 2009:7044 | 2009:7045 | 2009:7046 | 2009:7047 | 2009:7048 |
| 2010:7042 | 2010:7043 | 2010:7044 | 2010:7045 | 2010:7046 | 2010:7047 | 2010:7048 |
| 3001:7042 | 3001:7043 | 3001:7044 | 3001:7045 | 3001:7046 | 3001:7047 | 3001:7048 |
| 3002:7042 | 3002:7043 | 3002:7044 | 3002:7045 | 3002:7046 | 3002:7047 | 3002:7048 |
| 3003:7042 | 3003:7043 | 3003:7044 | 3003:7045 | 3003:7046 | 3003:7047 | 3003:7048 |
| 3004:7042 | 3004:7043 | 3004:7044 | 3004:7045 | 3004:7046 | 3004:7047 | 3004:7048 |
| 3005:7042 | 3005:7043 | 3005:7044 | 3005:7045 | 3005:7046 | 3005:7047 | 3005:7048 |
| 3006:7042 | 3006:7043 | 3006:7044 | 3006:7045 | 3006:7046 | 3006:7047 | 3006:7048 |
| 3007:7042 | 3007:7043 | 3007:7044 | 3007:7045 | 3007:7046 | 3007:7047 | 3007:7048 |
| 3008:7042 | 3008:7043 | 3008:7044 | 3008:7045 | 3008:7046 | 3008:7047 | 3008:7048 |
| 4001:7042 | 4001:7043 | 4001:7044 | 4001:7045 | 4001:7046 | 4001:7047 | 4001:7048 |
| 4002:7042 | 4002:7043 | 4002:7044 | 4002:7045 | 4002:7046 | 4002:7047 | 4002:7048 |
| 4003:7042 | 4003:7043 | 4003:7044 | 4003:7045 | 4003:7046 | 4003:7047 | 4003:7048 |
| 4004:7042 | 4004:7043 | 4004:7044 | 4004:7045 | 4004:7046 | 4004:7047 | 4004:7048 |
| 4005:7042 | 4005:7043 | 4005:7044 | 4005:7045 | 4005:7046 | 4005:7047 | 4005:7048 |
| 5001:7042 | 5001:7043 | 5001:7044 | 5001:7045 | 5001:7046 | 5001:7047 | 5001:7048 |
| 5002:7042 | 5002:7043 | 5002:7044 | 5002:7045 | 5002:7046 | 5002:7047 | 5002:7048 |
| X:Y | X:Y | X:Y | X:Y | X:Y | X:Y | X:Y |
| 1001:7049 | 1001:7050 | 1001:7051 | 1001:7052 | 1001:7053 | 1001:7054 | 1001:7055 |
| 1002:7049 | 1002:7050 | 1002:7051 | 1002:7052 | 1002:7053 | 1002:7054 | 1002:7055 |
| 1003:7049 | 1003:7050 | 1003:7051 | 1003:7052 | 1003:7053 | 1003:7054 | 1003:7055 |
| 1004:7049 | 1004:7050 | 1004:7051 | 1004:7052 | 1004:7053 | 1004:7054 | 1004:7055 |
| 1005:7049 | 1005:7050 | 1005:7051 | 1005:7052 | 1005:7053 | 1005:7054 | 1005:7055 |
| 1006:7049 | 1006:7050 | 1006:7051 | 1006:7052 | 1006:7053 | 1006:7054 | 1006:7055 |
| 1007:7049 | 1007:7050 | 1007:7051 | 1007:7052 | 1007:7053 | 1007:7054 | 1007:7055 |
| 1008:7049 | 1008:7050 | 1008:7051 | 1008:7052 | 1008:7053 | 1008:7054 | 1008:7055 |
| 1009:7049 | 1009:7050 | 1009:7051 | 1009:7052 | 1009:7053 | 1009:7054 | 1009:7055 |
| 1010:7049 | 1010:7050 | 1010:7051 | 1010:7052 | 1010:7053 | 1010:7054 | 1010:7055 |
| 1011:7049 | 1011:7050 | 1011:7051 | 1011:7052 | 1011:7053 | 1011:7054 | 1011:7055 |
| 1012:7049 | 1012:7050 | 1012:7051 | 1012:7052 | 1012:7053 | 1012:7054 | 1012:7055 |
| 1013:7049 | 1013:7050 | 1013:7051 | 1013:7052 | 1013:7053 | 1013:7054 | 1013:7055 |
| 1014:7049 | 1014:7050 | 1014:7051 | 1014:7052 | 1014:7053 | 1014:7054 | 1014:7055 |
| 2001:7049 | 2001:7050 | 2001:7051 | 2001:7052 | 2001:7053 | 2001:7054 | 2001:7055 |
| 2002:7049 | 2002:7050 | 2002:7051 | 2002:7052 | 2002:7053 | 2002:7054 | 2002:7055 |
| 2003:7049 | 2003:7050 | 2003:7051 | 2003:7052 | 2003:7053 | 2003:7054 | 2003:7055 |
| 2004:7049 | 2004:7050 | 2004:7051 | 2004:7052 | 2004:7053 | 2004:7054 | 2004:7055 |
| 2005:7049 | 2005:7050 | 2005:7051 | 2005:7052 | 2005:7053 | 2005:7054 | 2005:7055 |
| 2006:7049 | 2006:7050 | 2006:7051 | 2006:7052 | 2006:7053 | 2006:7054 | 2006:7055 |
| 2007:7049 | 2007:7050 | 2007:7051 | 2007:7052 | 2007:7053 | 2007:7054 | 2007:7055 |
| 2008:7049 | 2008:7050 | 2008:7051 | 2008:7052 | 2008:7053 | 2008:7054 | 2008:7055 |
| 2009:7049 | 2009:7050 | 2009:7051 | 2009:7052 | 2009:7053 | 2009:7054 | 2009:7055 |
| 2010:7049 | 2010:7050 | 2010:7051 | 2010:7052 | 2010:7053 | 2010:7054 | 2010:7055 |
| 3001:7049 | 3001:7050 | 3001:7051 | 3001:7052 | 3001:7053 | 3001:7054 | 3001:7055 |
| 3002:7049 | 3002:7050 | 3002:7051 | 3002:7052 | 3002:7053 | 3002:7054 | 3002:7055 |
| 3003:7049 | 3003:7050 | 3003:7051 | 3003:7052 | 3003:7053 | 3003:7054 | 3003:7055 |
| 3004:7049 | 3004:7050 | 3004:7051 | 3004:7052 | 3004:7053 | 3004:7054 | 3004:7055 |
| 3005:7049 | 3005:7050 | 3005:7051 | 3005:7052 | 3005:7053 | 3005:7054 | 3005:7055 |
| 3006:7049 | 3006:7050 | 3006:7051 | 3006:7052 | 3006:7053 | 3006:7054 | 3006:7055 |
| 3007:7049 | 3007:7050 | 3007:7051 | 3007:7052 | 3007:7053 | 3007:7054 | 3007:7055 |
| 3008:7049 | 3008:7050 | 3008:7051 | 3008:7052 | 3008:7053 | 3008:7054 | 3008:7055 |
| 4001:7049 | 4001:7050 | 4001:7051 | 4001:7052 | 4001:7053 | 4001:7054 | 4001:7055 |
| 4002:7049 | 4002:7050 | 4002:7051 | 4002:7052 | 4002:7053 | 4002:7054 | 4002:7055 |
| 4003:7049 | 4003:7050 | 4003:7051 | 4003:7052 | 4003:7053 | 4003:7054 | 4003:7055 |
| 4004:7049 | 4004:7050 | 4004:7051 | 4004:7052 | 4004:7053 | 4004:7054 | 4004:7055 |
| 4005:7049 | 4005:7050 | 4005:7051 | 4005:7052 | 4005:7053 | 4005:7054 | 4005:7055 |
| 5001:7049 | 5001:7050 | 5001:7051 | 5001:7052 | 5001:7053 | 5001:7054 | 5001:7055 |
| 5002:7049 | 5002:7050 | 5002:7051 | 5002:7052 | 5002:7053 | 5002:7054 | 5002:7055 |
| X:Y | X:Y | X:Y | X:Y | X:Y | X:Y | X:Y |
| 1001:7056 | 1001:7057 | 1001:7058 | 1001:7059 | 1001:7060 | 1001:7061 | 1001:7062 |
| 1002:7056 | 1002:7057 | 1002:7058 | 1002:7059 | 1002:7060 | 1002:7061 | 1002:7062 |
| 1003:7056 | 1003:7057 | 1003:7058 | 1003:7059 | 1003:7060 | 1003:7061 | 1003:7062 |
| 1004:7056 | 1004:7057 | 1004:7058 | 1004:7059 | 1004:7060 | 1004:7061 | 1004:7062 |
| 1005:7056 | 1005:7057 | 1005:7058 | 1005:7059 | 1005:7060 | 1005:7061 | 1005:7062 |
| 1006:7056 | 1006:7057 | 1006:7058 | 1006:7059 | 1006:7060 | 1006:7061 | 1006:7062 |
| 1007:7056 | 1007:7057 | 1007:7058 | 1007:7059 | 1007:7060 | 1007:7061 | 1007:7062 |
| 1008:7056 | 1008:7057 | 1008:7058 | 1008:7059 | 1008:7060 | 1008:7061 | 1008:7062 |
| 1009:7056 | 1009:7057 | 1009:7058 | 1009:7059 | 1009:7060 | 1009:7061 | 1009:7062 |
| 1010:7056 | 1010:7057 | 1010:7058 | 1010:7059 | 1010:7060 | 1010:7061 | 1010:7062 |
| 1011:7056 | 1011:7057 | 1011:7058 | 1011:7059 | 1011:7060 | 1011:7061 | 1011:7062 |
| 1012:7056 | 1012:7057 | 1012:7058 | 1012:7059 | 1012:7060 | 1012:7061 | 1012:7062 |
| 1013:7056 | 1013:7057 | 1013:7058 | 1013:7059 | 1013:7060 | 1013:7061 | 1013:7062 |
| 1014:7056 | 1014:7057 | 1014:7058 | 1014:7059 | 1014:7060 | 1014:7061 | 1014:7062 |
| 2001:7056 | 2001:7057 | 2001:7058 | 2001:7059 | 2001:7060 | 2001:7061 | 2001:7062 |
| 2002:7056 | 2002:7057 | 2002:7058 | 2002:7059 | 2002:7060 | 2002:7061 | 2002:7062 |
| 2003:7056 | 2003:7057 | 2003:7058 | 2003:7059 | 2003:7060 | 2003:7061 | 2003:7062 |
| 2004:7056 | 2004:7057 | 2004:7058 | 2004:7059 | 2004:7060 | 2004:7061 | 2004:7062 |
| 2005:7056 | 2005:7057 | 2005:7058 | 2005:7059 | 2005:7060 | 2005:7061 | 2005:7062 |
| 2006:7056 | 2006:7057 | 2006:7058 | 2006:7059 | 2006:7060 | 2006:7061 | 2006:7062 |
| 2007:7056 | 2007:7057 | 2007:7058 | 2007:7059 | 2007:7060 | 2007:7061 | 2007:7062 |
| 2008:7056 | 2008:7057 | 2008:7058 | 2008:7059 | 2008:7060 | 2008:7061 | 2008:7062 |
| 2009:7056 | 2009:7057 | 2009:7058 | 2009:7059 | 2009:7060 | 2009:7061 | 2009:7062 |
| 2010:7056 | 2010:7057 | 2010:7058 | 2010:7059 | 2010:7060 | 2010:7061 | 2010:7062 |
| 3001:7056 | 3001:7057 | 3001:7058 | 3001:7059 | 3001:7060 | 3001:7061 | 3001:7062 |
| 3002:7056 | 3002:7057 | 3002:7058 | 3002:7059 | 3002:7060 | 3002:7061 | 3002:7062 |
| 3003:7056 | 3003:7057 | 3003:7058 | 3003:7059 | 3003:7060 | 3003:7061 | 3003:7062 |
| 3004:7056 | 3004:7057 | 3004:7058 | 3004:7059 | 3004:7060 | 3004:7061 | 3004:7062 |
| 3005:7056 | 3005:7057 | 3005:7058 | 3005:7059 | 3005:7060 | 3005:7061 | 3005:7062 |
| 3006:7056 | 3006:7057 | 3006:7058 | 3006:7059 | 3006:7060 | 3006:7061 | 3006:7062 |
| 3007:7056 | 3007:7057 | 3007:7058 | 3007:7059 | 3007:7060 | 3007:7061 | 3007:7062 |
| 3008:7056 | 3008:7057 | 3008:7058 | 3008:7059 | 3008:7060 | 3008:7061 | 3008:7062 |
| 4001:7056 | 4001:7057 | 4001:7058 | 4001:7059 | 4001:7060 | 4001:7061 | 4001:7062 |
| 4002:7056 | 4002:7057 | 4002:7058 | 4002:7059 | 4002:7060 | 4002:7061 | 4002:7062 |
| 4003:7056 | 4003:7057 | 4003:7058 | 4003:7059 | 4003:7060 | 4003:7061 | 4003:7062 |
| 4004:7056 | 4004:7057 | 4004:7058 | 4004:7059 | 4004:7060 | 4004:7061 | 4004:7062 |
| 4005:7056 | 4005:7057 | 4005:7058 | 4005:7059 | 4005:7060 | 4005:7061 | 4005:7062 |
| 5001:7056 | 5001:7057 | 5001:7058 | 5001:7059 | 5001:7060 | 5001:7061 | 5001:7062 |
| 5002:7056 | 5002:7057 | 5002:7058 | 5002:7059 | 5002:7060 | 5002:7061 | 5002:7062 |
| X:Y | X:Y | X:Y | X:Y | X:Y | X:Y | X:Y |
| 1001:7063 | 1001:7064 | 1001:7065 | 1001:7066 | 1001:7067 | 1001:7068 | 1001:7069 |
| 1002:7063 | 1002:7064 | 1002:7065 | 1002:7066 | 1002:7067 | 1002:7068 | 1002:7069 |
| 1003:7063 | 1003:7064 | 1003:7065 | 1003:7066 | 1003:7067 | 1003:7068 | 1003:7069 |
| 1004:7063 | 1004:7064 | 1004:7065 | 1004:7066 | 1004:7067 | 1004:7068 | 1004:7069 |
| 1005:7063 | 1005:7064 | 1005:7065 | 1005:7066 | 1005:7067 | 1005:7068 | 1005:7069 |
| 1006:7063 | 1006:7064 | 1006:7065 | 1006:7066 | 1006:7067 | 1006:7068 | 1006:7069 |
| 1007:7063 | 1007:7064 | 1007:7065 | 1007:7066 | 1007:7067 | 1007:7068 | 1007:7069 |
| 1008:7063 | 1008:7064 | 1008:7065 | 1008:7066 | 1008:7067 | 1008:7068 | 1008:7069 |
| 1009:7063 | 1009:7064 | 1009:7065 | 1009:7066 | 1009:7067 | 1009:7068 | 1009:7069 |
| 1010:7063 | 1010:7064 | 1010:7065 | 1010:7066 | 1010:7067 | 1010:7068 | 1010:7069 |
| 1011:7063 | 1011:7064 | 1011:7065 | 1011:7066 | 1011:7067 | 1011:7068 | 1011:7069 |
| 1012:7063 | 1012:7064 | 1012:7065 | 1012:7066 | 1012:7067 | 1012:7068 | 1012:7069 |
| 1013:7063 | 1013:7064 | 1013:7065 | 1013:7066 | 1013:7067 | 1013:7068 | 1013:7069 |
| 1014:7063 | 1014:7064 | 1014:7065 | 1014:7066 | 1014:7067 | 1014:7068 | 1014:7069 |
| 2001:7063 | 2001:7064 | 2001:7065 | 2001:7066 | 2001:7067 | 2001:7068 | 2001:7069 |
| 2002:7063 | 2002:7064 | 2002:7065 | 2002:7066 | 2002:7067 | 2002:7068 | 2002:7069 |
| 2003:7063 | 2003:7064 | 2003:7065 | 2003:7066 | 2003:7067 | 2003:7068 | 2003:7069 |
| 2004:7063 | 2004:7064 | 2004:7065 | 2004:7066 | 2004:7067 | 2004:7068 | 2004:7069 |
| 2005:7063 | 2005:7064 | 2005:7065 | 2005:7066 | 2005:7067 | 2005:7068 | 2005:7069 |
| 2006:7063 | 2006:7064 | 2006:7065 | 2006:7066 | 2006:7067 | 2006:7068 | 2006:7069 |
| 2007:7063 | 2007:7064 | 2007:7065 | 2007:7066 | 2007:7067 | 2007:7068 | 2007:7069 |
| 2008:7063 | 2008:7064 | 2008:7065 | 2008:7066 | 2008:7067 | 2008:7068 | 2008:7069 |
| 2009:7063 | 2009:7064 | 2009:7065 | 2009:7066 | 2009:7067 | 2009:7068 | 2009:7069 |
| 2010:7063 | 2010:7064 | 2010:7065 | 2010:7066 | 2010:7067 | 2010:7068 | 2010:7069 |
| 3001:7063 | 3001:7064 | 3001:7065 | 3001:7066 | 3001:7067 | 3001:7068 | 3001:7069 |
| 3002:7063 | 3002:7064 | 3002:7065 | 3002:7066 | 3002:7067 | 3002:7068 | 3002:7069 |
| 3003:7063 | 3003:7064 | 3003:7065 | 3003:7066 | 3003:7067 | 3003:7068 | 3003:7069 |
| 3004:7063 | 3004:7064 | 3004:7065 | 3004:7066 | 3004:7067 | 3004:7068 | 3004:7069 |
| 3005:7063 | 3005:7064 | 3005:7065 | 3005:7066 | 3005:7067 | 3005:7068 | 3005:7069 |
| 3006:7063 | 3006:7064 | 3006:7065 | 3006:7066 | 3006:7067 | 3006:7068 | 3006:7069 |
| 3007:7063 | 3007:7064 | 3007:7065 | 3007:7066 | 3007:7067 | 3007:7068 | 3007:7069 |
| 3008:7063 | 3008:7064 | 3008:7065 | 3008:7066 | 3008:7067 | 3008:7068 | 3008:7069 |
| 4001:7063 | 4001:7064 | 4001:7065 | 4001:7066 | 4001:7067 | 4001:7068 | 4001:7069 |
| 4002:7063 | 4002:7064 | 4002:7065 | 4002:7066 | 4002:7067 | 4002:7068 | 4002:7069 |
| 4003:7063 | 4003:7064 | 4003:7065 | 4003:7066 | 4003:7067 | 4003:7068 | 4003:7069 |
| 4004:7063 | 4004:7064 | 4004:7065 | 4004:7066 | 4004:7067 | 4004:7068 | 4004:7069 |
| 4005:7063 | 4005:7064 | 4005:7065 | 4005:7066 | 4005:7067 | 4005:7068 | 4005:7069 |
| 5001:7063 | 5001:7064 | 5001:7065 | 5001:7066 | 5001:7067 | 5001:7068 | 5001:7069 |
| 5002:7063 | 5002:7064 | 5002:7065 | 5002:7066 | 5002:7067 | 5002:7068 | 5002:7069 |
| X:Y | X:Y | X:Y | X:Y | X:Y | X:Y | X:Y |
| 1001:7070 | 1001:7071 | 1001:7072 | 1001:7073 | 1001:7074 | 1001:7075 | 1001:7076 |
| 1002:7070 | 1002:7071 | 1002:7072 | 1002:7073 | 1002:7074 | 1002:7075 | 1002:7076 |
| 1003:7070 | 1003:7071 | 1003:7072 | 1003:7073 | 1003:7074 | 1003:7075 | 1003:7076 |
| 1004:7070 | 1004:7071 | 1004:7072 | 1004:7073 | 1004:7074 | 1004:7075 | 1004:7076 |
| 1005:7070 | 1005:7071 | 1005:7072 | 1005:7073 | 1005:7074 | 1005:7075 | 1005:7076 |
| 1006:7070 | 1006:7071 | 1006:7072 | 1006:7073 | 1006:7074 | 1006:7075 | 1006:7076 |
| 1007:7070 | 1007:7071 | 1007:7072 | 1007:7073 | 1007:7074 | 1007:7075 | 1007:7076 |
| 1008:7070 | 1008:7071 | 1008:7072 | 1008:7073 | 1008:7074 | 1008:7075 | 1008:7076 |
| 1009:7070 | 1009:7071 | 1009:7072 | 1009:7073 | 1009:7074 | 1009:7075 | 1009:7076 |
| 1010:7070 | 1010:7071 | 1010:7072 | 1010:7073 | 1010:7074 | 1010:7075 | 1010:7076 |
| 1011:7070 | 1011:7071 | 1011:7072 | 1011:7073 | 1011:7074 | 1011:7075 | 1011:7076 |
| 1012:7070 | 1012:7071 | 1012:7072 | 1012:7073 | 1012:7074 | 1012:7075 | 1012:7076 |
| 1013:7070 | 1013:7071 | 1013:7072 | 1013:7073 | 1013:7074 | 1013:7075 | 1013:7076 |
| 1014:7070 | 1014:7071 | 1014:7072 | 1014:7073 | 1014:7074 | 1014:7075 | 1014:7076 |
| 2001:7070 | 2001:7071 | 2001:7072 | 2001:7073 | 2001:7074 | 2001:7075 | 2001:7076 |
| 2002:7070 | 2002:7071 | 2002:7072 | 2002:7073 | 2002:7074 | 2002:7075 | 2002:7076 |
| 2003:7070 | 2003:7071 | 2003:7072 | 2003:7073 | 2003:7074 | 2003:7075 | 2003:7076 |
| 2004:7070 | 2004:7071 | 2004:7072 | 2004:7073 | 2004:7074 | 2004:7075 | 2004:7076 |
| 2005:7070 | 2005:7071 | 2005:7072 | 2005:7073 | 2005:7074 | 2005:7075 | 2005:7076 |
| 2006:7070 | 2006:7071 | 2006:7072 | 2006:7073 | 2006:7074 | 2006:7075 | 2006:7076 |
| 2007:7070 | 2007:7071 | 2007:7072 | 2007:7073 | 2007:7074 | 2007:7075 | 2007:7076 |
| 2008:7070 | 2008:7071 | 2008:7072 | 2008:7073 | 2008:7074 | 2008:7075 | 2008:7076 |
| 2009:7070 | 2009:7071 | 2009:7072 | 2009:7073 | 2009:7074 | 2009:7075 | 2009:7076 |
| 2010:7070 | 2010:7071 | 2010:7072 | 2010:7073 | 2010:7074 | 2010:7075 | 2010:7076 |
| 3001:7070 | 3001:7071 | 3001:7072 | 3001:7073 | 3001:7074 | 3001:7075 | 3001:7076 |
| 3002:7070 | 3002:7071 | 3002:7072 | 3002:7073 | 3002:7074 | 3002:7075 | 3002:7076 |
| 3003:7070 | 3003:7071 | 3003:7072 | 3003:7073 | 3003:7074 | 3003:7075 | 3003:7076 |
| 3004:7070 | 3004:7071 | 3004:7072 | 3004:7073 | 3004:7074 | 3004:7075 | 3004:7076 |
| 3005:7070 | 3005:7071 | 3005:7072 | 3005:7073 | 3005:7074 | 3005:7075 | 3005:7076 |
| 3006:7070 | 3006:7071 | 3006:7072 | 3006:7073 | 3006:7074 | 3006:7075 | 3006:7076 |
| 3007:7070 | 3007:7071 | 3007:7072 | 3007:7073 | 3007:7074 | 3007:7075 | 3007:7076 |
| 3008:7070 | 3008:7071 | 3008:7072 | 3008:7073 | 3008:7074 | 3008:7075 | 3008:7076 |
| 4001:7070 | 4001:7071 | 4001:7072 | 4001:7073 | 4001:7074 | 4001:7075 | 4001:7076 |
| 4002:7070 | 4002:7071 | 4002:7072 | 4002:7073 | 4002:7074 | 4002:7075 | 4002:7076 |
| 4003:7070 | 4003:7071 | 4003:7072 | 4003:7073 | 4003:7074 | 4003:7075 | 4003:7076 |
| 4004:7070 | 4004:7071 | 4004:7072 | 4004:7073 | 4004:7074 | 4004:7075 | 4004:7076 |
| 4005:7070 | 4005:7071 | 4005:7072 | 4005:7073 | 4005:7074 | 4005:7075 | 4005:7076 |
| 5001:7070 | 5001:7071 | 5001:7072 | 5001:7073 | 5001:7074 | 5001:7075 | 5001:7076 |
| 5002:7070 | 5002:7071 | 5002:7072 | 5002:7073 | 5002:7074 | 5002:7075 | 5002:7076 |
| X:Y | X:Y | X:Y | X:Y | X:Y | X:Y | X:Y |
| 1001:7077 | 1011:7077 | 2007:7077 | 3007:7077 | — | — | — |
| 1002:7077 | 1012:7077 | 2008:7077 | 3008:7077 | | | |
| 1003:7077 | 1013:7077 | 2009:7077 | 4001:7077 | | | |
| 1004:7077 | 1014:7077 | 2010:7077 | 4002:7077 | | | |
| 1005:7077 | 2001:7077 | 3001:7077 | 4003:7077 | | | |
| 1006:7077 | 2002:7077 | 3002:7077 | 4004:7077 | | | |
| 1007:7077 | 2003:7077 | 3003:7077 | 4005:7077 | | | |
| 1008:7077 | 2004:7077 | 3004:7077 | 5001:7077 | | | |
| 1009:7077 | 2005:7077 | 3005:7077 | 5002:7077 | | | |
| 1010:7077 | 2006:7077 | 3006:7077 | — | | | |

TABLE B
Example combinations of a compound X with a compound Y.

| X:Y | X:Y | X:Y | X:Y | X:Y | X:Y | X:Y |
|---|---|---|---|---|---|---|
| 6000:7000 | 6000:7001 | 6000:7002 | 6000:7003 | 6000:7004 | 6000:7005 | 6000:7006 |
| 6001:7000 | 6001:7001 | 6001:7002 | 6001:7003 | 6001:7004 | 6001:7005 | 6001:7006 |
| 6002:7000 | 6002:7001 | 6002:7002 | 6002:7003 | 6002:7004 | 6002:7005 | 6002:7006 |
| 6003:7000 | 6003:7001 | 6003:7002 | 6003:7003 | 6003:7004 | 6003:7005 | 6003:7006 |
| 6004:7000 | 6004:7001 | 6004:7002 | 6004:7003 | 6004:7004 | 6004:7005 | 6004:7006 |
| 6005:7000 | 6005:7001 | 6005:7002 | 6005:7003 | 6005:7004 | 6005:7005 | 6005:7006 |
| 6006:7000 | 6006:7001 | 6006:7002 | 6006:7003 | 6006:7004 | 6006:7005 | 6006:7006 |
| 6007:7000 | 6007:7001 | 6007:7002 | 6007:7003 | 6007:7004 | 6007:7005 | 6007:7006 |
| 6008:7000 | 6008:7001 | 6008:7002 | 6008:7003 | 6008:7004 | 6008:7005 | 6008:7006 |
| 6009:7000 | 6009:7001 | 6009:7002 | 6009:7003 | 6009:7004 | 6009:7005 | 6009:7006 |
| 6010:7000 | 6010:7001 | 6010:7002 | 6010:7003 | 6010:7004 | 6010:7005 | 6010:7006 |
| 6011:7000 | 6011:7001 | 6011:7002 | 6011:7003 | 6011:7004 | 6011:7005 | 6011:7006 |
| 6012:7000 | 6012:7001 | 6012:7002 | 6012:7003 | 6012:7004 | 6012:7005 | 6012:7006 |
| 6013:7000 | 6013:7001 | 6013:7002 | 6013:7003 | 6013:7004 | 6013:7005 | 6013:7006 |
| 6014:7000 | 6014:7001 | 6014:7002 | 6014:7003 | 6014:7004 | 6014:7005 | 6014:7006 |
| 6015:7000 | 6015:7001 | 6015:7002 | 6015:7003 | 6015:7004 | 6015:7005 | 6015:7006 |
| 6016:7000 | 6016:7001 | 6016:7002 | 6016:7003 | 6016:7004 | 6016:7005 | 6016:7006 |
| 6017:7000 | 6017:7001 | 6017:7002 | 6017:7003 | 6017:7004 | 6017:7005 | 6017:7006 |
| 6018:7000 | 6018:7001 | 6018:7002 | 6018:7003 | 6018:7004 | 6018:7005 | 6018:7006 |
| 6019:7000 | 6019:7001 | 6019:7002 | 6019:7003 | 6019:7004 | 6019:7005 | 6019:7006 |
| 6020:7000 | 6020:7001 | 6020:7002 | 6020:7003 | 6020:7004 | 6020:7005 | 6020:7006 |
| 6000:7007 | 6000:7008 | 6000:7009 | 6000:7010 | 6000:7011 | 6000:7012 | 6000:7013 |
| 6001:7007 | 6001:7008 | 6001:7009 | 6001:7010 | 6001:7011 | 6001:7012 | 6001:7013 |
| 6002:7007 | 6002:7008 | 6002:7009 | 6002:7010 | 6002:7011 | 6002:7012 | 6002:7013 |
| 6003:7007 | 6003:7008 | 6003:7009 | 6003:7010 | 6003:7011 | 6003:7012 | 6003:7013 |
| 6004:7007 | 6004:7008 | 6004:7009 | 6004:7010 | 6004:7011 | 6004:7012 | 6004:7013 |
| 6005:7007 | 6005:7008 | 6005:7009 | 6005:7010 | 6005:7011 | 6005:7012 | 6005:7013 |
| 6006:7007 | 6006:7008 | 6006:7009 | 6006:7010 | 6006:7011 | 6006:7012 | 6006:7013 |
| 6007:7007 | 6007:7008 | 6007:7009 | 6007:7010 | 6007:7011 | 6007:7012 | 6007:7013 |
| 6008:7007 | 6008:7008 | 6008:7009 | 6008:7010 | 6008:7011 | 6008:7012 | 6008:7013 |
| 6009:7007 | 6009:7008 | 6009:7009 | 6009:7010 | 6009:7011 | 6009:7012 | 6009:7013 |
| 6010:7007 | 6010:7008 | 6010:7009 | 6010:7010 | 6010:7011 | 6010:7012 | 6010:7013 |
| 6011:7007 | 6011:7008 | 6011:7009 | 6011:7010 | 6011:7011 | 6011:7012 | 6011:7013 |
| 6012:7007 | 6012:7008 | 6012:7009 | 6012:7010 | 6012:7011 | 6012:7012 | 6012:7013 |
| 6013:7007 | 6013:7008 | 6013:7009 | 6013:7010 | 6013:7011 | 6013:7012 | 6013:7013 |
| 6014:7007 | 6014:7008 | 6014:7009 | 6014:7010 | 6014:7011 | 6014:7012 | 6014:7013 |
| 6015:7007 | 6015:7008 | 6015:7009 | 6015:7010 | 6015:7011 | 6015:7012 | 6015:7013 |
| 6016:7007 | 6016:7008 | 6016:7009 | 6016:7010 | 6016:7011 | 6016:7012 | 6016:7013 |
| 6017:7007 | 6017:7008 | 6017:7009 | 6017:7010 | 6017:7011 | 6017:7012 | 6017:7013 |
| 6018:7007 | 6018:7008 | 6018:7009 | 6018:7010 | 6018:7011 | 6018:7012 | 6018:7013 |
| 6019:7007 | 6019:7008 | 6019:7009 | 6019:7010 | 6019:7011 | 6019:7012 | 6019:7013 |
| 6020:7007 | 6020:7008 | 6020:7009 | 6020:7010 | 6020:7011 | 6020:7012 | 6020:7013 |
| 6000:7014 | 6000:7015 | 6000:7016 | 6000:7017 | 6000:7018 | 6000:7019 | 6000:7020 |
| 6001:7014 | 6001:7015 | 6001:7016 | 6001:7017 | 6001:7018 | 6001:7019 | 6001:7020 |
| 6002:7014 | 6002:7015 | 6002:7016 | 6002:7017 | 6002:7018 | 6002:7019 | 6002:7020 |
| 6003:7014 | 6003:7015 | 6003:7016 | 6003:7017 | 6003:7018 | 6003:7019 | 6003:7020 |
| 6004:7014 | 6004:7015 | 6004:7016 | 6004:7017 | 6004:7018 | 6004:7019 | 6004:7020 |
| 6005:7014 | 6005:7015 | 6005:7016 | 6005:7017 | 6005:7018 | 6005:7019 | 6005:7020 |
| 6006:7014 | 6006:7015 | 6006:7016 | 6006:7017 | 6006:7018 | 6006:7019 | 6006:7020 |
| 6007:7014 | 6007:7015 | 6007:7016 | 6007:7017 | 6007:7018 | 6007:7019 | 6007:7020 |
| 6008:7014 | 6008:7015 | 6008:7016 | 6008:7017 | 6008:7018 | 6008:7019 | 6008:7020 |
| 6009:7014 | 6009:7015 | 6009:7016 | 6009:7017 | 6009:7018 | 6009:7019 | 6009:7020 |
| 6010:7014 | 6010:7015 | 6010:7016 | 6010:7017 | 6010:7018 | 6010:7019 | 6010:7020 |
| 6011:7014 | 6011:7015 | 6011:7016 | 6011:7017 | 6011:7018 | 6011:7019 | 6011:7020 |
| 6012:7014 | 6012:7015 | 6012:7016 | 6012:7017 | 6012:7018 | 6012:7019 | 6012:7020 |
| 6013:7014 | 6013:7015 | 6013:7016 | 6013:7017 | 6013:7018 | 6013:7019 | 6013:7020 |
| 6014:7014 | 6014:7015 | 6014:7016 | 6014:7017 | 6014:7018 | 6014:7019 | 6014:7020 |
| 6015:7014 | 6015:7015 | 6015:7016 | 6015:7017 | 6015:7018 | 6015:7019 | 6015:7020 |
| 6016:7014 | 6016:7015 | 6016:7016 | 6016:7017 | 6016:7018 | 6016:7019 | 6016:7020 |
| 6017:7014 | 6017:7015 | 6017:7016 | 6017:7017 | 6017:7018 | 6017:7019 | 6017:7020 |
| 6018:7014 | 6018:7015 | 6018:7016 | 6018:7017 | 6018:7018 | 6018:7019 | 6018:7020 |
| 6019:7014 | 6019:7015 | 6019:7016 | 6019:7017 | 6019:7018 | 6019:7019 | 6019:7020 |
| 6020:7014 | 6020:7015 | 6020:7016 | 6020:7017 | 6020:7018 | 6020:7019 | 6020:7020 |
| 6000:7021 | 6000:7022 | 6000:7023 | 6000:7024 | 6000:7025 | 6000:7026 | 6000:7027 |
| 6001:7021 | 6001:7022 | 6001:7023 | 6001:7024 | 6001:7025 | 6001:7026 | 6001:7027 |
| 6002:7021 | 6002:7022 | 6002:7023 | 6002:7024 | 6002:7025 | 6002:7026 | 6002:7027 |
| 6003:7021 | 6003:7022 | 6003:7023 | 6003:7024 | 6003:7025 | 6003:7026 | 6003:7027 |
| 6004:7021 | 6004:7022 | 6004:7023 | 6004:7024 | 6004:7025 | 6004:7026 | 6004:7027 |
| 6005:7021 | 6005:7022 | 6005:7023 | 6005:7024 | 6005:7025 | 6005:7026 | 6005:7027 |
| 6006:7021 | 6006:7022 | 6006:7023 | 6006:7024 | 6006:7025 | 6006:7026 | 6006:7027 |
| 6007:7021 | 6007:7022 | 6007:7023 | 6007:7024 | 6007:7025 | 6007:7026 | 6007:7027 |
| 6008:7021 | 6008:7022 | 6008:7023 | 6008:7024 | 6008:7025 | 6008:7026 | 6008:7027 |
| 6009:7021 | 6009:7022 | 6009:7023 | 6009:7024 | 6009:7025 | 6009:7026 | 6009:7027 |
| 6010:7021 | 6010:7022 | 6010:7023 | 6010:7024 | 6010:7025 | 6010:7026 | 6010:7027 |
| 6011:7021 | 6011:7022 | 6011:7023 | 6011:7024 | 6011:7025 | 6011:7026 | 6011:7027 |
| 6012:7021 | 6012:7022 | 6012:7023 | 6012:7024 | 6012:7025 | 6012:7026 | 6012:7027 |
| 6013:7021 | 6013:7022 | 6013:7023 | 6013:7024 | 6013:7025 | 6013:7026 | 6013:7027 |
| 6014:7021 | 6014:7022 | 6014:7023 | 6014:7024 | 6014:7025 | 6014:7026 | 6014:7027 |
| 6015:7021 | 6015:7022 | 6015:7023 | 6015:7024 | 6015:7025 | 6015:7026 | 6015:7027 |
| 6016:7021 | 6016:7022 | 6016:7023 | 6016:7024 | 6016:7025 | 6016:7026 | 6016:7027 |
| 6017:7021 | 6017:7022 | 6017:7023 | 6017:7024 | 6017:7025 | 6017:7026 | 6017:7027 |
| 6018:7021 | 6018:7022 | 6018:7023 | 6018:7024 | 6018:7025 | 6018:7026 | 6018:7027 |
| 6019:7021 | 6019:7022 | 6019:7023 | 6019:7024 | 6019:7025 | 6019:7026 | 6019:7027 |
| 6020:7021 | 6020:7022 | 6020:7023 | 6020:7024 | 6020:7025 | 6020:7026 | 6020:7027 |
| 6000:7028 | 6000:7029 | 6000:7030 | 6000:7031 | 6000:7032 | 6000:7033 | 6000:7034 |
| 6001:7028 | 6001:7029 | 6001:7030 | 6001:7031 | 6001:7032 | 6001:7033 | 6001:7034 |
| 6002:7028 | 6002:7029 | 6002:7030 | 6002:7031 | 6002:7032 | 6002:7033 | 6002:7034 |
| 6003:7028 | 6003:7029 | 6003:7030 | 6003:7031 | 6003:7032 | 6003:7033 | 6003:7034 |
| 6004:7028 | 6004:7029 | 6004:7030 | 6004:7031 | 6004:7032 | 6004:7033 | 6004:7034 |
| 6005:7028 | 6005:7029 | 6005:7030 | 6005:7031 | 6005:7032 | 6005:7033 | 6005:7034 |
| 6006:7028 | 6006:7029 | 6006:7030 | 6006:7031 | 6006:7032 | 6006:7033 | 6006:7034 |
| 6007:7028 | 6007:7029 | 6007:7030 | 6007:7031 | 6007:7032 | 6007:7033 | 6007:7034 |
| 6008:7028 | 6008:7029 | 6008:7030 | 6008:7031 | 6008:7032 | 6008:7033 | 6008:7034 |
| 6009:7028 | 6009:7029 | 6009:7030 | 6009:7031 | 6009:7032 | 6009:7033 | 6009:7034 |
| 6010:7028 | 6010:7029 | 6010:7030 | 6010:7031 | 6010:7032 | 6010:7033 | 6010:7034 |
| 6011:7028 | 6011:7029 | 6011:7030 | 6011:7031 | 6011:7032 | 6011:7033 | 6011:7034 |
| 6012:7028 | 6012:7029 | 6012:7030 | 6012:7031 | 6012:7032 | 6012:7033 | 6012:7034 |
| 6013:7028 | 6013:7029 | 6013:7030 | 6013:7031 | 6013:7032 | 6013:7033 | 6013:7034 |
| 6014:7028 | 6014:7029 | 6014:7030 | 6014:7031 | 6014:7032 | 6014:7033 | 6014:7034 |
| 6015:7028 | 6015:7029 | 6015:7030 | 6015:7031 | 6015:7032 | 6015:7033 | 6015:7034 |
| 6016:7028 | 6016:7029 | 6016:7030 | 6016:7031 | 6016:7032 | 6016:7033 | 6016:7034 |
| 6017:7028 | 6017:7029 | 6017:7030 | 6017:7031 | 6017:7032 | 6017:7033 | 6017:7034 |
| 6018:7028 | 6018:7029 | 6018:7030 | 6018:7031 | 6018:7032 | 6018:7033 | 6018:7034 |
| 6019:7028 | 6019:7029 | 6019:7030 | 6019:7031 | 6019:7032 | 6019:7033 | 6019:7034 |
| 6020:7028 | 6020:7029 | 6020:7030 | 6020:7031 | 6020:7032 | 6020:7033 | 6020:7034 |
| 6000:7035 | 6000:7036 | 6000:7037 | 6000:7038 | 6000:7039 | 6000:7040 | 6000:7041 |
| 6001:7035 | 6001:7036 | 6001:7037 | 6001:7038 | 6001:7039 | 6001:7040 | 6001:7041 |
| 6002:7035 | 6002:7036 | 6002:7037 | 6002:7038 | 6002:7039 | 6002:7040 | 6002:7041 |
| 6003:7035 | 6003:7036 | 6003:7037 | 6003:7038 | 6003:7039 | 6003:7040 | 6003:7041 |
| 6004:7035 | 6004:7036 | 6004:7037 | 6004:7038 | 6004:7039 | 6004:7040 | 6004:7041 |
| 6005:7035 | 6005:7036 | 6005:7037 | 6005:7038 | 6005:7039 | 6005:7040 | 6005:7041 |
| 6006:7035 | 6006:7036 | 6006:7037 | 6006:7038 | 6006:7039 | 6006:7040 | 6006:7041 |
| 6007:7035 | 6007:7036 | 6007:7037 | 6007:7038 | 6007:7039 | 6007:7040 | 6007:7041 |
| 6008:7035 | 6008:7036 | 6008:7037 | 6008:7038 | 6008:7039 | 6008:7040 | 6008:7041 |
| 6009:7035 | 6009:7036 | 6009:7037 | 6009:7038 | 6009:7039 | 6009:7040 | 6009:7041 |
| 6010:7035 | 6010:7036 | 6010:7037 | 6010:7038 | 6010:7039 | 6010:7040 | 6010:7041 |
| 6011:7035 | 6011:7036 | 6011:7037 | 6011:7038 | 6011:7039 | 6011:7040 | 6011:7041 |
| 6012:7035 | 6012:7036 | 6012:7037 | 6012:7038 | 6012:7039 | 6012:7040 | 6012:7041 |
| 6013:7035 | 6013:7036 | 6013:7037 | 6013:7038 | 6013:7039 | 6013:7040 | 6013:7041 |
| 6014:7035 | 6014:7036 | 6014:7037 | 6014:7038 | 6014:7039 | 6014:7040 | 6014:7041 |
| 6015:7035 | 6015:7036 | 6015:7037 | 6015:7038 | 6015:7039 | 6015:7040 | 6015:7041 |
| 6016:7035 | 6016:7036 | 6016:7037 | 6016:7038 | 6016:7039 | 6016:7040 | 6016:7041 |
| 6017:7035 | 6017:7036 | 6017:7037 | 6017:7038 | 6017:7039 | 6017:7040 | 6017:7041 |
| 6018:7035 | 6018:7036 | 6018:7037 | 6018:7038 | 6018:7039 | 6018:7040 | 6018:7041 |
| 6019:7035 | 6019:7036 | 6019:7037 | 6019:7038 | 6019:7039 | 6019:7040 | 6019:7041 |
| 6020:7035 | 6020:7036 | 6020:7037 | 6020:7038 | 6020:7039 | 6020:7040 | 6020:7041 |
| 6000:7042 | 6000:7043 | 6000:7044 | 6000:7045 | 6000:7046 | 6000:7047 | 6000:7048 |
| 6001:7042 | 6001:7043 | 6001:7044 | 6001:7045 | 6001:7046 | 6001:7047 | 6001:7048 |
| 6002:7042 | 6002:7043 | 6002:7044 | 6002:7045 | 6002:7046 | 6002:7047 | 6002:7048 |
| 6003:7042 | 6003:7043 | 6003:7044 | 6003:7045 | 6003:7046 | 6003:7047 | 6003:7048 |
| 6004:7042 | 6004:7043 | 6004:7044 | 6004:7045 | 6004:7046 | 6004:7047 | 6004:7048 |
| 6005:7042 | 6005:7043 | 6005:7044 | 6005:7045 | 6005:7046 | 6005:7047 | 6005:7048 |
| 6006:7042 | 6006:7043 | 6006:7044 | 6006:7045 | 6006:7046 | 6006:7047 | 6006:7048 |
| 6007:7042 | 6007:7043 | 6007:7044 | 6007:7045 | 6007:7046 | 6007:7047 | 6007:7048 |
| 6008:7042 | 6008:7043 | 6008:7044 | 6008:7045 | 6008:7046 | 6008:7047 | 6008:7048 |
| 6009:7042 | 6009:7043 | 6009:7044 | 6009:7045 | 6009:7046 | 6009:7047 | 6009:7048 |
| 6010:7042 | 6010:7043 | 6010:7044 | 6010:7045 | 6010:7046 | 6010:7047 | 6010:7048 |
| 6011:7042 | 6011:7043 | 6011:7044 | 6011:7045 | 6011:7046 | 6011:7047 | 6011:7048 |
| 6012:7042 | 6012:7043 | 6012:7044 | 6012:7045 | 6012:7046 | 6012:7047 | 6012:7048 |
| 6013:7042 | 6013:7043 | 6013:7044 | 6013:7045 | 6013:7046 | 6013:7047 | 6013:7048 |
| 6014:7042 | 6014:7043 | 6014:7044 | 6014:7045 | 6014:7046 | 6014:7047 | 6014:7048 |
| 6015:7042 | 6015:7043 | 6015:7044 | 6015:7045 | 6015:7046 | 6015:7047 | 6015:7048 |
| 6016:7042 | 6016:7043 | 6016:7044 | 6016:7045 | 6016:7046 | 6016:7047 | 6016:7048 |
| 6017:7042 | 6017:7043 | 6017:7044 | 6017:7045 | 6017:7046 | 6017:7047 | 6017:7048 |
| 6018:7042 | 6018:7043 | 6018:7044 | 6018:7045 | 6018:7046 | 6018:7047 | 6018:7048 |
| 6019:7042 | 6019:7043 | 6019:7044 | 6019:7045 | 6019:7046 | 6019:7047 | 6019:7048 |
| 6020:7042 | 6020:7043 | 6020:7044 | 6020:7045 | 6020:7046 | 6020:7047 | 6020:7048 |
| 6000:7049 | 6000:7050 | 6000:7051 | 6000:7052 | 6000:7053 | 6000:7054 | 6000:7055 |
| 6001:7049 | 6001:7050 | 6001:7051 | 6001:7052 | 6001:7053 | 6001:7054 | 6001:7055 |
| 6002:7049 | 6002:7050 | 6002:7051 | 6002:7052 | 6002:7053 | 6002:7054 | 6002:7055 |
| 6003:7049 | 6003:7050 | 6003:7051 | 6003:7052 | 6003:7053 | 6003:7054 | 6003:7055 |
| 6004:7049 | 6004:7050 | 6004:7051 | 6004:7052 | 6004:7053 | 6004:7054 | 6004:7055 |
| 6005:7049 | 6005:7050 | 6005:7051 | 6005:7052 | 6005:7053 | 6005:7054 | 6005:7055 |
| 6006:7049 | 6006:7050 | 6006:7051 | 6006:7052 | 6006:7053 | 6006:7054 | 6006:7055 |
| 6007:7049 | 6007:7050 | 6007:7051 | 6007:7052 | 6007:7053 | 6007:7054 | 6007:7055 |
| 6008:7049 | 6008:7050 | 6008:7051 | 6008:7052 | 6008:7053 | 6008:7054 | 6008:7055 |
| 6009:7049 | 6009:7050 | 6009:7051 | 6009:7052 | 6009:7053 | 6009:7054 | 6009:7055 |
| 6010:7049 | 6010:7050 | 6010:7051 | 6010:7052 | 6010:7053 | 6010:7054 | 6010:7055 |
| 6011:7049 | 6011:7050 | 6011:7051 | 6011:7052 | 6011:7053 | 6011:7054 | 6011:7055 |
| 6012:7049 | 6012:7050 | 6012:7051 | 6012:7052 | 6012:7053 | 6012:7054 | 6012:7055 |
| 6013:7049 | 6013:7050 | 6013:7051 | 6013:7052 | 6013:7053 | 6013:7054 | 6013:7055 |
| 6014:7049 | 6014:7050 | 6014:7051 | 6014:7052 | 6014:7053 | 6014:7054 | 6014:7055 |
| 6015:7049 | 6015:7050 | 6015:7051 | 6015:7052 | 6015:7053 | 6015:7054 | 6015:7055 |
| 6016:7049 | 6016:7050 | 6016:7051 | 6016:7052 | 6016:7053 | 6016:7054 | 6016:7055 |
| 6017:7049 | 6017:7050 | 6017:7051 | 6017:7052 | 6017:7053 | 6017:7054 | 6017:7055 |
| 6018:7049 | 6018:7050 | 6018:7051 | 6018:7052 | 6018:7053 | 6018:7054 | 6018:7055 |
| 6019:7049 | 6019:7050 | 6019:7051 | 6019:7052 | 6019:7053 | 6019:7054 | 6019:7055 |
| 6020:7049 | 6020:7050 | 6020:7051 | 6020:7052 | 6020:7053 | 6020:7054 | 6020:7055 |
| 6000:7056 | 6000:7057 | 6000:7058 | 6000:7059 | 6000:7060 | 6000:7061 | 6000:7062 |
| 6001:7056 | 6001:7057 | 6001:7058 | 6001:7059 | 6001:7060 | 6001:7061 | 6001:7062 |
| 6002:7056 | 6002:7057 | 6002:7058 | 6002:7059 | 6002:7060 | 6002:7061 | 6002:7062 |
| 6003:7056 | 6003:7057 | 6003:7058 | 6003:7059 | 6003:7060 | 6003:7061 | 6003:7062 |
| 6004:7056 | 6004:7057 | 6004:7058 | 6004:7059 | 6004:7060 | 6004:7061 | 6004:7062 |
| 6005:7056 | 6005:7057 | 6005:7058 | 6005:7059 | 6005:7060 | 6005:7061 | 6005:7062 |
| 6006:7056 | 6006:7057 | 6006:7058 | 6006:7059 | 6006:7060 | 6006:7061 | 6006:7062 |
| 6007:7056 | 6007:7057 | 6007:7058 | 6007:7059 | 6007:7060 | 6007:7061 | 6007:7062 |
| 6008:7056 | 6008:7057 | 6008:7058 | 6008:7059 | 6008:7060 | 6008:7061 | 6008:7062 |
| 6009:7056 | 6009:7057 | 6009:7058 | 6009:7059 | 6009:7060 | 6009:7061 | 6009:7062 |
| 6010:7056 | 6010:7057 | 6010:7058 | 6010:7059 | 6010:7060 | 6010:7061 | 6010:7062 |
| 6011:7056 | 6011:7057 | 6011:7058 | 6011:7059 | 6011:7060 | 6011:7061 | 6011:7062 |
| 6012:7056 | 6012:7057 | 6012:7058 | 6012:7059 | 6012:7060 | 6012:7061 | 6012:7062 |
| 6013:7056 | 6013:7057 | 6013:7058 | 6013:7059 | 6013:7060 | 6013:7061 | 6013:7062 |
| 6014:7056 | 6014:7057 | 6014:7058 | 6014:7059 | 6014:7060 | 6014:7061 | 6014:7062 |
| 6015:7056 | 6015:7057 | 6015:7058 | 6015:7059 | 6015:7060 | 6015:7061 | 6015:7062 |
| 6016:7056 | 6016:7057 | 6016:7058 | 6016:7059 | 6016:7060 | 6016:7061 | 6016:7062 |
| 6017:7056 | 6017:7057 | 6017:7058 | 6017:7059 | 6017:7060 | 6017:7061 | 6017:7062 |
| 6018:7056 | 6018:7057 | 6018:7058 | 6018:7059 | 6018:7060 | 6018:7061 | 6018:7062 |
| 6019:7056 | 6019:7057 | 6019:7058 | 6019:7059 | 6019:7060 | 6019:7061 | 6019:7062 |
| 6020:7056 | 6020:7057 | 6020:7058 | 6020:7059 | 6020:7060 | 6020:7061 | 6020:7062 |
| 6000:7063 | 6000:7064 | 6000:7065 | 6000:7066 | 6000:7067 | 6000:7068 | 6000:7069 |
| 6001:7063 | 6001:7064 | 6001:7065 | 6001:7066 | 6001:7067 | 6001:7068 | 6001:7069 |
| 6002:7063 | 6002:7064 | 6002:7065 | 6002:7066 | 6002:7067 | 6002:7068 | 6002:7069 |
| 6003:7063 | 6003:7064 | 6003:7065 | 6003:7066 | 6003:7067 | 6003:7068 | 6003:7069 |
| 6004:7063 | 6004:7064 | 6004:7065 | 6004:7066 | 6004:7067 | 6004:7068 | 6004:7069 |
| 6005:7063 | 6005:7064 | 6005:7065 | 6005:7066 | 6005:7067 | 6005:7068 | 6005:7069 |
| 6006:7063 | 6006:7064 | 6006:7065 | 6006:7066 | 6006:7067 | 6006:7068 | 6006:7069 |
| 6007:7063 | 6007:7064 | 6007:7065 | 6007:7066 | 6007:7067 | 6007:7068 | 6007:7069 |
| 6008:7063 | 6008:7064 | 6008:7065 | 6008:7066 | 6008:7067 | 6008:7068 | 6008:7069 |
| 6009:7063 | 6009:7064 | 6009:7065 | 6009:7066 | 6009:7067 | 6009:7068 | 6009:7069 |
| 6010:7063 | 6010:7064 | 6010:7065 | 6010:7066 | 6010:7067 | 6010:7068 | 6010:7069 |
| 6011:7063 | 6011:7064 | 6011:7065 | 6011:7066 | 6011:7067 | 6011:7068 | 6011:7069 |
| 6012:7063 | 6012:7064 | 6012:7065 | 6012:7066 | 6012:7067 | 6012:7068 | 6012:7069 |
| 6013:7063 | 6013:7064 | 6013:7065 | 6013:7066 | 6013:7067 | 6013:7068 | 6013:7069 |
| 6014:7063 | 6014:7064 | 6014:7065 | 6014:7066 | 6014:7067 | 6014:7068 | 6014:7069 |
| 6015:7063 | 6015:7064 | 6015:7065 | 6015:7066 | 6015:7067 | 6015:7068 | 6015:7069 |
| 6016:7063 | 6016:7064 | 6016:7065 | 6016:7066 | 6016:7067 | 6016:7068 | 6016:7069 |
| 6017:7063 | 6017:7064 | 6017:7065 | 6017:7066 | 6017:7067 | 6017:7068 | 6017:7069 |
| 6018:7063 | 6018:7064 | 6018:7065 | 6018:7066 | 6018:7067 | 6018:7068 | 6018:7069 |
| 6019:7063 | 6019:7064 | 6019:7065 | 6019:7066 | 6019:7067 | 6019:7068 | 6019:7069 |
| 6020:7063 | 6020:7064 | 6020:7065 | 6020:7066 | 6020:7067 | 6020:7068 | 6020:7069 |
| 6000:7070 | 6000:7071 | 6000:7072 | 6000:7073 | 6000:7074 | 6000:7075 | 6000:7076 |
| 6001:7070 | 6001:7071 | 6001:7072 | 6001:7073 | 6001:7074 | 6001:7075 | 6001:7076 |
| 6002:7070 | 6002:7071 | 6002:7072 | 6002:7073 | 6002:7074 | 6002:7075 | 6002:7076 |
| 6003:7070 | 6003:7071 | 6003:7072 | 6003:7073 | 6003:7074 | 6003:7075 | 6003:7076 |
| 6004:7070 | 6004:7071 | 6004:7072 | 6004:7073 | 6004:7074 | 6004:7075 | 6004:7076 |
| 6005:7070 | 6005:7071 | 6005:7072 | 6005:7073 | 6005:7074 | 6005:7075 | 6005:7076 |
| 6006:7070 | 6006:7071 | 6006:7072 | 6006:7073 | 6006:7074 | 6006:7075 | 6006:7076 |
| 6007:7070 | 6007:7071 | 6007:7072 | 6007:7073 | 6007:7074 | 6007:7075 | 6007:7076 |
| 6008:7070 | 6008:7071 | 6008:7072 | 6008:7073 | 6008:7074 | 6008:7075 | 6008:7076 |
| 6009:7070 | 6009:7071 | 6009:7072 | 6009:7073 | 6009:7074 | 6009:7075 | 6009:7076 |
| 6010:7070 | 6010:7071 | 6010:7072 | 6010:7073 | 6010:7074 | 6010:7075 | 6010:7076 |
| 6011:7070 | 6011:7071 | 6011:7072 | 6011:7073 | 6011:7074 | 6011:7075 | 6011:7076 |
| 6012:7070 | 6012:7071 | 6012:7072 | 6012:7073 | 6012:7074 | 6012:7075 | 6012:7076 |
| 6013:7070 | 6013:7071 | 6013:7072 | 6013:7073 | 6013:7074 | 6013:7075 | 6013:7076 |
| 6014:7070 | 6014:7071 | 6014:7072 | 6014:7073 | 6014:7074 | 6014:7075 | 6014:7076 |
| 6015:7070 | 6015:7071 | 6015:7072 | 6015:7073 | 6015:7074 | 6015:7075 | 6015:7076 |
| 6016:7070 | 6016:7071 | 6016:7072 | 6016:7073 | 6016:7074 | 6016:7075 | 6016:7076 |
| 6017:7070 | 6017:7071 | 6017:7072 | 6017:7073 | 6017:7074 | 6017:7075 | 6017:7076 |
| 6018:7070 | 6018:7071 | 6018:7072 | 6018:7073 | 6018:7074 | 6018:7075 | 6018:7076 |
| 6019:7070 | 6019:7071 | 6019:7072 | 6019:7073 | 6019:7074 | 6019:7075 | 6019:7076 |
| 6020:7070 | 6020:7071 | 6020:7072 | 6020:7073 | 6020:7074 | 6020:7075 | 6020:7076 |
| 6000:7077 | 6021:7000 | 6021:7001 | 6021:7002 | 6021:7003 | 6021:7004 | 6021:7005 |
| 6001:7077 | 6022:7000 | 6022:7001 | 6022:7002 | 6022:7003 | 6022:7004 | 6022:7005 |
| 6002:7077 | 6023:7000 | 6023:7001 | 6023:7002 | 6023:7003 | 6023:7004 | 6023:7005 |
| 6003:7077 | 6024:7000 | 6024:7001 | 6024:7002 | 6024:7003 | 6024:7004 | 6024:7005 |
| 6004:7077 | 6025:7000 | 6025:7001 | 6025:7002 | 6025:7003 | 6025:7004 | 6025:7005 |
| 6005:7077 | 6026:7000 | 6026:7001 | 6026:7002 | 6026:7003 | 6026:7004 | 6026:7005 |
| 6006:7077 | 6027:7000 | 6027:7001 | 6027:7002 | 6027:7003 | 6027:7004 | 6027:7005 |
| 6007:7077 | 6028:7000 | 6028:7001 | 6028:7002 | 6028:7003 | 6028:7004 | 6028:7005 |
| 6008:7077 | 6029:7000 | 6029:7001 | 6029:7002 | 6029:7003 | 6029:7004 | 6029:7005 |
| 6009:7077 | 6030:7000 | 6030:7001 | 6030:7002 | 6030:7003 | 6030:7004 | 6030:7005 |
| 6010:7077 | 6031:7000 | 6031:7001 | 6031:7002 | 6031:7003 | 6031:7004 | 6031:7005 |
| 6011:7077 | 6032:7000 | 6032:7001 | 6032:7002 | 6032:7003 | 6032:7004 | 6032:7005 |
| 6012:7077 | 6033:7000 | 6033:7001 | 6033:7002 | 6033:7003 | 6033:7004 | 6033:7005 |
| 6013:7077 | 6034:7000 | 6034:7001 | 6034:7002 | 6034:7003 | 6034:7004 | 6034:7005 |
| 6014:7077 | 6035:7000 | 6035:7001 | 6035:7002 | 6035:7003 | 6035:7004 | 6035:7005 |
| 6015:7077 | 6036:7000 | 6036:7001 | 6036:7002 | 6036:7003 | 6036:7004 | 6036:7005 |
| 6016:7077 | 6037:7000 | 6037:7001 | 6037:7002 | 6037:7003 | 6037:7004 | 6037:7005 |
| 6017:7077 | 6038:7000 | 6038:7001 | 6038:7002 | 6038:7003 | 6038:7004 | 6038:7005 |
| 6018:7077 | 6039:7000 | 6039:7001 | 6039:7002 | 6039:7003 | 6039:7004 | 6039:7005 |
| 6019:7077 | 6040:7000 | 6040:7001 | 6040:7002 | 6040:7003 | 6040:7004 | 6040:7005 |
| 6020:7077 | | | | | | |
| 6021:7006 | 6021:7007 | 6021:7008 | 6021:7009 | 6021:7010 | 6021:7011 | 6021:7012 |
| 6022:7006 | 6022:7007 | 6022:7008 | 6022:7009 | 6022:7010 | 6022:7011 | 6022:7012 |
| 6023:7006 | 6023:7007 | 6023:7008 | 6023:7009 | 6023:7010 | 6023:7011 | 6023:7012 |
| 6024:7006 | 6024:7007 | 6024:7008 | 6024:7009 | 6024:7010 | 6024:7011 | 6024:7012 |
| 6025:7006 | 6025:7007 | 6025:7008 | 6025:7009 | 6025:7010 | 6025:7011 | 6025:7012 |
| 6026:7006 | 6026:7007 | 6026:7008 | 6026:7009 | 6026:7010 | 6026:7011 | 6026:7012 |
| 6027:7006 | 6027:7007 | 6027:7008 | 6027:7009 | 6027:7010 | 6027:7011 | 6027:7012 |
| 6028:7006 | 6028:7007 | 6028:7008 | 6028:7009 | 6028:7010 | 6028:7011 | 6028:7012 |
| 6029:7006 | 6029:7007 | 6029:7008 | 6029:7009 | 6029:7010 | 6029:7011 | 6029:7012 |
| 6030:7006 | 6030:7007 | 6030:7008 | 6030:7009 | 6030:7010 | 6030:7011 | 6030:7012 |
| 6031:7006 | 6031:7007 | 6031:7008 | 6031:7009 | 6031:7010 | 6031:7011 | 6031:7012 |
| 6032:7006 | 6032:7007 | 6032:7008 | 6032:7009 | 6032:7010 | 6032:7011 | 6032:7012 |
| 6033:7006 | 6033:7007 | 6033:7008 | 6033:7009 | 6033:7010 | 6033:7011 | 6033:7012 |
| 6034:7006 | 6034:7007 | 6034:7008 | 6034:7009 | 6034:7010 | 6034:7011 | 6034:7012 |
| 6035:7006 | 6035:7007 | 6035:7008 | 6035:7009 | 6035:7010 | 6035:7011 | 6035:7012 |
| 6036:7006 | 6036:7007 | 6036:7008 | 6036:7009 | 6036:7010 | 6036:7011 | 6036:7012 |
| 6037:7006 | 6037:7007 | 6037:7008 | 6037:7009 | 6037:7010 | 6037:7011 | 6037:7012 |
| 6038:7006 | 6038:7007 | 6038:7008 | 6038:7009 | 6038:7010 | 6038:7011 | 6038:7012 |
| 6039:7006 | 6039:7007 | 6039:7008 | 6039:7009 | 6039:7010 | 6039:7011 | 6039:7012 |
| 6040:7006 | 6040:7007 | 6040:7008 | 6040:7009 | 6040:7010 | 6040:7011 | 6040:7012 |
| 6021:7013 | 6021:7014 | 6021:7015 | 6021:7016 | 6021:7017 | 6021:7018 | 6021:7019 |
| 6022:7013 | 6022:7014 | 6022:7015 | 6022:7016 | 6022:7017 | 6022:7018 | 6022:7019 |
| 6023:7013 | 6023:7014 | 6023:7015 | 6023:7016 | 6023:7017 | 6023:7018 | 6023:7019 |
| 6024:7013 | 6024:7014 | 6024:7015 | 6024:7016 | 6024:7017 | 6024:7018 | 6024:7019 |
| 6025:7013 | 6025:7014 | 6025:7015 | 6025:7016 | 6025:7017 | 6025:7018 | 6025:7019 |
| 6026:7013 | 6026:7014 | 6026:7015 | 6026:7016 | 6026:7017 | 6026:7018 | 6026:7019 |
| 6027:7013 | 6027:7014 | 6027:7015 | 6027:7016 | 6027:7017 | 6027:7018 | 6027:7019 |
| 6028:7013 | 6028:7014 | 6028:7015 | 6028:7016 | 6028:7017 | 6028:7018 | 6028:7019 |
| 6029:7013 | 6029:7014 | 6029:7015 | 6029:7016 | 6029:7017 | 6029:7018 | 6029:7019 |
| 6030:7013 | 6030:7014 | 6030:7015 | 6030:7016 | 6030:7017 | 6030:7018 | 6030:7019 |
| 6031:7013 | 6031:7014 | 6031:7015 | 6031:7016 | 6031:7017 | 6031:7018 | 6031:7019 |
| 6032:7013 | 6032:7014 | 6032:7015 | 6032:7016 | 6032:7017 | 6032:7018 | 6032:7019 |
| 6033:7013 | 6033:7014 | 6033:7015 | 6033:7016 | 6033:7017 | 6033:7018 | 6033:7019 |
| 6034:7013 | 6034:7014 | 6034:7015 | 6034:7016 | 6034:7017 | 6034:7018 | 6034:7019 |
| 6035:7013 | 6035:7014 | 6035:7015 | 6035:7016 | 6035:7017 | 6035:7018 | 6035:7019 |
| 6036:7013 | 6036:7014 | 6036:7015 | 6036:7016 | 6036:7017 | 6036:7018 | 6036:7019 |
| 6037:7013 | 6037:7014 | 6037:7015 | 6037:7016 | 6037:7017 | 6037:7018 | 6037:7019 |
| 6038:7013 | 6038:7014 | 6038:7015 | 6038:7016 | 6038:7017 | 6038:7018 | 6038:7019 |
| 6039:7013 | 6039:7014 | 6039:7015 | 6039:7016 | 6039:7017 | 6039:7018 | 6039:7019 |
| 6040:7013 | 6040:7014 | 6040:7015 | 6040:7016 | 6040:7017 | 6040:7018 | 6040:7019 |
| 6021:7020 | 6021:7021 | 6021:7022 | 6021:7023 | 6021:7024 | 6021:7025 | 6021:7026 |
| 6022:7020 | 6022:7021 | 6022:7022 | 6022:7023 | 6022:7024 | 6022:7025 | 6022:7026 |
| 6023:7020 | 6023:7021 | 6023:7022 | 6023:7023 | 6023:7024 | 6023:7025 | 6023:7026 |
| 6024:7020 | 6024:7021 | 6024:7022 | 6024:7023 | 6024:7024 | 6024:7025 | 6024:7026 |
| 6025:7020 | 6025:7021 | 6025:7022 | 6025:7023 | 6025:7024 | 6025:7025 | 6025:7026 |
| 6026:7020 | 6026:7021 | 6026:7022 | 6026:7023 | 6026:7024 | 6026:7025 | 6026:7026 |
| 6027:7020 | 6027:7021 | 6027:7022 | 6027:7023 | 6027:7024 | 6027:7025 | 6027:7026 |
| 6028:7020 | 6028:7021 | 6028:7022 | 6028:7023 | 6028:7024 | 6028:7025 | 6028:7026 |
| 6029:7020 | 6029:7021 | 6029:7022 | 6029:7023 | 6029:7024 | 6029:7025 | 6029:7026 |
| 6030:7020 | 6030:7021 | 6030:7022 | 6030:7023 | 6030:7024 | 6030:7025 | 6030:7026 |
| 6031:7020 | 6031:7021 | 6031:7022 | 6031:7023 | 6031:7024 | 6031:7025 | 6031:7026 |
| 6032:7020 | 6032:7021 | 6032:7022 | 6032:7023 | 6032:7024 | 6032:7025 | 6032:7026 |
| 6033:7020 | 6033:7021 | 6033:7022 | 6033:7023 | 6033:7024 | 6033:7025 | 6033:7026 |
| 6034:7020 | 6034:7021 | 6034:7022 | 6034:7023 | 6034:7024 | 6034:7025 | 6034:7026 |
| 6035:7020 | 6035:7021 | 6035:7022 | 6035:7023 | 6035:7024 | 6035:7025 | 6035:7026 |
| 6036:7020 | 6036:7021 | 6036:7022 | 6036:7023 | 6036:7024 | 6036:7025 | 6036:7026 |
| 6037:7020 | 6037:7021 | 6037:7022 | 6037:7023 | 6037:7024 | 6037:7025 | 6037:7026 |
| 6038:7020 | 6038:7021 | 6038:7022 | 6038:7023 | 6038:7024 | 6038:7025 | 6038:7026 |
| 6039:7020 | 6039:7021 | 6039:7022 | 6039:7023 | 6039:7024 | 6039:7025 | 6039:7026 |
| 6040:7020 | 6040:7021 | 6040:7022 | 6040:7023 | 6040:7024 | 6040:7025 | 6040:7026 |
| 6021:7027 | 6021:7028 | 6021:7029 | 6021:7030 | 6021:7031 | 6021:7032 | 6021:7033 |
| 6022:7027 | 6022:7028 | 6022:7029 | 6022:7030 | 6022:7031 | 6022:7032 | 6022:7033 |
| 6023:7027 | 6023:7028 | 6023:7029 | 6023:7030 | 6023:7031 | 6023:7032 | 6023:7033 |
| 6024:7027 | 6024:7028 | 6024:7029 | 6024:7030 | 6024:7031 | 6024:7032 | 6024:7033 |
| 6025:7027 | 6025:7028 | 6025:7029 | 6025:7030 | 6025:7031 | 6025:7032 | 6025:7033 |
| 6026:7027 | 6026:7028 | 6026:7029 | 6026:7030 | 6026:7031 | 6026:7032 | 6026:7033 |
| 6027:7027 | 6027:7028 | 6027:7029 | 6027:7030 | 6027:7031 | 6027:7032 | 6027:7033 |
| 6028:7027 | 6028:7028 | 6028:7029 | 6028:7030 | 6028:7031 | 6028:7032 | 6028:7033 |
| 6029:7027 | 6029:7028 | 6029:7029 | 6029:7030 | 6029:7031 | 6029:7032 | 6029:7033 |
| 6030:7027 | 6030:7028 | 6030:7029 | 6030:7030 | 6030:7031 | 6030:7032 | 6030:7033 |
| 6031:7027 | 6031:7028 | 6031:7029 | 6031:7030 | 6031:7031 | 6031:7032 | 6031:7033 |
| 6032:7027 | 6032:7028 | 6032:7029 | 6032:7030 | 6032:7031 | 6032:7032 | 6032:7033 |
| 6033:7027 | 6033:7028 | 6033:7029 | 6033:7030 | 6033:7031 | 6033:7032 | 6033:7033 |
| 6034:7027 | 6034:7028 | 6034:7029 | 6034:7030 | 6034:7031 | 6034:7032 | 6034:7033 |
| 6035:7027 | 6035:7028 | 6035:7029 | 6035:7030 | 6035:7031 | 6035:7032 | 6035:7033 |
| 6036:7027 | 6036:7028 | 6036:7029 | 6036:7030 | 6036:7031 | 6036:7032 | 6036:7033 |
| 6037:7027 | 6037:7028 | 6037:7029 | 6037:7030 | 6037:7031 | 6037:7032 | 6037:7033 |
| 6038:7027 | 6038:7028 | 6038:7029 | 6038:7030 | 6038:7031 | 6038:7032 | 6038:7033 |
| 6039:7027 | 6039:7028 | 6039:7029 | 6039:7030 | 6039:7031 | 6039:7032 | 6039:7033 |
| 6040:7027 | 6040:7028 | 6040:7029 | 6040:7030 | 6040:7031 | 6040:7032 | 6040:7033 |
| 6021:7034 | 6021:7035 | 6021:7036 | 6021:7037 | 6021:7038 | 6021:7039 | 6021:7040 |
| 6022:7034 | 6022:7035 | 6022:7036 | 6022:7037 | 6022:7038 | 6022:7039 | 6022:7040 |
| 6023:7034 | 6023:7035 | 6023:7036 | 6023:7037 | 6023:7038 | 6023:7039 | 6023:7040 |
| 6024:7034 | 6024:7035 | 6024:7036 | 6024:7037 | 6024:7038 | 6024:7039 | 6024:7040 |
| 6025:7034 | 6025:7035 | 6025:7036 | 6025:7037 | 6025:7038 | 6025:7039 | 6025:7040 |
| 6026:7034 | 6026:7035 | 6026:7036 | 6026:7037 | 6026:7038 | 6026:7039 | 6026:7040 |
| 6027:7034 | 6027:7035 | 6027:7036 | 6027:7037 | 6027:7038 | 6027:7039 | 6027:7040 |
| 6028:7034 | 6028:7035 | 6028:7036 | 6028:7037 | 6028:7038 | 6028:7039 | 6028:7040 |
| 6029:7034 | 6029:7035 | 6029:7036 | 6029:7037 | 6029:7038 | 6029:7039 | 6029:7040 |
| 6030:7034 | 6030:7035 | 6030:7036 | 6030:7037 | 6030:7038 | 6030:7039 | 6030:7040 |
| 6031:7034 | 6031:7035 | 6031:7036 | 6031:7037 | 6031:7038 | 6031:7039 | 6031:7040 |
| 6032:7034 | 6032:7035 | 6032:7036 | 6032:7037 | 6032:7038 | 6032:7039 | 6032:7040 |
| 6033:7034 | 6033:7035 | 6033:7036 | 6033:7037 | 6033:7038 | 6033:7039 | 6033:7040 |
| 6034:7034 | 6034:7035 | 6034:7036 | 6034:7037 | 6034:7038 | 6034:7039 | 6034:7040 |
| 6035:7034 | 6035:7035 | 6035:7036 | 6035:7037 | 6035:7038 | 6035:7039 | 6035:7040 |
| 6036:7034 | 6036:7035 | 6036:7036 | 6036:7037 | 6036:7038 | 6036:7039 | 6036:7040 |
| 6037:7034 | 6037:7035 | 6037:7036 | 6037:7037 | 6037:7038 | 6037:7039 | 6037:7040 |
| 6038:7034 | 6038:7035 | 6038:7036 | 6038:7037 | 6038:7038 | 6038:7039 | 6038:7040 |
| 6039:7034 | 6039:7035 | 6039:7036 | 6039:7037 | 6039:7038 | 6039:7039 | 6039:7040 |
| 6040:7034 | 6040:7035 | 6040:7036 | 6040:7037 | 6040:7038 | 6040:7039 | 6040:7040 |
| 6021:7041 | 6021:7042 | 6021:7043 | 6021:7044 | 6021:7045 | 6021:7046 | 6021:7047 |
| 6022:7041 | 6022:7042 | 6022:7043 | 6022:7044 | 6022:7045 | 6022:7046 | 6022:7047 |
| 6023:7041 | 6023:7042 | 6023:7043 | 6023:7044 | 6023:7045 | 6023:7046 | 6023:7047 |
| 6024:7041 | 6024:7042 | 6024:7043 | 6024:7044 | 6024:7045 | 6024:7046 | 6024:7047 |
| 6025:7041 | 6025:7042 | 6025:7043 | 6025:7044 | 6025:7045 | 6025:7046 | 6025:7047 |
| 6026:7041 | 6026:7042 | 6026:7043 | 6026:7044 | 6026:7045 | 6026:7046 | 6026:7047 |
| 6027:7041 | 6027:7042 | 6027:7043 | 6027:7044 | 6027:7045 | 6027:7046 | 6027:7047 |
| 6028:7041 | 6028:7042 | 6028:7043 | 6028:7044 | 6028:7045 | 6028:7046 | 6028:7047 |
| 6029:7041 | 6029:7042 | 6029:7043 | 6029:7044 | 6029:7045 | 6029:7046 | 6029:7047 |
| 6030:7041 | 6030:7042 | 6030:7043 | 6030:7044 | 6030:7045 | 6030:7046 | 6030:7047 |
| 6031:7041 | 6031:7042 | 6031:7043 | 6031:7044 | 6031:7045 | 6031:7046 | 6031:7047 |
| 6032:7041 | 6032:7042 | 6032:7043 | 6032:7044 | 6032:7045 | 6032:7046 | 6032:7047 |
| 6033:7041 | 6033:7042 | 6033:7043 | 6033:7044 | 6033:7045 | 6033:7046 | 6033:7047 |
| 6034:7041 | 6034:7042 | 6034:7043 | 6034:7044 | 6034:7045 | 6034:7046 | 6034:7047 |
| 6035:7041 | 6035:7042 | 6035:7043 | 6035:7044 | 6035:7045 | 6035:7046 | 6035:7047 |
| 6036:7041 | 6036:7042 | 6036:7043 | 6036:7044 | 6036:7045 | 6036:7046 | 6036:7047 |
| 6037:7041 | 6037:7042 | 6037:7043 | 6037:7044 | 6037:7045 | 6037:7046 | 6037:7047 |
| 6038:7041 | 6038:7042 | 6038:7043 | 6038:7044 | 6038:7045 | 6038:7046 | 6038:7047 |
| 6039:7041 | 6039:7042 | 6039:7043 | 6039:7044 | 6039:7045 | 6039:7046 | 6039:7047 |
| 6040:7041 | 6040:7042 | 6040:7043 | 6040:7044 | 6040:7045 | 6040:7046 | 6040:7047 |
| 6021:7048 | 6021:7049 | 6021:7050 | 6021:7051 | 6021:7052 | 6021:7053 | 6021:7054 |
| 6022:7048 | 6022:7049 | 6022:7050 | 6022:7051 | 6022:7052 | 6022:7053 | 6022:7054 |
| 6023:7048 | 6023:7049 | 6023:7050 | 6023:7051 | 6023:7052 | 6023:7053 | 6023:7054 |
| 6024:7048 | 6024:7049 | 6024:7050 | 6024:7051 | 6024:7052 | 6024:7053 | 6024:7054 |
| 6025:7048 | 6025:7049 | 6025:7050 | 6025:7051 | 6025:7052 | 6025:7053 | 6025:7054 |
| 6026:7048 | 6026:7049 | 6026:7050 | 6026:7051 | 6026:7052 | 6026:7053 | 6026:7054 |
| 6027:7048 | 6027:7049 | 6027:7050 | 6027:7051 | 6027:7052 | 6027:7053 | 6027:7054 |
| 6028:7048 | 6028:7049 | 6028:7050 | 6028:7051 | 6028:7052 | 6028:7053 | 6028:7054 |
| 6029:7048 | 6029:7049 | 6029:7050 | 6029:7051 | 6029:7052 | 6029:7053 | 6029:7054 |
| 6030:7048 | 6030:7049 | 6030:7050 | 6030:7051 | 6030:7052 | 6030:7053 | 6030:7054 |
| 6031:7048 | 6031:7049 | 6031:7050 | 6031:7051 | 6031:7052 | 6031:7053 | 6031:7054 |
| 6032:7048 | 6032:7049 | 6032:7050 | 6032:7051 | 6032:7052 | 6032:7053 | 6032:7054 |
| 6033:7048 | 6033:7049 | 6033:7050 | 6033:7051 | 6033:7052 | 6033:7053 | 6033:7054 |
| 6034:7048 | 6034:7049 | 6034:7050 | 6034:7051 | 6034:7052 | 6034:7053 | 6034:7054 |
| 6035:7048 | 6035:7049 | 6035:7050 | 6035:7051 | 6035:7052 | 6035:7053 | 6035:7054 |
| 6036:7048 | 6036:7049 | 6036:7050 | 6036:7051 | 6036:7052 | 6036:7053 | 6036:7054 |
| 6037:7048 | 6037:7049 | 6037:7050 | 6037:7051 | 6037:7052 | 6037:7053 | 6037:7054 |
| 6038:7048 | 6038:7049 | 6038:7050 | 6038:7051 | 6038:7052 | 6038:7053 | 6038:7054 |
| 6039:7048 | 6039:7049 | 6039:7050 | 6039:7051 | 6039:7052 | 6039:7053 | 6039:7054 |
| 6040:7048 | 6040:7049 | 6040:7050 | 6040:7051 | 6040:7052 | 6040:7053 | 6040:7054 |
| 6021:7055 | 6021:7056 | 6021:7057 | 6021:7058 | 6021:7059 | 6021:7060 | 6021:7061 |
| 6022:7055 | 6022:7056 | 6022:7057 | 6022:7058 | 6022:7059 | 6022:7060 | 6022:7061 |
| 6023:7055 | 6023:7056 | 6023:7057 | 6023:7058 | 6023:7059 | 6023:7060 | 6023:7061 |
| 6024:7055 | 6024:7056 | 6024:7057 | 6024:7058 | 6024:7059 | 6024:7060 | 6024:7061 |
| 6025:7055 | 6025:7056 | 6025:7057 | 6025:7058 | 6025:7059 | 6025:7060 | 6025:7061 |
| 6026:7055 | 6026:7056 | 6026:7057 | 6026:7058 | 6026:7059 | 6026:7060 | 6026:7061 |
| 6027:7055 | 6027:7056 | 6027:7057 | 6027:7058 | 6027:7059 | 6027:7060 | 6027:7061 |
| 6028:7055 | 6028:7056 | 6028:7057 | 6028:7058 | 6028:7059 | 6028:7060 | 6028:7061 |
| 6029:7055 | 6029:7056 | 6029:7057 | 6029:7058 | 6029:7059 | 6029:7060 | 6029:7061 |
| 6030:7055 | 6030:7056 | 6030:7057 | 6030:7058 | 6030:7059 | 6030:7060 | 6030:7061 |
| 6031:7055 | 6031:7056 | 6031:7057 | 6031:7058 | 6031:7059 | 6031:7060 | 6031:7061 |
| 6032:7055 | 6032:7056 | 6032:7057 | 6032:7058 | 6032:7059 | 6032:7060 | 6032:7061 |
| 6033:7055 | 6033:7056 | 6033:7057 | 6033:7058 | 6033:7059 | 6033:7060 | 6033:7061 |
| 6034:7055 | 6034:7056 | 6034:7057 | 6034:7058 | 6034:7059 | 6034:7060 | 6034:7061 |
| 6035:7055 | 6035:7056 | 6035:7057 | 6035:7058 | 6035:7059 | 6035:7060 | 6035:7061 |
| 6036:7055 | 6036:7056 | 6036:7057 | 6036:7058 | 6036:7059 | 6036:7060 | 6036:7061 |
| 6037:7055 | 6037:7056 | 6037:7057 | 6037:7058 | 6037:7059 | 6037:7060 | 6037:7061 |
| 6038:7055 | 6038:7056 | 6038:7057 | 6038:7058 | 6038:7059 | 6038:7060 | 6038:7061 |
| 6039:7055 | 6039:7056 | 6039:7057 | 6039:7058 | 6039:7059 | 6039:7060 | 6039:7061 |
| 6040:7055 | 6040:7056 | 6040:7057 | 6040:7058 | 6040:7059 | 6040:7060 | 6040:7061 |
| 6021:7062 | 6021:7063 | 6021:7064 | 6021:7065 | 6021:7066 | 6021:7067 | 6021:7068 |
| 6022:7062 | 6022:7063 | 6022:7064 | 6022:7065 | 6022:7066 | 6022:7067 | 6022:7068 |
| 6023:7062 | 6023:7063 | 6023:7064 | 6023:7065 | 6023:7066 | 6023:7067 | 6023:7068 |
| 6024:7062 | 6024:7063 | 6024:7064 | 6024:7065 | 6024:7066 | 6024:7067 | 6024:7068 |
| 6025:7062 | 6025:7063 | 6025:7064 | 6025:7065 | 6025:7066 | 6025:7067 | 6025:7068 |
| 6026:7062 | 6026:7063 | 6026:7064 | 6026:7065 | 6026:7066 | 6026:7067 | 6026:7068 |
| 6027:7062 | 6027:7063 | 6027:7064 | 6027:7065 | 6027:7066 | 6027:7067 | 6027:7068 |
| 6028:7062 | 6028:7063 | 6028:7064 | 6028:7065 | 6028:7066 | 6028:7067 | 6028:7068 |
| 6029:7062 | 6029:7063 | 6029:7064 | 6029:7065 | 6029:7066 | 6029:7067 | 6029:7068 |
| 6030:7062 | 6030:7063 | 6030:7064 | 6030:7065 | 6030:7066 | 6030:7067 | 6030:7068 |
| 6031:7062 | 6031:7063 | 6031:7064 | 6031:7065 | 6031:7066 | 6031:7067 | 6031:7068 |
| 6032:7062 | 6032:7063 | 6032:7064 | 6032:7065 | 6032:7066 | 6032:7067 | 6032:7068 |
| 6033:7062 | 6033:7063 | 6033:7064 | 6033:7065 | 6033:7066 | 6033:7067 | 6033:7068 |
| 6034:7062 | 6034:7063 | 6034:7064 | 6034:7065 | 6034:7066 | 6034:7067 | 6034:7068 |
| 6035:7062 | 6035:7063 | 6035:7064 | 6035:7065 | 6035:7066 | 6035:7067 | 6035:7068 |
| 6036:7062 | 6036:7063 | 6036:7064 | 6036:7065 | 6036:7066 | 6036:7067 | 6036:7068 |
| 6037:7062 | 6037:7063 | 6037:7064 | 6037:7065 | 6037:7066 | 6037:7067 | 6037:7068 |
| 6038:7062 | 6038:7063 | 6038:7064 | 6038:7065 | 6038:7066 | 6038:7067 | 6038:7068 |
| 6039:7062 | 6039:7063 | 6039:7064 | 6039:7065 | 6039:7066 | 6039:7067 | 6039:7068 |
| 6040:7062 | 6040:7063 | 6040:7064 | 6040:7065 | 6040:7066 | 6040:7067 | 6040:7068 |
| 6021:7069 | 6021:7070 | 6021:7071 | 6021:7072 | 6021:7073 | 6021:7074 | 6021:7075 |
| 6022:7069 | 6022:7070 | 6022:7071 | 6022:7072 | 6022:7073 | 6022:7074 | 6022:7075 |
| 6023:7069 | 6023:7070 | 6023:7071 | 6023:7072 | 6023:7073 | 6023:7074 | 6023:7075 |
| 6024:7069 | 6024:7070 | 6024:7071 | 6024:7072 | 6024:7073 | 6024:7074 | 6024:7075 |
| 6025:7069 | 6025:7070 | 6025:7071 | 6025:7072 | 6025:7073 | 6025:7074 | 6025:7075 |
| 6026:7069 | 6026:7070 | 6026:7071 | 6026:7072 | 6026:7073 | 6026:7074 | 6026:7075 |
| 6027:7069 | 6027:7070 | 6027:7071 | 6027:7072 | 6027:7073 | 6027:7074 | 6027:7075 |
| 6028:7069 | 6028:7070 | 6028:7071 | 6028:7072 | 6028:7073 | 6028:7074 | 6028:7075 |
| 6029:7069 | 6029:7070 | 6029:7071 | 6029:7072 | 6029:7073 | 6029:7074 | 6029:7075 |
| 6030:7069 | 6030:7070 | 6030:7071 | 6030:7072 | 6030:7073 | 6030:7074 | 6030:7075 |
| 6031:7069 | 6031:7070 | 6031:7071 | 6031:7072 | 6031:7073 | 6031:7074 | 6031:7075 |
| 6032:7069 | 6032:7070 | 6032:7071 | 6032:7072 | 6032:7073 | 6032:7074 | 6032:7075 |
| 6033:7069 | 6033:7070 | 6033:7071 | 6033:7072 | 6033:7073 | 6033:7074 | 6033:7075 |
| 6034:7069 | 6034:7070 | 6034:7071 | 6034:7072 | 6034:7073 | 6034:7074 | 6034:7075 |
| 6035:7069 | 6035:7070 | 6035:7071 | 6035:7072 | 6035:7073 | 6035:7074 | 6035:7075 |
| 6036:7069 | 6036:7070 | 6036:7071 | 6036:7072 | 6036:7073 | 6036:7074 | 6036:7075 |
| 6037:7069 | 6037:7070 | 6037:7071 | 6037:7072 | 6037:7073 | 6037:7074 | 6037:7075 |
| 6038:7069 | 6038:7070 | 6038:7071 | 6038:7072 | 6038:7073 | 6038:7074 | 6038:7075 |
| 6039:7069 | 6039:7070 | 6039:7071 | 6039:7072 | 6039:7073 | 6039:7074 | 6039:7075 |
| 6040:7069 | 6040:7070 | 6040:7071 | 6040:7072 | 6040:7073 | 6040:7074 | 6040:7075 |
| 6021:7076 | 6021:7077 | 6041:7000 | 6041:7001 | 6041:7002 | 6041:7003 | 6041:7004 |
| 6022:7076 | 6022:7077 | 6042:7000 | 6042:7001 | 6042:7002 | 6042:7003 | 6042:7004 |
| 6023:7076 | 6023:7077 | 6043:7000 | 6043:7001 | 6043:7002 | 6043:7003 | 6043:7004 |
| 6024:7076 | 6024:7077 | 6044:7000 | 6044:7001 | 6044:7002 | 6044:7003 | 6044:7004 |
| 6025:7076 | 6025:7077 | 6045:7000 | 6045:7001 | 6045:7002 | 6045:7003 | 6045:7004 |
| 6026:7076 | 6026:7077 | 6046:7000 | 6046:7001 | 6046:7002 | 6046:7003 | 6046:7004 |
| 6027:7076 | 6027:7077 | 6047:7000 | 6047:7001 | 6047:7002 | 6047:7003 | 6047:7004 |
| 6028:7076 | 6028:7077 | 6048:7000 | 6048:7001 | 6048:7002 | 6048:7003 | 6048:7004 |
| 6029:7076 | 6029:7077 | 6049:7000 | 6049:7001 | 6049:7002 | 6049:7003 | 6049:7004 |
| 6030:7076 | 6030:7077 | 6050:7000 | 6050:7001 | 6050:7002 | 6050:7003 | 6050:7004 |
| 6031:7076 | 6031:7077 | 6051:7000 | 6051:7001 | 6051:7002 | 6051:7003 | 6051:7004 |
| 6032:7076 | 6032:7077 | 6052:7000 | 6052:7001 | 6052:7002 | 6052:7003 | 6052:7004 |
| 6033:7076 | 6033:7077 | 6053:7000 | 6053:7001 | 6053:7002 | 6053:7003 | 6053:7004 |
| 6034:7076 | 6034:7077 | 6054:7000 | 6054:7001 | 6054:7002 | 6054:7003 | 6054:7004 |
| 6035:7076 | 6035:7077 | 6055:7000 | 6055:7001 | 6055:7002 | 6055:7003 | 6055:7004 |
| 6036:7076 | 6036:7077 | 6056:7000 | 6056:7001 | 6056:7002 | 6056:7003 | 6056:7004 |
| 6037:7076 | 6037:7077 | 6057:7000 | 6057:7001 | 6057:7002 | 6057:7003 | 6057:7004 |
| 6038:7076 | 6038:7077 | 6058:7000 | 6058:7001 | 6058:7002 | 6058:7003 | 6058:7004 |
| 6039:7076 | 6039:7077 | 6059:7000 | 6059:7001 | 6059:7002 | 6059:7003 | 6059:7004 |
| 6040:7076 | 6040:7077 | 6060:7000 | 6060:7001 | 6060:7002 | 6060:7003 | 6060:7004 |
| 6041:7005 | 6041:7006 | 6041:7007 | 6041:7008 | 6041:7009 | 6041:7010 | 6041:7011 |
| 6042:7005 | 6042:7006 | 6042:7007 | 6042:7008 | 6042:7009 | 6042:7010 | 6042:7011 |
| 6043:7005 | 6043:7006 | 6043:7007 | 6043:7008 | 6043:7009 | 6043:7010 | 6043:7011 |
| 6044:7005 | 6044:7006 | 6044:7007 | 6044:7008 | 6044:7009 | 6044:7010 | 6044:7011 |
| 6045:7005 | 6045:7006 | 6045:7007 | 6045:7008 | 6045:7009 | 6045:7010 | 6045:7011 |
| 6046:7005 | 6046:7006 | 6046:7007 | 6046:7008 | 6046:7009 | 6046:7010 | 6046:7011 |
| 6047:7005 | 6047:7006 | 6047:7007 | 6047:7008 | 6047:7009 | 6047:7010 | 6047:7011 |
| 6048:7005 | 6048:7006 | 6048:7007 | 6048:7008 | 6048:7009 | 6048:7010 | 6048:7011 |
| 6049:7005 | 6049:7006 | 6049:7007 | 6049:7008 | 6049:7009 | 6049:7010 | 6049:7011 |
| 6050:7005 | 6050:7006 | 6050:7007 | 6050:7008 | 6050:7009 | 6050:7010 | 6050:7011 |
| 6051:7005 | 6051:7006 | 6051:7007 | 6051:7008 | 6051:7009 | 6051:7010 | 6051:7011 |
| 6052:7005 | 6052:7006 | 6052:7007 | 6052:7008 | 6052:7009 | 6052:7010 | 6052:7011 |
| 6053:7005 | 6053:7006 | 6053:7007 | 6053:7008 | 6053:7009 | 6053:7010 | 6053:7011 |
| 6054:7005 | 6054:7006 | 6054:7007 | 6054:7008 | 6054:7009 | 6054:7010 | 6054:7011 |
| 6055:7005 | 6055:7006 | 6055:7007 | 6055:7008 | 6055:7009 | 6055:7010 | 6055:7011 |
| 6056:7005 | 6056:7006 | 6056:7007 | 6056:7008 | 6056:7009 | 6056:7010 | 6056:7011 |
| 6057:7005 | 6057:7006 | 6057:7007 | 6057:7008 | 6057:7009 | 6057:7010 | 6057:7011 |
| 6058:7005 | 6058:7006 | 6058:7007 | 6058:7008 | 6058:7009 | 6058:7010 | 6058:7011 |
| 6059:7005 | 6059:7006 | 6059:7007 | 6059:7008 | 6059:7009 | 6059:7010 | 6059:7011 |
| 6060:7005 | 6060:7006 | 6060:7007 | 6060:7008 | 6060:7009 | 6060:7010 | 6060:7011 |
| 6041:7012 | 6041:7013 | 6041:7014 | 6041:7015 | 6041:7016 | 6041:7017 | 6041:7018 |
| 6042:7012 | 6042:7013 | 6042:7014 | 6042:7015 | 6042:7016 | 6042:7017 | 6042:7018 |
| 6043:7012 | 6043:7013 | 6043:7014 | 6043:7015 | 6043:7016 | 6043:7017 | 6043:7018 |
| 6044:7012 | 6044:7013 | 6044:7014 | 6044:7015 | 6044:7016 | 6044:7017 | 6044:7018 |
| 6045:7012 | 6045:7013 | 6045:7014 | 6045:7015 | 6045:7016 | 6045:7017 | 6045:7018 |
| 6046:7012 | 6046:7013 | 6046:7014 | 6046:7015 | 6046:7016 | 6046:7017 | 6046:7018 |
| 6047:7012 | 6047:7013 | 6047:7014 | 6047:7015 | 6047:7016 | 6047:7017 | 6047:7018 |
| 6048:7012 | 6048:7013 | 6048:7014 | 6048:7015 | 6048:7016 | 6048:7017 | 6048:7018 |
| 6049:7012 | 6049:7013 | 6049:7014 | 6049:7015 | 6049:7016 | 6049:7017 | 6049:7018 |
| 6050:7012 | 6050:7013 | 6050:7014 | 6050:7015 | 6050:7016 | 6050:7017 | 6050:7018 |
| 6051:7012 | 6051:7013 | 6051:7014 | 6051:7015 | 6051:7016 | 6051:7017 | 6051:7018 |
| 6052:7012 | 6052:7013 | 6052:7014 | 6052:7015 | 6052:7016 | 6052:7017 | 6052:7018 |
| 6053:7012 | 6053:7013 | 6053:7014 | 6053:7015 | 6053:7016 | 6053:7017 | 6053:7018 |
| 6054:7012 | 6054:7013 | 6054:7014 | 6054:7015 | 6054:7016 | 6054:7017 | 6054:7018 |
| 6055:7012 | 6055:7013 | 6055:7014 | 6055:7015 | 6055:7016 | 6055:7017 | 6055:7018 |
| 6056:7012 | 6056:7013 | 6056:7014 | 6056:7015 | 6056:7016 | 6056:7017 | 6056:7018 |
| 6057:7012 | 6057:7013 | 6057:7014 | 6057:7015 | 6057:7016 | 6057:7017 | 6057:7018 |
| 6058:7012 | 6058:7013 | 6058:7014 | 6058:7015 | 6058:7016 | 6058:7017 | 6058:7018 |
| 6059:7012 | 6059:7013 | 6059:7014 | 6059:7015 | 6059:7016 | 6059:7017 | 6059:7018 |
| 6060:7012 | 6060:7013 | 6060:7014 | 6060:7015 | 6060:7016 | 6060:7017 | 6060:7018 |
| 6041:7019 | 6041:7020 | 6041:7021 | 6041:7022 | 6041:7023 | 6041:7024 | 6041:7025 |
| 6042:7019 | 6042:7020 | 6042:7021 | 6042:7022 | 6042:7023 | 6042:7024 | 6042:7025 |
| 6043:7019 | 6043:7020 | 6043:7021 | 6043:7022 | 6043:7023 | 6043:7024 | 6043:7025 |
| 6044:7019 | 6044:7020 | 6044:7021 | 6044:7022 | 6044:7023 | 6044:7024 | 6044:7025 |
| 6045:7019 | 6045:7020 | 6045:7021 | 6045:7022 | 6045:7023 | 6045:7024 | 6045:7025 |
| 6046:7019 | 6046:7020 | 6046:7021 | 6046:7022 | 6046:7023 | 6046:7024 | 6046:7025 |
| 6047:7019 | 6047:7020 | 6047:7021 | 6047:7022 | 6047:7023 | 6047:7024 | 6047:7025 |
| 6048:7019 | 6048:7020 | 6048:7021 | 6048:7022 | 6048:7023 | 6048:7024 | 6048:7025 |
| 6049:7019 | 6049:7020 | 6049:7021 | 6049:7022 | 6049:7023 | 6049:7024 | 6049:7025 |
| 6050:7019 | 6050:7020 | 6050:7021 | 6050:7022 | 6050:7023 | 6050:7024 | 6050:7025 |
| 6051:7019 | 6051:7020 | 6051:7021 | 6051:7022 | 6051:7023 | 6051:7024 | 6051:7025 |
| 6052:7019 | 6052:7020 | 6052:7021 | 6052:7022 | 6052:7023 | 6052:7024 | 6052:7025 |
| 6053:7019 | 6053:7020 | 6053:7021 | 6053:7022 | 6053:7023 | 6053:7024 | 6053:7025 |
| 6054:7019 | 6054:7020 | 6054:7021 | 6054:7022 | 6054:7023 | 6054:7024 | 6054:7025 |
| 6055:7019 | 6055:7020 | 6055:7021 | 6055:7022 | 6055:7023 | 6055:7024 | 6055:7025 |
| 6056:7019 | 6056:7020 | 6056:7021 | 6056:7022 | 6056:7023 | 6056:7024 | 6056:7025 |
| 6057:7019 | 6057:7020 | 6057:7021 | 6057:7022 | 6057:7023 | 6057:7024 | 6057:7025 |
| 6058:7019 | 6058:7020 | 6058:7021 | 6058:7022 | 6058:7023 | 6058:7024 | 6058:7025 |
| 6059:7019 | 6059:7020 | 6059:7021 | 6059:7022 | 6059:7023 | 6059:7024 | 6059:7025 |
| 6060:7019 | 6060:7020 | 6060:7021 | 6060:7022 | 6060:7023 | 6060:7024 | 6060:7025 |
| 6041:7026 | 6041:7027 | 6041:7028 | 6041:7029 | 6041:7030 | 6041:7031 | 6041:7032 |
| 6042:7026 | 6042:7027 | 6042:7028 | 6042:7029 | 6042:7030 | 6042:7031 | 6042:7032 |
| 6043:7026 | 6043:7027 | 6043:7028 | 6043:7029 | 6043:7030 | 6043:7031 | 6043:7032 |
| 6044:7026 | 6044:7027 | 6044:7028 | 6044:7029 | 6044:7030 | 6044:7031 | 6044:7032 |
| 6045:7026 | 6045:7027 | 6045:7028 | 6045:7029 | 6045:7030 | 6045:7031 | 6045:7032 |
| 6046:7026 | 6046:7027 | 6046:7028 | 6046:7029 | 6046:7030 | 6046:7031 | 6046:7032 |
| 6047:7026 | 6047:7027 | 6047:7028 | 6047:7029 | 6047:7030 | 6047:7031 | 6047:7032 |
| 6048:7026 | 6048:7027 | 6048:7028 | 6048:7029 | 6048:7030 | 6048:7031 | 6048:7032 |
| 6049:7026 | 6049:7027 | 6049:7028 | 6049:7029 | 6049:7030 | 6049:7031 | 6049:7032 |
| 6050:7026 | 6050:7027 | 6050:7028 | 6050:7029 | 6050:7030 | 6050:7031 | 6050:7032 |
| 6051:7026 | 6051:7027 | 6051:7028 | 6051:7029 | 6051:7030 | 6051:7031 | 6051:7032 |
| 6052:7026 | 6052:7027 | 6052:7028 | 6052:7029 | 6052:7030 | 6052:7031 | 6052:7032 |
| 6053:7026 | 6053:7027 | 6053:7028 | 6053:7029 | 6053:7030 | 6053:7031 | 6053:7032 |
| 6054:7026 | 6054:7027 | 6054:7028 | 6054:7029 | 6054:7030 | 6054:7031 | 6054:7032 |
| 6055:7026 | 6055:7027 | 6055:7028 | 6055:7029 | 6055:7030 | 6055:7031 | 6055:7032 |
| 6056:7026 | 6056:7027 | 6056:7028 | 6056:7029 | 6056:7030 | 6056:7031 | 6056:7032 |
| 6057:7026 | 6057:7027 | 6057:7028 | 6057:7029 | 6057:7030 | 6057:7031 | 6057:7032 |
| 6058:7026 | 6058:7027 | 6058:7028 | 6058:7029 | 6058:7030 | 6058:7031 | 6058:7032 |
| 6059:7026 | 6059:7027 | 6059:7028 | 6059:7029 | 6059:7030 | 6059:7031 | 6059:7032 |
| 6060:7026 | 6060:7027 | 6060:7028 | 6060:7029 | 6060:7030 | 6060:7031 | 6060:7032 |
| 6041:7033 | 6041:7034 | 6041:7035 | 6041:7036 | 6041:7037 | 6041:7038 | 6041:7039 |
| 6042:7033 | 6042:7034 | 6042:7035 | 6042:7036 | 6042:7037 | 6042:7038 | 6042:7039 |
| 6043:7033 | 6043:7034 | 6043:7035 | 6043:7036 | 6043:7037 | 6043:7038 | 6043:7039 |
| 6044:7033 | 6044:7034 | 6044:7035 | 6044:7036 | 6044:7037 | 6044:7038 | 6044:7039 |
| 6045:7033 | 6045:7034 | 6045:7035 | 6045:7036 | 6045:7037 | 6045:7038 | 6045:7039 |
| 6046:7033 | 6046:7034 | 6046:7035 | 6046:7036 | 6046:7037 | 6046:7038 | 6046:7039 |
| 6047:7033 | 6047:7034 | 6047:7035 | 6047:7036 | 6047:7037 | 6047:7038 | 6047:7039 |
| 6048:7033 | 6048:7034 | 6048:7035 | 6048:7036 | 6048:7037 | 6048:7038 | 6048:7039 |
| 6049:7033 | 6049:7034 | 6049:7035 | 6049:7036 | 6049:7037 | 6049:7038 | 6049:7039 |
| 6050:7033 | 6050:7034 | 6050:7035 | 6050:7036 | 6050:7037 | 6050:7038 | 6050:7039 |
| 6051:7033 | 6051:7034 | 6051:7035 | 6051:7036 | 6051:7037 | 6051:7038 | 6051:7039 |
| 6052:7033 | 6052:7034 | 6052:7035 | 6052:7036 | 6052:7037 | 6052:7038 | 6052:7039 |
| 6053:7033 | 6053:7034 | 6053:7035 | 6053:7036 | 6053:7037 | 6053:7038 | 6053:7039 |
| 6054:7033 | 6054:7034 | 6054:7035 | 6054:7036 | 6054:7037 | 6054:7038 | 6054:7039 |
| 6055:7033 | 6055:7034 | 6055:7035 | 6055:7036 | 6055:7037 | 6055:7038 | 6055:7039 |
| 6056:7033 | 6056:7034 | 6056:7035 | 6056:7036 | 6056:7037 | 6056:7038 | 6056:7039 |
| 6057:7033 | 6057:7034 | 6057:7035 | 6057:7036 | 6057:7037 | 6057:7038 | 6057:7039 |
| 6058:7033 | 6058:7034 | 6058:7035 | 6058:7036 | 6058:7037 | 6058:7038 | 6058:7039 |
| 6059:7033 | 6059:7034 | 6059:7035 | 6059:7036 | 6059:7037 | 6059:7038 | 6059:7039 |
| 6060:7033 | 6060:7034 | 6060:7035 | 6060:7036 | 6060:7037 | 6060:7038 | 6060:7039 |
| 6041:7040 | 6041:7041 | 6041:7042 | 6041:7043 | 6041:7044 | 6041:7045 | 6041:7046 |
| 6042:7040 | 6042:7041 | 6042:7042 | 6042:7043 | 6042:7044 | 6042:7045 | 6042:7046 |
| 6043:7040 | 6043:7041 | 6043:7042 | 6043:7043 | 6043:7044 | 6043:7045 | 6043:7046 |
| 6044:7040 | 6044:7041 | 6044:7042 | 6044:7043 | 6044:7044 | 6044:7045 | 6044:7046 |
| 6045:7040 | 6045:7041 | 6045:7042 | 6045:7043 | 6045:7044 | 6045:7045 | 6045:7046 |
| 6046:7040 | 6046:7041 | 6046:7042 | 6046:7043 | 6046:7044 | 6046:7045 | 6046:7046 |
| 6047:7040 | 6047:7041 | 6047:7042 | 6047:7043 | 6047:7044 | 6047:7045 | 6047:7046 |
| 6048:7040 | 6048:7041 | 6048:7042 | 6048:7043 | 6048:7044 | 6048:7045 | 6048:7046 |
| 6049:7040 | 6049:7041 | 6049:7042 | 6049:7043 | 6049:7044 | 6049:7045 | 6049:7046 |
| 6050:7040 | 6050:7041 | 6050:7042 | 6050:7043 | 6050:7044 | 6050:7045 | 6050:7046 |
| 6051:7040 | 6051:7041 | 6051:7042 | 6051:7043 | 6051:7044 | 6051:7045 | 6051:7046 |
| 6052:7040 | 6052:7041 | 6052:7042 | 6052:7043 | 6052:7044 | 6052:7045 | 6052:7046 |
| 6053:7040 | 6053:7041 | 6053:7042 | 6053:7043 | 6053:7044 | 6053:7045 | 6053:7046 |
| 6054:7040 | 6054:7041 | 6054:7042 | 6054:7043 | 6054:7044 | 6054:7045 | 6054:7046 |
| 6055:7040 | 6055:7041 | 6055:7042 | 6055:7043 | 6055:7044 | 6055:7045 | 6055:7046 |
| 6056:7040 | 6056:7041 | 6056:7042 | 6056:7043 | 6056:7044 | 6056:7045 | 6056:7046 |
| 6057:7040 | 6057:7041 | 6057:7042 | 6057:7043 | 6057:7044 | 6057:7045 | 6057:7046 |
| 6058:7040 | 6058:7041 | 6058:7042 | 6058:7043 | 6058:7044 | 6058:7045 | 6058:7046 |
| 6059:7040 | 6059:7041 | 6059:7042 | 6059:7043 | 6059:7044 | 6059:7045 | 6059:7046 |
| 6060:7040 | 6060:7041 | 6060:7042 | 6060:7043 | 6060:7044 | 6060:7045 | 6060:7046 |
| 6041:7047 | 6041:7048 | 6041:7049 | 6041:7050 | 6041:7051 | 6041:7052 | 6041:7053 |
| 6042:7047 | 6042:7048 | 6042:7049 | 6042:7050 | 6042:7051 | 6042:7052 | 6042:7053 |
| 6043:7047 | 6043:7048 | 6043:7049 | 6043:7050 | 6043:7051 | 6043:7052 | 6043:7053 |
| 6044:7047 | 6044:7048 | 6044:7049 | 6044:7050 | 6044:7051 | 6044:7052 | 6044:7053 |
| 6045:7047 | 6045:7048 | 6045:7049 | 6045:7050 | 6045:7051 | 6045:7052 | 6045:7053 |
| 6046:7047 | 6046:7048 | 6046:7049 | 6046:7050 | 6046:7051 | 6046:7052 | 6046:7053 |
| 6047:7047 | 6047:7048 | 6047:7049 | 6047:7050 | 6047:7051 | 6047:7052 | 6047:7053 |
| 6048:7047 | 6048:7048 | 6048:7049 | 6048:7050 | 6048:7051 | 6048:7052 | 6048:7053 |
| 6049:7047 | 6049:7048 | 6049:7049 | 6049:7050 | 6049:7051 | 6049:7052 | 6049:7053 |
| 6050:7047 | 6050:7048 | 6050:7049 | 6050:7050 | 6050:7051 | 6050:7052 | 6050:7053 |
| 6051:7047 | 6051:7048 | 6051:7049 | 6051:7050 | 6051:7051 | 6051:7052 | 6051:7053 |
| 6052:7047 | 6052:7048 | 6052:7049 | 6052:7050 | 6052:7051 | 6052:7052 | 6052:7053 |
| 6053:7047 | 6053:7048 | 6053:7049 | 6053:7050 | 6053:7051 | 6053:7052 | 6053:7053 |
| 6054:7047 | 6054:7048 | 6054:7049 | 6054:7050 | 6054:7051 | 6054:7052 | 6054:7053 |
| 6055:7047 | 6055:7048 | 6055:7049 | 6055:7050 | 6055:7051 | 6055:7052 | 6055:7053 |
| 6056:7047 | 6056:7048 | 6056:7049 | 6056:7050 | 6056:7051 | 6056:7052 | 6056:7053 |
| 6057:7047 | 6057:7048 | 6057:7049 | 6057:7050 | 6057:7051 | 6057:7052 | 6057:7053 |
| 6058:7047 | 6058:7048 | 6058:7049 | 6058:7050 | 6058:7051 | 6058:7052 | 6058:7053 |
| 6059:7047 | 6059:7048 | 6059:7049 | 6059:7050 | 6059:7051 | 6059:7052 | 6059:7053 |
| 6060:7047 | 6060:7048 | 6060:7049 | 6060:7050 | 6060:7051 | 6060:7052 | 6060:7053 |
| 6041:7054 | 6041:7055 | 6041:7056 | 6041:7057 | 6041:7058 | 6041:7059 | 6041:7060 |
| 6042:7054 | 6042:7055 | 6042:7056 | 6042:7057 | 6042:7058 | 6042:7059 | 6042:7060 |
| 6043:7054 | 6043:7055 | 6043:7056 | 6043:7057 | 6043:7058 | 6043:7059 | 6043:7060 |
| 6044:7054 | 6044:7055 | 6044:7056 | 6044:7057 | 6044:7058 | 6044:7059 | 6044:7060 |
| 6045:7054 | 6045:7055 | 6045:7056 | 6045:7057 | 6045:7058 | 6045:7059 | 6045:7060 |
| 6046:7054 | 6046:7055 | 6046:7056 | 6046:7057 | 6046:7058 | 6046:7059 | 6046:7060 |
| 6047:7054 | 6047:7055 | 6047:7056 | 6047:7057 | 6047:7058 | 6047:7059 | 6047:7060 |
| 6048:7054 | 6048:7055 | 6048:7056 | 6048:7057 | 6048:7058 | 6048:7059 | 6048:7060 |
| 6049:7054 | 6049:7055 | 6049:7056 | 6049:7057 | 6049:7058 | 6049:7059 | 6049:7060 |
| 6050:7054 | 6050:7055 | 6050:7056 | 6050:7057 | 6050:7058 | 6050:7059 | 6050:7060 |
| 6051:7054 | 6051:7055 | 6051:7056 | 6051:7057 | 6051:7058 | 6051:7059 | 6051:7060 |
| 6052:7054 | 6052:7055 | 6052:7056 | 6052:7057 | 6052:7058 | 6052:7059 | 6052:7060 |
| 6053:7054 | 6053:7055 | 6053:7056 | 6053:7057 | 6053:7058 | 6053:7059 | 6053:7060 |
| 6054:7054 | 6054:7055 | 6054:7056 | 6054:7057 | 6054:7058 | 6054:7059 | 6054:7060 |
| 6055:7054 | 6055:7055 | 6055:7056 | 6055:7057 | 6055:7058 | 6055:7059 | 6055:7060 |
| 6056:7054 | 6056:7055 | 6056:7056 | 6056:7057 | 6056:7058 | 6056:7059 | 6056:7060 |
| 6057:7054 | 6057:7055 | 6057:7056 | 6057:7057 | 6057:7058 | 6057:7059 | 6057:7060 |
| 6058:7054 | 6058:7055 | 6058:7056 | 6058:7057 | 6058:7058 | 6058:7059 | 6058:7060 |
| 6059:7054 | 6059:7055 | 6059:7056 | 6059:7057 | 6059:7058 | 6059:7059 | 6059:7060 |
| 6060:7054 | 6060:7055 | 6060:7056 | 6060:7057 | 6060:7058 | 6060:7059 | 6060:7060 |
| 6041:7061 | 6041:7062 | 6041:7063 | 6041:7064 | 6041:7065 | 6041:7066 | 6041:7067 |
| 6042:7061 | 6042:7062 | 6042:7063 | 6042:7064 | 6042:7065 | 6042:7066 | 6042:7067 |
| 6043:7061 | 6043:7062 | 6043:7063 | 6043:7064 | 6043:7065 | 6043:7066 | 6043:7067 |
| 6044:7061 | 6044:7062 | 6044:7063 | 6044:7064 | 6044:7065 | 6044:7066 | 6044:7067 |
| 6045:7061 | 6045:7062 | 6045:7063 | 6045:7064 | 6045:7065 | 6045:7066 | 6045:7067 |
| 6046:7061 | 6046:7062 | 6046:7063 | 6046:7064 | 6046:7065 | 6046:7066 | 6046:7067 |
| 6047:7061 | 6047:7062 | 6047:7063 | 6047:7064 | 6047:7065 | 6047:7066 | 6047:7067 |
| 6048:7061 | 6048:7062 | 6048:7063 | 6048:7064 | 6048:7065 | 6048:7066 | 6048:7067 |
| 6049:7061 | 6049:7062 | 6049:7063 | 6049:7064 | 6049:7065 | 6049:7066 | 6049:7067 |
| 6050:7061 | 6050:7062 | 6050:7063 | 6050:7064 | 6050:7065 | 6050:7066 | 6050:7067 |
| 6051:7061 | 6051:7062 | 6051:7063 | 6051:7064 | 6051:7065 | 6051:7066 | 6051:7067 |
| 6052:7061 | 6052:7062 | 6052:7063 | 6052:7064 | 6052:7065 | 6052:7066 | 6052:7067 |
| 6053:7061 | 6053:7062 | 6053:7063 | 6053:7064 | 6053:7065 | 6053:7066 | 6053:7067 |
| 6054:7061 | 6054:7062 | 6054:7063 | 6054:7064 | 6054:7065 | 6054:7066 | 6054:7067 |
| 6055:7061 | 6055:7062 | 6055:7063 | 6055:7064 | 6055:7065 | 6055:7066 | 6055:7067 |
| 6056:7061 | 6056:7062 | 6056:7063 | 6056:7064 | 6056:7065 | 6056:7066 | 6056:7067 |
| 6057:7061 | 6057:7062 | 6057:7063 | 6057:7064 | 6057:7065 | 6057:7066 | 6057:7067 |
| 6058:7061 | 6058:7062 | 6058:7063 | 6058:7064 | 6058:7065 | 6058:7066 | 6058:7067 |
| 6059:7061 | 6059:7062 | 6059:7063 | 6059:7064 | 6059:7065 | 6059:7066 | 6059:7067 |
| 6060:7061 | 6060:7062 | 6060:7063 | 6060:7064 | 6060:7065 | 6060:7066 | 6060:7067 |
| 6041:7068 | 6041:7069 | 6041:7070 | 6041:7071 | 6041:7072 | 6041:7073 | 6041:7074 |
| 6042:7068 | 6042:7069 | 6042:7070 | 6042:7071 | 6042:7072 | 6042:7073 | 6042:7074 |
| 6043:7068 | 6043:7069 | 6043:7070 | 6043:7071 | 6043:7072 | 6043:7073 | 6043:7074 |
| 6044:7068 | 6044:7069 | 6044:7070 | 6044:7071 | 6044:7072 | 6044:7073 | 6044:7074 |
| 6045:7068 | 6045:7069 | 6045:7070 | 6045:7071 | 6045:7072 | 6045:7073 | 6045:7074 |
| 6046:7068 | 6046:7069 | 6046:7070 | 6046:7071 | 6046:7072 | 6046:7073 | 6046:7074 |
| 6047:7068 | 6047:7069 | 6047:7070 | 6047:7071 | 6047:7072 | 6047:7073 | 6047:7074 |
| 6048:7068 | 6048:7069 | 6048:7070 | 6048:7071 | 6048:7072 | 6048:7073 | 6048:7074 |
| 6049:7068 | 6049:7069 | 6049:7070 | 6049:7071 | 6049:7072 | 6049:7073 | 6049:7074 |
| 6050:7068 | 6050:7069 | 6050:7070 | 6050:7071 | 6050:7072 | 6050:7073 | 6050:7074 |
| 6051:7068 | 6051:7069 | 6051:7070 | 6051:7071 | 6051:7072 | 6051:7073 | 6051:7074 |
| 6052:7068 | 6052:7069 | 6052:7070 | 6052:7071 | 6052:7072 | 6052:7073 | 6052:7074 |
| 6053:7068 | 6053:7069 | 6053:7070 | 6053:7071 | 6053:7072 | 6053:7073 | 6053:7074 |
| 6054:7068 | 6054:7069 | 6054:7070 | 6054:7071 | 6054:7072 | 6054:7073 | 6054:7074 |
| 6055:7068 | 6055:7069 | 6055:7070 | 6055:7071 | 6055:7072 | 6055:7073 | 6055:7074 |
| 6056:7068 | 6056:7069 | 6056:7070 | 6056:7071 | 6056:7072 | 6056:7073 | 6056:7074 |
| 6057:7068 | 6057:7069 | 6057:7070 | 6057:7071 | 6057:7072 | 6057:7073 | 6057:7074 |
| 6058:7068 | 6058:7069 | 6058:7070 | 6058:7071 | 6058:7072 | 6058:7073 | 6058:7074 |
| 6059:7068 | 6059:7069 | 6059:7070 | 6059:7071 | 6059:7072 | 6059:7073 | 6059:7074 |
| 6060:7068 | 6060:7069 | 6060:7070 | 6060:7071 | 6060:7072 | 6060:7073 | 6060:7074 |
| 6041:7075 | 6041:7076 | 6041:7077 | | | | |
| 6042:7075 | 6042:7076 | 6042:7077 | 6061:7000 | 6061:7001 | 6061:7002 | 6061:7003 |
| 6043:7075 | 6043:7076 | 6043:7077 | 6062:7000 | 6062:7001 | 6062:7002 | 6062:7003 |
| 6044:7075 | 6044:7076 | 6044:7077 | 6063:7000 | 6063:7001 | 6063:7002 | 6063:7003 |
| 6045:7075 | 6045:7076 | 6045:7077 | 6064:7000 | 6064:7001 | 6064:7002 | 6064:7003 |
| 6046:7075 | 6046:7076 | 6046:7077 | 6065:7000 | 6065:7001 | 6065:7002 | 6065:7003 |
| 6047:7075 | 6047:7076 | 6047:7077 | 6066:7000 | 6066:7001 | 6066:7002 | 6066:7003 |
| 6048:7075 | 6048:7076 | 6048:7077 | 6067:7000 | 6067:7001 | 6067:7002 | 6067:7003 |
| 6049:7075 | 6049:7076 | 6049:7077 | 6068:7000 | 6068:7001 | 6068:7002 | 6068:7003 |
| 6050:7075 | 6050:7076 | 6050:7077 | 6069:7000 | 6069:7001 | 6069:7002 | 6069:7003 |
| 6051:7075 | 6051:7076 | 6051:7077 | 6070:7000 | 6070:7001 | 6070:7002 | 6070:7003 |
| 6052:7075 | 6052:7076 | 6052:7077 | 6071:7000 | 6071:7001 | 6071:7002 | 6071:7003 |
| 6053:7075 | 6053:7076 | 6053:7077 | 6072:7000 | 6072:7001 | 6072:7002 | 6072:7003 |
| 6054:7075 | 6054:7076 | 6054:7077 | 6073:7000 | 6073:7001 | 6073:7002 | 6073:7003 |
| 6055:7075 | 6055:7076 | 6055:7077 | 6074:7000 | 6074:7001 | 6074:7002 | 6074:7003 |
| 6056:7075 | 6056:7076 | 6056:7077 | 6075:7000 | 6075:7001 | 6075:7002 | 6075:7003 |
| 6057:7075 | 6057:7076 | 6057:7077 | 6076:7000 | 6076:7001 | 6076:7002 | 6076:7003 |
| 6058:7075 | 6058:7076 | 6058:7077 | 6077:7000 | 6077:7001 | 6077:7002 | 6077:7003 |
| 6059:7075 | 6059:7076 | 6059:7077 | 6078:7000 | 6078:7001 | 6078:7002 | 6078:7003 |
| 6060:7075 | 6060:7076 | 6060:7077 | | | | |
| 6061:7004 | 6061:7005 | 6061:7006 | 6061:7007 | 6061:7008 | 6061:7009 | 6061:7010 |
| 6062:7004 | 6062:7005 | 6062:7006 | 6062:7007 | 6062:7008 | 6062:7009 | 6062:7010 |
| 6063:7004 | 6063:7005 | 6063:7006 | 6063:7007 | 6063:7008 | 6063:7009 | 6063:7010 |
| 6064:7004 | 6064:7005 | 6064:7006 | 6064:7007 | 6064:7008 | 6064:7009 | 6064:7010 |
| 6065:7004 | 6065:7005 | 6065:7006 | 6065:7007 | 6065:7008 | 6065:7009 | 6065:7010 |
| 6066:7004 | 6066:7005 | 6066:7006 | 6066:7007 | 6066:7008 | 6066:7009 | 6066:7010 |
| 6067:7004 | 6067:7005 | 6067:7006 | 6067:7007 | 6067:7008 | 6067:7009 | 6067:7010 |
| 6068:7004 | 6068:7005 | 6068:7006 | 6068:7007 | 6068:7008 | 6068:7009 | 6068:7010 |
| 6069:7004 | 6069:7005 | 6069:7006 | 6069:7007 | 6069:7008 | 6069:7009 | 6069:7010 |
| 6070:7004 | 6070:7005 | 6070:7006 | 6070:7007 | 6070:7008 | 6070:7009 | 6070:7010 |
| 6071:7004 | 6071:7005 | 6071:7006 | 6071:7007 | 6071:7008 | 6071:7009 | 6071:7010 |
| 6072:7004 | 6072:7005 | 6072:7006 | 6072:7007 | 6072:7008 | 6072:7009 | 6072:7010 |
| 6073:7004 | 6073:7005 | 6073:7006 | 6073:7007 | 6073:7008 | 6073:7009 | 6073:7010 |
| 6074:7004 | 6074:7005 | 6074:7006 | 6074:7007 | 6074:7008 | 6074:7009 | 6074:7010 |
| 6075:7004 | 6075:7005 | 6075:7006 | 6075:7007 | 6075:7008 | 6075:7009 | 6075:7010 |
| 6076:7004 | 6076:7005 | 6076:7006 | 6076:7007 | 6076:7008 | 6076:7009 | 6076:7010 |
| 6077:7004 | 6077:7005 | 6077:7006 | 6077:7007 | 6077:7008 | 6077:7009 | 6077:7010 |
| 6078:7004 | 6078:7005 | 6078:7006 | 6078:7007 | 6078:7008 | 6078:7009 | 6078:7010 |
| 6061:7011 | 6061:7012 | 6061:7013 | 6061:7014 | 6061:7015 | 6061:7016 | 6061:7017 |
| 6062:7011 | 6062:7012 | 6062:7013 | 6062:7014 | 6062:7015 | 6062:7016 | 6062:7017 |
| 6063:7011 | 6063:7012 | 6063:7013 | 6063:7014 | 6063:7015 | 6063:7016 | 6063:7017 |
| 6064:7011 | 6064:7012 | 6064:7013 | 6064:7014 | 6064:7015 | 6064:7016 | 6064:7017 |
| 6065:7011 | 6065:7012 | 6065:7013 | 6065:7014 | 6065:7015 | 6065:7016 | 6065:7017 |
| 6066:7011 | 6066:7012 | 6066:7013 | 6066:7014 | 6066:7015 | 6066:7016 | 6066:7017 |
| 6067:7011 | 6067:7012 | 6067:7013 | 6067:7014 | 6067:7015 | 6067:7016 | 6067:7017 |
| 6068:7011 | 6068:7012 | 6068:7013 | 6068:7014 | 6068:7015 | 6068:7016 | 6068:7017 |
| 6069:7011 | 6069:7012 | 6069:7013 | 6069:7014 | 6069:7015 | 6069:7016 | 6069:7017 |
| 6070:7011 | 6070:7012 | 6070:7013 | 6070:7014 | 6070:7015 | 6070:7016 | 6070:7017 |
| 6071:7011 | 6071:7012 | 6071:7013 | 6071:7014 | 6071:7015 | 6071:7016 | 6071:7017 |
| 6072:7011 | 6072:7012 | 6072:7013 | 6072:7014 | 6072:7015 | 6072:7016 | 6072:7017 |
| 6073:7011 | 6073:7012 | 6073:7013 | 6073:7014 | 6073:7015 | 6073:7016 | 6073:7017 |
| 6074:7011 | 6074:7012 | 6074:7013 | 6074:7014 | 6074:7015 | 6074:7016 | 6074:7017 |
| 6075:7011 | 6075:7012 | 6075:7013 | 6075:7014 | 6075:7015 | 6075:7016 | 6075:7017 |
| 6076:7011 | 6076:7012 | 6076:7013 | 6076:7014 | 6076:7015 | 6076:7016 | 6076:7017 |
| 6077:7011 | 6077:7012 | 6077:7013 | 6077:7014 | 6077:7015 | 6077:7016 | 6077:7017 |
| 6078:7011 | 6078:7012 | 6078:7013 | 6078:7014 | 6078:7015 | 6078:7016 | 6078:7017 |
| 6061:7018 | 6061:7019 | 6061:7020 | 6061:7021 | 6061:7022 | 6061:7023 | 6061:7024 |
| 6062:7018 | 6062:7019 | 6062:7020 | 6062:7021 | 6062:7022 | 6062:7023 | 6062:7024 |
| 6063:7018 | 6063:7019 | 6063:7020 | 6063:7021 | 6063:7022 | 6063:7023 | 6063:7024 |
| 6064:7018 | 6064:7019 | 6064:7020 | 6064:7021 | 6064:7022 | 6064:7023 | 6064:7024 |
| 6065:7018 | 6065:7019 | 6065:7020 | 6065:7021 | 6065:7022 | 6065:7023 | 6065:7024 |
| 6066:7018 | 6066:7019 | 6066:7020 | 6066:7021 | 6066:7022 | 6066:7023 | 6066:7024 |
| 6067:7018 | 6067:7019 | 6067:7020 | 6067:7021 | 6067:7022 | 6067:7023 | 6067:7024 |
| 6068:7018 | 6068:7019 | 6068:7020 | 6068:7021 | 6068:7022 | 6068:7023 | 6068:7024 |
| 6069:7018 | 6069:7019 | 6069:7020 | 6069:7021 | 6069:7022 | 6069:7023 | 6069:7024 |
| 6070:7018 | 6070:7019 | 6070:7020 | 6070:7021 | 6070:7022 | 6070:7023 | 6070:7024 |
| 6071:7018 | 6071:7019 | 6071:7020 | 6071:7021 | 6071:7022 | 6071:7023 | 6071:7024 |
| 6072:7018 | 6072:7019 | 6072:7020 | 6072:7021 | 6072:7022 | 6072:7023 | 6072:7024 |
| 6073:7018 | 6073:7019 | 6073:7020 | 6073:7021 | 6073:7022 | 6073:7023 | 6073:7024 |
| 6074:7018 | 6074:7019 | 6074:7020 | 6074:7021 | 6074:7022 | 6074:7023 | 6074:7024 |
| 6075:7018 | 6075:7019 | 6075:7020 | 6075:7021 | 6075:7022 | 6075:7023 | 6075:7024 |
| 6076:7018 | 6076:7019 | 6076:7020 | 6076:7021 | 6076:7022 | 6076:7023 | 6076:7024 |
| 6077:7018 | 6077:7019 | 6077:7020 | 6077:7021 | 6077:7022 | 6077:7023 | 6077:7024 |
| 6078:7018 | 6078:7019 | 6078:7020 | 6078:7021 | 6078:7022 | 6078:7023 | 6078:7024 |
| 6061:7025 | 6061:7026 | 6061:7027 | 6061:7028 | 6061:7029 | 6061:7030 | 6061:7031 |
| 6062:7025 | 6062:7026 | 6062:7027 | 6062:7028 | 6062:7029 | 6062:7030 | 6062:7031 |
| 6063:7025 | 6063:7026 | 6063:7027 | 6063:7028 | 6063:7029 | 6063:7030 | 6063:7031 |
| 6064:7025 | 6064:7026 | 6064:7027 | 6064:7028 | 6064:7029 | 6064:7030 | 6064:7031 |
| 6065:7025 | 6065:7026 | 6065:7027 | 6065:7028 | 6065:7029 | 6065:7030 | 6065:7031 |
| 6066:7025 | 6066:7026 | 6066:7027 | 6066:7028 | 6066:7029 | 6066:7030 | 6066:7031 |
| 6067:7025 | 6067:7026 | 6067:7027 | 6067:7028 | 6067:7029 | 6067:7030 | 6067:7031 |
| 6068:7025 | 6068:7026 | 6068:7027 | 6068:7028 | 6068:7029 | 6068:7030 | 6068:7031 |
| 6069:7025 | 6069:7026 | 6069:7027 | 6069:7028 | 6069:7029 | 6069:7030 | 6069:7031 |
| 6070:7025 | 6070:7026 | 6070:7027 | 6070:7028 | 6070:7029 | 6070:7030 | 6070:7031 |
| 6071:7025 | 6071:7026 | 6071:7027 | 6071:7028 | 6071:7029 | 6071:7030 | 6071:7031 |
| 6072:7025 | 6072:7026 | 6072:7027 | 6072:7028 | 6072:7029 | 6072:7030 | 6072:7031 |
| 6073:7025 | 6073:7026 | 6073:7027 | 6073:7028 | 6073:7029 | 6073:7030 | 6073:7031 |
| 6074:7025 | 6074:7026 | 6074:7027 | 6074:7028 | 6074:7029 | 6074:7030 | 6074:7031 |
| 6075:7025 | 6075:7026 | 6075:7027 | 6075:7028 | 6075:7029 | 6075:7030 | 6075:7031 |
| 6076:7025 | 6076:7026 | 6076:7027 | 6076:7028 | 6076:7029 | 6076:7030 | 6076:7031 |
| 6077:7025 | 6077:7026 | 6077:7027 | 6077:7028 | 6077:7029 | 6077:7030 | 6077:7031 |
| 6078:7025 | 6078:7026 | 6078:7027 | 6078:7028 | 6078:7029 | 6078:7030 | 6078:7031 |
| 6061:7032 | 6061:7033 | 6061:7034 | 6061:7035 | 6061:7036 | 6061:7037 | 6061:7038 |
| 6062:7032 | 6062:7033 | 6062:7034 | 6062:7035 | 6062:7036 | 6062:7037 | 6062:7038 |
| 6063:7032 | 6063:7033 | 6063:7034 | 6063:7035 | 6063:7036 | 6063:7037 | 6063:7038 |
| 6064:7032 | 6064:7033 | 6064:7034 | 6064:7035 | 6064:7036 | 6064:7037 | 6064:7038 |
| 6065:7032 | 6065:7033 | 6065:7034 | 6065:7035 | 6065:7036 | 6065:7037 | 6065:7038 |
| 6066:7032 | 6066:7033 | 6066:7034 | 6066:7035 | 6066:7036 | 6066:7037 | 6066:7038 |
| 6067:7032 | 6067:7033 | 6067:7034 | 6067:7035 | 6067:7036 | 6067:7037 | 6067:7038 |
| 6068:7032 | 6068:7033 | 6068:7034 | 6068:7035 | 6068:7036 | 6068:7037 | 6068:7038 |
| 6069:7032 | 6069:7033 | 6069:7034 | 6069:7035 | 6069:7036 | 6069:7037 | 6069:7038 |
| 6070:7032 | 6070:7033 | 6070:7034 | 6070:7035 | 6070:7036 | 6070:7037 | 6070:7038 |
| 6071:7032 | 6071:7033 | 6071:7034 | 6071:7035 | 6071:7036 | 6071:7037 | 6071:7038 |
| 6072:7032 | 6072:7033 | 6072:7034 | 6072:7035 | 6072:7036 | 6072:7037 | 6072:7038 |
| 6073:7032 | 6073:7033 | 6073:7034 | 6073:7035 | 6073:7036 | 6073:7037 | 6073:7038 |
| 6074:7032 | 6074:7033 | 6074:7034 | 6074:7035 | 6074:7036 | 6074:7037 | 6074:7038 |
| 6075:7032 | 6075:7033 | 6075:7034 | 6075:7035 | 6075:7036 | 6075:7037 | 6075:7038 |
| 6076:7032 | 6076:7033 | 6076:7034 | 6076:7035 | 6076:7036 | 6076:7037 | 6076:7038 |
| 6077:7032 | 6077:7033 | 6077:7034 | 6077:7035 | 6077:7036 | 6077:7037 | 6077:7038 |
| 6078:7032 | 6078:7033 | 6078:7034 | 6078:7035 | 6078:7036 | 6078:7037 | 6078:7038 |
| 6061:7039 | 6061:7040 | 6061:7041 | 6061:7042 | 6061:7043 | 6061:7044 | 6061:7045 |
| 6062:7039 | 6062:7040 | 6062:7041 | 6062:7042 | 6062:7043 | 6062:7044 | 6062:7045 |
| 6063:7039 | 6063:7040 | 6063:7041 | 6063:7042 | 6063:7043 | 6063:7044 | 6063:7045 |
| 6064:7039 | 6064:7040 | 6064:7041 | 6064:7042 | 6064:7043 | 6064:7044 | 6064:7045 |
| 6065:7039 | 6065:7040 | 6065:7041 | 6065:7042 | 6065:7043 | 6065:7044 | 6065:7045 |
| 6066:7039 | 6066:7040 | 6066:7041 | 6066:7042 | 6066:7043 | 6066:7044 | 6066:7045 |
| 6067:7039 | 6067:7040 | 6067:7041 | 6067:7042 | 6067:7043 | 6067:7044 | 6067:7045 |
| 6068:7039 | 6068:7040 | 6068:7041 | 6068:7042 | 6068:7043 | 6068:7044 | 6068:7045 |
| 6069:7039 | 6069:7040 | 6069:7041 | 6069:7042 | 6069:7043 | 6069:7044 | 6069:7045 |
| 6070:7039 | 6070:7040 | 6070:7041 | 6070:7042 | 6070:7043 | 6070:7044 | 6070:7045 |
| 6071:7039 | 6071:7040 | 6071:7041 | 6071:7042 | 6071:7043 | 6071:7044 | 6071:7045 |
| 6072:7039 | 6072:7040 | 6072:7041 | 6072:7042 | 6072:7043 | 6072:7044 | 6072:7045 |
| 6073:7039 | 6073:7040 | 6073:7041 | 6073:7042 | 6073:7043 | 6073:7044 | 6073:7045 |
| 6074:7039 | 6074:7040 | 6074:7041 | 6074:7042 | 6074:7043 | 6074:7044 | 6074:7045 |
| 6075:7039 | 6075:7040 | 6075:7041 | 6075:7042 | 6075:7043 | 6075:7044 | 6075:7045 |
| 6076:7039 | 6076:7040 | 6076:7041 | 6076:7042 | 6076:7043 | 6076:7044 | 6076:7045 |
| 6077:7039 | 6077:7040 | 6077:7041 | 6077:7042 | 6077:7043 | 6077:7044 | 6077:7045 |
| 6078:7039 | 6078:7040 | 6078:7041 | 6078:7042 | 6078:7043 | 6078:7044 | 6078:7045 |
| 6061:7046 | 6061:7047 | 6061:7048 | 6061:7049 | 6061:7050 | 6061:7051 | 6061:7052 |
| 6062:7046 | 6062:7047 | 6062:7048 | 6062:7049 | 6062:7050 | 6062:7051 | 6062:7052 |
| 6063:7046 | 6063:7047 | 6063:7048 | 6063:7049 | 6063:7050 | 6063:7051 | 6063:7052 |
| 6064:7046 | 6064:7047 | 6064:7048 | 6064:7049 | 6064:7050 | 6064:7051 | 6064:7052 |
| 6065:7046 | 6065:7047 | 6065:7048 | 6065:7049 | 6065:7050 | 6065:7051 | 6065:7052 |
| 6066:7046 | 6066:7047 | 6066:7048 | 6066:7049 | 6066:7050 | 6066:7051 | 6066:7052 |
| 6067:7046 | 6067:7047 | 6067:7048 | 6067:7049 | 6067:7050 | 6067:7051 | 6067:7052 |
| 6068:7046 | 6068:7047 | 6068:7048 | 6068:7049 | 6068:7050 | 6068:7051 | 6068:7052 |
| 6069:7046 | 6069:7047 | 6069:7048 | 6069:7049 | 6069:7050 | 6069:7051 | 6069:7052 |
| 6070:7046 | 6070:7047 | 6070:7048 | 6070:7049 | 6070:7050 | 6070:7051 | 6070:7052 |
| 6071:7046 | 6071:7047 | 6071:7048 | 6071:7049 | 6071:7050 | 6071:7051 | 6071:7052 |
| 6072:7046 | 6072:7047 | 6072:7048 | 6072:7049 | 6072:7050 | 6072:7051 | 6072:7052 |
| 6073:7046 | 6073:7047 | 6073:7048 | 6073:7049 | 6073:7050 | 6073:7051 | 6073:7052 |
| 6074:7046 | 6074:7047 | 6074:7048 | 6074:7049 | 6074:7050 | 6074:7051 | 6074:7052 |
| 6075:7046 | 6075:7047 | 6075:7048 | 6075:7049 | 6075:7050 | 6075:7051 | 6075:7052 |
| 6076:7046 | 6076:7047 | 6076:7048 | 6076:7049 | 6076:7050 | 6076:7051 | 6076:7052 |
| 6077:7046 | 6077:7047 | 6077:7048 | 6077:7049 | 6077:7050 | 6077:7051 | 6077:7052 |
| 6078:7046 | 6078:7047 | 6078:7048 | 6078:7049 | 6078:7050 | 6078:7051 | 6078:7052 |
| 6061:7053 | 6061:7054 | 6061:7055 | 6061:7056 | 6061:7057 | 6061:7058 | 6061:7059 |
| 6062:7053 | 6062:7054 | 6062:7055 | 6062:7056 | 6062:7057 | 6062:7058 | 6062:7059 |
| 6063:7053 | 6063:7054 | 6063:7055 | 6063:7056 | 6063:7057 | 6063:7058 | 6063:7059 |
| 6064:7053 | 6064:7054 | 6064:7055 | 6064:7056 | 6064:7057 | 6064:7058 | 6064:7059 |
| 6065:7053 | 6065:7054 | 6065:7055 | 6065:7056 | 6065:7057 | 6065:7058 | 6065:7059 |
| 6066:7053 | 6066:7054 | 6066:7055 | 6066:7056 | 6066:7057 | 6066:7058 | 6066:7059 |
| 6067:7053 | 6067:7054 | 6067:7055 | 6067:7056 | 6067:7057 | 6067:7058 | 6067:7059 |
| 6068:7053 | 6068:7054 | 6068:7055 | 6068:7056 | 6068:7057 | 6068:7058 | 6068:7059 |
| 6069:7053 | 6069:7054 | 6069:7055 | 6069:7056 | 6069:7057 | 6069:7058 | 6069:7059 |
| 6070:7053 | 6070:7054 | 6070:7055 | 6070:7056 | 6070:7057 | 6070:7058 | 6070:7059 |
| 6071:7053 | 6071:7054 | 6071:7055 | 6071:7056 | 6071:7057 | 6071:7058 | 6071:7059 |
| 6072:7053 | 6072:7054 | 6072:7055 | 6072:7056 | 6072:7057 | 6072:7058 | 6072:7059 |
| 6073:7053 | 6073:7054 | 6073:7055 | 6073:7056 | 6073:7057 | 6073:7058 | 6073:7059 |
| 6074:7053 | 6074:7054 | 6074:7055 | 6074:7056 | 6074:7057 | 6074:7058 | 6074:7059 |
| 6075:7053 | 6075:7054 | 6075:7055 | 6075:7056 | 6075:7057 | 6075:7058 | 6075:7059 |
| 6076:7053 | 6076:7054 | 6076:7055 | 6076:7056 | 6076:7057 | 6076:7058 | 6076:7059 |
| 6077:7053 | 6077:7054 | 6077:7055 | 6077:7056 | 6077:7057 | 6077:7058 | 6077:7059 |
| 6078:7053 | 6078:7054 | 6078:7055 | 6078:7056 | 6078:7057 | 6078:7058 | 6078:7059 |
| 6061:7060 | 6061:7061 | 6061:7062 | 6061:7063 | 6061:7064 | 6061:7065 | 6061:7066 |
| 6062:7060 | 6062:7061 | 6062:7062 | 6062:7063 | 6062:7064 | 6062:7065 | 6062:7066 |
| 6063:7060 | 6063:7061 | 6063:7062 | 6063:7063 | 6063:7064 | 6063:7065 | 6063:7066 |
| 6064:7060 | 6064:7061 | 6064:7062 | 6064:7063 | 6064:7064 | 6064:7065 | 6064:7066 |
| 6065:7060 | 6065:7061 | 6065:7062 | 6065:7063 | 6065:7064 | 6065:7065 | 6065:7066 |
| 6066:7060 | 6066:7061 | 6066:7062 | 6066:7063 | 6066:7064 | 6066:7065 | 6066:7066 |
| 6067:7060 | 6067:7061 | 6067:7062 | 6067:7063 | 6067:7064 | 6067:7065 | 6067:7066 |
| 6068:7060 | 6068:7061 | 6068:7062 | 6068:7063 | 6068:7064 | 6068:7065 | 6068:7066 |
| 6069:7060 | 6069:7061 | 6069:7062 | 6069:7063 | 6069:7064 | 6069:7065 | 6069:7066 |
| 6070:7060 | 6070:7061 | 6070:7062 | 6070:7063 | 6070:7064 | 6070:7065 | 6070:7066 |
| 6071:7060 | 6071:7061 | 6071:7062 | 6071:7063 | 6071:7064 | 6071:7065 | 6071:7066 |
| 6072:7060 | 6072:7061 | 6072:7062 | 6072:7063 | 6072:7064 | 6072:7065 | 6072:7066 |
| 6073:7060 | 6073:7061 | 6073:7062 | 6073:7063 | 6073:7064 | 6073:7065 | 6073:7066 |
| 6074:7060 | 6074:7061 | 6074:7062 | 6074:7063 | 6074:7064 | 6074:7065 | 6074:7066 |
| 6075:7060 | 6075:7061 | 6075:7062 | 6075:7063 | 6075:7064 | 6075:7065 | 6075:7066 |
| 6076:7060 | 6076:7061 | 6076:7062 | 6076:7063 | 6076:7064 | 6076:7065 | 6076:7066 |
| 6077:7060 | 6077:7061 | 6077:7062 | 6077:7063 | 6077:7064 | 6077:7065 | 6077:7066 |
| 6078:7060 | 6078:7061 | 6078:7062 | 6078:7063 | 6078:7064 | 6078:7065 | 6078:7066 |
| 6061:7067 | 6061:7068 | 6061:7069 | 6061:7070 | 6061:7071 | 6061:7072 | 6061:7073 |
| 6062:7067 | 6062:7068 | 6062:7069 | 6062:7070 | 6062:7071 | 6062:7072 | 6062:7073 |
| 6063:7067 | 6063:7068 | 6063:7069 | 6063:7070 | 6063:7071 | 6063:7072 | 6063:7073 |
| 6064:7067 | 6064:7068 | 6064:7069 | 6064:7070 | 6064:7071 | 6064:7072 | 6064:7073 |
| 6065:7067 | 6065:7068 | 6065:7069 | 6065:7070 | 6065:7071 | 6065:7072 | 6065:7073 |
| 6066:7067 | 6066:7068 | 6066:7069 | 6066:7070 | 6066:7071 | 6066:7072 | 6066:7073 |
| 6067:7067 | 6067:7068 | 6067:7069 | 6067:7070 | 6067:7071 | 6067:7072 | 6067:7073 |
| 6068:7067 | 6068:7068 | 6068:7069 | 6068:7070 | 6068:7071 | 6068:7072 | 6068:7073 |
| 6069:7067 | 6069:7068 | 6069:7069 | 6069:7070 | 6069:7071 | 6069:7072 | 6069:7073 |
| 6070:7067 | 6070:7068 | 6070:7069 | 6070:7070 | 6070:7071 | 6070:7072 | 6070:7073 |
| 6071:7067 | 6071:7068 | 6071:7069 | 6071:7070 | 6071:7071 | 6071:7072 | 6071:7073 |
| 6072:7067 | 6072:7068 | 6072:7069 | 6072:7070 | 6072:7071 | 6072:7072 | 6072:7073 |
| 6073:7067 | 6073:7068 | 6073:7069 | 6073:7070 | 6073:7071 | 6073:7072 | 6073:7073 |
| 6074:7067 | 6074:7068 | 6074:7069 | 6074:7070 | 6074:7071 | 6074:7072 | 6074:7073 |
| 6075:7067 | 6075:7068 | 6075:7069 | 6075:7070 | 6075:7071 | 6075:7072 | 6075:7073 |
| 6076:7067 | 6076:7068 | 6076:7069 | 6076:7070 | 6076:7071 | 6076:7072 | 6076:7073 |
| 6077:7067 | 6077:7068 | 6077:7069 | 6077:7070 | 6077:7071 | 6077:7072 | 6077:7073 |
| 6078:7067 | 6078:7068 | 6078:7069 | 6078:7070 | 6078:7071 | 6078:7072 | 6078:7073 |
| 6061:7074 | 6061:7075 | 6061:7076 | 6061:7077 | — | — | — |
| 6062:7074 | 6062:7075 | 6062:7076 | 6062:7077 | | | |
| 6063:7074 | 6063:7075 | 6063:7076 | 6063:7077 | | | |
| 6064:7074 | 6064:7075 | 6064:7076 | 6064:7077 | | | |
| 6065:7074 | 6065:7075 | 6065:7076 | 6065:7077 | | | |
| 6066:7074 | 6066:7075 | 6066:7076 | 6066:7077 | | | |
| 6067:7074 | 6067:7075 | 6067:7076 | 6067:7077 | | | |
| 6068:7074 | 6068:7075 | 6068:7076 | 6068:7077 | | | |
| 6069:7074 | 6069:7075 | 6069:7076 | 6069:7077 | | | |
| 6070:7074 | 6070:7075 | 6070:7076 | 6070:7077 | | | |
| 6071:7074 | 6071:7075 | 6071:7076 | 6071:7077 | | | |
| 6072:7074 | 6072:7075 | 6072:7076 | 6072:7077 | | | |
| 6073:7074 | 6073:7075 | 6073:7076 | 6073:7077 | | | |
| 6074:7074 | 6074:7075 | 6074:7076 | 6074:7077 | | | |
| 6075:7074 | 6075:7075 | 6075:7076 | 6075:7077 | | | |
| 6076:7074 | 6076:7075 | 6076:7076 | 6076:7077 | | | |
| 6077:7074 | 6077:7075 | 6077:7076 | 6077:7077 | | | |
| 6078:7074 | 6078:7075 | 6078:7076 | 6078:7077 | | | |

TABLE C
Example combinations of a compound X with a compound Y.

| X:Y | X:Y | X:Y | X:Y | X:Y | X:Y |
|---|---|---|---|---|---|
| 8000:7000 | 8000:7026 | 8000:7052 | 8001:7000 | 8001:7026 | 8001:7052 |
| 8000:7001 | 8000:7027 | 8000:7053 | 8001:7001 | 8001:7027 | 8001:7053 |
| 8000:7002 | 8000:7028 | 8000:7054 | 8001:7002 | 8001:7028 | 8001:7054 |
| 8000:7003 | 8000:7029 | 8000:7055 | 8001:7003 | 8001:7029 | 8001:7055 |
| 8000:7004 | 8000:7030 | 8000:7056 | 8001:7004 | 8001:7030 | 8001:7056 |
| 8000:7005 | 8000:7031 | 8000:7057 | 8001:7005 | 8001:7031 | 8001:7057 |
| 8000:7006 | 8000:7032 | 8000:7058 | 8001:7006 | 8001:7032 | 8001:7058 |
| 8000:7007 | 8000:7033 | 8000:7059 | 8001:7007 | 8001:7033 | 8001:7059 |
| 8000:7008 | 8000:7034 | 8000:7060 | 8001:7008 | 8001:7034 | 8001:7060 |
| 8000:7009 | 8000:7035 | 8000:7061 | 8001:7009 | 8001:7035 | 8001:7061 |
| 8000:7010 | 8000:7036 | 8000:7062 | 8001:7010 | 8001:7036 | 8001:7062 |
| 8000:7011 | 8000:7037 | 8000:7063 | 8001:7011 | 8001:7037 | 8001:7063 |
| 8000:7012 | 8000:7038 | 8000:7064 | 8001:7012 | 8001:7038 | 8001:7064 |
| 8000:7013 | 8000:7039 | 8000:7065 | 8001:7013 | 8001:7039 | 8001:7065 |
| 8000:7014 | 8000:7040 | 8000:7066 | 8001:7014 | 8001:7040 | 8001:7066 |
| 8000:7015 | 8000:7041 | 8000:7067 | 8001:7015 | 8001:7041 | 8001:7067 |
| 8000:7016 | 8000:7042 | 8000:7068 | 8001:7016 | 8001:7042 | 8001:7068 |
| 8000:7017 | 8000:7043 | 8000:7069 | 8001:7017 | 8001:7043 | 8001:7069 |
| 8000:7018 | 8000:7044 | 8000:7070 | 8001:7018 | 8001:7044 | 8001:7070 |
| 8000:7019 | 8000:7045 | 8000:7071 | 8001:7019 | 8001:7045 | 8001:7071 |
| 8000:7020 | 8000:7046 | 8000:7072 | 8001:7020 | 8001:7046 | 8001:7072 |
| 8000:7021 | 8000:7047 | 8000:7073 | 8001:7021 | 8001:7047 | 8001:7073 |
| 8000:7022 | 8000:7048 | 8000:7074 | 8001:7022 | 8001:7048 | 8001:7074 |
| 8000:7023 | 8000:7049 | 8000:7075 | 8001:7023 | 8001:7049 | 8001:7075 |
| 8000:7024 | 8000:7050 | 8000:7076 | 8001:7024 | 8001:7050 | 8001:7076 |
| 8000:7025 | 8000:7051 | 8000:7077 | 8001:7025 | 8001:7051 | 8001:7077 |
| 8002:7000 | 8002:7026 | 8002:7052 | 8003:7000 | 8003:7026 | 8003:7052 |
| 8002:7001 | 8002:7027 | 8002:7053 | 8003:7001 | 8003:7027 | 8003:7053 |
| 8002:7002 | 8002:7028 | 8002:7054 | 8003:7002 | 8003:7028 | 8003:7054 |
| 8002:7003 | 8002:7029 | 8002:7055 | 8003:7003 | 8003:7029 | 8003:7055 |
| 8002:7004 | 8002:7030 | 8002:7056 | 8003:7004 | 8003:7030 | 8003:7056 |
| 8002:7005 | 8002:7031 | 8002:7057 | 8003:7005 | 8003:7031 | 8003:7057 |
| 8002:7006 | 8002:7032 | 8002:7058 | 8003:7006 | 8003:7032 | 8003:7058 |
| 8002:7007 | 8002:7033 | 8002:7059 | 8003:7007 | 8003:7033 | 8003:7059 |
| 8002:7008 | 8002:7034 | 8002:7060 | 8003:7008 | 8003:7034 | 8003:7060 |
| 8002:7009 | 8002:7035 | 8002:7061 | 8003:7009 | 8003:7035 | 8003:7061 |
| 8002:7010 | 8002:7036 | 8002:7062 | 8003:7010 | 8003:7036 | 8003:7062 |
| 8002:7011 | 8002:7037 | 8002:7063 | 8003:7011 | 8003:7037 | 8003:7063 |
| 8002:7012 | 8002:7038 | 8002:7064 | 8003:7012 | 8003:7038 | 8003:7064 |
| 8002:7013 | 8002:7039 | 8002:7065 | 8003:7013 | 8003:7039 | 8003:7065 |
| 8002:7014 | 8002:7040 | 8002:7066 | 8003:7014 | 8003:7040 | 8003:7066 |
| 8002:7015 | 8002:7041 | 8002:7067 | 8003:7015 | 8003:7041 | 8003:7067 |
| 8002:7016 | 8002:7042 | 8002:7068 | 8003:7016 | 8003:7042 | 8003:7068 |
| 8002:7017 | 8002:7043 | 8002:7069 | 8003:7017 | 8003:7043 | 8003:7069 |
| 8002:7018 | 8002:7044 | 8002:7070 | 8003:7018 | 8003:7044 | 8003:7070 |
| 8002:7019 | 8002:7045 | 8002:7071 | 8003:7019 | 8003:7045 | 8003:7071 |
| 8002:7020 | 8002:7046 | 8002:7072 | 8003:7020 | 8003:7046 | 8003:7072 |
| 8002:7021 | 8002:7047 | 8002:7073 | 8003:7021 | 8003:7047 | 8003:7073 |
| 8002:7022 | 8002:7048 | 8002:7074 | 8003:7022 | 8003:7048 | 8003:7074 |
| 8002:7023 | 8002:7049 | 8002:7075 | 8003:7023 | 8003:7049 | 8003:7075 |
| 8002:7024 | 8002:7050 | 8002:7076 | 8003:7024 | 8003:7050 | 8003:7076 |
| 8002:7025 | 8002:7051 | 8002:7077 | 8003:7025 | 8003:7051 | 8003:7077 |
| 8004:7000 | 8004:7026 | 8004:7052 | 8005:7000 | 8005:7026 | 8005:7052 |
| 8004:7001 | 8004:7027 | 8004:7053 | 8005:7001 | 8005:7027 | 8005:7053 |
| 8004:7002 | 8004:7028 | 8004:7054 | 8005:7002 | 8005:7028 | 8005:7054 |
| 8004:7003 | 8004:7029 | 8004:7055 | 8005:7003 | 8005:7029 | 8005:7055 |
| 8004:7004 | 8004:7030 | 8004:7056 | 8005:7004 | 8005:7030 | 8005:7056 |
| 8004:7005 | 8004:7031 | 8004:7057 | 8005:7005 | 8005:7031 | 8005:7057 |
| 8004:7006 | 8004:7032 | 8004:7058 | 8005:7006 | 8005:7032 | 8005:7058 |
| 8004:7007 | 8004:7033 | 8004:7059 | 8005:7007 | 8005:7033 | 8005:7059 |
| 8004:7008 | 8004:7034 | 8004:7060 | 8005:7008 | 8005:7034 | 8005:7060 |
| 8004:7009 | 8004:7035 | 8004:7061 | 8005:7009 | 8005:7035 | 8005:7061 |
| 8004:7010 | 8004:7036 | 8004:7062 | 8005:7010 | 8005:7036 | 8005:7062 |
| 8004:7011 | 8004:7037 | 8004:7063 | 8005:7011 | 8005:7037 | 8005:7063 |
| 8004:7012 | 8004:7038 | 8004:7064 | 8005:7012 | 8005:7038 | 8005:7064 |
| 8004:7013 | 8004:7039 | 8004:7065 | 8005:7013 | 8005:7039 | 8005:7065 |
| 8004:7014 | 8004:7040 | 8004:7066 | 8005:7014 | 8005:7040 | 8005:7066 |
| 8004:7015 | 8004:7041 | 8004:7067 | 8005:7015 | 8005:7041 | 8005:7067 |
| 8004:7016 | 8004:7042 | 8004:7068 | 8005:7016 | 8005:7042 | 8005:7068 |
| 8004:7017 | 8004:7043 | 8004:7069 | 8005:7017 | 8005:7043 | 8005:7069 |
| 8004:7018 | 8004:7044 | 8004:7070 | 8005:7018 | 8005:7044 | 8005:7070 |
| 8004:7019 | 8004:7045 | 8004:7071 | 8005:7019 | 8005:7045 | 8005:7071 |
| 8004:7020 | 8004:7046 | 8004:7072 | 8005:7020 | 8005:7046 | 8005:7072 |
| 8004:7021 | 8004:7047 | 8004:7073 | 8005:7021 | 8005:7047 | 8005:7073 |
| 8004:7022 | 8004:7048 | 8004:7074 | 8005:7022 | 8005:7048 | 8005:7074 |
| 8004:7023 | 8004:7049 | 8004:7075 | 8005:7023 | 8005:7049 | 8005:7075 |
| 8004:7024 | 8004:7050 | 8004:7076 | 8005:7024 | 8005:7050 | 8005:7076 |
| 8004:7025 | 8004:7051 | 8004:7077 | 8005:7025 | 8005:7051 | 8005:7077 |
| 8006:7000 | 8006:7026 | 8006:7052 | 8007:7000 | 8007:7026 | 8007:7052 |
| 8006:7001 | 8006:7027 | 8006:7053 | 8007:7001 | 8007:7027 | 8007:7053 |
| 8006:7002 | 8006:7028 | 8006:7054 | 8007:7002 | 8007:7028 | 8007:7054 |
| 8006:7003 | 8006:7029 | 8006:7055 | 8007:7003 | 8007:7029 | 8007:7055 |
| 8006:7004 | 8006:7030 | 8006:7056 | 8007:7004 | 8007:7030 | 8007:7056 |
| 8006:7005 | 8006:7031 | 8006:7057 | 8007:7005 | 8007:7031 | 8007:7057 |
| 8006:7006 | 8006:7032 | 8006:7058 | 8007:7006 | 8007:7032 | 8007:7058 |
| 8006:7007 | 8006:7033 | 8006:7059 | 8007:7007 | 8007:7033 | 8007:7059 |
| 8006:7008 | 8006:7034 | 8006:7060 | 8007:7008 | 8007:7034 | 8007:7060 |
| 8006:7009 | 8006:7035 | 8006:7061 | 8007:7009 | 8007:7035 | 8007:7061 |
| 8006:7010 | 8006:7036 | 8006:7062 | 8007:7010 | 8007:7036 | 8007:7062 |
| 8006:7011 | 8006:7037 | 8006:7063 | 8007:7011 | 8007:7037 | 8007:7063 |
| 8006:7012 | 8006:7038 | 8006:7064 | 8007:7012 | 8007:7038 | 8007:7064 |
| 8006:7013 | 8006:7039 | 8006:7065 | 8007:7013 | 8007:7039 | 8007:7065 |
| 8006:7014 | 8006:7040 | 8006:7066 | 8007:7014 | 8007:7040 | 8007:7066 |
| 8006:7015 | 8006:7041 | 8006:7067 | 8007:7015 | 8007:7041 | 8007:7067 |
| 8006:7016 | 8006:7042 | 8006:7068 | 8007:7016 | 8007:7042 | 8007:7068 |
| 8006:7017 | 8006:7043 | 8006:7069 | 8007:7017 | 8007:7043 | 8007:7069 |
| 8006:7018 | 8006:7044 | 8006:7070 | 8007:7018 | 8007:7044 | 8007:7070 |
| 8006:7019 | 8006:7045 | 8006:7071 | 8007:7019 | 8007:7045 | 8007:7071 |
| 8006:7020 | 8006:7046 | 8006:7072 | 8007:7020 | 8007:7046 | 8007:7072 |
| 8006:7021 | 8006:7047 | 8006:7073 | 8007:7021 | 8007:7047 | 8007:7073 |
| 8006:7022 | 8006:7048 | 8006:7074 | 8007:7022 | 8007:7048 | 8007:7074 |
| 8006:7023 | 8006:7049 | 8006:7075 | 8007:7023 | 8007:7049 | 8007:7075 |
| 8006:7024 | 8006:7050 | 8006:7076 | 8007:7024 | 8007:7050 | 8007:7076 |
| 8006:7025 | 8006:7051 | 8006:7077 | 8007:7025 | 8007:7051 | 8007:7077 |
| 8008:7000 | 8008:7026 | 8008:7052 | 8009:7000 | 8009:7026 | 8009:7052 |
| 8008:7001 | 8008:7027 | 8008:7053 | 8009:7001 | 8009:7027 | 8009:7053 |
| 8008:7002 | 8008:7028 | 8008:7054 | 8009:7002 | 8009:7028 | 8009:7054 |
| 8008:7003 | 8008:7029 | 8008:7055 | 8009:7003 | 8009:7029 | 8009:7055 |
| 8008:7004 | 8008:7030 | 8008:7056 | 8009:7004 | 8009:7030 | 8009:7056 |
| 8008:7005 | 8008:7031 | 8008:7057 | 8009:7005 | 8009:7031 | 8009:7057 |
| 8008:7006 | 8008:7032 | 8008:7058 | 8009:7006 | 8009:7032 | 8009:7058 |
| 8008:7007 | 8008:7033 | 8008:7059 | 8009:7007 | 8009:7033 | 8009:7059 |
| 8008:7008 | 8008:7034 | 8008:7060 | 8009:7008 | 8009:7034 | 8009:7060 |
| 8008:7009 | 8008:7035 | 8008:7061 | 8009:7009 | 8009:7035 | 8009:7061 |
| 8008:7010 | 8008:7036 | 8008:7062 | 8009:7010 | 8009:7036 | 8009:7062 |
| 8008:7011 | 8008:7037 | 8008:7063 | 8009:7011 | 8009:7037 | 8009:7063 |
| 8008:7012 | 8008:7038 | 8008:7064 | 8009:7012 | 8009:7038 | 8009:7064 |
| 8008:7013 | 8008:7039 | 8008:7065 | 8009:7013 | 8009:7039 | 8009:7065 |
| 8008:7014 | 8008:7040 | 8008:7066 | 8009:7014 | 8009:7040 | 8009:7066 |
| 8008:7015 | 8008:7041 | 8008:7067 | 8009:7015 | 8009:7041 | 8009:7067 |
| 8008:7016 | 8008:7042 | 8008:7068 | 8009:7016 | 8009:7042 | 8009:7068 |
| 8008:7017 | 8008:7043 | 8008:7069 | 8009:7017 | 8009:7043 | 8009:7069 |
| 8008:7018 | 8008:7044 | 8008:7070 | 8009:7018 | 8009:7044 | 8009:7070 |
| 8008:7019 | 8008:7045 | 8008:7071 | 8009:7019 | 8009:7045 | 8009:7071 |
| 8008:7020 | 8008:7046 | 8008:7072 | 8009:7020 | 8009:7046 | 8009:7072 |
| 8008:7021 | 8008:7047 | 8008:7073 | 8009:7021 | 8009:7047 | 8009:7073 |
| 8008:7022 | 8008:7048 | 8008:7074 | 8009:7022 | 8009:7048 | 8009:7074 |
| 8008:7023 | 8008:7049 | 8008:7075 | 8009:7023 | 8009:7049 | 8009:7075 |
| 8008:7024 | 8008:7050 | 8008:7076 | 8009:7024 | 8009:7050 | 8009:7076 |
| 8008:7025 | 8008:7051 | 8008:7077 | 8009:7025 | 8009:7051 | 8009:7077 |
| 8010:7000 | 8010:7026 | 8010:7052 | 8011:7000 | 8011:7026 | 8011:7052 |
| 8010:7001 | 8010:7027 | 8010:7053 | 8011:7001 | 8011:7027 | 8011:7053 |
| 8010:7002 | 8010:7028 | 8010:7054 | 8011:7002 | 8011:7028 | 8011:7054 |
| 8010:7003 | 8010:7029 | 8010:7055 | 8011:7003 | 8011:7029 | 8011:7055 |
| 8010:7004 | 8010:7030 | 8010:7056 | 8011:7004 | 8011:7030 | 8011:7056 |
| 8010:7005 | 8010:7031 | 8010:7057 | 8011:7005 | 8011:7031 | 8011:7057 |
| 8010:7006 | 8010:7032 | 8010:7058 | 8011:7006 | 8011:7032 | 8011:7058 |
| 8010:7007 | 8010:7033 | 8010:7059 | 8011:7007 | 8011:7033 | 8011:7059 |
| 8010:7008 | 8010:7034 | 8010:7060 | 8011:7008 | 8011:7034 | 8011:7060 |
| 8010:7009 | 8010:7035 | 8010:7061 | 8011:7009 | 8011:7035 | 8011:7061 |
| 8010:7010 | 8010:7036 | 8010:7062 | 8011:7010 | 8011:7036 | 8011:7062 |
| 8010:7011 | 8010:7037 | 8010:7063 | 8011:7011 | 8011:7037 | 8011:7063 |
| 8010:7012 | 8010:7038 | 8010:7064 | 8011:7012 | 8011:7038 | 8011:7064 |
| 8010:7013 | 8010:7039 | 8010:7065 | 8011:7013 | 8011:7039 | 8011:7065 |
| 8010:7014 | 8010:7040 | 8010:7066 | 8011:7014 | 8011:7040 | 8011:7066 |
| 8010:7015 | 8010:7041 | 8010:7067 | 8011:7015 | 8011:7041 | 8011:7067 |
| 8010:7016 | 8010:7042 | 8010:7068 | 8011:7016 | 8011:7042 | 8011:7068 |
| 8010:7017 | 8010:7043 | 8010:7069 | 8011:7017 | 8011:7043 | 8011:7069 |
| 8010:7018 | 8010:7044 | 8010:7070 | 8011:7018 | 8011:7044 | 8011:7070 |
| 8010:7019 | 8010:7045 | 8010:7071 | 8011:7019 | 8011:7045 | 8011:7071 |
| 8010:7020 | 8010:7046 | 8010:7072 | 8011:7020 | 8011:7046 | 8011:7072 |
| 8010:7021 | 8010:7047 | 8010:7073 | 8011:7021 | 8011:7047 | 8011:7073 |
| 8010:7022 | 8010:7048 | 8010:7074 | 8011:7022 | 8011:7048 | 8011:7074 |
| 8010:7023 | 8010:7049 | 8010:7075 | 8011:7023 | 8011:7049 | 8011:7075 |
| 8010:7024 | 8010:7050 | 8010:7076 | 8011:7024 | 8011:7050 | 8011:7076 |
| 8010:7025 | 8010:7051 | 8010:7077 | 8011:7025 | 8011:7051 | 8011:7077 |
| 8012:7000 | 8012:7026 | 8012:7052 | — | — | — |
| 8012:7001 | 8012:7027 | 8012:7053 | | | |
| 8012:7002 | 8012:7028 | 8012:7054 | | | |
| 8012:7003 | 8012:7029 | 8012:7055 | | | |
| 8012:7004 | 8012:7030 | 8012:7056 | | | |
| 8012:7005 | 8012:7031 | 8012:7057 | | | |
| 8012:7006 | 8012:7032 | 8012:7058 | | | |
| 8012:7007 | 8012:7033 | 8012:7059 | | | |
| 8012:7008 | 8012:7034 | 8012:7060 | | | |
| 8012:7009 | 8012:7035 | 8012:7061 | | | |
| 8012:7010 | 8012:7036 | 8012:7062 | | | |
| 8012:7011 | 8012:7037 | 8012:7063 | | | |
| 8012:7012 | 8012:7038 | 8012:7064 | | | |
| 8012:7013 | 8012:7039 | 8012:7065 | | | |
| 8012:7014 | 8012:7040 | 8012:7066 | | | |
| 8012:7015 | 8012:7041 | 8012:7067 | | | |
| 8012:7016 | 8012:7042 | 8012:7068 | | | |
| 8012:7017 | 8012:7043 | 8012:7069 | | | |
| 8012:7018 | 8012:7044 | 8012:7070 | | | |
| 8012:7019 | 8012:7045 | 8012:7071 | | | |
| 8012:7020 | 8012:7046 | 8012:7072 | | | |
| 8012:7021 | 8012:7047 | 8012:7073 | | | |
| 8012:7022 | 8012:7048 | 8012:7074 | | | |
| 8012:7023 | 8012:7049 | 8012:7075 | | | |
| 8012:7024 | 8012:7050 | 8012:7076 | | | |
| 8012:7025 | 8012:7051 | 8012:7077 | | | |

TABLE D
Example combinations of a compound X with a compound Y.

| X:Y | X:Y | X:Y |
|---|---|---|
| 9000:7000 | 9000:7026 | 9000:7052 |
| 9000:7001 | 9000:7027 | 9000:7053 |
| 9000:7002 | 9000:7028 | 9000:7054 |
| 9000:7003 | 9000:7029 | 9000:7055 |
| 9000:7004 | 9000:7030 | 9000:7056 |
| 9000:7005 | 9000:7031 | 9000:7057 |
| 9000:7006 | 9000:7032 | 9000:7058 |
| 9000:7007 | 9000:7033 | 9000:7059 |
| 9000:7008 | 9000:7034 | 9000:7060 |
| 9000:7009 | 9000:7035 | 9000:7061 |
| 9000:7010 | 9000:7036 | 9000:7062 |
| 9000:7011 | 9000:7037 | 9000:7063 |
| 9000:7012 | 9000:7038 | 9000:7064 |
| 9000:7013 | 9000:7039 | 9000:7065 |
| 9000:7014 | 9000:7040 | 9000:7066 |
| 9000:7015 | 9000:7041 | 9000:7067 |
| 9000:7016 | 9000:7042 | 9000:7068 |
| 9000:7017 | 9000:7043 | 9000:7069 |
| 9000:7018 | 9000:7044 | 9000:7070 |
| 9000:7019 | 9000:7045 | 9000:7071 |
| 9000:7020 | 9000:7046 | 9000:7072 |
| 9000:7021 | 9000:7047 | 9000:7073 |
| 9000:7022 | 9000:7048 | 9000:7074 |
| 9000:7023 | 9000:7049 | 9000:7075 |
| 9000:7024 | 9000:7050 | 9000:7076 |
| 9000:7025 | 9000:7051 | 9000:7077 |

### EXAMPLES

Additional embodiments are disclosed in further detail in the following examples, which are not in any way intended to limit the scope of the claims.

### Example 1

### Preparation of 1-O-acetyl-2,3-O-dibenzoyl-5(S)—C-methyl-5-O-(4-nitrobenzoyl)-D-ribofuranose (P1)

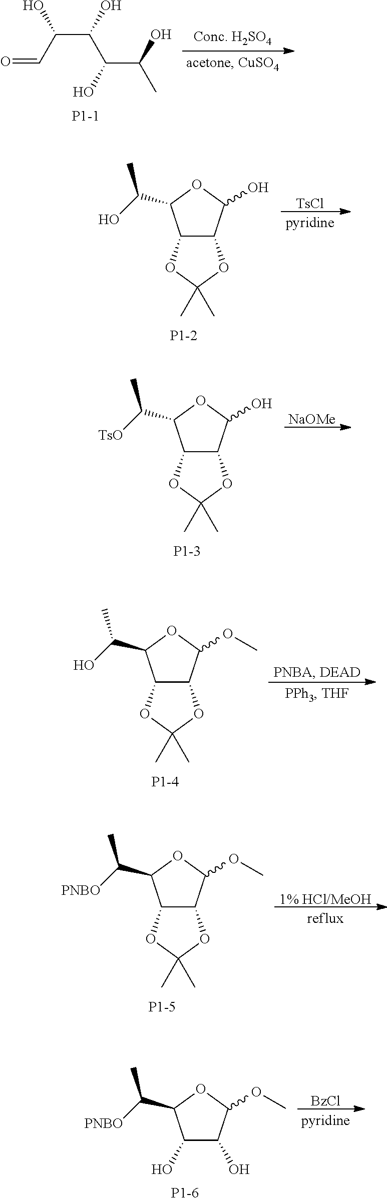

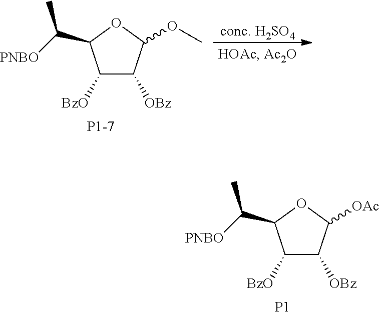

### Step 1. Preparation of 2,3-O-isopropylidene-L-rhamnofuranose (P1-2)

To a suspension of L-rhamnose hydrate (P1-1) (550 g×3, 3354 mmol×3) and anhydrous CuSO₄ (1000 g×3, 6250 mmol×3) in acetone (4000 mL×3) was added conc. H₂SO₄ (98%, 20 mL×3) dropwise. The mixture was stirred at RT (room temperature) for 20 h. The mixture was neutralized with saturated aq. ammonia, and the precipitate was removed by filtration on celite. The filtrate was concentrated to nearly dryness and then chloroform (5000 mL) was added. The mixture was stirred at RT for 2 h, and the precipitate was removed by filtration. The filtrate was concentrated to give crude P1-2 as light yellow oil (2010 g, 98%) which was used in the next step without further purification.

### Step 2. Preparation of 2,3-O-isopropylidene-5-O-tosyl-L-rhamnofuranose (P1-3)

To a solution of compound P1-2 (670 g×3, 3267 mmol×3) in anhydrous pyridine (1000 mL×3) was added a solution of TsCl (749 g×3, 3933 mmol×3) in dry CHCl₃ dropwise at 0° C. After addition, the mixture was warmed to RT and stirred for 20 h. The reaction was quenched with H₂O, and the solution was concentrated under reduced pressure. The residue was taken up to EA (ethyl acetate) and washed with water, cold H₂SO₄ (5%), saturated NaHCO₃ aqueous solution and brine in sequence. The organic phase was dried over Na₂SO₄ and concentrated to give a residue, which was subjected to crystallization in toluene and petroleum ether to give P1-3 as white solid (1800 g, 51%).

### Step 3. Preparation of 1-O,5(R)—C-dimethyl-2,3-O-isopropylidene-D-ribofuranose (P1-4)

To a stirred solution of compound P1-3 (450 g×4, 1257 mmol×4) in anhydrous MeOH (1000 mL×4) was added NaOMe (137 g×4, 2537 mmol×4) in portions at 0° C. The mixture was then stirred at RT for 20 h. The mixture was bubbled with CO₂ to adjust the pH value to about 8. The solvent was removed under reduced pressure. The residue was taken up to EA and washed with brine. The organic layer was dried over Na₂SO₄ and concentrated to give crude P1-4 (690 g), which was used in the next step without further purification.

### Step 4. Preparation of 1-O,5(S)—C-dimethyl-2,3-O-isopropylidene-5-O-(4-nitrobenzoyl)-D-ribofuranose (P1-5)

To a stirred solution of compound P1-4 (166 g×3, 761 mmol×3), p-nitrobenzoic acid (127 g×3, 761 mmol×3) and PPh₃ (600 g×3, 2290 mmol×3) in anhydrous THF (tetrahydrofuran) (1200 mL×3) was added DEAD (Diethyl azodicarboxylate) (400 g×3, 2290 mmol×3) dropwise at 0° C. After addition, the mixture was warmed to RT and stirred overnight. The solvent was removed, and the residue was re-dissolved in DCM (dichloromethane). The mixture was then treated with H₂O₂ (10% aqueous solution) at 0-5° C. The organic phase was concentrated and dissolved in MTBE (methyl tert-butyl ether). PPh₃O was filtered out, and 1600 g of the crude product was obtained. The crude product was then purified on silica gel column (pure PE (petroleum ether) to PE:EA=5:1 gradient) to give P1-5 as white solid (400 g, 48%).

### Step 5. Preparation of 1-O,5(S)—C-dimethyl-5-O-(4-nitrobenzoyl)-D-ribofuranose (P1-6)

Compound P1-5 (200 g×2, 545 mmol×2) was dissolved in conc. HCl and MeOH (2000 mL×2, 1% HCl in MeOH), and the mixture was refluxed for 8 h. The mixture was then cooled to RT and concentrated under reduced pressure. The residue was dissolved in DCM and washed with saturated NaHCO₃ aqueous solution, 5% H₂SO₄ and brine in sequence. The organic layer was dried over Na₂SO₄ and concentrated to give crude P1-6 (320 g), which was used in the next step without further purification.

### Step 6. Preparation of 2,3-O-dibenzoyl-1-O,5(S)—C-dimethyl-5-O-(4-nitrobenzoyl)-D-ribofuranose (P1-7)

To a stirred solution of crude P1-6 (160 g×2, 489 mmol×2) in dry pyridine (2000 mL×2) was added BzCl (212 g×2, 1504 mmol×2) at 0° C. dropwise. After addition, the mixture was stirred at RT for 20 h as checked by TLC. The reaction was quenched with H₂O and concentrated. The residue was taken up to EA and washed with saturated NaHCO₃ aqueous solution, 5% cold H₂SO₄ and brine in sequence. The organic phase was dried over Na₂SO₄ and concentrated to give crude P1-7 (520 g), which was used in the next step without further purification.

### Step 7. Preparation of 1-O-acetyl-2,3-O-dibenzoyl-5(S)—C-methyl-5-O-(4-nitrobenzoyl)-D-ribofuranose (P1)

To a stirred solution of crude P1-7 (130 g×4, 243 mmol×4) in HOAc (1000 mL×4) and Ac₂O (70 mL×4) was added conc. H₂SO₄ (70 mL×4) at 0° C. dropwise. After addition, the mixture was warmed to RT and stirred for 20 h as checked by TLC. The mixture was poured into ice-water with vigorous stirring. The precipitate was collected by filtration, and the filter cake was washed with water. The cake was then dissolved in EA and washed with saturated NaHCO₃ aqueous solution. The organic phase was dried over Na₂SO₄ and concentrated. The residue was purified on silica gel column (PE:EA=50:1 to 5:1) to give 1-O-acetyl-2,3-O-dibenzoyl-5(R)—C-methyl-5-O-(4-nitrobenzoyl)-D-ribofuranose (P1) as white foam (270 g, 49%); ¹H NMR (CDCl₃) δ 8.31-7.29 (m, 14H), 6.74 & 6.42 (d, J=4.8 Hz), brs, 1H), 5.85 (dd, J=4.8, 7.2 Hz, 1H), 5.74-5.43 (m, 2H), 4.65-4.61-5.43 (m, 1H), 2.19, 2.14 (2s, 3H), 1.55, 1.49 (2d, J=6.4 Hz, 3H), ESI-LCMS: m/z 586.2 [M+Na]⁺.

### Example 2

### Preparation of 1-O-acetyl-5(R)—C-methyl-2,3,5-O-tribenzoyl-D-ribofuranose (P2)

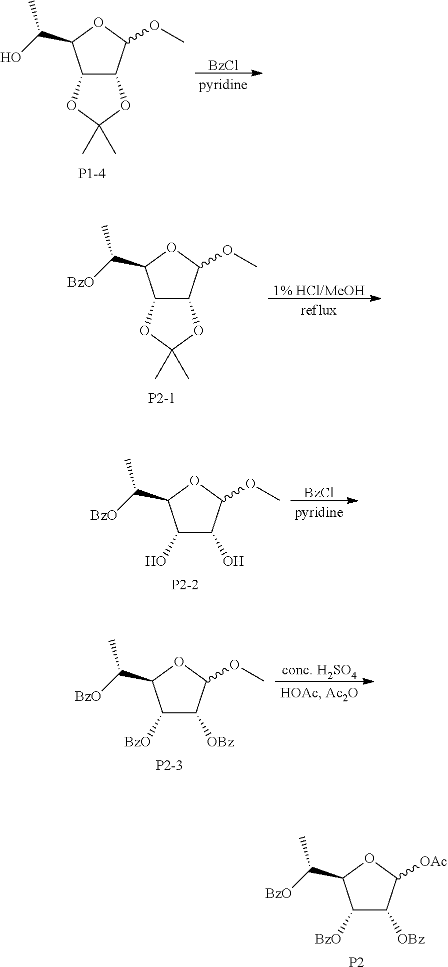

### Step 1. Preparation of 5-O-benzoyl-1-O, 5(R)—C-dimethyl-2,3-O-isopropylidene-D-ribofuranose (P2-1)

To a stirred solution of 1-O,5(R)—C-dimethyl-2,3-O-isopropylidene-D-ribofuranose (P1-4) (30 g, 137.61 mmol) in anhydrous pyridine (300 mL) was added BzCl (38.53 g, 275.23 mmol) dropwise at 0° C. The mixture was then stirred at RT for 20 h as checked by TLC. The reaction was quenched with water, and the solution was concentrated. The residue was diluted with EA and washed with saturated NaHCO₃ aqueous solution, cold 5% H₂SO₄ and brine in sequence. The organic phase was dried over Na₂SO₄ and concentrated to give crude P2-1 (40 g).

### Step 2. Preparation of 5-O-benzoyl-1-O,5(R)—C-dimethyl-D-ribofuranose (P2-2)

Compound P2-1 (40 g) was dissolved in conc. HCl and MeOH (300 mL, 1% HCl in MeOH). The mixture was refluxed for 4 h as checked by TLC. The mixture was then cooled to RT and concentrated. The residue was diluted with DCM and washed with saturated NaHCO₃ aqueous solution. The organic phase was dried over Na₂SO₄ and concentrated to give crude P2-2 (38 g), which was used in the next step without further purification.

### Step 3. Preparation of 1-O,5(R)—C-dimethyl-2,3,5-O-tribenzoyl-D-ribofuranose (P2-3)

To a stirred solution of crude P2-2 (38 g) in anhydrous pyridine (350 mL) was added BzCl (66.03 g, 471.63 mmol) dropwise at 0° C. After addition, the mixture was warmed to RT and stirred for 20 h as checked by TLC. The reaction was quenched with water, and the solution was concentrated. The residue was diluted with EA and washed with saturated NaHCO₃ aqueous solution, 5% H₂SO₄ and brine in sequence. The organic phase was dried over Na₂SO₄ and concentrated to give crude P2-3 (40 g), which was used in the next step without further purification.

### Step 4. Preparation of 1-O-acetyl-5(R)—C-methyl-2,3,5-O-tribenzoyl-D-ribofuranose (P2)

To a stirred solution of crude P2-3 (40 g) in HOAc (500 mL) and Ac₂O (35 mL) was added conc. H₂SO₄ (98%, 20 mL) dropwise at 0° C. After addition, the mixture was stirred at RT for 20 h as checked with TLC. The solution was poured into ice water with vigorous stirring. The precipitate was collected by filtration, and the filter cake was washed with water. The filter cake was then dissolved in EA and washed with saturated NaHCO₃ aqueous solution and brine. The organic phase was dried over Na₂SO₄ and concentrated. The residue was purified by column on silica gel (PE:EA=100:1 to 5:1) to give 1-O-acetyl-5(R)—C-methyl-2,3,5-O-tribenzoyl-D-ribofuranose (P2) (25 g, 59.12%); ¹H NMR (CDCl₃) δ 8.10-7.26 (m, 15H), 6.61 & 6.37 (2d, J=4.8, 0.8 Hz, 1H), 6.03-5.96 (m, 1H), 5.75, 5.59 (2dd, J=4.8, 0.8 & J=4.4, 6.4 Hz, 1H), 5.51-5.45 (m, 1H), 4.62-4.59 (m, 1H), 2.12, 1.81 (2s, 3H), 1.51, 1.45 (2d, J=6.4 Hz, 3H), ESI-LCMS: m/z 541.4 [M+Na]⁺.

### Example 3

### Preparation of 2′,3′-O-methoxymethylidene-N⁶-(4-methoxytrityl)-5′(S)—C-methyladenosine (P3)

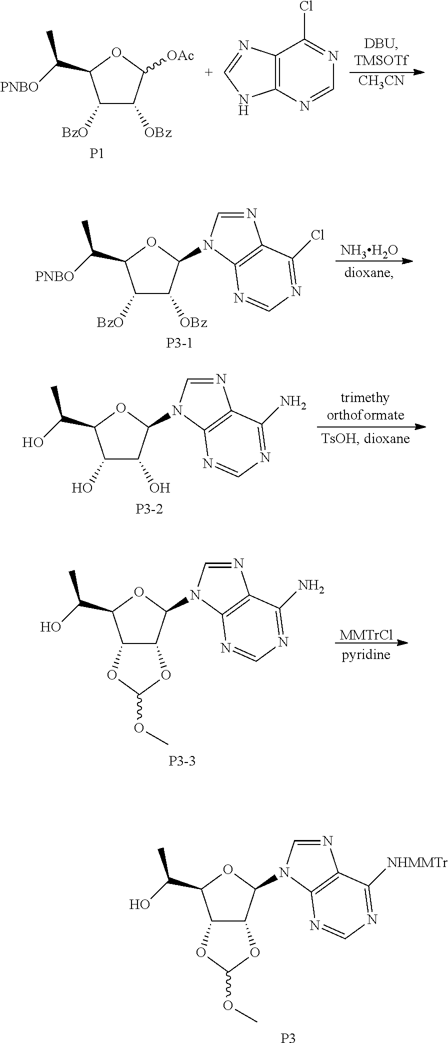

### Step 1. Preparation of 9-(2,3-O-dibenzoyl-5-O-4-nitrobenzoyl-5(S)—C-methyl-β-D-ribofuranosyl)-6-chloropurine (P3-1)

To a stirred suspension of 1-O-acetyl-2,3-O-dibenzoyl-5(S)—C-methyl-5-O-(4-nitribenzoyl)-D-ribofuranose (P1) (75 g×3, 133 mmol×3) and 6-chloro-9H-purine (20.9 g×3, 135 mmol×3) in anhydrous MeCN (400 mL×3) was added DBU (1,8-diazabicyclo(5.4.0)undec-7-ene) (61 g×3, 400 mmol×3) at 0° C. The mixture was stirred at 0° C. for 5 min and then TMSOTf (105 mL×3, 536 mmol×3) was added dropwise at 0° C. After addition, the mixture was stirred at 0° C. for 20 min until a clear solution achieved. Then the mixture was heated to 70° C. and stirred for 3 h. The reaction was cooled to room temperature and diluted with EA. The solution was washed with saturated NaHCO₃ and brine in sequence. The organic layer was dried over Na₂SO₄ and then concentrated. The residue was purified on silica gel column (PE:EA=4:1 to 3:1) to give P3-1 as light yellow foam (201 g, 76%).

### Step 2. Preparation of 5′(S)—C-methyladenosine (P3-2)

Compound P3-1 (100 g×2, 152 mmol×2) was dissolved in a (200 ml×2) of 1,4-dioxane and then saturated aqueous ammonia was added (200 mL×2). The mixture was stirred at 100° C. in a sealed vessel for 10 h. The mixture was cooled to room temperature and diluted with MeOH. The solvent was removed under reduced pressure, and the residue was purified column on silica gel column (MeOH:DCM=1:20 to 1:8) to give 5′ (S)—C-methyladenosine (P3-2) as white solid (76 g, 88%); ¹H NMR (CD₃OD) δ 8.31 (s, 1H), 8.17 (s, 1H), 5.95 (d, J=6.8 Hz, 1H), 4.73 (m, 1H), 4.27 (dd, J=5.2 Hz, 2.4 Hz, 1H), 4.07 (t, J=2.4 Hz, 1H), 3.96-3.91 (m, 1H), 3.30 (m, 1H), 1.25 (d, J=6.8 Hz, 3H); ESI-LCMS: m/z 282 [M+H]⁺.

### Step 3. Preparation of 2′,3′-O-methoxymethylidene-5′(S)—C-methyladenosine (P3-3)

A mixture of compound P3-2 (17 g, 60.5 mmol), trimethyl orthoformate (170 mL) and p-toluenesulfonic acid monohydrate (18 g, 94.7 mmol) in 1,4-dioxane (160 mL) was stirred at 50° C. for 12 h, cooled with ice and quenched by triethylamine (15 mL), The mixture was then concentrated. The residue was purified by chromatography on silica gel with 0-0.5% MeOH in EA gave product P3-3 as white solid (15 g, 77%).

### Step 4. Preparation of 2′,3′-O-methoxymethylidene-N⁶-(4′-methoxytrityl)-5′(S)—C-methyladenosine (P3)

A mixture of compound P3-3 (15 g, 46.4 mmol, co-evaporated with dry pyridine for twice) and MMTrCl (21 g, 68 mmol) were suspended in anhydrous pyridine (150 mL). The mixture was stirred at 50° C. for 12 h. The mixture was then quenched with H₂O and concentrated. The residue was purified by column on silica gel (PE/EA=3:1 to 1:1) to afford 2′,3′-O-methoxymethylidene-N⁶-(4-methoxytrityl)-5′(S)—C-methyladenosine (P3) as white foam (12 g, 44%).

### Example 4

### Preparation of 2′,3′-O-methoxymethylidene-N⁴-(4-methoxytrityl)-5′(S)—C-methylcytidine (P4)

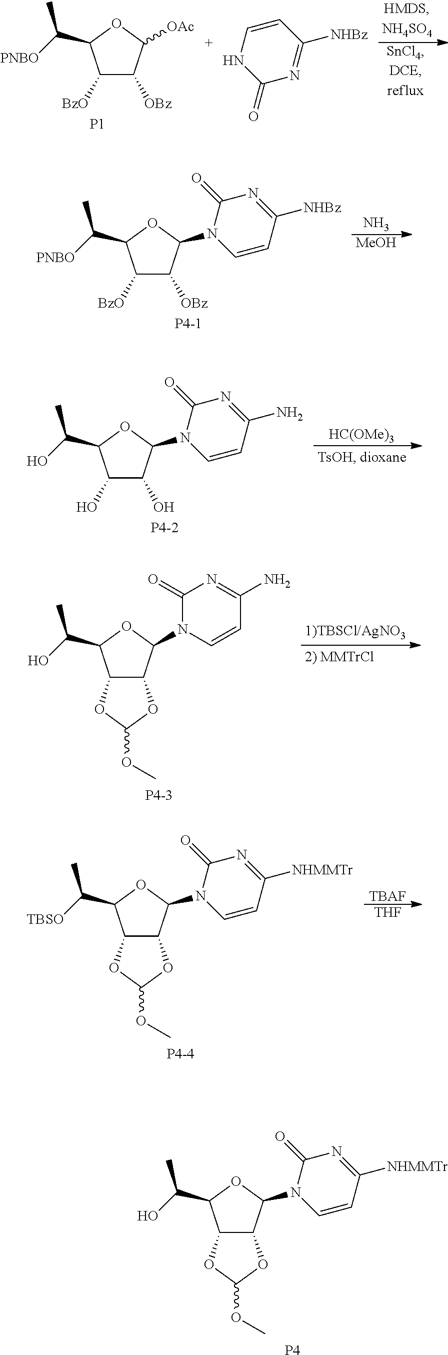

### Step 1. Preparation of 5′(S)—C-methyl-5′-O-(4-nitrobenzoyl)-2′,3′-O, N4-tribenzoylcytidine (P4-1)

N⁴-Benzoylcytosine (3.5 g, 16.87 mmol) in dry dichloroethane (100 mL) was treated with excess 1,1,1,3,3,3-hexamethyl-disilazane (15 mL) in the presence of ammonium sulfate (100 mg) under argon and refluxed at 125° C. for 2 h until all the solid dissolved. Excess solvent was evaporated under reduced pressure, and the resulting syrup was dissolved in dry dichloroethane (100 mL). Compound P1 (5 g, 8.88 mmol) was added, followed by addition of SnCl₄ (10 mL). The resulting mixture was heated under reflux overnight, cooled with ice, diluted with ethyl acetate, washed with aqueous sodium bicarbonate, dried over anhydrous Na₂SO₄ and concentrated. Chromatography on silica gel with 10-15% ethyl acetate in DCM gave 5.5 g of compound P4-1.

### Step 2. Preparation of 5′(S)—C-methylcytidine (P4-2)

Compound P4-1 (5.5 g, 7.66 mmol) in saturated ammonia in MeOH (200 mL) was stirred at RT overnight. The solvent was removed and the residue was re-dissolved in MeOH. Precipitation from MeOH/DCM gave P4-2 (1.5 g, 76.19%). ¹H NMR (400 MHz, MeOD): δ 8.08 (d, J=7.6 Hz, 1H), 5.85 (d, J=7.6 Hz, 1H), 5.82 (d, J=3.6 Hz, 1H), 4.11-4.13 (m, 1H), 4.05-4.08 (m, 1H), 3.89-3.94 (m, 1H), 3.79-3.81 (m, 1H), 1.27 (d, J=6.8 Hz, 3H); ESI-MS: m/z 515 [2M+H]⁺, 258 [M+H]⁺.

### Step 3. Preparation of 2,3′-O-methoxymethylidene-5′(R)—C-methylcytidine (P4-3)

A mixture of compound P4-2 (500 mg, 1.95 mmol), trimethyl orthoformate (3 mL) and p-toluenesulfonic acid monohydrate (450 mg, 2.33 mmol) in 1,4-dioxane (10 mL) was stirred at RT for 24 h, cooled with ice and quenched by adding triethylamine (5 mL) and concentrated. The residue was purified by column on silica gel with 5-6% MeOH in DCM gave compound P4-3 as white foam (450 mg, 77.36%).

### Step 4. Preparation of 5′-O-(tert-butyldimethylsilyl)-2′,3′-O-methoxymethylidene-N4-(4-methoxytrityl)-5′(S)—C-methylcytidine (P4-4)

To a stirred solution of compound P4-3 (450 mg 1.51 mmol) in pyridine (5 ml) was added TBSCl (t-butyldimethylsilyl chloride) (450 mg, 3.01 mmol) and AgNO₃ (0.51 g, 3.01 mmol). The mixture was stirred at 50-60° C. for 3 h. MMTrCl (0.93 g, 3.01 mmol) was then added. The mixture was stirred overnight at 50-60° C. until the reaction was complete, as determined by TLC. The reaction was cooled to RT and diluted with EA. The precipitate was removed by filtration, and the filtrate was washed with brine in sequence. The organic layer was dried over Na₂SO₄ and then concentrated to give 800 mg crude product of P4-4.

### Step 5. Preparation of 2,3′-O-methoxymethylidene-N4-(4-methoxytrityl)-5′(S)—C-methylcytidine (P4)

Compound P4-4 (800 mg crude) in 1M TBAF in THF (20 mL) was stirred at RT overnight. The solvent was removed and the residue was purified on silica gel column and then by prep. TLC to give P4 (100 mg), ¹H NMR (400 MHz, CDCl₃): δ 7.25-6.76 (m, 14H), 5.79 (d, 1H), 5.28-4.99 (m, 4H), 4.09 (m, 3H), 3.72 (s, 3H), 3.28, 3.21 (2s, 3H), 1.17 (brs, 3H); ESI-MS: m/z 572 [M+H]⁺.

### Example 5

### Synthesis of 2′,3′-O-methoxymethylidene-N4-(4-methoxytrityl)-5′(R)—C-methylcytidine (P5)

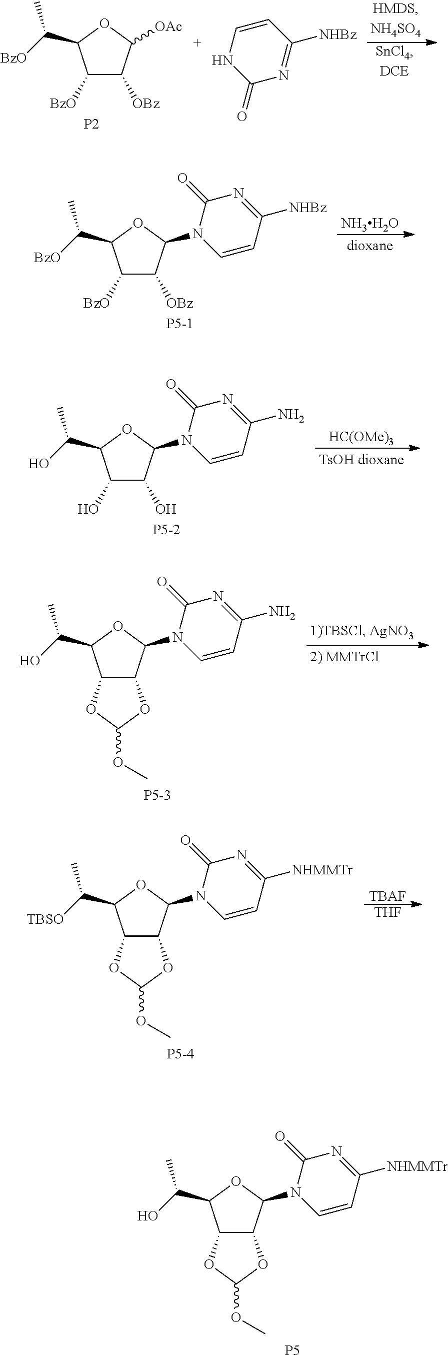

### Step 1. Preparation of 5′(R)—C-methyl-5′-O-(4-nitrobenzoyl)-2′,3′-O,N4-tribenzoylcytidine (P5-1)

N⁴-Benzoylcytosine (1.5 g, 6.95 mmol) in dry dichloroethane (100 mL) was treated with excess 1,1,1,3,3,3-hexamethyl-disilazane (15 mL) in the presence ammonium sulfate (75 mg) under argon and refluxed at 125° C. for 2 h until all the solid dissolved. Excess solvent was evaporated under reduced pressure, and the resulting syrup was dissolved in dry dichloroethane (100 mL). Compound P2 (3 g, 5.79 mmol) was added, followed by addition of SnCl₄ (5 mL). The resulting mixture was heated under reflux overnight, cooled with ice, diluted with ethyl acetate, washed with aqueous sodium bicarbonate, dried over anhydrous Na₂SO₄ and concentrated. Chromatography on silica gel with 10-15% ethyl acetate in DCM gave 2.8 g of compound P5-1.

### Step 2. Preparation of 5′(R)—C-methylcytidine (P5-2)

Compound P5-1 (2.8 g, 4.16 mmol) in dioxane (5 mL) and saturated ammonia in H₂O (30 mL) was stirred at 100° C. in a sealed vessel overnight. The solvent was removed, and the residue was re-dissolved in MeOH. Precipitation from MeOH/DCM gave 5′(R)—C-methylcytidine (P5-2) (750 mg, 70.1%). ¹H NMR (400 MHz, MeOD): δ 7.87 (d, J=7.6 Hz, 1H), 5.81 (d, J=7.2 Hz, 1H), 5.75 (d, J=4.8 Hz, 1H), 4.10-4.15 (m, 2H), 3.90-3.96 (m, 1H), 3.76-3.78 (m, 1H), 1.16 (d, J=6.8 Hz, 3H); ESI-LCMS: m/z 515 [2M+H]⁺, 258 [M+H]⁺.

### Step 3. Preparation of 2,3′-O-methoxymethylidene-5′(R)—C-methylcytidine (P5-3)

A mixture of compound P5-2 (750 mg, 2.92 mmol), trimethyl orthoformate (5 mL) and p-toluenesulfonic acid monohydrate (670 mg, 3.5 mmol) in 1,4-dioxane (10 mL) was stirred at RT for 24 h, cooled with ice, quenched by adding triethylamine (5 mL) and concentrated. The residue was purified by column on silica gel with 5-6% MeOH in DCM gave compound P5-3 as white foam (700 mg, 80.3%).

### Step 4. Preparation of 5′-O-(tert-butyldimethylsilyl)-2′,3′-O-methoxymethylidene-N4-(4-methoxytrityl)-5′ (R)—C-methylcytidine (P5-4)

To a stirred solution of compound P5-3 (700 mg 2.34 mmol) in pyridine (5 mL) was added TBSCl (700 mg, 4.68 mmol) and AgNO₃ (0.79 g, 4.68 mmol). The mixture was stirred at 50-60° C. for 3 h as checked by LCMS. MMTrCl (1.44 g, 4.68 mmol) was added. The mixture was stirred overnight at 50-60° C. The reaction was cooled to room temperature and diluted with EA. The precipitate was removed by filtration, and the filtrate was washed with brine. The organic layer was dried over Na₂SO₄ and then concentrated to give crude product of compound P5-4.

### Step 5. Preparation of 2,3′-O-methoxymethylidene-N4-(4-methoxytrityl)-5′ (R)—C-methylcytidine (P5)

Compound P5-4 (1.2 g crude) in 1M TBAF in THF (20 mL) was stirred at RT overnight. The solvent was removed, and the residue was purified by prep. TLC to give 220 mg of 2′,3′-O-methoxymethylidene-N⁴-(4-methoxytrityl)-5′(R)—C-methylcytidine (P5).

### Example 6

### Preparation of 2′,3′-O-methoxymethylidene-5′(S)—C-methyluridine (P6)

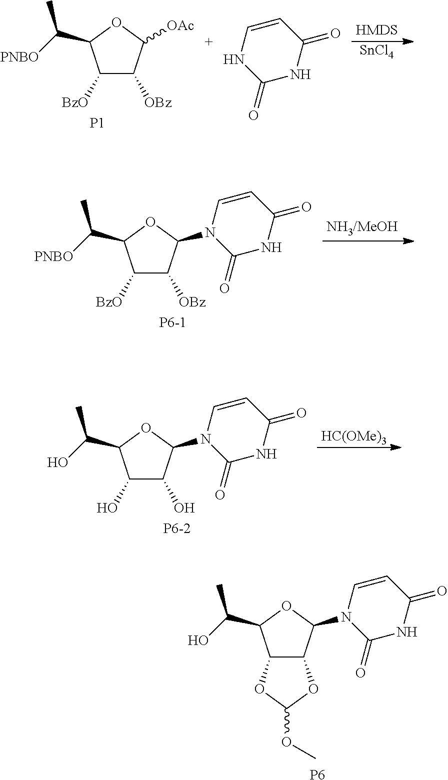

### Step 1. Preparation of 2,3′-O-dibenzoyl-5′(S)—C-methyl-5′-O-(4-nitrobenzoyl)uridine

Uracil (2 g, 8.25 mmol) in dry dichloroethane (50 mL) was treated with excess 1,1,1,3,3,3-hexamethyl-disilazane (20 mL) in the presence ammonium sulfate (100 mg) under argon. The mixture was refluxed at 125° C. for 2 h until all the solid had dissolved. Excess solvent was evaporated under reduced pressure, and the resulting syrup was dissolved in dry dichloroethane (50 mL). 1-O-acetyl-2,3-O-dibenzoyl-5(5)-C-methyl-5-O-(4-nitro-benzoyl)-D-ribofuranose (P1) (4 g, 7.10 mmol) was added, followed by addition of SnCl₄ (10 mL). The resulting mixture was heated under reflux overnight, cooled with ice, diluted with ethyl acetate, washed with aqueous sodium bicarbonate, dried over anhydrous Na₂SO₄ and concentrated. Chromatography on silica gel with 10-15% ethyl acetate in DCM gave 4 g of P6-1.

### Step 2. Preparation of 5′(S)—C-methyluridine (P6-2)

2′,3′-O-dibenzoyl-5′(S)—C-methyl-5′-O-(4-nitrobenzoyl)uridine (P6-1) (4 g, 6.51 mmol) in methanol (100 mL) and saturated ammonia in MeOH (200 mL) was stirred at RT overnight. The solvent was removed, and the residue was re-dissolved in MeOH. Precipitation from MeOH/DCM gave 1.5 g of 5′(S)—C-methyluridine (P6-2) as a white solid. ¹H NMR (400 MHz, CD₃OD): δ 8.07 (d, J=8.0 Hz, 1H), 5.88 (d, J=5.2 Hz, 1H), 5.67 (d, J=8.0 Hz, 1H), 4.15 (s, 1H), 4.10-4.08 (m, 1H), 3.92-3.90 (m, 1H), 3.80 (dd, J₁=4.4 Hz, J₂=2.4 Hz, 1H), 1.25 (d, J=6.4 Hz, 3H); ESI-LCMS: m/z 281 [M+Na]⁺, 259 [M+H]⁺.

### Step 3. Preparation of 2′,3′-O-methoxymethylidene-5′(S)—C-methyluridine (P6)

A mixture of 5′(S)—C-methyluridine (P6-2) (500 mg, 1.8 mmol), trimethyl orthoformate (2.5 mL) and p-toluenesulfonic acid monohydrate (500 mg, 0.6 mmol) in THF (100 mL) was stirred at RT for 24 h, the crude product was purified by HPLC to give 300 mg of 2′,3′-O-methoxymethylidene-5′(S)—C-methyluridine (P6); ¹H NMR (400 MHz, CD₃OD): δ 8.04 (brs, 1H), 7.30, 7.25 (2×d, J=8.0 Hz, 1H), 5.88, 5.92 (2×s, 1H), 5.70, 5.68 (dd, J=2.8, 8.0 Hz, 1H), 5.6, 5.52 (2×d, J=3.2 Hz, 1H), 5.02 (m, 1H), 4.87-4.93 (m, 1H), 4.10-3.91 (m, 2H), 3.34 (s, 3H), 2.51, 2.38 (2×d, J=6.8, 5.6 Hz, 1H), 1.23, 1.21 (2×d, J=2.4, 2.8 Hz, 3H); ESI-LCMS: m/z 323.08 [M+Na]⁺.

### Example 7

### Preparation of 2′,3′-O-methoxymethylidene-5′(R)—C-methyluridine (P7)

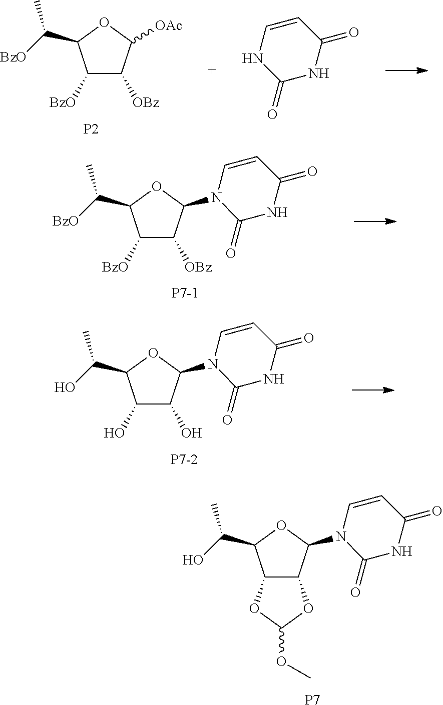

### Step 1. Preparation of 2′,3′,5′-O-tribenzoyl-5′(R)—C-methyluridine (P7)

Uracil (2 g, 8.9 mmol) in dry dichloroethane (50 mL) was treated with excess 1,1,1,3,3,3-hexamethyl-disilazane (20 mL) in the presence of ammonium sulfate (100 mg) under argon. The mixture was refluxed at 125° C. for 2 h until all the solid had dissolved. Excess solvent was evaporated under reduced pressure, and the resulting syrup was dissolved in dry dichloroethane (50 mL). 1-O-acetyl-2,3,5-O-tribenzoyl-5(R)—C-methyl-D-ribofuranose (P2) (2.3 g, 4.5 mmol) was added, followed by addition of SnCl₄ (5 mL). The resulting mixture was heated under reflux overnight, cooled with ice, diluted with ethyl acetate, washed with aqueous sodium bicarbonate, dried over anhydrous Na₂SO₄ and concentrated. Chromatography on silica gel with 10-15% ethyl acetate in DCM gave 1.2 g of 2′,3′,5′-O-tribenzoyl-5′(R)—C-methyluridine (P7-1).

### Step 2. Preparation of 5′(R)—C-methyluridine (P7-2)

2′,3′,5′-O-tribenzoyl-5′(R)—C-methyl-uridine (P7-1) (1.2 g, 2.1 mmol) in methanol (100 mL) and saturated ammonia in MeOH (200 mL) was stirred at 100° C. in a sealed vessel for 10 h. The mixture was cooled to RT and diluted with MeOH. The solvent was removed under reduced pressure, and the residue was purified by column on silica gel (MeOH:DCM=1:20 to 1:8) to give 400 mg of P7-2 as white solid; ¹H NMR (400 MHz, CD3OD): δ7.95 (d, J=8.4 Hz, 1H), 5.89 (d, J=6 Hz, 1H), 5.69 (d, J=8.4 Hz, 1H), 4.21-4.15 (m, 2H), 3.97-3.95 (m, 1H), 3.80 (t, J=3.2 Hz, 1H), 1.23 (d, J=6.8 Hz, 3H); MS: m/z 259 [M+H]⁺.

### Step 3. Preparation of 2′,3′-O-methoxymethylidene-5′(R)—C-methyluridine (P7)

A mixture of 5′(R)—C-methyluridine (P7-2) (500 mg, 1.8 mmol), trimethyl orthoformate (2.5 mL) and p-toluenesulfonic acid monohydrate (500 mg, 0.6 mmol) in THF (100 mL) was stirred at RT for 24 h, the crude product was purified by reverse-phase HPLC (HCOOH) to gave 320 mg of 2′,3′-O-methoxymethylidene-5′(R)—C-methyluridine (P7); ¹H NMR (400 MHz, CD₃OD): δ 9.04, 8.98 (2×brs, 1H), 7.30, 7.26 (2×d, J=8.0 Hz, 1H), 5.97, 5.91 (2×s, 1H), 5.73 (d, J=8.0 Hz, 1H), 5.58, 5.48 (2×d, J=2.8 Hz, 1H), 5.16-5.08 (m, 2H), 4.15-3.97 (m, 2H), 3.37, 3.31 (2×s, 3H), 1.26, 1.25 (2×d, J=2.4, 2.8 Hz, 3H); ESI-LCMS: m/z 301.1 [M+H]⁺.

### Example 8

### Preparation of 2′-deoxy-2′-α-fluoro-3′-O, N4-di(4-methoxytrityl)-2′-β,5′(S)—C-dimethylcytidine (P8)

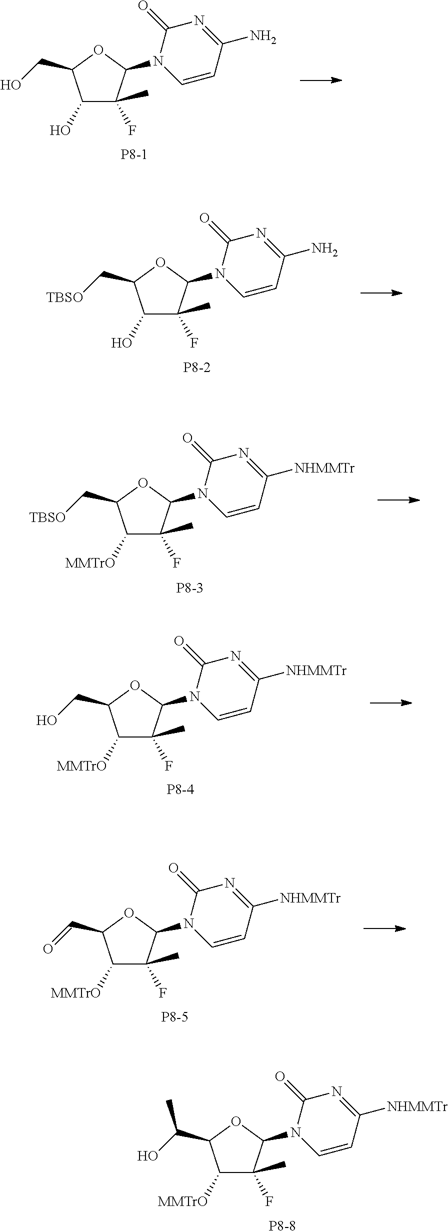

### Step 1. Preparation of 5′-O-(tert-butyldimethylsilyl)-2′-deoxy-2′-α-fluoro-2′-β-C-methylcytidine (P8-2)

To an ice-cold solution of 2′-α-fluoro-2′-β-C-methylcytidine (P8-1) (2.5 g, 9.6 mmol) in anhydrous pyridine (20 mL) was added TBSCl (1.6 g, 10.6 mmol) in small portions under N₂. The reaction mixture was stirred at RT overnight. LCMS showed the reaction was completed. The solvent was removed under vacuum. The residue was diluted with EA (100 mL), washed with water and brine. The organic layer was separated, dried over anhydrous Na₂SO₄ and filtered. The filtrate was concentrated in vacuum to give crude compound P8-2 (3.5 g) without further purification.

### Step 2. Preparation of 5′-O-(tert-butyldimethylsilyl)-2′-deoxy-2′-α-fluoro-2′-β-C-methylcytidine (P8-3)

To a mixture of crude P8-2 (3.5 g, 9.38 mmol), AgNO₃ (3.1 g, 18.7 mmol) and collidine (3.4 g, 28.1 mmol) in anhydrous DCM (300 mL) was added MMTrCl (6.1 g, 20 mmol) in small portions under N₂. The reaction mixture was stirred at RT overnight under N₂. The reaction mixture was filtered on celite. The filtrate was washed with saturated NaHCO₃ solution and followed by brine. The organic layer was separated, dried over anhydrous Na₂SO₄ and filtered. The filtrate was concentrated in vacuum to give the crude P8-3 (4.8 g), which was used in the next step without further purification.

### Step 3: Preparation of 2′-deoxy-2′-O,N4-di(4-methoxytrityl)-2′-β-C-methylcytidine (P8-4)

To an ice-cold crude P8-3 (4.8 g, 5.2 mmol) was added TBAF (1M solution in THF, 26 mmol) dropwise under N₂. The reaction mixture was stirred at RT overnight. The solvent was removed, and the residue was dissolved in EA (200 mL) and washed with water and brine. The organic layer was separated, dried over anhydrous Na₂SO₄ and filtered. The filtrate was concentrated in vacuum to give a residue, which was purified on silica gel column (PE/EA=6/1 to 2/1) to give compound P8-4 (4.8 g, 62%).

### Step 4: Preparation of 2′-deoxy-5′-C,5′-O-didehydro-3′-O,N4-di(4-methoxytrityl)-2′-α-fluoro-2′-β-C-methylcytidine (P8-5)

To a stirred solution of anhydrous pyridine (567 mg, 7.2 mmol) in anhydrous DMSO (10 mL) was added TFA (trifluoroacetic acid) (681 mg, 5.98 mmol) 0-5° C. The mixture was stirred at RT until a clear solution formed. The solution was then added to a mixture of compound P8-4 (4.8 g, 5.98 mmol) and DCC(N-dicyclohexylcarbodiimide) (4.9 g, 17.9 mmol) in 15 mL anhydrous DMSO under N₂. The reaction mixture was stirred at RT overnight. The reaction mixture was diluted with EA (200 mL), and washed with water and brine. The organic layer was separated, dried over anhydrous Na₂SO₄ and filtered. The filtrate was concentrated in vacuum to give an oil which was purified by silica gel column (PE/EA=10/1 to 2/1) to give compound P8-5 (3.5 g, 72%).

### Step 5: Preparation of 2′-deoxy-3′-O,N⁴-di(4-methoxytrityl)-2′-β,5′(S)—C-dimethyl-2′-α-fluorocytidine (P8)

To a solution of compound P8-5 (3.5 g, 4.3 mmol) in anhydrous THF (10 mL) was added MeMgBr (3 M solution in ether) (4.4 mL, 13.1 mmol) dropwise under N₂ at −78° C. The reaction mixture was stirred at RT overnight as monitored by TLC. The mixture was cooled to 0° C. The mixture was then quenched with saturated NH₄Cl and extracted with EA (100 mL×2). The combined organic layer was dried over anhydrous Na₂SO₄ and concentrated. The crude product was purified on silica gel column (PE/EA=3/1 to 1/1) to give 1.5 g (42.8%) of 2′-deoxy-3′-O,N⁴-di(4-methoxytrityl)-2′-β,5′(S)—C-dimethyl-2′-α-fluoro-cytidine (P8). Further purification by prep. HPLC afforded pure compound P8; ¹H NMR (400 Hz, CDCl₃): 7.45-6.78 (m, 30H), 6.19 (m, 1H), 4.90 (d, J=7.6 Hz, 1H), 4.08 (d, J=9.6 Hz, 1H), 3.81 (s, 3H), 3.76 (s, 3H), 3.50-3.52 (m, 1H), 1.15 (d, J=6.8 Hz, 3H), 0.78 (d, J=22 Hz, 3H); MS: m/z 918 [M+H]⁺.

### Example 9

### Preparation of 2′-deoxy-3′-O, N⁴-di(4-methoxytrityl)-2′-β,5′-(R)—C-dimethyl-2′-α-fluorocytidine (P9)

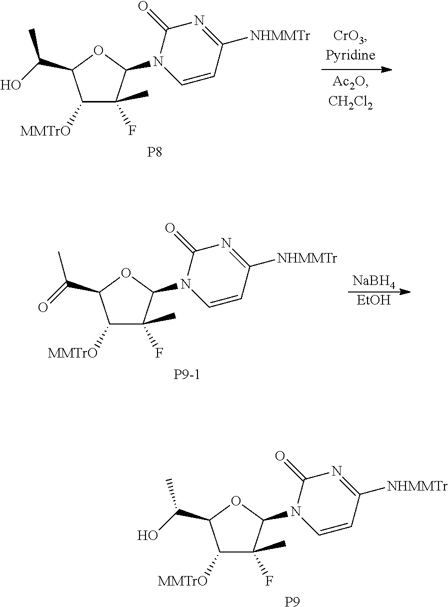

### Step 1. Preparation of 2′-deoxy-5′-C,5′-O-didehydro-3′-O, N⁴-di(4-methoxytrityl)-2′-β,5′(R)—C-dimethyl-12′-α-fluorocytidine (P9-1)

To an ice-cooled suspension of CrO₃ (100 mg, 1 mmol) in anhydrous DCM (5 mL) was added anhydrous pyridine (0.14 mL, 1.8 mmol) and Ac₂O (0.1 mL, 0.8 mmol) under N₂. The mixture was stirred at RT for about 10 min until the mixture became homogeneous. The mixture was cooled to 0° C., and a solution of compound P8 (240 mg, 0.3 mmol) in anhydrous DCM (5 mL) was added. The resulting mixture was stirred at RT for 1 h. The reaction went to completion as determined by TLC. The reaction mixture was diluted with DCM (50 mL), washed with NaHCO₃ solution twice and brine. The organic layer was separated, dried over anhydrous Na₂SO₄ and filtered. The filtrate was concentrated in vacuum to give P9-1 (200 mg, 83%) without further purification.

### Step 2. Preparation of 2′-deoxy-3′-O, N⁴-di(4-methoxytrityl)-2′-β,5′(R)—C-dimethyl-2′-α-fluorocytidine (P9)

To an ice-cold solution of compound P9-1 (200 mg, 0.25 mmol) in anhydrous EtOH (10 mL) was added NaBH₄ (19 mg, 0.5 mmol) under N₂. The reaction mixture was stirred at RT overnight. The reaction went to completion as determined by TLC. The solvent was evaporated. The residue was diluted with EA (30 mL), washed with saturated NaHCO₃ and brine. The organic layer was separated, dried over anhydrous Na₂SO₄ and concentrated. Purification by preparative TLC gave 2′-deoxy-3′-O,N⁴-di(4-methoxytrityl)-2′-β,5′(R)—C-dimethyl-2′-α-fluorocytidine (P9) (190 mg, 95%).

### Example 10

### Preparation of 5′(R)—C-methyl-2′,3′-O, N4-tri(4-methoxytrityl)arabinocytidine (P10)

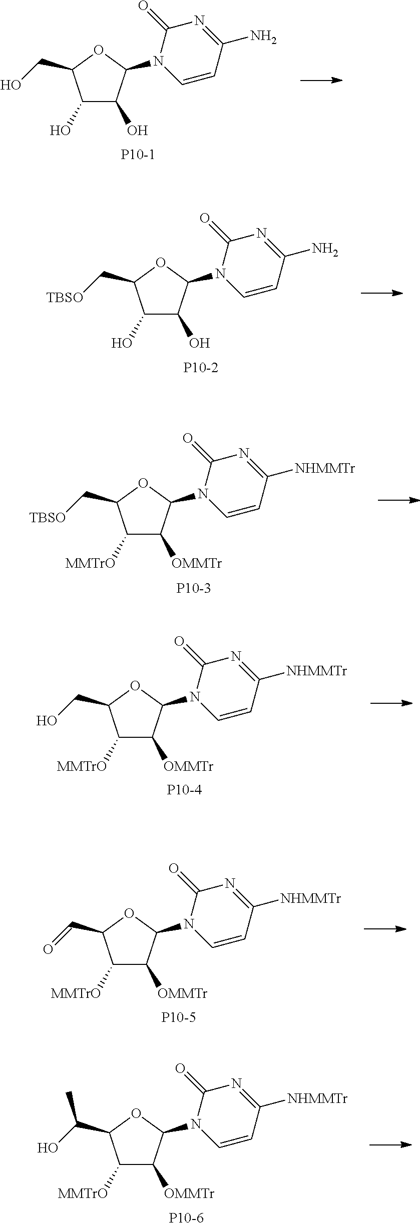

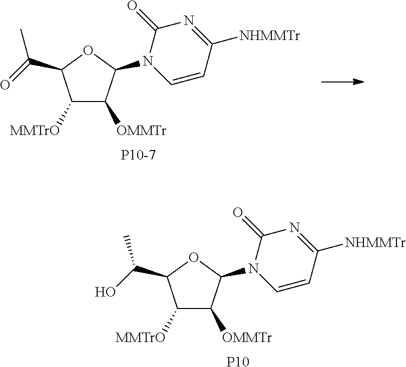

### Step 1. Preparation of 5′-O-(tert-butyldimethylsilyl)arabinocytidine (P10-1)

To an ice-cooled solution of arabinocytidine (P10-1) (20.0 g, 82.2 mmol) in anhydrous pyridine (200 mL) was added TBSCl (14.9 g, 98.7 mmol) in small portions under N₂. The reaction mixture was stirred at RT overnight. The solvent was removed under vacuum, and the residue was diluted with EA (300 mL), washed with water and brine. The organic layer was separated, dried over anhydrous Na₂SO₄ and filtered. The filtrate was concentrated in vacuum to give compound P10-2 (25.1 g, 85.4%) as a white solid, which was used without further purification.

### Step 2. Preparation of 5′-O-(tert-butyldimethylsilyl)-2′,3′-O,N4-tri(4-methoxytrityl)arabinocytidine (P10-3)

To a mixture of compound P10-2 (15.0 g, 41.96 mmol), AgNO₃ (43.5 g, 252 mmol) and collidine (61 g, 503.5 mmol) in anhydrous DCM (300 mL) was added MMTrCl (77.7 g, 252 mmol) in small portions under N₂. The reaction mixture was stirred at RT for two days under N₂. The reaction mixture was filtered with celite. The filtrate was washed with saturated NaHCO₃ solution and followed by brine. The organic layer was separated, dried over anhydrous Na₂SO₄ and filtered. The filtrate was concentrated in vacuum to give the residue which was purified on silica gel column (PE/EA=2/1) to give compound P10-3 (33.5 g, 67.9%).

### Step 3. Preparation of 2,3′-O,N4-tri(4-methoxytrityl)arabinocytidine (P10-4)

To an ice-cooled solution compound P10-3 (10.45 g, 8.9 mmol) in anhydrous THF (50 mL) was added TBAF (1M solution in THF) (49.8 mL, 49.8 mmol) dropwise under N₂. The reaction mixture was stirred at RT overnight. The solvent was removed, and the residue was dissolved in EA (180 mL) and washed with water and brine. The organic layer was separated, dried over anhydrous Na₂SO₄ and filtered. The filtrate was concentrated in vacuum to give a residue, which was purified by silica gel column (PE/EA=5/1 to 1/1) to give compound P10-4 (6.15 g, 97.0% and 4.17 g, 70%).

### Step 4. Preparation of 5′-C, 5′-O-didehydro-2′,3′-O,N4-tri(4-methoxytrityl)arabinocytidine (P10-5)

To a Stirred Solution of Dry Pyridine (588 Mg, 7.44 Mmol) in Anhydrous DMSO (12 mL) was added TFA (707 mg, 5.79 mmol) at about 5° C. The mixture was stirred at RT for 30 min until a clear solution formed. The solution was added to a solution of DCC (5.2 g, 25.2 mmol) and compound P10-4 (6.55 g, 6.17 mmol) in DMSO (18 mL) dropwise. The mixture was stirred at RT overnight. The reaction was quenched with H₂O, and the precipitate was removed by filtration. The filtrate was diluted with EA and washed with brine. The organic layer was dried over Na₂SO₄ and concentrated. The residue was purified on silica gel (PE:EA=5:1 to 1:2) to give compound P10-5 (5.06 g, 77%).

### Step 5. Preparation of 5′(S)—C-methyl-2,3′-O,N4-tri(4-methoxytrityl)arabinocytidine (P10-6)

To a solution of compound P10-5 (3.348 g, 3.16 mmol) in anhydrous THF (20 mL) was added MeMgBr (3M solution in ether) (6.27 mL, 15.8 mmol) dropwise at −78° C. The reaction mixture was stirred at RT overnight. After the reaction was complete, the mixture was cooled to 0° C. and quenched by saturated NH₄Cl. The product was extracted with EA (150 mL×2). The combined organic layer was dried over anhydrous Na₂SO₄ and concentrated to give 3.24 g (95%) of crude P10-6, which was further purified by chromatography on silica gel (PE/EA=10:1 to 1:1).

### Step 6. Preparation of 5′-C, 5′-O-didehydro-5′(S)—C-methyl-2,3′-O,N4-tri(4-methoxytrityl)arabinocytidine (P10-7)

To an ice-cooled suspension of CrO₃ (697.5 mg, 6.98 mmol) in anhydrous DCM (12.5 mL) was added anhydrous pyridine (1.125 mL, 13.98 mmol) and Ac₂O (0.7 mL, 6.98 mmol) under N₂. The mixture was stirred at RT for about 10 min until the mixture became homogeneous. The mixture was cooled to 0° C., and a solution of compound P10-6 (2.5 g, 2.33 mmol) in anhydrous DCM (12.5 mL) was added. The resultant mixture was stirred at RT overnight. The reaction mixture was diluted with EA (100 mL), washed with NaHCO₃ solution twice and brine. The organic layer was separated, dried over anhydrous Na₂SO₄ and filtered. The filtrate was concentrated under vacuum to give a crude product P10-7 (2.5 g).

### Step 7. Preparation of 5′ (R)—C-methyl-2′,3′-O,N4-tri(4-methoxytrityl)arabinocytidine (P10)

To an ice-cold solution of compound P10-7 (2.5 g, 2.33 mmol) in anhydrous EtOH (50 mL) was added NaBH₄ (250 mg, 6.47 mmol) under N₂. The reaction mixture was stirred at RT overnight. The solvent was evaporated. The residue was diluted with EA (30 mL), washed with saturated NaHCO₃ and brine. The organic layer was separated, dried over anhydrous Na₂SO₄ and concentrated to give the crude product. The crude was further purified by prep. TLC to give 5′(R)—C-methyl-2′,3′-O,N⁴-tri(4-methoxytrityl)arabinocytidine (P10) (1.4 g, 95% purity). ESI-MS: m/z 1074.2 [M+H]⁺.

### Example 11

### Preparation of 2′,3′-O-methoxymethylidene-N²-(4-methoxytrityl)-5′(S)—C-methylguanosine (P11)

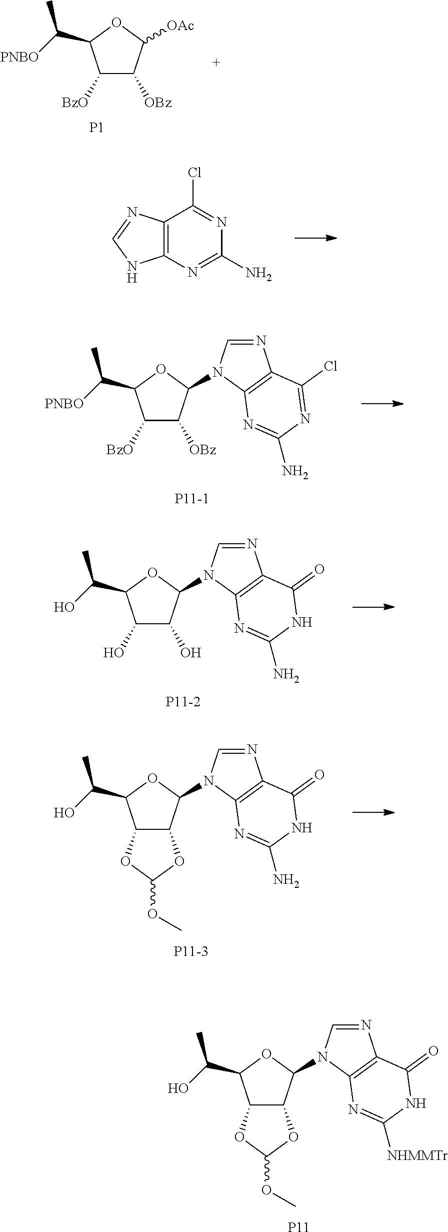

### Step 1. Preparation of 9-(2′,3′-O-dibenzoyl-5′(S)—C-methyl-5′-O-(4-nitrobenzoyl)-β-D-ribofuranosyl)-2-amino-6-chloropurine (P11-1)

To a stirred suspension of P1 (10 g, 17.8 mmol) and 2 (3.1 g, 18.2 mmol) in anhydrous MeCN (200 mL) was added DBU (8.1 g, 53.4 mmol) at 0° C. The mixture was stirred at 0° C. for 5 min. TMSOTf (13.9 mL, 71.2 mmol) was added dropwise at 0° C. After addition, the mixture was stirred at 0° C. for 20 min until a clear solution achieved. The mixture was heated to 70° C. and stirred for 3 h. The reaction was cooled to room temperature and diluted with EA. The solution was washed with saturated NaHCO₃ and brine in sequence. The organic layer was dried over Na₂SO₄ and then concentrated. The residue was purified by chromatography on silica gel (PE:EA=4:1 to 2:1) to give compound P11-1 as light yellow foam (7 g, 58%).

### Step 2. Preparation of 5′(S)—C-methylguanosine (P11-2)

Compound P11-1 (7 g, 10.4 mmol) was treated with 2-meraptoethanol (4.6 ml, 64.4 mmol) and sodium methoxide (3.5 g, 64.8 mmol) in MeOH (200 mL). The mixture was refluxed at 70-80° C. for 24 h. The reaction mixture was cooled to room temperature, and the pH was adjusted to 7.0 by using glacial acetic acid. The solvent was evaporated, and the crude product was purified by HPLC to give compound P11-2 (2.4 g, 77%). ¹H NMR (400 MHz, CD₃OD): δ 7.89 (s, 1H), 5.76 (d, J=7.2 Hz, 1H), 4.63 (dd, J=7.2, 5.6 Hz, 1H), 4.29 (dd, J=5.2, 1.6 Hz, 1H), 4.02 (m, 1H), 3.93 (dd, J=3.2, 1.6 Hz, 1H), 1.27 (d, J=6.4 Hz, 3H).

### Step 3. Preparation of 2,3′-O-methoxymethylidene-5′(S)—C-methylguanosine (P11-3)

A mixture of compound P11-2 (1.0 g, 3.4 mmol), trimethyl orthoformate (5.0 mL) and p-toluenesulfonic acid monohydrate (1.0 g, 5.8 mmol) in 1,4-dioxane (130 mL) was stirred at RT for 24 h, cooled with ice, quenched by adding triethylamine (4 mL) and concentrated. The residue was purified by HPLC to give compound P11-3 as white foam (500 mg, 44%).

### Step 4. Preparation of 2,3′-O-methoxymethylidene-N2-(4-methoxytrityl)-5′(S)—C-methylguanosine (P11)

A solution of compound P11-3 (500 mg, 1.47 mmol) and 4-methoxytrityl chloride (500 mg, 1.62 mmol) in pyridine (10 mL) was stirred at 20° C. for 48 h. The solution was then diluted with ethyl acetate and washed with brine three times. Solvent was evaporated, and the residue was chromatographed on silica gel with 1-2% methanol in dichloromethane to give 187 mg of 2′,3′-O-methoxymethylidene-N²-(4-methoxytrityl)-5′(S)—C-methylguanosine (P11) as foam solid.

### Example 12

### Preparation of 2′,3′-O-methoxymethylidene-N²-(4-methoxytrityl)-5′(R)—C-methylguanosine (P12)

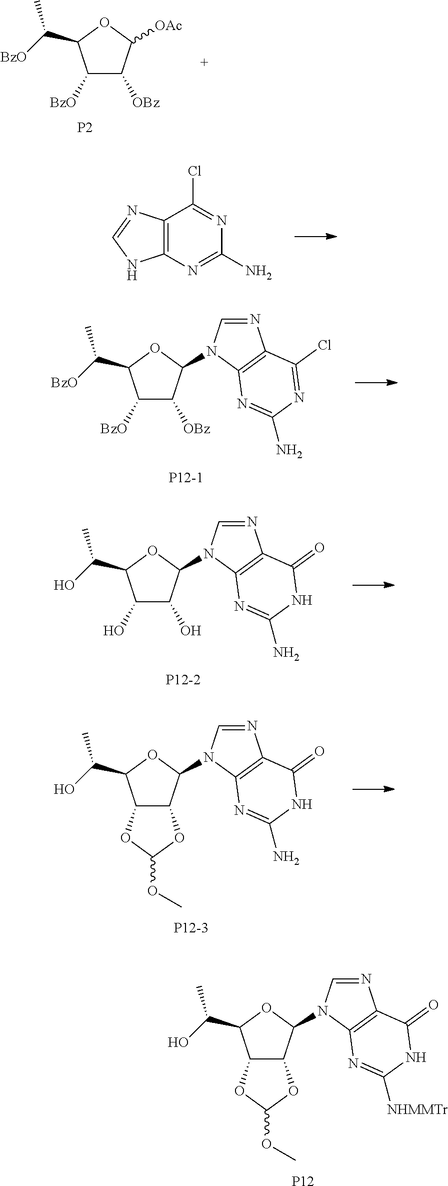

### Step 1. Preparation of 9-(2′,3′,5′-O-tribenzoyl-5′(R)—C-methyl-β-D-ribofuranosyl)-2-amino-6-chloropurine (P12-1)

To a stirred suspension of P2 (8 g, 15.4 mmol) and 2-amino-6-chloropurine (2.7 g, 15.8 mmol) in anhydrous MeCN (150 mL) was added DBU (7 g, 46.1 mmol) at 0° C. The mixture was stirred at 0° C. for 5 min and then TMSOTf (12.1 mL, 62 mmol) was added dropwise at 0° C. After addition, the mixture was stirred at 0° C. for 20 min until a clear solution was achieved. The mixture was heated to 70° C. and stirred for 3 h. The reaction was cooled to RT and diluted with EA. The solution was washed with saturated NaHCO₃ and brine in sequence. The organic layer was dried over Na₂SO₄ and concentrated. The residue was purified by chromatography on silica gel (PE:EA=4:1 to 2:1) to give P12-1 as light yellow foam (5.5 g, 57%).

### Step 2. Preparation of 5′(R)-methylguanosine (P12-2)

Compound P12-1 (3.5 g, 17.1 mmol) was treated with 2-meraptoethanol (2.5 ml, 35 mmol) and sodium methoxide (1.8 g, 33.3 mmol) in MeOH (100 mL), and the mixture was refluxed for 24 h. The reaction mixture was then cooled to RT, and the pH was adjusted to 7.0 by using acetic acid. The solvent was evaporated, and the crude product was purified by HPLC to give product P12-2 (1.1 g, 67%); ¹H NMR (400 MHz, CD3OD): δ 7.89 (s, 1H), 5.76 (d, J=7.2 Hz, 1H), 4.63 (dd, J=7.2, 5.6 Hz, 1H), 4.29 (dd, J=5.2, 1.6 Hz, 1H), 4.02 (m, 1H), 3.93 (dd, J=3.2, 1.6 Hz, 1H), 1.270 (d, J=6.4 Hz, 3H).

### Step 3. Preparation of 2′,3′-O-methoxymethylidene-5′(R)—C-methylguanosine (P12-3)

A mixture of compound P12-2 (1.1 g, 3.7 mmol), trimethyl orthoformate (5 mL) and p-toluenesulfonic acid monohydrate (1.1 g, 6.4 mmol) in 1,4-dioxane (150 mL) was stirred at RT for 24 h, cooled with ice, quenched by adding triethylamine (4 mL) and concentrated. The residue was purified by HPLC to give product P12-3 as white foam (700 mg, 56%).

### Step 4. Preparation of 2′,3′-O-methoxymethylidene-N2-(4-methoxytrityl)-5′(R)—C-methylguanosine (P12)

A solution of compound P12-3 (700 mg, 2.06 mmol) and 4-methoxytrityl chloride (700 mg, 2.27 mmol) in pyridine (10 mL) was stirred at 20° C. for 48 h. The mixture was diluted with ethyl acetate and washed with brine three times. Solvent was evaporated, and the residue was chromatographed on silica gel with 1-2% methanol in dichloromethane to give 317 mg of 2′,3′-O-methoxymethylidene-N²-(4-methoxytrityl)-5′(R)—C-methylguanosine (P12) as foam solid. MS m/z 611.9 (MH⁺).

### Example 13

### Preparation of 2′,3′-O-methoxymethylidene-5′(S)—C-methylinosine (P13)

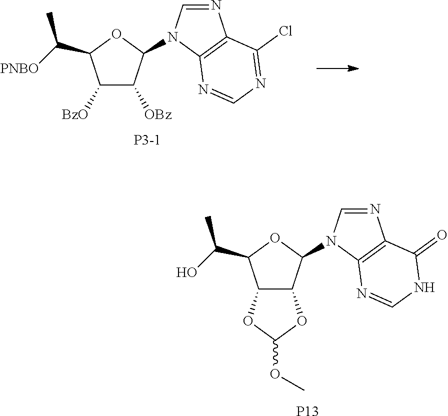

A solution of compound P3-1 (2 g, 2.97 mmol), 2-mercaptoethanol (1.3 mL, 18.2 mmol) and sodium methoxide (1.0 g, 18.5 mmol) in MeOH (100 mL) was refluxed for 24 h. The reaction mixture was cooled to RT and neutralized to pH 7.0 with acetic acid. The solvent was evaporated, and the crude product was purified by reverse-phase HPLC to give 657 mg (77%) of 5′(S)—C-methylinosine as white solid; ¹H NMR (CD₃OD) δ 8.37 (s, 1H), 8.06 (s, 1H), 4.01 (d, J=6.0 Hz, 1H), 4.61 (t, J=5.6 Hz, 1H), 4.28 (dd, J=5.2, 3.2 Hz, 1H), 4.02 (m, 2H), 1.26 (d, J=6.4 Hz, 3H).

A mixture of 5′(S)—C-methylinosine (657 mg, 2.3 mmol), trimethyl orthoformate (5.0 mL) and p-toluenesulfonic acid monohydrate (1.0 g, 5.8 mmol) in 1,4-dioxane (130 mL) was stirred at RT for 24 h. The mixture was then cooled with ice, quenched by adding triethylamine (4 mL) and concentrated. The residue was purified by reverse-phase HPLC to give 128 mg (17%) of 2′,3′-O-methoxymethylidene-5′(S)—C-methylinosine (P13) as white foam; ¹H NMR (CD₃OD) δ8.37, 8.36 (2s, 1H), 8.06, 8.04 (2s, 1H), 6.34, 6.18 (2d, J=3.2 Hz, 1H), 6.08, 5.98 (2s, 1H), 5.28, 5.23 (2m, 1H), 5.04, 4.96 (2m, 1H), 4.21, 4.09 (2m, 1H), 2.95 (m, 1H), 1.21, 1.17 (2d, J=6.4 Hz, 3H). MS m/z 324.8 (MH⁺).

### Example 14

### Preparation of 2′-deoxy-2′,2′-difluoro-3′4-(4-methoxytrityl)-5′(S)—C-methyluridine

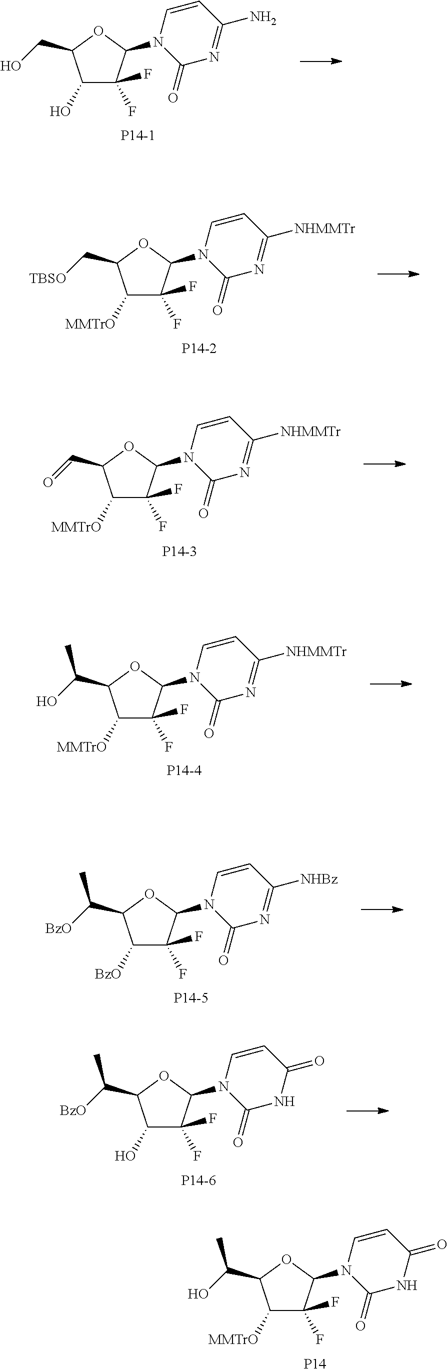

### Preparation of 5′-O-(t-butyldimethylsilyl)-2′-deoxy-2′,2′-difluoro-3′-O,N4-di(4-methoxytrityl) cytidine (P14-2)

To a solution of gemcitabine (P14-1) (48.3 g, 162 mmol) in anhydrous pyridine (500 mL) was added TBSCl (29.2 g, 194.4 mmol) in small portions at 0° C. under N₂. The reaction mixture was stirred at RT overnight. The solvent was removed under vacuum, and the residue was diluted with EA (1000 mL), washed with water and brine. The organic layer was separated, dried over anhydrous Na₂SO₄ and filtered. The filtrate was concentrated to give 62 g (92%) of 3′-O-(t-butyldimethylsilyl)-2′-deoxy-2′,2′-difluorocytidine as a white solid, which was used without further purification.

To a mixture of 5′-O-(t-butyldimethylsilyl)-2′-deoxy-2′,2′-difluorocytidine (60 g, 160 mmol), AgNO₃ (77.8 g, 510 mmol) and sym-collidine (159.8 g, 1.32 mol) in anhydrous DCM (800 mL) was added MMTrCl (156.8 g, 510 mmol) in small portions under N₂. The reaction mixture was stirred at RT overnight. The reaction mixture was then filtered through Buchner funnel. The filtrate was washed with saturated NaHCO₃ solution and followed by brine. The organic layer was separated, dried over anhydrous Na₂SO₄, filtered and concentrated. Chromatography on silica gel (PE/EA=3/1 to 2/1) gave 200 g of 5′-O-(t-butyldimethylsilyl)-2′-deoxy-2′,2′-difluoro-3′-O,N⁴-di(4-methoxytrityl) cytidine (P14-2) contaminated with collidine.

### Preparation of 2′-deoxy-5′-C, 5′-O-didehydro-2′,2′-O,N4-di(4-methoxytrityl)cytidine (P14-3)

To a solution of compound P14-2 (200 g, crude) in anhydrous THF (322 mL) was added TBAF (1M solution in THF) (85.3 g, 330 mmol) dropwise at 0° C. under N₂. The reaction mixture was stirred at RT overnight. The solvent was removed. The residue was dissolved in EA (800 mL) and washed with water and brine. The organic layer was separated, dried over anhydrous Na₂SO₄, filtered and concentrated. Chromatography on silica gel column (CH₂Cl₂/EA=10/1 to 5/1) gave 128 g of 2′-deoxy-2′,2′-difluoro-3′-O,N⁴-di(4-methoxytrityl) cytidine.

To a solution of pyridine (2.85 g, 36 mmol) in anhydrous DMSO (30 mL) at 10° C. was added TFA (2.05 g, 18 mmol) dropwise. After addition, the mixture was stirred at RT until a clear solution formed. The solution was then added to a solution of 2′-deoxy-2′,2′-difluoro-3′-O,N⁴-di(4-methoxytrityl) cytidine (24.2 g, 30 mmol) and DCC (18.6 g, 90 mmol) in anhydrous DMSO at 10° C. dropwise. Stirring was continued at RT for 12 h. Water (200 mL) was then added, and the mixture was stirred at RT for another hour. The precipitate was removed by filtration, and the filtrate was extracted with EtOAc (1000 mL). The organic layer was washed with brine (200 mL) and then dried over Na₂SO₄. The solvent was removed, and the residue was purified on silica gel column (EA:PE=1/1 to 2/1) to give 21.0 g (88%) of 2′-deoxy-5′-C,5′-O-didehydro-2′,2′-difluoro-3′-O,N⁴-di(4-methoxytrityl)cytidine (P14-3).

### Preparation of 2′-deoxy-2′,2′-O,N4-di(4-methoxytrityl)-5′(S)—C-methylcytidine (P14-4)

To a stirred solution of compound P14-3 (26 g, 32.3 mmol) in anhydrous THF (250 mL) was added MeMgBr (3 M solution in ether) (80 mL, 161.5 mmol) dropwise at −78° C. under N₂. The reaction mixture was stirred at RT overnight. The reaction was quenched by saturated NH₄Cl, and the mixture was extracted with EA (500 mL×3). The combined organic layer was dried over anhydrous Na₂SO₄ and concentrated. The resulting residue was purified by silica gel column (EA:PE=10/1 to 3/2) two times to give 8 g (44%) of crude 2′-deoxy-2′,2′-difluoro-3′-O,N⁴-di(4-methoxytrityl)-5′(S)—C-methylcytidine (P14-4). ¹H NMR (400 Hz, CDCl₃) 7.44-7.48 (m, 4H), 7.08-7.37 (m, 21H), 6.92 (br, 1H), 6.81-6.84 (m, 4H), 6.28 (t, J=8.4 Hz, 1H), 4.99 (d, J=7.6 Hz, 1H), 4.20-4.25 (m, 1H), 3.81 (s, 1H), 3.80 (s, 3H), 3.77 (s, 3H), 3.07-3.12 (m, 1H), 1.05 (d, J=6.4 Hz, 3H); ESI-MS: 822 [M+H]⁺.

### Preparation of 2′-deoxy-2′,2′-difluoro-5′(S)—C-methyl-3′,5′-O,N4-tribenzoylcytidine (P14-5)

Compound P14-4 (8 g, 9.73 mmol) was dissolved in 125 mL AcOH/H₂O (v/v=4:1). The mixture was stirred at 60° C. for 6 h. The solvent was removed, and the residue was purified on silica gel column (CH₂Cl₂:MeOH=100/1 to 10/1 with 0.5% TEA) two times to give 2.0 g of 2′-deoxy-2′,2′-difluoro-5′(S)—C-methylcytidine as white solid. ¹H NMR (CD₃OD) δ 7.87 (d, J=7.6 Hz, 1H), 6.18 (t, J=7.6 Hz, 1H), 5.90 (d, J=7.6 Hz, 1H), 4.25-4.17 (m, 1H), 3.97 (dd, J=6.4 Hz, 3.6 Hz, 1H), 3.68 (dd, J=8.4 Hz, 2.8 Hz, 1H), 1.31 (d, J=6.4 Hz, 3H); ¹³C NMR (100 Hz, CD₃OD): δ 166.3, 156.5, 141.2, 122.6 (t, J=267 Hz), 94.9, 84.6 (t, J=30.6 Hz), 83.4 (t, J=25 Hz), 70.2 (t, J=23 Hz), 65.0, 18.2; ESI-MS: 555 [2M+H]⁺, 278 [M+H]⁺.

To a stirred solution of 2′-deoxy-2′,2′-difluoro-5′(S)—C-methylcytidine (0.975 g, 3.5 mmol, co-evaporated with dry pyridine for three times) in anhydrous pyridine (40 mL) was added BzCl (1.73 g, 12 mmol) dropwise at 0° C. under N₂. After addition, the mixture was warmed to RT and stirred for 3 h. The reaction was quenched with H₂O, and the solvent was removed under reduced pressure. The residue was taken up into DCM and washed with saturated NaHCO₃, 1% H₂SO₄ and brine in sequence. The organic layer was dried over Na₂SO₄ and concentrated. The residue was purified on silica gel (PE:EA=10:1 to 3:1) to afford 1.43 g (69%) of 2′-deoxy-2′,2′-difluoro-5′(S)—C-methyl-3′,5′-O,N⁴-tribenzoylcytidine (P14-5) as white solid.

### Preparation of 5′-O-benzoyl-2′-deoxy-2′,2′-difluoro-5′(S)—C-methyluridine (P14-6)

Compound P14-5 (1.43 g) was dissolved in a mixture of DME (dimethoxyethane) (36 mL) and H₂O (24 mL), and the resulting solution in a sealed vessel was then stirred at 125° C. overnight. The solvent was removed under reduced pressure, and the residue was purified on silica gel (PE:EA=10:1 to 3:1) to give 0.98 g (85%) of 2′-deoxy-3′,5′-O-dibenzoyl-2′,2′-difluoro-5′(S)—C-methyluridine as white solid, which was dissolved in methanol (30 mL). Aqueous ammonia 25%, 30 mL) was added, and the resulting mixture was stirred at RT for 3 h. The solvent was removed, and the residue was purified by column chromatography on silica gel eluting with a mixture of PE:EA=10:1-3:2 to afford 0.58 g (75%) of 5′-O-benzoyl-2′-deoxy-2′,2′-difluoro-5′(S)—C-methyluridine (P14-6).

### Preparation of 2′-deoxy-2′,2′-difluoro-5′ (S)—C-methyl-3′-O-(4-methoxymethyl)uridine (P14)

To a solution of compound P14-6 (0.53 g, 1.39 mmol) in dry DCM (25 mL) were added AgNO₃ (0.29 g, 1.67 mmol) and 2,4,6-collidine (0.22 g, 1.8 mmol). A solution of MMTrCl (0.51 g, 1.67 mmol) in dry DCM (15 mL) was then added. The resulting mixture was stirred at room temperature overnight and filtered through celite. The cake was washed with EA (300 mL). Combined organic phase was washed with brine, dried over Na₂SO₄, filtered and concentrated. The residue was purified by column chromatography on silica gel eluting with a mixture of PE:EA=10:1-3:1 to afford 0.8 g (88%) of 5′-O-benzoyl-2′-deoxy-2′,2′-difluoro-5′(S)—C-methyl-3′-O-(4-methoxytrityl)uridine, which was dissolved in MeOH (40 mL). The resulting solution was bubbled with ammonia gas for 30 min at −78° C. Another 30 mL of aq. ammonia was added to the mixture and heated at 40-50° C. overnight. The mixture was concentrated and purified by column chromatography on silica gel eluting with a mixture of PE:EA=5:1-2:1 to give 0.2 g, (29%) of 2′-deoxy-2′,2′-difluoro-5′ (S)—C-methyl-3′-O-(4-methoxymethyl)uridine (P14) as white solid; ¹H NMR (CDCl₃, 400 MHz): δ 8.52 (s, 1H), 7.50-7.47 (m, 5H), 7.37 (d, J=8.8 Hz, 2H), 7.32-7.26 (m, 4H), 6.85 (d, J=8.8 Hz, 1H), 6.19 (t, J=9.6 Hz, 1H), 5.63 (d, J=7.6 Hz, 1H), 4.27 (dd, J=11.6, 18.4 Hz, 1H), 3.84 (d, J=6.8 Hz, 1H), 3.18 (br s, 1H); ESI-MS: m/z 573 [M+Na]⁺.

### Example 15

### Preparation of 5′(S)—C-methyladenosine 5′-[1-naphthyl(cyclohexoxy-L-alaninyl)]phosphate (A1)

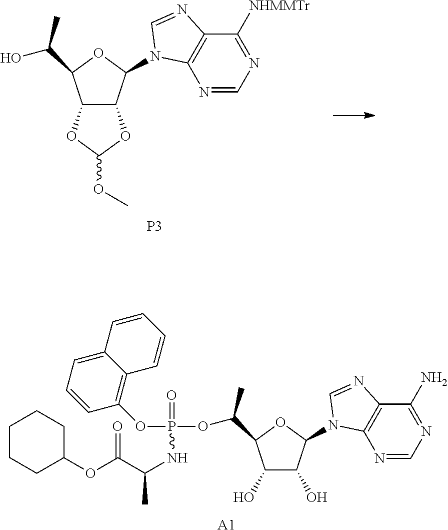

To a solution of 2′,3′-O-methoxymethylidene-N⁶-(4-methoxytrityl)-5′(S)-methyladenosine (P3) (595 mg, 1.0 mmol) in THF (8 mL) under argon was added dropwise 1.0 M tert-BuMgBr in THF (3.0 mL). The resulting solution was stirred at RT for 30 min. 1-naphth-yl(cyclohexoxy-L-alaninyl) phosphorochloridate was prepared according to a general procedure (McGuigan et al. J. Med. Chem. 2008, 51, 5807) (0.95 M in THF, 4.0 mL) was added. The reaction mixture was stirred at RT for 3 days, cooled with ice, quenched with water, diluted with ethyl acetate, washed with brine three times, dried over sodium sulfate, and concentrated. Chromatography on silica gel with ethyl acetate/hexanes (3:2 to 4:1) gave a mixture of four isomers. The mixture was dissolved in 80% formic acid (25 mL), and the resulting solution stood at RT overnight. Solvent was evaporated at RT and co-evaporated with MeOH/toluene three times. Chromatography on silica gel with 5-8% MeOH in DCM gave 112 mg of pure 5′(S)—C-methyladenosine 5′-[1-naphthyl(cyclohexoxy-L-alaninyl)]phosphate (A1) as white solid. The second chromatography of the impure portion gave 322 mg of the pure product, and then the third chromatography gave 101 mg of the pure product. Total yield of 5′(S)—C-methyladenosine 5′-[1-naphthyl(cyclohexoxy-L-alaninyl)]phosphate was 535 mg as white solid; ¹H NMR (CD₃OD, two isomers) δ 1.20, 1.23 (2dd, J=7.2, 1.2 Hz, 3H), 1.41, 1.56 (2d, J=6.4 Hz, 3H), 1.16-1.76 (m, 10H), 3.88-3.99 (m, 1H), 4.05-4.08 (m, 1H), 4.02-4.06 (m, 1H), 4.32-4.65 (m, 3H), 4.82-4.97 (m, 1H), 5.96, 6.03 (2d, J=4.4 Hz, 1H), 7.31, 7.38 (2t, J=8.0 Hz, 1H), 7.39-7.53 (m, 3H), 7.62, 7.68 (2d, J=8.0 Hz, 1H), 7.81, 8.06 (2m, 1H), 7.87, 8.12 (2m, 1H), 8.09, 8.20 (2s, 1H), 8.15, 8.28 (2s, 1H); ³¹P NMR (CD₃OD, two isomers) δ 3.31 (s), 3.48 (s). MS m/z 641.4 (MH⁺).

### Example 16

### Preparation of 5′(S)—C-methyladenosine 5′-[1-naphthyl(neopentoxy-L-alaninyl)]phosphate (A2)

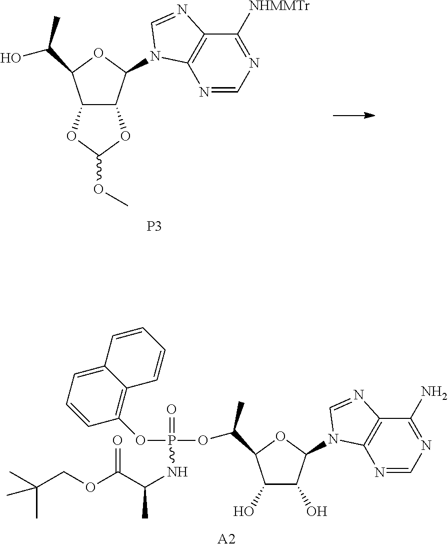

Following the general procedure described for 5′(S)—C-methyladenosine, 5′-[1-naphthyl(cyclohexoxy-L-alaninyl)]phosphate (A1), 509 mg of 5′(S)—C-methyladenosine 5′-[1-naphthyl(neopentoxy-L-alaninyl)]phosphate (A2) was obtained as white solid from 714 mg (1.2 mmol) of 2′,3′-O-methoxymethylidene-N⁶-(4-methoxytrityl)-5′(S)-methyladenosine (P3) and 1-naphthyl(neopentoxy-L-alaninyl)]phosphorochloridate prepared according to a general procedure (McGuigan et al. J. Med. Chem. 2008, 51, 5807). ¹H NMR (CD₃OD, two isomers) δ0.83, 0.87 (2s, 9H), 1.23, 1.26 (2dd, J=7.2, 0.8 Hz, 3H), 1.40, 1.66 (2d, J=6.4 Hz, 3H), 3.64, 3.71 (2AB, J=35.2/29.2, 10.4 Hz, 2H), 3.95-4.06 (m, 2H), 4.42-4.58 (m, 2H), 4.81-4.96 (m, 1H), 5.96, 6.02 (2d, J=4.4 Hz, 1H), 7.31, 7.38 (2t, J=8.0 Hz, 1H), 7.39-7.53 (m, 3H), 7.62, 7.68 (2d, J=8.0 Hz, 1H), 7.81, 8.05 (2d, J=8.0 Hz, 1H), 7.86, 8.12 (2m, 1H), 8.10, 8.20 (2s, 1H), 8.15, 8.27 (2s, 1H); ³¹P NMR (CD₃OD, two isomers) δ 3.36 (s), 3.48 (s). MS m/z 629.5 (MH⁺).

### Example 17

### Preparation of 2′,3′-O-carbonyl-5′(S)—C-methyladenosine 5′-[1-naphthyl(cyclohexoxy-L-alaninyl)]phosphate (A3)

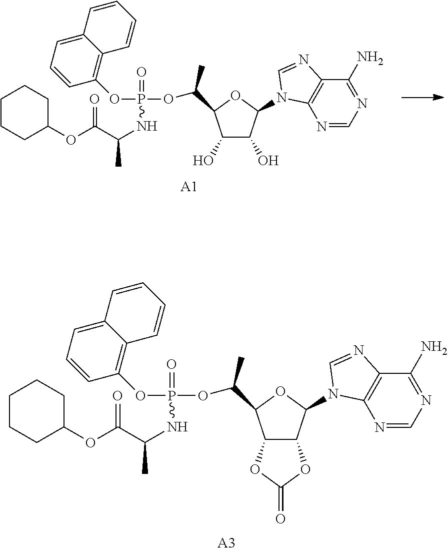

A solution of 5′(S)—C-methyladenosine 5′-[1-naphthyl(cyclohexoxy-L-alaninyl)]phosphate (A1) (159 mg, 0.248 mmol) and carbonyldiimidazole (97 mg, 0.6 mmol) in anhydrous DMF (2.5 mL) was stirred at RT for 5 h and then evaporated at RT under high vacuum. The crude was purified on silica gel with 5-8% MeOH in DCM. The collected fractions were concentrated and purified on reverse-phase HPLC (C-18) with acetonitrile/water system. The collected fractions were concentrated again and subjected to a chromatography on silica gel with 6-8% EtOAc in DCM. The collected fractions were concentrated and purified on silica gel with 6-9% isopropanol in DCM to give 58 mg of 2′,3′-O-carbonyl-5′(S)—C-methyladenosine 5′-[1-naphthyl(cyclohexoxy-L-alaninyl)]phosphate (A3) as white solid; ¹H NMR (CDCl₃, two isomers) δ 1.20, 1.26 (2d, J=7.2 Hz, 3H), 1.31, 1.53 (2d, J=6.4 Hz, 3H), 1.28-1.81 (m, 10H), 3.87, 4.38 (2t, J=10.4 Hz, 1H), 3.92-4.08 (m, 1H), 4.30-4.34 (m, 1H), 4.67-4.75 (m, 1H), 4.80-4.92 (m, 1H), 5.15, 5.23 (2dd, J=7.6, 3.6/2.4 Hz, 1H), 5.64, 5.95 (2dd, J=7.6, 3.6/2.0, Hz, 1H), 5.90, 6.13 (2s, br, 2H), 6.07, 6.20 (2d, J=6.8/6.0 Hz, 1H), 7.32, 7.33 (2t, J=8.0/7.6 Hz, 1H), 7.35-7.51 (m, 3H), 7.62 (d, J=7.6 Hz, 1H), 7.72, 8.07 (2s, 1H), 7.78-7.83 (m, 1H); 7.87-7.91 (m, 1H), 7.95-8.0 (m, 1H), 8.25, 8.29 (2s, 1H); ³¹P NMR (CD₃OD, two isomers) δ 2.41 (s), 2.80 (s).

### Example 18

### Preparation of 2′,3′-O-carbonyl-5′(S)—C-methyladenosine 5′-[1-naphthyl(neopentoxy-L-alaninyl)]phosphate (A4)

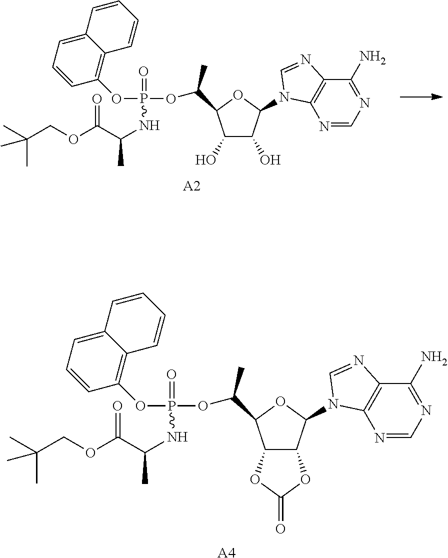

Following the general procedure described for 2′,3′-O-carbonyl-5′(S)—C-methyladenosine 5′-[1-naphthyl(cyclohexoxy-L-alaninyl)]phosphate (A3), 67 mg (two isomers) of 2′,3′-O-carbonyl-5′(S)—C-methyladenosine 5′-[1-naphthyl(neopentoxy-L-alaninyl)]phosphate (A4) was obtained as white solid from 126 mg (0.2 mmol) of 5′ (S)—C-methyladenosine 5′-[1-naphthyl(neopentoxy-L-alaninyl)]phosphate (A2) and 1-naphthyl(neopentoxy-L-alaninyl) phosphorochloridate. ¹H NMR (CDCl₃, two isomers) δ 0.88, 0.92 (2s, 9H), (2d, J=7.2 Hz, 3H), 1.25, 1.54 (2d, J=6.8 Hz, 3H), 1.27-1.30 (2d, J=7.2 Hz, 1H), 3.70, 3.84 (2dd, J=11.6/16.0, 10.4 Hz, 1H), 3.82, 4.52 (2t, J=10.0 Hz, 1H), 3.96-4.15 (m, 1H), 4.30-4.36 (m, 1H), 4.82-4.93 (m, 1H), 5.21, 5.30 (2dd, J=7.6, 3.6/2.8 Hz, 1H), 5.63, 5.93 (2dd, J=7.2, 4.0/2.4, Hz, 1H), 5.99, 6.29 (2s, br, 2H), 6.09, 6.19 (2d, J=2.8/2.0 Hz, 1H), 7.32, 7.33 (2t, J=8.0/7.6 Hz, 1H), 7.35-7.51 (m, 3H), 7.62 (d, J=7.6 Hz, 1H), 7.76, 8.10 (2s, 1H), 7.79-8.0 (m, 2H); 8.24, 8.29 (2s, 1H); ³¹P NMR (CD₃OD, two isomers) δ 2.39 (s), 2.80 (s).

### Example 19

### Preparation of 2′,3′-O-carbonyl-N6-methoxycarbonyl-5′(S)—C-methyladenosine 5′-[1-naphthyl(cyclohexoxy-L-alaninyl)]phosphate (A5) and N6-methoxycarbonyl-5′(S)—C-methyladenosine 5′-[1-naphthyl(cyclohexoxy-L-alaninyl)]phosphate (A6)

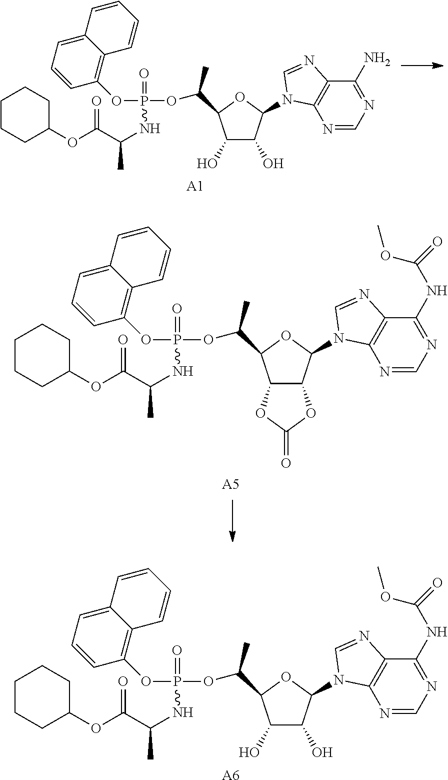

A solution of 5′(S)—C-methyladenosine 5′-[1-naphthyl(cyclohexoxy-L-alaninyl)]phosphate (A1) (130 mg, 0.20 mmol) and carbonyldiimidazole (810 mg, 5.0 mmol) in anhydrous DMF (12 mL) was stirred at RT for 5 h and then evaporated at RT under high vacuum. The crude was purified on silica gel with 5-8% MeOH in DCM. The higher R_{f} fractions were collected and evaporated. A portion of the higher R_{f} minor product was further purified by chromatography on silica gel to give 17 mg of 2′,3′-O-carbonyl-N⁶-methoxycarbonyl-5′(S)—C-methyladenosine 5′-[1-naphthyl(cyclohexoxy-L-alaninyl)]phosphate (A5) as white solid; ¹H NMR (acetonitrile-d₃, two isomers) δ 1.13, 1.19 (2dd, J=7.2, 0.8 Hz, 3H), 1.41, 1.55 (2d, J=6.4 Hz, 3H), 1.21-1.76 (m, 10H), 3.79-4.93 (m, 1H), 3.798, 3.803 (2s, 3H), 4.13, 4.25 (2t, J=10.4 Hz, 1H), 4.46, 4.50 (2dd, J=6.4/4.8, 4.4 Hz, 1H), 4.55-4.66 (m, 1H), 4.82-4.98 (m, 1H), 5.50, 5.60 (2dd, J=8.0, 4.0 Hz, 1H), 5.63, 5.87 (2dd, J=8.0, 2.4/2.0 Hz, 1H), 6.38, 6.46 (2d, J=2.4/2.0 Hz, 1H), 7.28-7.57 (m, 4H), 7.3, 7.69 (2dd, J=8.0, 0.8 Hz, 1H), 7.82-8.03 (m, 2H), 8.19, 8.29 (2s, 1H), 8.54, 8.61 (2s, 1H); ³¹P NMR (CD₃OD, two isomers) δ 2.50 (s), 2.87 (s). MS m/z 725.3 (MH⁺).

The remainder of the higher R_{f} product was dissolved in acetonitrile/water, and the resulting solution stood at RT for 5 days. Chromatography on silica gel with 6-10% i-PrOH in DCM gave 15.5 mg of N⁶-methoxycarbonyl-5′(S)—C-methyladenosine 5′-[1-naphthyl(cyclohexoxy-L-alaninyl)]phosphate (A6) as white solid; ¹H NMR (CD₃OD, two isomers) δ1.18, 1.21 (2dd, J=7.2, 1.2 Hz, 3H), 1.42, 1.57 (2d, J=6.4 Hz, 3H), 1.2-1.78 (m, 10H), 3.84 (s, 3H), 3.85-3.97 (m, 1H), 4.02-4.09 (m, 1H), 4.37, 4.48 (2t, J=5.2 Hz, 1H), 4.48 (2dd, J=5.2, 4.4 Hz, 1H), 4.48-4.64 (m, 1H), 4.82-4.98 (m, 1H), 6.04, 6.11 (2d, J=4.8/4.4 Hz, 1H), 7.29, 7.37 (2t, J=8.0/7.6 Hz, 1H), 7.36-7.52 (m, 3H), 7.59, 7.67 (2d, J=8.0 Hz, 1H), 7.79, 8.00 (2m, 1H), 7.83-7.87, 8.08-8.13 (2m, 1H), 8.31, 8.52 (2s, 1H), 8.47, 8.58 (2s, 1H); ³¹P NMR (CD₃OD, two isomers) δ 3.23 (s), 3.43 (s). MS m/z 699.4 (MH⁺), 828.5 (MH⁺+6-methyl-2-heptylamine).

### Example 20

### Preparation of 2′,3′-O-carbonyl-N6-methoxycarbonyl-5′(S)—C-methyladenosine 5′-[1-naphthyl(neopentoxy-L-alaninyl)]phosphate (A7)

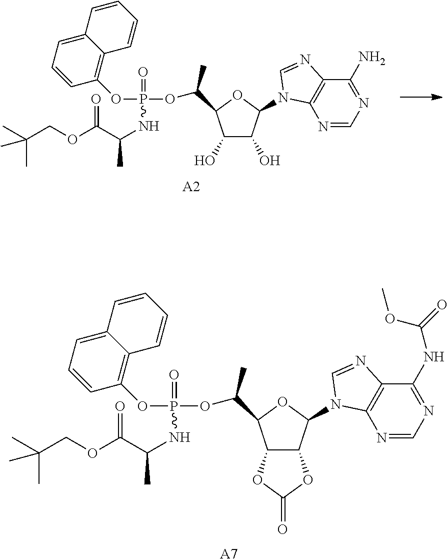

A solution of 5′(S)—C-methyladenosine 5′-[1-naphthyl(neopentoxy-L-alaninyl)]phosphate (A2) (126 mg, 0.20 mmol) and carbonyldiimidazole (810 mg, 5.0 mmol) in anhydrous DMF (12 mL) was stirred at RT for 5 h and then evaporated at RT under high vacuum. The crude was purified on silica gel with 5-8% MeOH in DCM. The higher R_{f} fractions were collected and re-purified by chromatography on silica gel to give 11 mg of 2′,3′-O-carbonyl-N⁶-methoxycarbonyl-5′(S)—C-methyladenosine 5′-[1-naphthyl(neopentoxy-L-alaninyl)]phosphate (A7) as white solid; ¹H NMR (acetonitrile-d₃, two isomers) δ 0.87, 0.90 (2s, 9H), 1.17, 1.22 (2dd, J=7.2, 0.8/1.2 Hz, 3H), 1.41, 1.55 (2d, J=6.4 Hz, 3H), 3.68, 3.72 (2AB, J=18.4/37.2, 10.4 Hz, 2H), 3.802, 3.807 (2s, 3H), 3.88-4.04 (m, 1H), 4.17, 4.28 (2t, J=10.4 Hz, 1H), 4.46, 4.51 (2dd, J=6.0/4.8, 4.4/4.0 Hz, 1H), 4.82-4.98 (m, 1H), 5.52, 5.60 (2dd, J=8.0, 4.0 Hz, 1H), 5.64, 5.87 (2dd, J=8.0, 2.4 Hz, 1H), 6.39, 6.46 (2d, J=2.4/2.8 Hz, 1H), 7.29-7.57 (m, 4H), 7.63, 7.70 (2d, J=8.0 Hz, 1H), 7.82-8.03 (m, 2H), 8.22, 8.62 (2s, 1H), 8.31, 8.54 (2s, 1H); ³¹P NMR (CD₃OD, two isomers) δ 2.58 (s), 2.83 (s). MS m/z 713.4 (MH⁺).

### Example 21

### Preparation of 5′(S)—C-methyladenosine 5′-[phenyl(isopropoxy-L-alaninyl)]phosphate (A8)

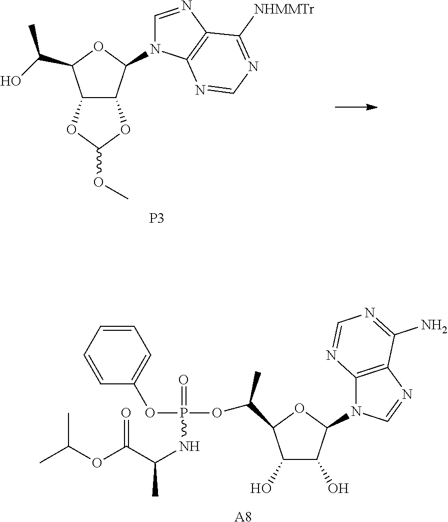

### Step 1. Preparation of phenyl(isopropoxy-L-alaninyl) phosphorochloridate

A solution of triethylamine (5.7 g, 56.4 mmol) in anhydrous dichloromethane (50 mL) was added dropwise to a solution of phenyl phosphorodichloridate (6 g, 28.4 mmol) and isopropyl L-alaninate hydrochloride (4.7 g, 28.1 mmol) in dichloromethane (120 mL) with vigorous stirring at −78° C. over 2 h. After addition, the reaction was allowed to warm to RT gradually and stirred for 2 h. The solvent was removed under vacuum and anhydrous ether (20 mL) was added. The precipitated salt was filtered, and the filtrate was washed with ether. The combined filtrate was concentrated and purified by flash chromatography on silica gel (DCM) to give phenyl(isopropoxy-L-alaninyl) phosphorochloridate as colorless syrup.

### Step 2. Preparation of 5′(S)—C-methyladenosine 5′-[phenyl(isopropoxy-L-alaninyl)]phosphate (A8)

To a solution of 2′,3′-O-methoxymethylene-N⁶-(4-methoxytrityl)-5′(5)-methyladenosine (P3) (1.0 g, 16.8 mmol) in THF (30 mL) under argon was added 1.0 M t-BuMgBr in THF (5.0 mL, 5.0 mmol) at 0° C. The resulting solution was stirred at RT for 30 min and phenyl(isopropoxy-L-alaninyl) phosphorochloridate (5 mL, 1M in THF) was added at 0° C. The reaction mixture was stirred at RT for 20 h, cooled with ice, quenched with water, diluted with ethyl acetate, washed with brine, extracted with ethyl acetate three times, and dried over MgSO₄. After concentration, the residue was purified by chromatography on silica gel (PE:EA=2:1 to 1:1) to give 1.3 g (89%) of a coupling product, which was dissolved in 80% formic acid (25 mL). The resulting solution was stirred at RT overnight, solvent evaporated at RT, and the residue purified by chromatography on silica gel with 10-15% MeOH in DCM. Re-purification by reverse-phase HPLC with acetonitrile/water with HCOOH, gave 5′(S)—C-methyladenosine 5′-[phenyl(isopropoxy-L-alaninyl)]phosphate (A8) as a mixture of two P-isomers (370 mg, 36%); ¹H NMR (CD₃OD, two isomers) δ 1.13, 1.19 (2dd, 6H), 1.223, 1.226 (2d, J=7.2, 3H), 1.42, 1.51 (2d, J=6.4 Hz, 3H), 3.8-3.9 (m, 2H), 4.0-4.05 (m, 1H), 4.33, 4.42 (2t, J=5.2 Hz, 1H), 4.52, 4.56 (2t, J=5.2 Hz, 1H), 4.75-4.85 (m, 1H), 4.94 (m, 1H), 6.02 6.03 (2s, 1H), 7.19-7.34 (m, 5H), 8.18, 8.20 (2s, 1H), 8.26, 8.30 (2s, 1H); ³¹P NMR (CD₃OD, two isomers) δ 0.78 (s), 0.85 (s). MS m/z 550.9 (MH⁺).

### Example 22

### Preparation of 2′,3′-carbonyl-5′(S)—C-methyladenosine 5′-[phenyl(isopropoxy-L-alaninyl)]phosphate (A9)

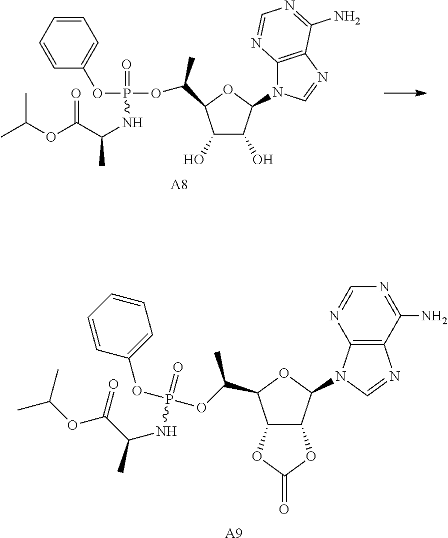

A solution of compound A8 (110 mg, 0.2 mmol) in anhydrous dichloromethane (20 mL) was added CDI (1,1′-carbonyldiimidazole) (100 mg, 0.6 mmol) at RT. The mixture was stirred for about 2 h. The solvent was removed under vacuum at 0° C. and purified by preparative HPLC to give 46 mg (40%) of 2′,3′-carbonyl-5′(S)—C-methyladenosine 5′-[phenyl(isopropoxy-L-alaninyl)]phosphate (A9) as a mixture of two P-isomers; ³¹P NMR (CD₃OD, two isomers) δ 1.95 (s), 2.32 (s). MS m/z 577.1 (MH⁺).

### Example 23

### Preparation of 5′(S)—C-methyladenosine 5′-[phenyl(cyclohexoxy-L-alaninyl)]phosphate (A10)

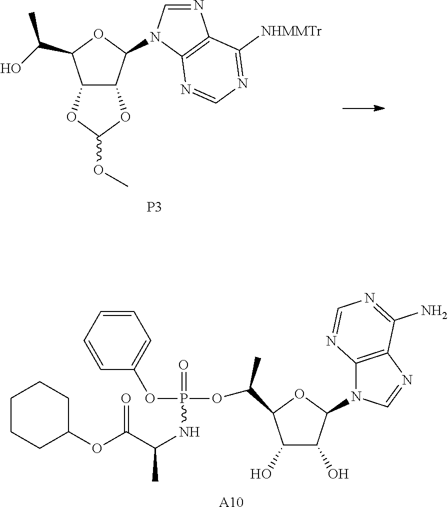

### Step 1. Preparation of phenyl(cyclohexoxy-L-alaninyl) phosphorochloridate

To a stirred solution of phenyl phosphorodichloridate (6.33 g, 30 mmol) and cyclohexyl alaninate hydrochloride (6.24 g, 30 mmol) in anhydrous DCM (130 mL) was added a solution of TEA (triethylamine) (8.3 mL, 60 mmol) in DCM (20 mL) dropwise at −78° C. After addition, the mixture was warmed to RT gradually and stirred overnight. The solvent was removed, and the residue was dissolved in methyl-butyl ether. The precipitate was removed by filtration, and the filtrate was concentrated. The residue was purified by column on silica gel with DCM to give pure phenyl(cyclohexoxy-L-alaninyl) phosphorochloridate (7.20 g, 70%).

### Step 2. Preparation of 5′(S)—C-methyladenosine 5′-[phenyl(cyclohexoxy-L-alaninyl)]phosphate (A10)

To a stirred solution of compound P3 (850 mg, 1.43 mmol) in anhydrous THF (20 mL) was added a solution of t-BuMgCl (4 mL, 1M in THF) dropwise at 0° C. The mixture was then stirred at RT for 40 min and re-cooled to 0° C. A solution of phenyl(cyclohexoxy-L-alaninyl) phosphorochloridate (4 mL, 1.0 M in THF) was added dropwise. After addition, the mixture was stirred at RT for 40 h. The reaction was quenched with H₂O and extracted EA. The organic layer was dried over Na₂SO₄ and concentrated. The residue was purified by column on silica gel (PE:EA=2:1 to 1:1) to give 1.1 g of a protected form of A10 (85%).

The protected form of A10 (810 mg) was dissolved in 80% HCOOH aqueous solution, and the mixture was stirred at RT for 50 h. The solvent was removed, and the residue was purified by RP HPLC (HCOOH system) to give 5′(S)—C-methyladenosine 5′-[phenyl(cyclohexoxy-L-alaninyl)]phosphate (A10) as a mixture of two P-isomers (370 mg, 59%); ³¹P NMR (CD₃OD, two isomers) δ 0.74 (s), 0.80 (s). MS m/z 591.0 (MH⁺).

### Example 24

### Preparation of 5′(S)—C-methyladenosine 5′-[phenyl(neopentoxy-L-alaninyl)]phosphate (A11)

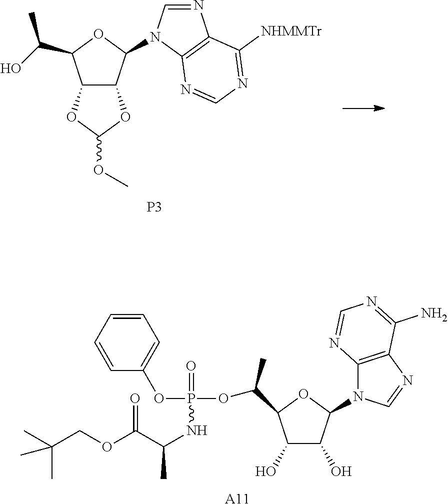

### Step 1. Preparation of phenyl(neopentoxy-L-alaninyl) phosphorochloridate

A solution of triethylamine (6 g, 59.4 mmol) in anhydrous dichloromethane (50 mL) was added dropwise to a solution of phenyl phosphorodichloridate (5.5 g, 28.2 mmol) and neopentyl alaninate hydrochloride (6 g, 28.4 mmol) in DCM (120 mL) with vigorous stirring at −78° C. over a period of 2 h. After addition, the reaction temperature was allowed to warm to RT gradually and stirred for about 2 h. The solvent was removed under vacuum and anhydrous ether 20 mL was added. The precipitated salt was filtered, and the precipitate was washed with ether. The combined organic phase was concentrated and purified by column chromatography to give the colorless oil of phenyl(neopentoxy-L-alaninyl) phosphorochloridate.

### Step 2. Preparation of 5′(S)—C-methyladenosine 5′-[phenyl(neopentoxy-L-alaninyl)]phosphate (A11)

To a solution of compound P3 (850 mg, 1.43 mmol) in THF (30 mL) under argon was added 1.0 M t-BuMgBr in THF (4.3 mL, 4.3 mmol) at 0° C. The resulting solution was stirred at RT for 30 min and phenyl(neopentoxy-L-alaninyl) phosphorochloridate (4.3 mL, 1M in THF) was added at 0° C. The reaction mixture was stirred at RT for 20 h, cooled with ice, quenched with water, diluted with ethyl acetate, washed with brine, extracted with ethyl acetate three times, and dried over MgSO₄. After concentration, the residue was purified by column on silica gel (PE:EA=2:1 to 1:1) to give 1.1 g of a protected product of A11, which was dissolved in 80% formic acid (25 mL) and stirred at RT overnight. The solvent was evaporated at RT, and the residue was purified by chromatography on silica gel with 10-15% MeOH in DCM. The residue was then re-purification by reverse-phase HPLC with acetonitrile/water, to give 5′(S)—C-methyladenosine 5′-[phenyl(neopentoxy-L-alaninyl)]phosphate (A11) as a mixture of two P-isomers (240 mg, 34%); ³¹P NMR (CD₃OD, two isomers) δ 2.26 (s), 2.36 (s). MS m/z 578.9 (MH⁺).

### Example 25

### Preparation of 2′,3′-O-carbonyl-5′(S)—C-methyladenosine 5′-[phenyl(neopentoxy-L-alaninyl)]phosphate (A12)

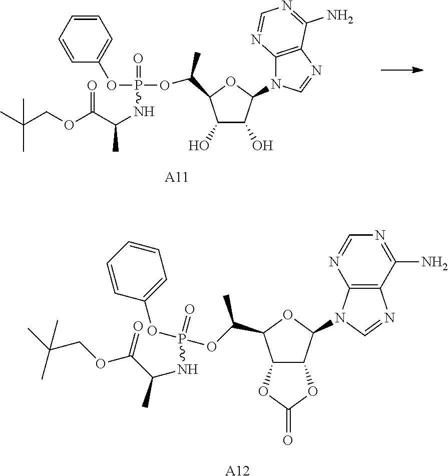

A solution of compound A11 (132 mg, 0.23 mmol) in anhydrous dichloromethane (20 mL) was added CDI (120 mg, 0.70 mmol) at RT, and stirred about 2 h. The solvent was removed under vacuum at 0° C. and purified by prep. HPLC (neutral) to give mg (47%) of 2′,3′-O-carbonyl-5′(S)—C-methyladenosine 5′-[phenyl(neopentoxy-L-alaninyl)]phosphate (A12) as a mixture of 2 P-isomers; ³¹P NMR (CD₃OD, two isomers) δ 2.03 (s), 2.44 (s). MS m/z 605.2 (MH⁺).

### Example 26

### Preparation of 2′,3′-O-carbonyl-5′(S)—C-methyladenosine 5′-[phenyl(cyclohexoxy-L-alaninyl)]phosphate (A13)

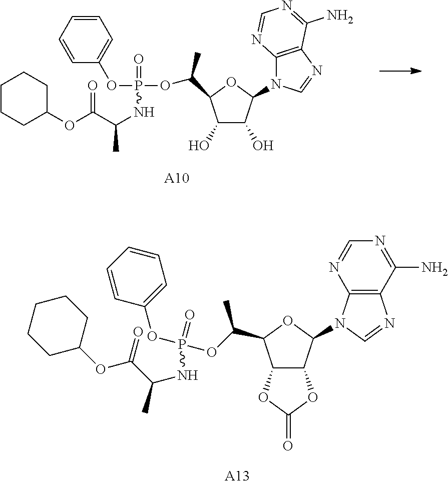

A solution of compound A10 (120 mg, 0.30 mmol) in anhydrous dichloromethane (20 mL) was added CDI (150 mg, 0.90 mmol) at RT. The mixture was stirred for about 2 h. The solvent was removed under vacuum at 0° C. and purified by prep. HPLC (neutral) to give 60 mg (32%) of 2′,3′-O-carbonyl-5′(S)—C-methyladenosine 5′-[phenyl(cyclohexoxy-L-alaninyl)]phosphate (A13) as a mixture of two P-isomers; ³¹P NMR (160 MHz, CDCl₃): δ1.98 (s), 2.35 (s). MS m/z 617.1 (MH⁺).

### Example 27

### Preparation of 2′,3′-O-dipropionyl-5′(S)—C-methyladenosine 5′-[phenyl(isopropoxy-L-alaninyl)]phosphate (A14)

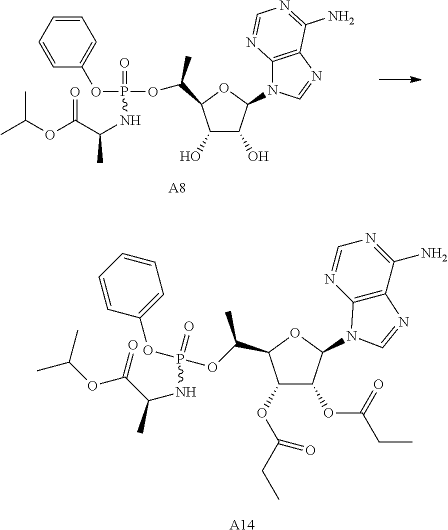

To a solution of compound A8 (150 mg, 0.27 mmol) in anhydrous pyridine (5 mL) was added propionic anhydride (150 mg, 1.15 mmol) and DMAP (4-dimethylaminopyridine) (50 mg, 0.41 mmol) at RT. The mixture was stirred for about 18 h. The solvent was removed under vacuum at 0° C. and purified by column chromatography to give 120 mg (67%) of 2′,3′-O-dipropionyl-5′(S)—C-methyladenosine 5′-[phenyl(isopropoxy-L-alaninyl)]phosphate (A14) as a mixture of 2 P-isomers; ³¹P NMR (160 MHz, CDCl₃): δ1.98 (s), 2.35 (s). MS m/z 663.2 (MH⁺).

### Example 28

### Preparation of 5′(S)—C-methyladenosine 5′-[phenylmethoxy-L-alaninyl)]phosphate (A15) and 2′,3′-O-carbonyl-5′(S)—C-methyladenosine 5′-[phenylmethoxy-L-alaninyl)]phosphate (A16)

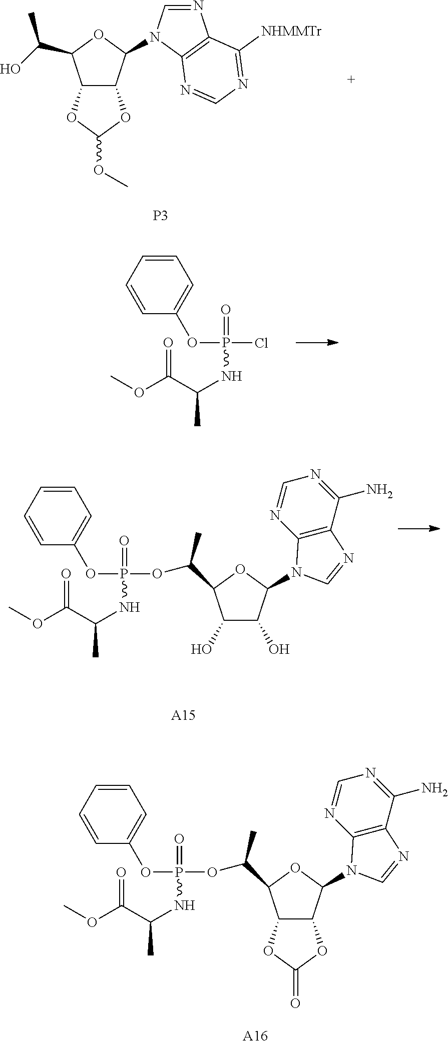

### Step 1. Preparation of phenyl(methoxy-L-alaninyl) phosphorochloridate

A solution of TEA (6 g, 59.4 mmol) in anhydrous dichloromethane (50 mL) was added dropwise to a solution of phenyl phosphorodichloridate (6 g, 28.4 mmol) and methyl alaninate hydrochloride (4 g, 28.8 mmol) in DCM (120 mL) with vigorous stirring at −78° C. over a period of 2 h. After addition, the reaction temperature was allowed to warm to RT gradually and stirred about 2 h. The solvent was removed under vacuum. Anhydrous ether 20 mL was added. The precipitated salt was filtered, and the precipitate was washed with ether. The combined organic phase was concentrated and purified by column chromatography to give phenyl(methoxy-L-alaninyl) phosphorochloridate as colorless syrup.

### Step 2. Preparation of 5′(S)—C-methyladenosine 5′-[phenyl(methoxy-L-alaninyl)]phosphate (A15)

To a solution of 2′,3′-O-methoxymethylene-N⁶-(4-methoxytrityl)-5′(S)-methyladenosine (P3) (500 mg, 0.84 mmol) in THF (30 mL) under argon was added 1.0 M t-BuMgBr in THF (2.1 mL, 2.1 mmol) at 0° C. The resulting solution was stirred at RT for 30 min and phenyl(methyl-L-alaninyl) phosphorochloridate (700 mg, 2.5 mmol) was added at 0° C. The reaction mixture was stirred at RT for 20 h, cooled with ice, quenched with water, diluted with ethyl acetate, washed with brine, extracted with ethyl acetate three times, and dried over MgSO₄. After concentration of organic layer, a protected product of A15 was obtained as a solid. The protect product of A15 was dissolved in 80% formic acid (25 mL) and stirred at RT overnight. Solvent was evaporated at RT and co-evaporated with MeOH/toluene three times. Chromatography on silica gel with 10-15% MeOH in DCM, followed by re-purification on reverse-phase HPLC with acetonitrile/water, gave 110 mg of 5′(S)—C-methyladenosine 5′-[phenyl(methoxy-L-alaninyl)]phosphate (A15) as white solid (two separated P-isomers A15-1 and A-15-2); ¹H NMR (major isomer A15-1, CD₃OD) δ 1.24 (d, J=6.8 Hz, 3H), 1.43 (d, J=6.4 Hz, 3H), 3.58 (s, 3H), 3.88-3.95 (m, 1H), 4.02-4.05 (m, 1H), 4.42 (t, J=4.4 Hz, 1H), 4.58 (t, J=4.8 Hz, 1H), 4.74-4.83 (m, 1H), 6.04 (d, J=4.8 Hz, 1H), 7.15-7.36 (m, 5H), 8.21 (s, 1H), 8.31 (s, 1H); MS m/z 522.8 (MH⁺); ¹H NMR (CD₃OD, minor isomer A15-2) δ 1.24 (d, J=6.8 Hz, 3H), 1.52 (d, J=6.4 Hz, 3H), 3.66 (s, 3H), 3.91-3.95 (m, 1H), 4.06-4.08 (m, 1H), 4.35 (t, J=4.4 Hz, 1H), 4.52 (t, J=5.2 Hz, 1H), 4.82-4.85 (m, 1H), 6.05 (d, J=5.2 Hz, 1H), 7.13-7.31 (m, 5H), 8.20 (s, 1H), 8.29 (s, 1H); MS m/z 522.9 (MH⁺).

### Step 3. Preparation of 2,3′-O-carbonyl-5′ (S)—C-methyladenosine 5′-[phenyl(methoxy-L-alaninyl)]phosphate (A16)

A solution of compound A15-1 (200 g, 0.38 mmol) in anhydrous dichloromethane (20 mL) was added CDI (200 g, 1.23 mmol) at RT and stirred about 2 h. The solvent was removed under vacuum at 0° C. and purified by prep. HPLC (neutral) to give mg (10%) of 2′,3′-O-carbonyl-5′(S)—C-methyladenosine 5′-[phenyl(methoxy-L-alaninyl)]phosphate (A16) as white solid; ¹H NMR (CDCl₃) δ1.26 (d, J=6.8 Hz, 3H), 1.50 (d, J=6.4 Hz, 3H), 3.68 (s, 3H), 3.71-3.76 (m, 1H), 3.92-3.98 (m, 1H), 4.37 (t, J=4 Hz, 1H), 4.77-4.82 (m, 1H), 5.42 (dd, J₁=7.6 Hz, J₂=3.6 Hz, 1H), 5.67 (dd, J₁=7.6 Hz, J₂=2.0 Hz, 1H), 5.77 (s, 2H), 6.05 (d, J=4.8 Hz, 1H), 6.95-6.98 (m, 2H), 7.08-7.12 (m, 1H), 7.19-7.23 (m, 2H), 7.91 (s, 1H), 8.30 (s, 1H); ³¹P NMR (160 MHz, CDCl₃): δ 1.70 (s). MS m/z 549.0 (MH⁺).

### Example 29

### Preparation of 2′,3′-O-dipropionyl-5′(S)—C-methyladenosine 5′-[phenyl(neopentoxy-L-alaninyl)]phosphate (A17)

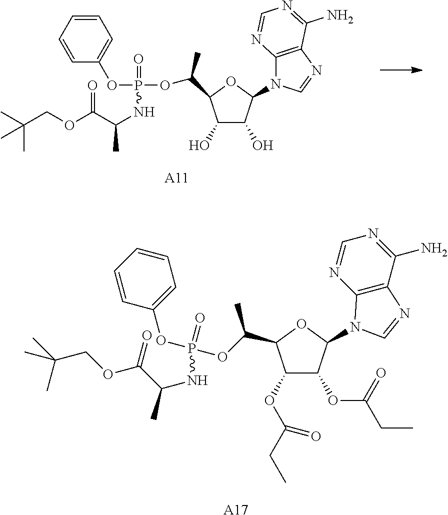

To a solution of compound A11 (200 mg, 0.35 mmol) in anhydrous pyridine (10 mL) were added propionic anhydride (182 mg, 1.4 mmol) and DMAP (68 mg, 0.52 mmol) at RT. The mixture was stirred about 18 h as checked with LCMS. The solvent was removed under reduced pressure at RT, and the residue was purified by reverse-phase HPLC to give 102 mg (43%) of 2′,3′-O-dipropionyl-5′(S)—C-methyladenosine 5′-[phenyl(neopentoxy-L-alaninyl)]phosphate (A17) as a mixture of two P-isomers; ³¹P NMR (160 MHz, CDCl₃): δ1.88 (s). MS m/z 690.9 (MH⁺).

### Example 30

### Preparation of 2′,3′-O-dipropionyl-F(S)—C-methyladenosine 5′-[phenyl(neopentoxy-L-alaninyl)]phosphate (A18)

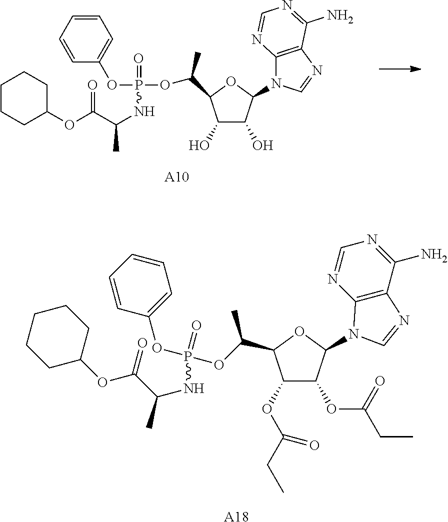

To a solution of compound A10 (270 mg, 0.46 mmol) in anhydrous pyridine (10 mL) were added propionic anhydride (270 mg, 2.07 mmol) and DMAP (65 mg, 0.53 mmol) at RT. The resulting mixture was stirred about 18 h. The solvent was removed under vacuum at RT and purified by reverse-phase HPLC to give 110 mg (34%) of 2′,3′-O-dipropionyl-5′(S)—C-methyladenosine 5′-[phenyl(neopentoxy-L-alaninyl)]phosphate (A18) as a mixture of two P-isomers, ³¹P NMR (160 MHz, CDCl₃): δ1.92 (s). MS m/z 703.5 (MH⁺).

### Example 31

### Preparation of 5′(S)—C-methyladenosine 5′-[phenyl(ethoxy-L-alaninyl)]phosphate (A19)

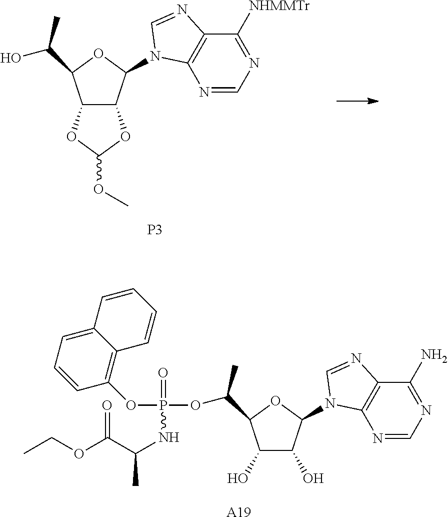

Following the general procedure for 5′(S)—C-methyladenosine 5′-[1-naphthyl(cyclohexoxy-L-alaninyl)]phosphate, 50 mg of 5′(S)—C-methyladenosine 5′-[1-naphthyl(ethoxy-L-alaninyl)]phosphate (A19) was obtained as white solid from 112 mg of 2′,3′-O-methoxymethylidene-N⁶-(4-methoxytrityl)-5′(S)-methyladenosine (P3). ³¹P NMR (CD₃OD, two isomers) δ 3.32 (s), 3.48 (s). MS m/z 587.2 (MH⁺).

### Example 32

### Preparation of 5′(S)—C-methyladenosine 5′-[phenyl(isopropoxy-L-alaninyl)]phosphate (A20)

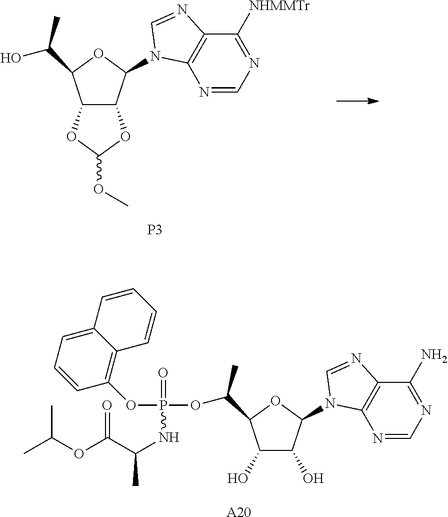

Following the general procedure for 5′(S)—C-methyladenosine 5′-[1-naphthyl(cyclohexoxy-L-alaninyl)]phosphate, 20.3 mg of 5′(S)—C-methyladenosine 5′-[1-naphthyl(isopropoxy-L-alaninyl)]phosphate (A20) was obtained as white solid from 121 mg of 2′,3′-O-methoxymethylidene-N⁶-(4-methoxytrityl)-5′(S)-methyladenosine (P3). ³¹P NMR (CD₃OD, two isomers) δ 3.41 (s), 3.51 (s). MS m/z 601.2 (MH⁺).

### Example 33

### Preparation of 5′(S)—C-methyladenosine 5′-[phenyl(benzyloxy-L-alaninyl)]phosphate (A21)

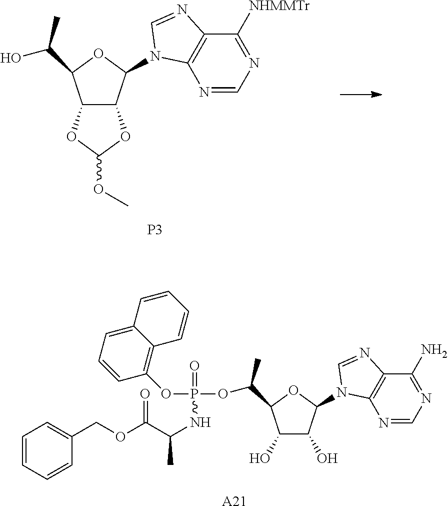

Following the general procedure for 5′(S)—C-methyladenosine 5′-[1-naphthyl(cyclohexoxy-L-alaninyl)]phosphate, 50 mg of 5′(S)—C-methyladenosine 5′-[1-naphthyl(benzyloxy-L-alaninyl)]phosphate (A21) was obtained as white solid from 87 mg of 2′,3′-O-methoxymethylidene-N⁶-(4-methoxytrityl)-5′(S)-methyladenosine (P3). ³¹P NMR (CD₃OD, two isomers) δ 5.88 (s), 5.90 (s). MS m/z 647.4 (M⁻).

### Example 34

### Preparation of 2′,3′-O-dipropionyl-5′-(S)—C-methyladenosine 5′-[1-naphthyl(isopropoxy-L-alaninyl)]phosphate (A22)

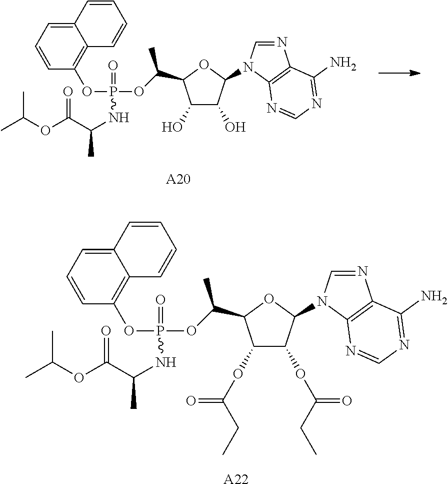

To a solution of 5′-(S)—C-methyladenosine-5′-[1-naphthyl-(isopropoxy-L-alaninyl)]phosphate (A20) (148 mg, 0.25 mmol) in DMF (3 mL), were added DCC (153 mg, 0.74 mmol), propionic acid (55 μl, 0.74 mmol), DMAP (30 mg, 0.25 mmol). The mixture was stirred to RT for overnight. Reaction mixture was filtered, and filtrate was concentrated with a rotary evaporator until half of its original volume. EA was added to the reaction mixture. The reaction mixture was then washed with water, followed by brine, dried over anhydrous Na₂SO₄ and concentrated in vacuo. The residue which was purified by silica gel with DCM/MeOH=95:5 to give 110.0 mg (62%) of 2′,3′-O-dipropionyl-5′-(S)—C-methyladenosine 5′-[1-naphthyl(isopropoxy-L-alaninyl)]phosphate (A22) as white foam after lyophilization; ¹H NMR (DMSO-d₆, two isomers) δ 1.01-1.16 (m, 10H), 1.26, 1.42 (2d, J=6.4 Hz, 2H), 1.27, 1.42 (2d, J=6.4 Hz, 3H), 2.24-2.31 (m, 4H), 3.82-3.87 (m, 1H), 4.24-4.26 (m, 1H), 4.7-4.4.81 (m, 2H), 5.69-4.5.7 (m, 1H), 5.85-5.89 (m, 1H), 6.10 (dd, J=10.4, 11.2 Hz, 1H), 7.34 (br s, 2H), 7.38-7.54 (m, 4H), 7.88-7.92 (m, 1H), 8.05-8.09 (m, 1H), 8.10, 8.13 (2s, 1H), 8.16, 8.27 (2s, 1H); ³¹P NMR (DMSO-d6, two isomers) δ 3.36 (s), 4.03 (s); MS m/z 713.4 (MH⁺).

### Example 35

### Preparation of 2′,3′-O-carbonyl-5(S)—C-methyladenosine 5′-[1-naphthyl(isopropoxy-L-alaninyl)]phosphate (A23)

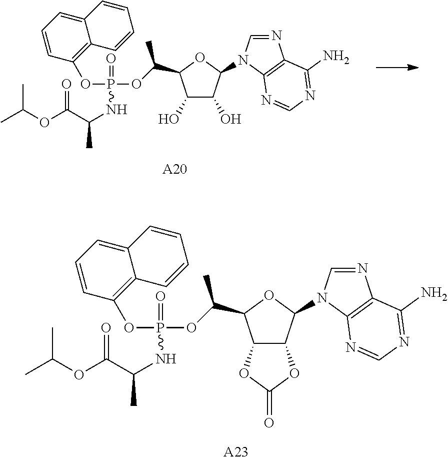

To a solution of 5′(S)—C-methyladenosine-5′-[1-naphthyl-(isopropyloxy-L-alaninyl)]phosphate (A20) (100 mg, 0.17 mmol) in DMF (2 mL) at 0-5° C., was added DCC (62 mg, 0.38 mmol). The mixture was allowed to warm to RT and was stirred for 2 h. The solvent was removed with a rotary evaporator, and the residue was subjected to column chromatography on silica gel with 5-8% MeOH in DCM, and gave 18 mg of pure 2′,3′-carbonyl-5′-(S)—C-methyladenosine 5′-[1-naphthyl(isopropoxy-L-alaninyl)]phosphate. Re-purification of the impure fractions on silica gel with 5-10% isopropanol in DCM gave 61 mg of 2′,3′-carbonate-5′-(S)—C-methyladenosine-5′-[1-naphthyl(isopropoxy-L-alaninyl)]phosphate (A23) as white foam. (total amount=79 mg, 74%); ¹H NMR (DMSO-d₆, two isomers) δ 1.01-1.16 (m, 10H), 1.26, 1.42 (2d, J=6.4 Hz, 2H), 3.76-3.83 (m, 1H), 4.41-4.46 (m, 1H), 4.72-4.86 (m, 1H), 5.46, 5.24 (2×dd, J=8.8, 14.0 Hz, 1H), 5.77-5.87 (m, 1H), 5.92, 6.08 (2×dd, J=3.2, 10.0 Hz, 1H), 6.45, 6.47 (2×d, J=3.6 Hz, 1H), 7.35 (br s, 2H), 7.38-7.70 (m, 7H), 7.85-7.95 (m, 2H), 8.11, 8.22 (2s, 1H), 8.24, 8.26 (2s, 1H); ³¹P NMR (DMSO-d₆, two isomers) δ 3.05 (s), 3.93 (s). MS m/z 627.3 (MH⁺).

### Example 36

### Preparation of 5′(S)—C-methylguanosine 5′-[phenyl(methoxy-L-alaninyl)]phosphate (B1)

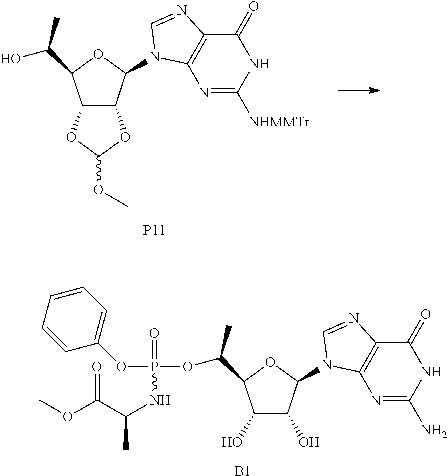

To a solution of 2′,3′-O-methoxymethylidene-N⁶-(4-methoxytrityl)-5′(5)-methylguanosine (P11) (79 mg, 0.13 mmol) in THF (1.3 mL) under argon was added dropwise 1.0 M tert-BuMgBr in THF (0.52 mL). The resulting solution was stirred at RT for 30 min and phenyl(methoxy-L-alaninyl) phosphorochloridate (1.0 M in THF, 0.65 mL) was added. The reaction mixture was stirred at RT for 3 days. The mixture was then cooled with ice, quenched with aqueous ammonium chloride, diluted with ethyl acetate, washed with aqueous ammonium three times, dried over sodium sulfate, and concentrated. Chromatography on silica gel with 5-7% MeOH in DCM gave a mixture of four isomers. The mixture was dissolved in 80% formic acid (9 mL), and the resulting solution stood at RT overnight. Solvent was evaporated at RT and co-evaporated with MeOH/toluene three times. Purification on reverse-phase HPLC (C18) using 1% formic acid in acetonitrile and water, followed by lyophilization, gave 5′(S)—C-methylguanosine 5′-[phenyl(methoxy-L-alaninyl)]phosphate (B1) (9.9 mg of major isomer and 2.2 mg of minor isomer) as white solid; ¹H NMR (CD₃OD, major isomer) δ 1.17 (dd, J=7.2, 1.2 Hz, 3H), 1.43 (d, J=6.4 Hz, 3H), 3.58 (s, 3H), 3.81-3.89 (m, 1H), 3.93 (m, 1H), 4.24 (dd, J=5.2, 4.0 Hz, 1H), 4.33 (t, J=5.2 Hz, 1H), 4.70-4.78 (m, 1H), 5.78 (d, J=5.6 Hz, 1H), 7.03-7.10 (m, 3H), 7.19-7.23 (m, 2H), 7.78 (s, 1H); ³¹P NMR (CD₃OD, major isomer) δ 3.09 (s). MS m/z 539.3 (MH⁺).

### Example 37

### Preparation of 5′(R)—C-methylguanosine 5′-[phenylmethoxy-L-alaninyl)]phosphate (B2)

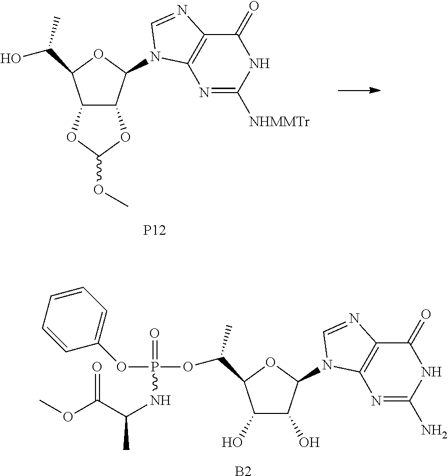

Following the general procedure for 5′(S)—C-methylguanosine 5′-[phenyl(methoxy-L-alaninyl)]phosphate (B1), 20.1 mg of 5′(R)—C-methylguanosine 5′-[phenyl(methoxy-L-alaninyl)]phosphate (B2) was obtained as white solid from 120 mg (0.2 mmol) of 2′,3′-O-methoxymethylidene-N²-(4-methoxytrityl)-5′(R)-methylguanosine (P12). ¹H NMR (CD₃OD, two isomers) δ 1.12, 1.20 (2dd, J=6.4, 1.2/0.8 Hz, 3H), 1.34 (d, J=6.4 Hz, 3H), 3.53, 3.57 (2s, 3H), 3.73-3.89 (m, 2H), 4.32, 4.46 (2dd, J=5.6, 3.2/4.0 Hz, 1H), 4.62, 4.63 (2t, J=6.0/5.6 Hz, 1H), 4.78-4.87 (m, 1H), 5.69, 5.73 (2d, J=6.0/5.6 Hz, 1H), 7.05-7.15 (m, 3H), 7.22-7.29 (m, 2H), 7.78 (2s, 1H); ³¹P NMR (CD₃OD, two isomers) δ 2.93 (s), 3.23 (s). MS m/z 539.0 (MH⁺).

### Example 38

### Preparation of 5′(R)—C-methylguanosine 5′-[1-naphthyl(isopropoxy-L-alaninyl)]phosphate (B3)

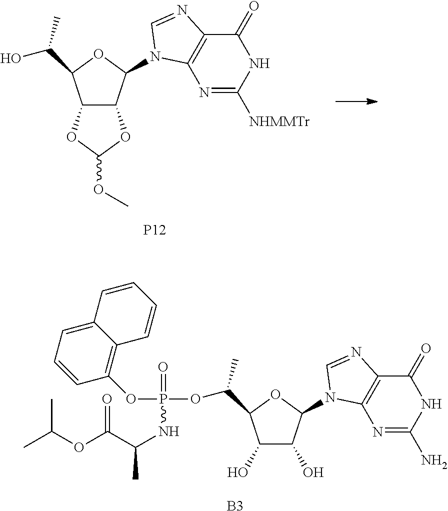

Following the general procedure for 5′(S)—C-methylguanosine 5′-[phenyl(methoxy-L-alaninyl)]phosphate (B1), 11 mg (two isomers) of 5′ (R)—C-methylguanosine 5′-[1-naphthyl(isopropoxy-L-alaninyl)]phosphate (B3) was obtained as white solid from 73 mg (0.12 mmol) of 2′,3′-O-methoxymethylidene-N²-(4-methoxytrityl)-5′(R)—C-methylguanosine (P12) and 1-naphthyl(isopropoxy-L-alaninyl) phosphorochloridate. ¹H NMR (CD₃OD, two isomers) δ 1.12-1.20 (m, 6H), 1.22, 1.26 (2dd, J=7.2, 1.2 Hz, 3H), 1.35, 1.46 (2d, J=6.8/6.4 Hz, 3H), 3.89-3.99 (m, 2H), 4.41, 4.51 (2dd, J=5.6, 3.2 Hz, 1H), 4.63, 4.71 (2t, J=6.0 Hz, 1H), 4.87-5.02 (m, 2H), 5.77, 5.79 (2d, J=6.0 Hz, 1H), 7.35-7.54 (m, 4H), 7.67, 7.05 (2d, J=8.0 Hz, 1H), 7.83-7.89 (m, 1H); 7.85 (s, 1H), 8.09-8.16 (m, 1H); ³¹P NMR (CD₃OD, two isomers) δ 3.32 (s), 3.58 (s). MS m/z 746.6 (MH⁺+6-methyl-2-heptylamine).

### Example 39

### Preparation of 5′(S)—C-methylguanosine 5′-[1-naphthyl(isopropoxy-L-alaninyl)]phosphate (B4)

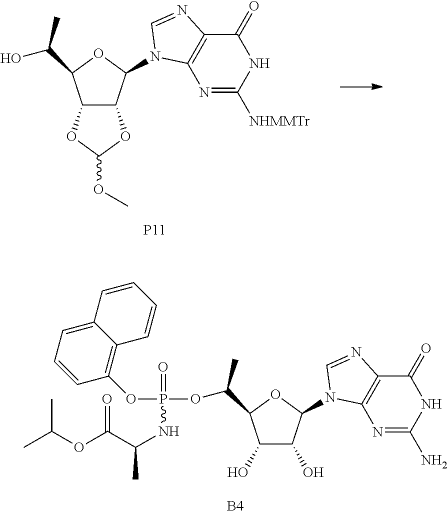

Following the general procedure for 5′(S)—C-methylguanosine 5′-[phenyl(methoxy-L-alaninyl)]phosphate (B1), 8.5 mg (two separated P-isomers) of 5′(S)—C-methylguanosine 5′-[1-naphthyl(isopropoxy-L-alaninyl)]phosphate (B4) was obtained as white solid from 73 mg (0.12 mmol) of 2′,3′-O-methoxymethylidene-N²-(4-methoxytrityl)-5′(S)—C-methylguanosine (P11) and 1-naphthyl(isopropoxy-L-alaninyl) phosphorochloridate. ¹H NMR (CD₃OD, isomer I) δ 1.09, 1.11 (2d, J=6.4 Hz, 6H), 1.21 (dd, J=7.2, 0.8 Hz, 3H), 1.40 (d, J=6.4 Hz, 3H), 3.86-3.95 (m, 1H), 3.98 (m, 1H), 4.42-4.50 (m, 2H), 4.79-4.89 (m, 2H), 5.85 (d, J=4.8 Hz, 1H), 7.40 (t, J=8.0 Hz, 1H), 7.45-7.56 (m, 3H), 7.69 (d, J=8.0 Hz, 1H), 7.85-7.89 (m, 1H); 7.89 (s, 1H), 8.12-8.17 (m, 1H); ³¹P NMR (CD₃OD, isomer I) δ 3.62 (s). MS m/z 746.5 (MH⁺+6-methyl-2-heptylamine). ¹H NMR (CD₃OD, isomer II) δ 1.13, 1.15 (2d, J=6.4 Hz, 6H), 1.23 (dd, J=7.2, 1.2 Hz, 3H), 1.55 (d, J=6.4 Hz, 3H), 3.89-3.98 (m, 1H), 4.00 (m, 1H), 4.22 (t, J=5.6 Hz, 1H), 4.29 (dd, J=5.6, 4.0 Hz, 1H), 4.85-4.96 (m, 2H), 5.77 (d, J=5.6 Hz, 1H), 7.27-7.51 (m, 4H), 7.63 (d, J=8.0 Hz, 1H), 7.71 (s, 1H), 7.81-7.84 (m, 1H); 8.04-8.08 (m, 1H); ³¹P NMR (CD₃OD, isomer II) δ 3.46 (s). MS m/z 746.5 (MH⁺+6-methyl-2-heptylamine).

### Example 40

### Preparation of 5′(S)—C-methylinosine 5′-[1-naphthyl(isopropoxy-L-alaninyl)]phosphate (B5)

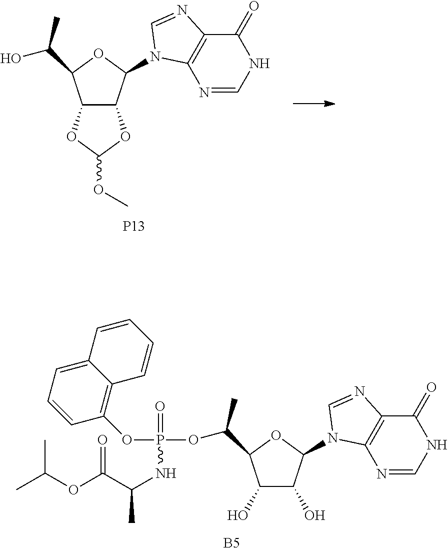

Following the general procedure for 5′(S)—C-methylguanosine 5′-[phenyl(methoxy-L-alaninyl)]phosphate (B1), 44.4 mg (two separated P-isomers) of 5′(S)—C-methylinosine 5′-[1-naphthyl(isopropoxy-L-alaninyl)]phosphate (B5) was obtained as white solid from 45.5 mg (0.14 mmol) of 2′,3′-O-methoxymethylidene-5′(S)—C-methylinosine (P13) and 1-naphthyl(isopropoxy-L-alaninyl) phosphorochloridate. ¹H NMR (CD₃OD, P-isomer I) δ 1.13, 1.14 (2d, J=6.0 Hz, 6H), 1.22 (dd, J=7.2, 1.2 Hz, 3H), 1.56 (d, J=6.4 Hz, 3H), 3.88-3.96 (m, 1H), 4.04 (dt, J=4.0, 1.2 Hz, 1H), 4.32 (dd, J=5.6, 4.0 Hz, 1H), 4.36 (t, 5.6 Hz, 1H), 4.87 9q, J=6.0 Hz, 1H), 4.90-4.98 (m, 1H), 5.95 (d, J=4.8 Hz, 1H), 7.31 (t, J=8.0 Hz, 1H), 7.42-7.51 (m, 3H), 7.63 (d, J=8.0 Hz, 1H), 7.80-7.84 (m, 1H); 7.92 (s, 1H), 8.03-8.05 (m, 1H), 8.06 (s, 1H); ³¹P NMR (CD₃OD, isomer I) δ 3.38 (s). MS m/z 731.5 (MH⁺+6-methyl-2-heptylamine). ¹H NMR (CD₃OD, P-isomer II) δ 1.09, 1.11 (2d, J=6.4 Hz, 6H), 1.21 (dd, J=7.2, 0.8 Hz, 3H), 1.40 (d, J=6.4 Hz, 3H), 3.85-3.93 (m, 1H), 4.03 (dt, J=4.4, 1.2 Hz, 1H), 4.45 (t, J=5.2 Hz, 1H), 4.57 (t, 5.2 Hz, 1H), 4.79-4.90 (m, 2H), 6.03 (d, J=5.2 Hz, 1H), 7.39 (t, J=8.0 Hz, 1H), 7.46-7.55 (m, 3H), 7.69 (dd, J=8.0, 0.8 Hz, 1H), 7.85-7.89 (m, 1H); 8.02 (s, 1H), 8.11-8.16 (m, 1H), 8.24 (s, 1H); ³¹P NMR (CD₃OD, isomer II) δ 3.55 (s). MS m/z 731.4 (MH⁺+6-methyl-2-heptylamine).

### Example 41

### Preparation of 2′-deoxy-2′-β,5′(S)—C-dimethyl-2′-α-fluorocytidine 5′-[phenyl(methoxy-L-alaninyl)]phosphate (C1)

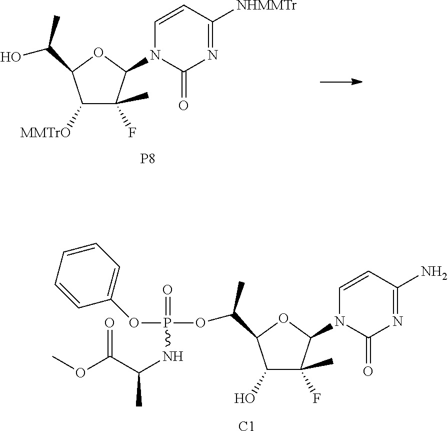

To a solution of 2′-deoxy-3′-O,N⁴-di(4-methoxytrityl)-2′-β,5′(S)—C-dimethyl-2′-α-fluorocytidine (P8) (75 mg, 0.09 mmol) in THF (1 mL) under argon was added dropwise 1.0 M tert-BuMgBr in THF (0.45 mL). The resulting solution was stirred at RT for 30 min and phenyl(methoxy-L-alaninyl) phosphorochloridate (1.0 M in THF, 0.50 mL) was added. The reaction mixture was stirred at RT for 5 days. The mixture was then cooled with ice, quenched with aqueous NH₄Cl, diluted with ethyl acetate, washed with aqueous ammonium three times, dried over sodium sulfate, and concentrated. Chromatography on silica gel with EtOAc/hexanes (2:1 to 9:1) gave 2′-deoxy-3′-O,N⁴-di(4-methoxytrityl)-2′-β,5′(S)—C-dimethyl-2′-α-fluorocytidine 5′-[phenyl(methoxy-L-alaninyl)]phosphate (18 mg of P-isomer 1 and 48 mg of P-isomer II). The P-isomer II was dissolved in 80% formic acid (3 mL), and the resulting solution stood at RT overnight and then 40° C. for 2 h. Solvent was evaporated and co-evaporated with MeOH/toluene three times. Purification on reverse-phase HPLC (C18) using 1% formic acid in acetonitrile and water, followed by lyophilization, gave 10.2 mg of 2′-deoxy-2′-β,5′(S)—C-dimethyl-2′-α-fluorocytidine 5′-[phenyl(methoxy-L-alaninyl)]phosphate (C1) as white solid; ¹H NMR (DMSO-d₆, P-isomer II) δ 1.19 (d, J=22.4 Hz, 3H), 1.25 (d, J=7.2 Hz, 3H), 1.38 (d, J=6.4 Hz, 3H), 3.55 (s, 3H), 3.75-3.96 (m, 3H), 4.69 (m, 1H), 5.74 (d, J=7.6 Hz, 1H), 5.95 (s, br, 1H), 6.14 (d, br, J=20 Hz, 1H), 7.16-7.24 (m, 4H), 7.32 (s, br, 1H), 7.36-7.41 (m, 2H), 7.53 (d, J=7.6 Hz, 1H); ³¹P NMR (DMSO-d₆, major isomer) δ 3.63 (s). MS m/z 644.3 (MH⁺+6-methyl-2-heptylamine).

### Example 42

### Preparation of 5′(S)—C-methylcytidine-[naphthyl(isopropoxy-L-alaninyl)]phosphate (C2)

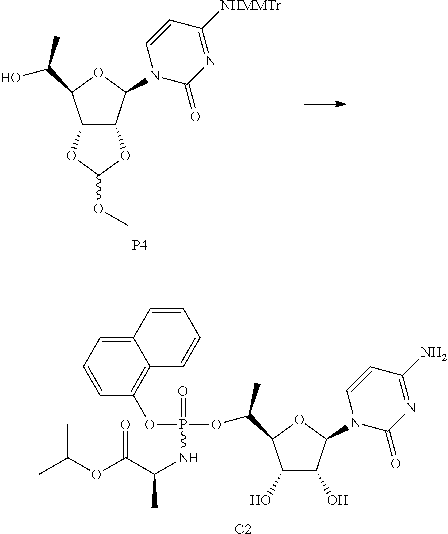

5′-(S)—C-Methylcytidine-[naphthyl(isopropoxy-L-alaninyl)]phosphate (C2) (20 mg) were prepared from 57 mg of 5′-C—(S)-methyl-2′,3′-O-methoxymethylene-N⁴-methoxytrityl)cytidine (P4) using procedure for synthesis of compound B1. ¹H NMR (CD₃OD, two P-isomers) δ8.38 (2H, bs); 8.10-8.04 (1H, m), 7.82-7.78 (1H, m), 7.63-7.58 (2H, m), 7.47-7.26 (5H, m), 5.78-5.74 (1H, two d), 5.71-5.56 (1H, two d), 5.05-4.95 (2H, m), 4.06-3.85 (6H, m), 1.49-1.34 (3H, two d), 1.22-1.18 (4H, m), 1.09-1.04 (6H, m). ³¹P NMR (CD₃OD, two isomers): δ 3.70 (s), 3.43 (s) MS: m/z 706.4 (M+H+129).

### Example 43

### Preparation of 2′-deoxy-2′-β,5′(R)—C-dimethyl-2′-α-fluorocytidine 5′-[1-naphthyl(isopropoxy-L-alaninyl)]phosphate (C3)

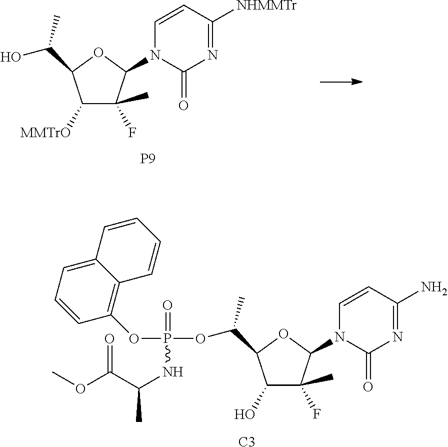

Following the general procedure for 2′-deoxy-2′-β-C-,5′(S)—C-dimethyl-2′-α-fluorocytidine 5′-[phenyl(methoxy-L-alaninyl)]phosphate, 41 mg (two P-isomers) of 2′-deoxy-2′-β,5′(R)—C-dimethyl-2′-α-fluorocytidine 5′-[phenyl(methoxy-L-alaninyl)]phosphate (C3) was obtained as white solid from 122 mg (0.15 mmol) of 2′-deoxy-3′-O,N⁴-di(4-methoxytrityl)-2′-β,5′(S)—C-dimethyl-2′-α-fluorocytidine (P9) and 1-naphthyl(methoxy-L-alaninyl) phosphorochloridate. ¹H NMR (CD₃OD, two P-isomers)δ 1.14, 1.17 (2d, J=6.0 Hz, 6H), 1.20 (d, J=22.4 Hz, 3H), 1.24, 1.30 (2dd, J=7.6, 1.2 Hz, 3H), 1.58 (d, J=6.8 Hz, 1H), 3.88-4.16 (m, 3H), 4.87-4.97 (m, 1H), 5.05-5.17 (m, 1H), 5.55, 5.73 (2d, J=7.6 Hz, 1H), 6.20 (d, br, J=20.4 Hz, 1H), 7.24, 7.43 (2t, J=8.0 Hz, 1H), 7.49-7.56 (m, 3H), 7.61, 7.75 (2d, J=7.6 Hz, 1H), 7.69-7.4 (m, 1H), 7.85-7.91 (m, 1H), 8.16-8.21 (m, 1H); ³¹P NMR (CD₃OD) δ 3.21 (s), 3.38 (s). MS m/z 722.3 (MH⁺+6-methyl-2-heptylamine).

### Example 44

### Preparation of 2′-deoxy-2′,2′-difluoro-5′(S)—C-methylcytidine 5′-[phenylmethoxy-L-alaninyl)]phosphate (C5)

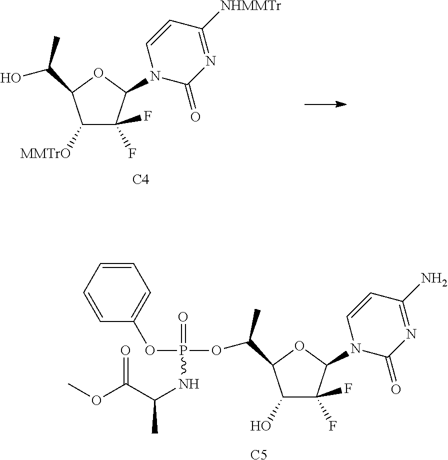

2′-Deoxy-2′,2′-difluoro-5′(S)—C-methylcytidine 5′-[phenyl(methoxy-L-alaninyl)]phosphate (C5) (5 mg) was prepared from 82 mg of 2′-deoxy-2′,2′-difluoro-3′-O,N⁴-di(4-methoxytrityl)-5′(S)—C-methylcytidine (C4) using procedure described for synthesis of 2′-deoxy-2′-β,5′(S)—C-dimethyl-2′-α-fluorocytidine 5′-[phenyl(methoxy-L-alaninyl)]phosphate. ¹H NMR (CD₃OD, two P-isomers): δ 67.53-7.51 (1H, two d); 7.47-7.10 (5H, m); 6.15-6.08 (1H, m); 5.85-5.79 (1H, two d); 4.20-3.72 (3H, m); 3.60-3.58 (3H, two s), 1.48-1.21 (6H, m). ³¹P NMR (CD₃OD, two isomers): δ 63.08 (bs). MS m/z 517.5 (M−1).

### Example 45

### Preparation of 5′(S)—C-methylcytidine 5′-[phenylmethoxy-L-alaninyl)]phosphate (C6)

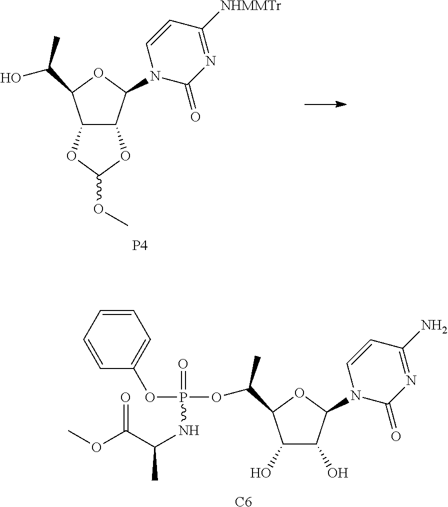

5′(S)—C-Methylcytidine 5′-[phenyl(methoxy-L-alaninyl)]phosphate (C6) (12 mg) was prepared from 86 mg of 5′-C—(S)-methyl-2′,3′-O-methoxymethylene-N⁴-(4-methoxytrityl)cytidine using procedure for synthesis of 5′(S)—C-methyladenosine 5′-[1-naphthyl(cyclohexoxy-L-alaninyl)]phosphate. ¹H NMR (CD₃OD, two isomers): δ 7.71-7.68 (1H, t); 7.29-7.06 (5H, m); 5.81-5.74 (2H, m); 4.72-3.62 (1H, m); 4.04-3.82 (4H, m); 3.60-3.58 (3H, two s), 1.46-1.19 (6H, m). ³¹P NMR (CD₃OD, two isomers): δ 3.15, 2.96 (1:1) MS: m/z 628.4 (MH⁺+2-methylheptylamine).

### Example 46

### Preparation of 5′(R)—C-methylcytidine 5′-[phenylmethoxy-L-alaninyl)]phosphate (C7)

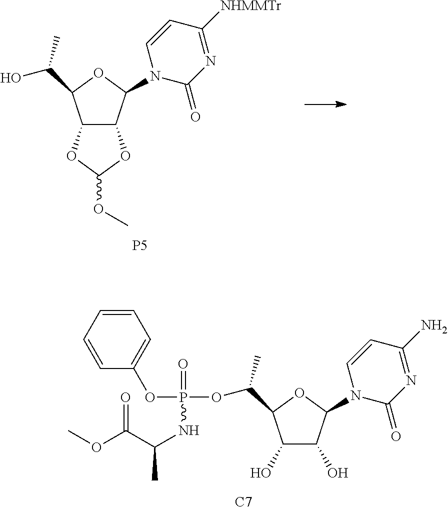

5′(R)—C-Methylcytidine 5′-[phenyl(methoxy-L-alaninyl)]phosphate (C7) (6.7 mg) was prepared from 57 mg of 5′-C—(R)-methyl-2′,3′-O-methoxymethylidene-N⁴-methoxytrityl)cytidine (P5) using procedure for synthesis of 5′(S)—C-methyladenosine 5′-[1-naphthyl(cyclohexoxy-L-alaninyl)]phosphate. ¹H NMR (CD₃OD, two isomers): δ 7.82, 7.61 (0.8H, two bs); 7.49-7.41 (1H, d); 7.02-6.81 (5H, m); 5.55-5.54 (1H, d); 5.45-5.43 (1H, d); 4.72-3.62 (1H, m); 3.88-3.85 (1H, m); 3.62-3.58 (3H, m), 3.30-3.29 (3H, s); 1.12-1.11 (3H, two s), 0.97-0.96 (3H, two s). ³¹P NMR (CD₃OD, two isomers): δ 2.86 MS: m/z 497.3 (M−H).

### Example 47

### Preparation of 5′(S)—C-methylcytidine 5′-[phenyl(isopropoxy-L-alaninyl)]phosphate (C8)

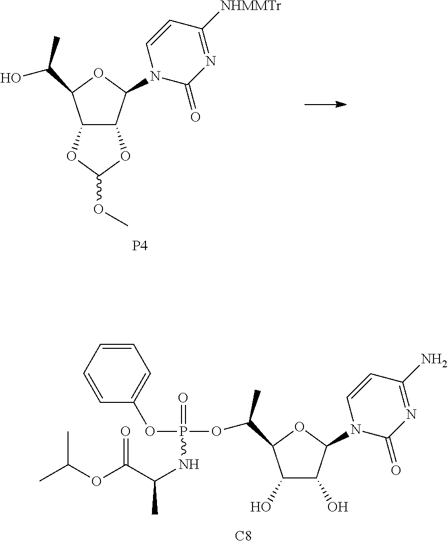

5′(S)—C-Methylcytidine 5′-[phenyl(isopropoxy-L-alaninyl)]phosphate (C8) (6.4 mg) was prepared from 57 mg of 5′-C—(S)-methyl-2′,3′-O-methoxymethylidene-N⁴-(4-methoxytrityl)cytidine (P4) using procedure for synthesis of 5′(S)—C-methyladenosine 5′-[1-naphthyl(cyclohexoxy-L-alaninyl)]phosphate. ¹H NMR (CD₃OD, two P-isomers): δ 7.79-7.78 (1H, d); 7.53-7.14 (5H, m); 5.93-5.88 (2H, m); 5.00-4.80 (1H, m); 4.25-3.85 (4H, m); 1.53-1.44 (3H, two d); 1.32-1.05 (7H, m). ³¹P NMR (CD₃OD, two isomers): δ 3.32, 2.97 (1:1) MS: m/z 656.4 (M+H+129).

### Example 48

### Preparation of 2′-deoxy-2′-C-β-fluoro-5′(R/S)—C-methylcytidine-5′-[phenyl-(methoxy-L-alaninyl)]phosphate (C9)

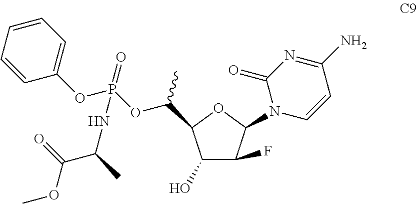

According to the procedure described for Example 41, 20.7 mg of 2′-deoxy-2′-C-β-fluoro-5′(R/S)—C-methylcytidine-5′-[phenyl-(methoxy-L-alaninyl)]phosphate (C9) was synthesized from 80.0 mg (0.1 mmol) of 2′-deoxy-3′-O,N⁴-di(4-methoxytrityl)-2′-C-β-fluoro-5′-(R/S)—C-methylcytidine. ¹H NMR (CD₃OD, four isomers) δ 1.21, 1.24, 1.27 (3d, J=7.2, 6.8, 7.2 Hz, 3H), 1.31, 1.39, 1.44 (4d, J=6.4, 6.4, 6.8, 6.4 Hz, 3H), 3.55, 3.58 (2s, 3H), 3.75-3.78 (m, 1H), 3.86-3.91 (m, 1H), 4.19-4.26 (m, 1H), 4.61-4.66 (m, 1H), 4.83, 4.97 (2dd, 1H), 5.67, 5.79 (2d, 1H), 7.09-7.17 (m, 3H), 7.18-7.28 (m, 2H), 7.74 (dd, J=7.6 Hz 1H); ¹⁹F NMR (CD₃OD) δ −200.56 to −200.85 (m); ³¹P NMR (CD₃OD, 4 isomer) δ 2.59 (s), 2.78 (s), 2.9 (s), 2.99 (s); MS m/z 499.4 (MH⁺).

### Example 49

### Preparation of 2′-deoxy-2′-C-β-methyl-5′(R/S)—C-methylcytidine 5′-[1-naphthyl (isopropoxy-L-alaninyl)]phosphate (C10)

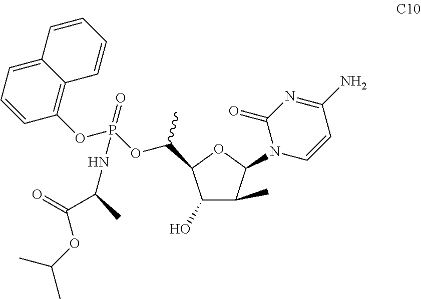

### Step 1. Preparation of 5′-O-(t-butyldimethylsilyl)-2′-deoxy-3′-O-(4-methoxytrityl)-2′-β,5′(R/S)—C-dimethyl-3′-O-(4-methoxytrityl)uridine (C12)

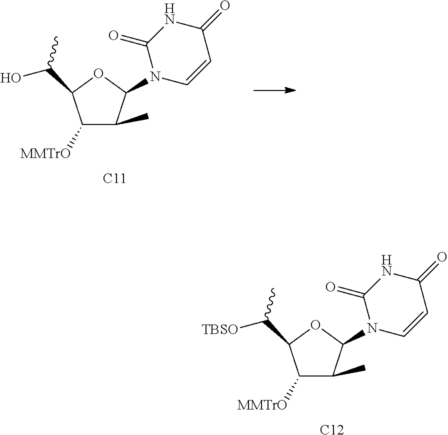

To a solution of 2′-deoxy-2′-β,5′(R/S)—C-dimethyl-3′-O-(4-methoxytrityl)uridine (C11) (390 mg, 0.74 mmol) in DMF (10 mL), were added imidazole (251 mg, 3.7 mmol), TBSCl (334 mg, 2.21 mmol), DMAP (180 mg, 1.47 mmol) successively. The reaction mixture was at stirred at 65° C. under N₂ for overnight. The reaction was monitored to completion by TLC. The reaction mixture was then cooled, diluted with EA, washed with water and brine, dried over anhydrous Na₂SO₄ and concentrated in vacuo. The residue was purified by silica gel (DCM/MeOH; 95:5) to give 5′-O-(t-butyldimethylsilyl)-2′-deoxy-2′-β,5′ (R/S)—C-dimethyl-3′-O-(4-methoxytrityl)uridine (C12) (416 g, 88%) as a white solid.

### Step 2. Preparation of 5′-O-(t-butyldimethylsilyl)-2′-deoxy-2′-β,5′(R/S)—C-dimethyl-3′-O-(4-methoxytrityl)cytidine (C13)

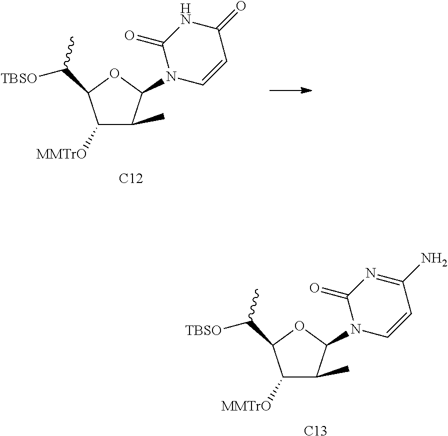

To a solution of 5′-O-(t-butyldimethylsilyl)-2′-deoxy-2′-β,5′ (R/S)—C-dimethyl-3′-O-(4-methoxytrityl)uridine (C12) (160 mg, 0.25 mmol) in anhydrous CH₃CN (3.0 mL), TEA (0.11 mL, 0.75 mmol), N-methylpiperidine (50 μL, 0.5 mmol) and TsCl (143 mg, 0.75 mmol) were added successively. The resulting mixture was stirred at RT for 2 h. After cooling the reaction to 0° C., 29% NH₄OH (2.5 mL) was then added. The resulting mixture was stirred for 2 h at RT and evaporated. The residue was purified by silica gel column chromatography (DCM/MeOH; 95:5-93:7) to give 5′-O-(t-butyldimethylsilyl)-2′-deoxy-2′-β,5′ (R/S)—C-dimethyl-3′-O-(4-methoxytrityl)cytidine (C13) (131 mg, 82%) as a white solid.

### Step 3. Preparation of 2′-deoxy-3′-O,N4-di(4-methoxytrityl)-2′-β,5′(R/S)—C-dimethylcytidine (C15)

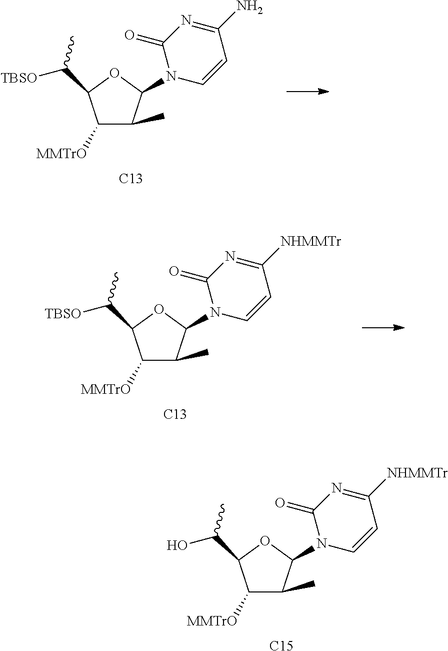

MMTrCl (452 mg, 1.47 mmol) was added to a solution of 5′-O-(t-butyldimethylsilyl)-2′-deoxy-2′-β,5′ (R/S)—C-dimethyl-3′-O-(4-methoxytrityl)cytidine (C13) (378 mg, 0.49 mmol) in anhydrous DCM (6 mL). AgNO₃ (250.0 mg, 1.47 mmol) and collidine (178 mg, 1.47 mmol) were added. The reaction mixture was stirred at RT overnight under N₂. The reaction was monitored by TLC. The reaction mixture was filtered. The mixture was then washed with saturated NaHCO₃ and brine. The organic layer was dried over Na₂SO₄ and concentrated in vacuo. The residue was purified by silica gel DCM/MeOH; 95:5) to give 5′-O-(t-butyldimethylsilyl)-2′-deoxy-3′-O,N⁴-di(4-methoxytrityl)-2′-β,5′(R/S)—C-dimethylcytidine (C14) (527 mg).

TBAF (tetra-n-butylammonium fluoride) (1.0M solution in THF) (1.1 ml, 1.1 mmol) was added to a solution of 5′-O-(t-butyldimethylsilyl)-2′-deoxy-3′-O,N⁴-di-(4-methoxytrityl)-2′-C-(β)-methyl-5′(R/S)—C-methylcytidine (500 mg, 0.55 mmol) in anhydrous THF (10 mL). The reaction mixture was stirred at RT overnight, and the reaction was monitored by TLC. EA was added to the reaction mixture. The mixture was then washed with water and brine, dried over anhydrous Na₂SO₄ and concentrated in vacuo. The residue was purified by silica gel (DCM/MeOH=95:5) to give 2′-deoxy-3′-O,N⁴-di(4-methoxytrityl)-2′-β,5′(R/S)—C-dimethylcytidine (C15) (414 mg, 94%).

### Step 4. Preparation of 2′-deoxy-2′-C-β-methyl-5′(R/S)—C-methylcytidine-5-[1-naphthyl(isopropoxy-L-alaninyl)]phosphate (C10)

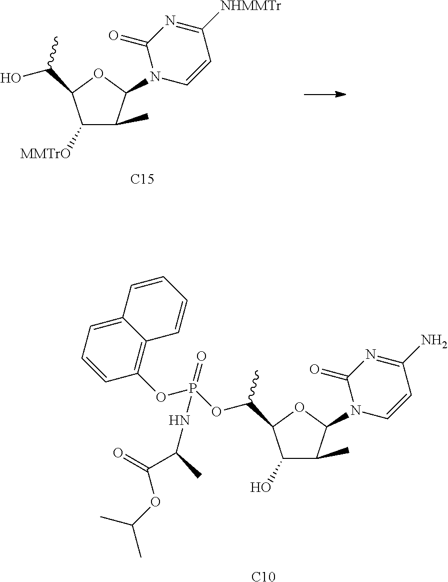

According to the procedure described for Example 41, 13.3 mg of 2′-deoxy-2′-C-β-methyl-5′ (R/S)—C-methylcytidine-5′-[1-naphthyl(isopropoxy-L-alaninyl)]phosphate (C10) was synthesized from 111 mg (0.14 mmol) of 2′-deoxy-3′-O,N⁴-di(4-methoxytrityl)-2′-β,5′ (R/S)—C-dimethylcytidine (C15). ¹H NMR (CD₃OD, two isomers) δ0.83 (d, J=7.2 Hz, 3H), 1.13-1.15 & 1-16-1.19 (2m, 6H), 1.31 (d, J=6.8 Hz, 3H), 1.44, 1.57 (2d, J=each 6.4 Hz, 3H), 2.50-2.56 (m, 1H), 3.67-3.7 (m, 1H), 3.78 (t, J=6.4 Hz, 1H), 3.96 (dd, J=6.8, 9.6 Hz, 1H), 4.85-4.88 (m, 1H), 5.77, 6.2 (d, J=7.2, 7.6 Hz, 1H), 7.43 (d, J=8.0 Hz, 1H), 7.51-7.54 (m, 4H), 7.66 (d, J=8.0 Hz, 1H), 7.72 (d, J=8.4 Hz, 1H), 7.88-7.90 (m, 1H), 8.16-8.18 (m, 1H); ³¹P NMR (CD₃OD, major isomer) δ 3.49 (s); MS m/z 704.5 (MH⁺+2-methylheptylamine).

### Example 50

### Preparation of 2′O,5′(R)—C-dimethylcytidine 5′-[phenyl(isopropoxy-L-alaninyl)]phosphate (C16)

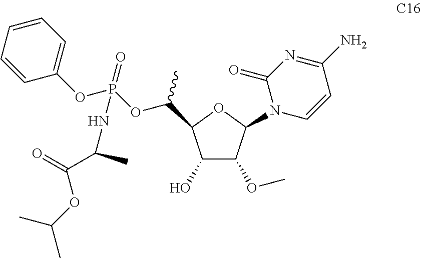

### Step 1. Preparation of 5′-O-(t-butyldimethylsilyl)-2′-O,5′(R)—C-dimethyl-3′-O-(4-methoxytrityl)uridine (C18)

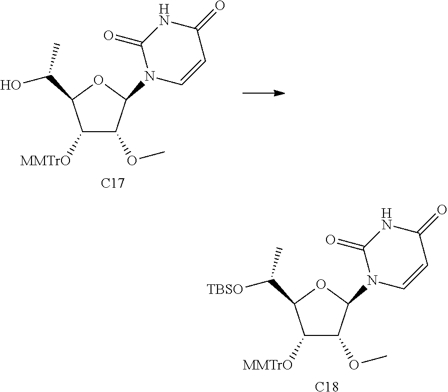

To a solution of compound C17 (140 mg, 0.26 mmol in DMF (2.5 mL), imidazole (87 mg, 1.28 mmol), TBSCl (194 mg, 1.28 mmol), DMAP (4-dimethylaminopyridine) (156 mg, 1.28 mmol) were added successively. The reaction mixture was at stirred at 80° C. under N₂ for overnight. TLC showed the reaction was complete. The reaction mixture was cooled, and diluted with EA, washed with water and brine, dried over anhydrous Na₂SO₄ and concentrated in vacuo. The residue was purified by silica gel (DCM/MeOH; 95:5) to give 5′-O-(t-butyldimethylsilyl)-2′-O,5′(R)—C-dimethyl-3′-O-(4-methoxytrityl)uridine (C18) (101 mg, 59%) as a white solid.

### Step 2. Preparation of 5′-O-(t-butyldimethylsilyl)-2′-O,5′(R)—C-dimethyl-3′-O-(4-methoxytrityl)cytidine (C19)

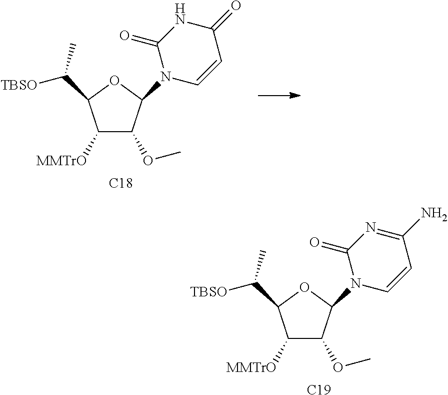

To a solution of compound C18 (160 mg, 0.24 mmol) in anhydrous CH₃CN (2.0 mL), TEA (0.105 mL, 0.72 mmol), N-methylpiperidine (49 μL, 0.48 mmol), TsCl (139 mg, 0.72 mmol) were added successively. The resulting mixture was stirred at RT for 2 h. After cooling the reaction to 0° C., 29% NH₄OH (1.5 mL) was then added. The resulting mixture was stirred for 2 h at RT and evaporated. The residue was purified by silica gel column chromatography (DCM/MeOH; 95:5-93:7) to give 5′-O-(t-butyldimethylsilyl)-2′-O,5′(R)—C-dimethyl-3′-O-(4-methoxytrityl)cytidine (C19) (131 mg, 82%) as a white solid.

### Step 3. Preparation of 3′-O,N4-di(4-methoxytrityl)-2′-O,5′(R)—C-dimethylcytidine (C21)

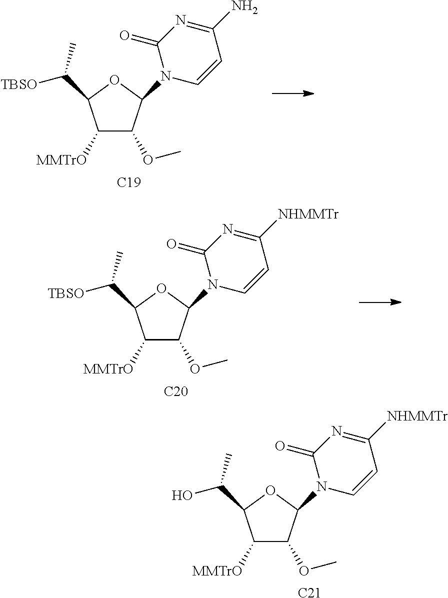

MMTrCl (184 mg, 0.6 mmol) was added to a solution of compound C19 (131 mg, 0.2 mmol) in anhydrous DCM (4 mL). AgNO₃ (102 mg, 0.6 mmol) and collidine (73 μL, 0.6 mmol) were added. The reaction mixture was stirred at RT overnight under N₂. The reaction was monitored by TLC. The reaction mixture was filtered and washed with saturated NaHCO₃ solution and brine. The organic layer was dried over Na₂SO₄ and concentrated in vacuo. The residue was purified by silica gel DCM/MeOH; 95:5) to give 5′-O-(t-butyldimethylsilyl)-3′-O,N⁴-di(4-methoxytrityl)-2′-O,5′(R)—C-dimethylcytidine (C20) (180 mg, 97%).

TBAF (1.0M solution in THF) (0.6 ml, 0.6 mmol) was added to a solution of compound C20 (180 mg, 0.19 mmol) in anhydrous THF (2 mL) and stirred at RT overnight. TLC showed the reaction was complete. EA was added to the reaction mixture and washed with water, followed by brine, dried over anhydrous Na₂SO₄ and concentrated in vacuo to give the residue which was purified by silica gel (DCM/MeOH=95:5) to give 3′-O,N⁴-di(4-methoxytrityl)-2′-O,5′(R)—C-dimethylcytidine (C21) (100.4 mg, 65%).

### Step 4. Preparation of 2′-O,5′(R)—C-dimethylcytidine 5′-[phenyl(isopropoxy-L-alaninyl)]phosphate (C16)

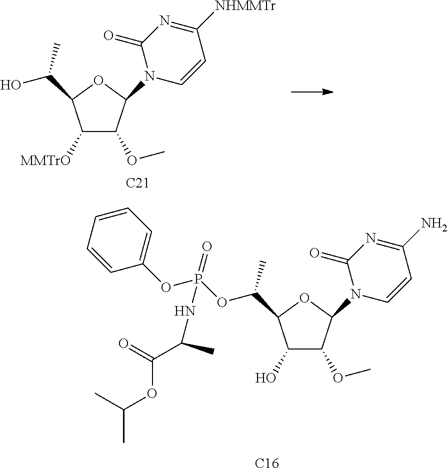

According to the procedure described for Example 41, 4.7 mg of 2′-O,5′(R)—C-dimethylcytidine 5′-[phenyl(isopropoxy-L-alaninyl)]phosphate (C16) was prepared from 100 mg (0.12 mmol) of 3′-O, N⁴-di(4-methoxytrityl)-2′-O,5′(R)—C-dimethylcytidine (C21). ¹H NMR (CD₃OD, two isomers) δ 1.18-1.37 (m, 9H), 1.47 (d, J=6.8 Hz, 3H), 3.46, 3.49 (2s, 3H), 3.71-3.94 (m, 3H), 4.28, 4.37 (each t, J=5.6, 6.0 Hz, 1H), 4.91-4.96 (m, 1H), 5.78-5.91 (m, 1H), 5.95, 5.98 (2d, J=4.4, 4.4 Hz, 1H), 7.16-7.26 (m, 3H), 7.33-7.37 (m, 2H), 7.53, 7.79 (2d, J=7.2, 7.6 Hz, 1H); ³¹P NMR (CD₃OD, two isomer) δ 2.95 (s), 3.11 (s). MS m/z 670.5 (MH⁺+diisopropylethylamine).

### Example 51

### Preparation of 5′(R)—C-methylarabinocytidine 5′-[phenyl(methoxy-L-alaninyl)]phosphate (C22)

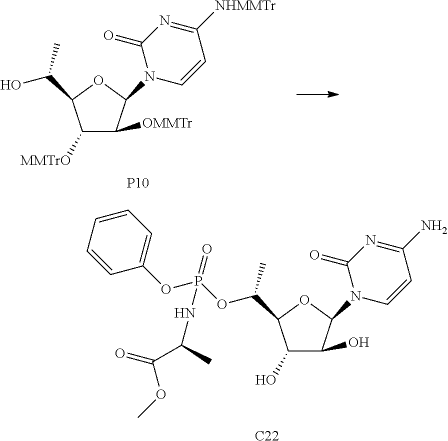

According to the procedure described for Example 41, 5.8 mg of 5′ (R)—C-methylarabinocytidine 5′-[phenyl(methoxy-L-alaninyl)]phosphate (C22) was prepared from 100 mg (0.09 mmol) of 5′(R)—C-methyl-2′,3′-O,N⁴-tri(4-methoxytrityl)arabinocytidine (P10). ¹H NMR (CD₃OD, two isomers) δ 1.28, 1.33 (2d, J=each 7.2 Hz, 3H), 1.43, 1.47 (2d, J=6.4, 6.8 Hz, 3H), 3.65, 3.66 (2s, 3H), 3.74-3.77 (m, 1H), 3.92-3.97 (m, 1H), 4.13-4.18 (m, 1H), 4.28-4.29 (m, 1H), 5.77, 5.82 (2d, J=7.6, 7.2 Hz, 1H), 6.15, 6.17 (2d, J=3.6, 4.0 Hz, 1H), 7.16-7.25 (m, 3H), 7.32-7.37 (m, 2H), 7.71 (d, J=7.6 Hz, 1H); ³¹P NMR (CD₃OD, major isomer) δ 2.38 (s), 2.65 (s). MS m/z 497.3 (MH⁺).

### Example 52

### Preparation of 5′(R)—C-methylarabinouridine 5′-[phenylmethoxy-L-alaninyl)]phosphate (D1)

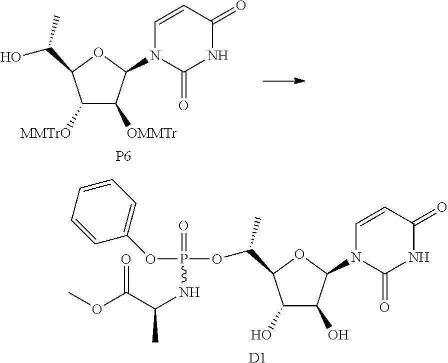

Following the general procedure for 2′-deoxy-2′-β-C-,5′(S)—C-dimethyl-2′-α-fluorocytidine 5′-[phenyl(methoxy-L-alaninyl)]phosphate, 24.7 mg (two isomers) of 5′(R)—C-methylarabinouridine 5′-[phenyl(methoxy-L-alaninyl)]phosphate (D1) was obtained as white solid from 160 mg (0.2 mmol) of 2′,3′-O-di(4-methoxytrityl)-5′(R)—C-methylarabinouridine (P6). ¹H NMR (DMSO-d₆, major isomer) δ 1.22 (d, J=6.8, Hz, 3H), 1.28 (d, J=6.0 Hz, 3H), 3.6 (s, 3H), 3.66 (dd, J=7.2, 3.2 Hz, 1H), 3.81-3.94 (m, 2H), 3.97-4.01 (m, 1H), 4.61-4.70 (m, 1H), 5.40 (d, J=8.0 Hz, 1H), 5.58 (d, J=4.8 Hz, 1H, OH), 5.66 (d, J=4.4 Hz, 1H, OH), 5.85 (dd, J=12.4, 10.0 Hz, 1H, NH), 7.15-7.23 (m, 3H), 7.35-7.40 (m, 2H), 7.3 (d, J=8.0 Hz, 1H); ³¹P NMR (DMSO-d₆, major isomer) δ 3.54 (s). MS m/z 629.4 (MH⁺+6-methyl-2-heptylamine).

### Example 53

### Preparation of 5′(S)—C-methylarabinouridine 5′-[phenylmethoxy-L-alaninyl)]phosphate (D2)

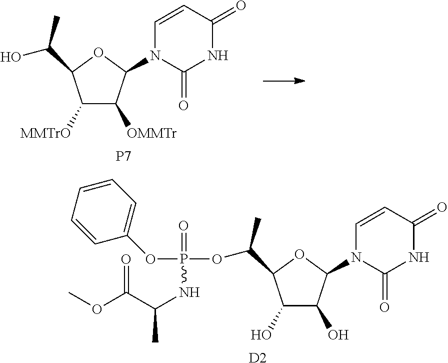

Following the general procedure for 2′-deoxy-2′-β-C-,5′(S)—C-dimethyl-2′-α-fluorocytidine 5′-[phenyl(methoxy-L-alaninyl)]phosphate, 3.1 mg (two isomers) of 5′ (S)—C-methylarabinouridine 5′-[phenyl(methoxy-L-alaninyl)]phosphate (D2) was obtained as white solid from 160 mg (0.2 mmol) of 2′,3′-O-di(4-methoxytrityl)-5′(S)—C-methylarabinouridine (P7). ¹H NMR (CD₃OD, two isomers) δ 1.29, 1.31 (2dd, J=7.2, 1.2/0.8 Hz, 3H), 1.45, 1.49 (2d, J=6.4/6.0 Hz, 3H), 3.65, 3.66 (2s, 3H), 3.73-3.78 (m, 1H), 4.12, 4.17 (2dd, J=4.0, 2.0 Hz, 1H), 4.26, 4.30 (2dd, J=3.6/4.0, 2.4 Hz, 1H), 4.78-4.90 (m, 1H), 5.57, 6.20 (2d, J=8.0 Hz, 1H), 6.12, 6.14 (2d, J=4.0/4.4 Hz, 1H), 7.16-7.26 (m, 3H), 7.33-7.38 (m, 2H), 7.69, 7.70 (2d, J=8.0 Hz, 1H); ³¹P NMR (CD₃OD, two isomers) δ 2.41 (s), 2.62 (s). MS m/z 629.4 (MH⁺+6-methyl-2-heptylamine).

### Example 54

### Preparation of 5′(S)—C-methyluridine 5′-[1-phenylmethoxy-L-alaninyl)]phosphate (D3)

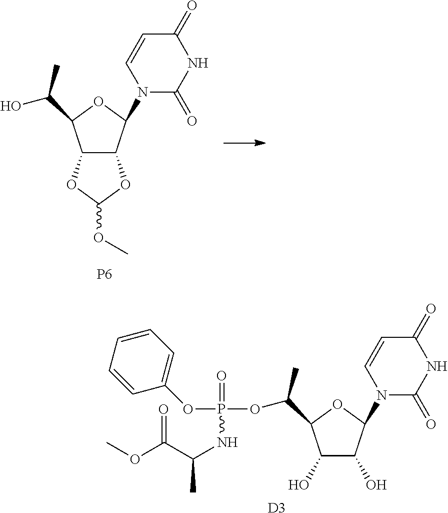

To a solution of 2′,3′-O-methoxymethylidene-5′(S)-methyluridine (P6) (106.2 mg) in 2 mL THF under argon at 0° C. was added t-BuMgCl (0.88 mL, 1 M in THF) dropwise over 5 min. After 15 min, a solution of phenyl(methoxy-L-alaninyl) phosphorochloridate (1.0 mL, 1.0 M in THF) was added. The reaction was allowed to warm to ambient temperature and was stirred for 2 days. After cooling to 0° C., the reaction was quenched with saturated NH₄Cl, and the desired product extracted with ethyl acetate. The solvents were removed, and the resultant intermediate taken up in 80% aqueous formic acid and warmed briefly to 60° C. The solvent was evaporated. The residue was co-evaporated with MeOH/toluene three times. The resultant material was subjected to silica gel chromatography, eluting with a gradient of 3% to 10% methanol in methylene chloride. 40 mg of 5′(S)—C-methyluridine 5′-[1-phenyl(methyl-L-alaninyl)]phosphate (D3) was obtained. ³¹P NMR (CDCl₃, two isomers) δ 2.30 (s), 2.52 (s). MS m/z 498.3 (M−1)⁻.

### Example 55

### Preparation of 2′-deoxy-2′,2′-difluoro-5′(S)—C-methyluridine 5′-[phenylmethoxy-L-alaninyl)]phosphate (D4)

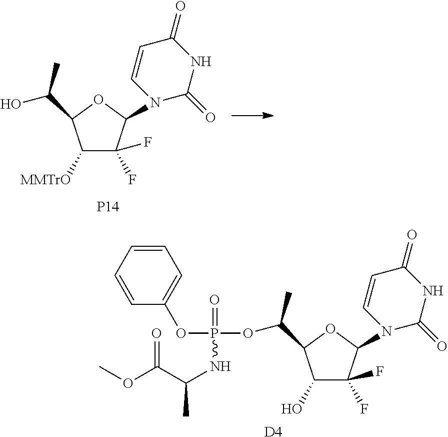

2′-Deoxy-2′,2′-difluoro-5′(S)—C-methyluridine 5′-[phenyl(methoxy-L-alaninyl)]phosphate (D4) (7.5 mg) was prepared from 55 mg of 2′-deoxy-2′,2′-difluoro-3′-(4-O-methoxytrityl)-5′(S)—C-methyluridine using procedure for synthesis of 2′-deoxy-2′-β-C-,5′(S)—C-dimethyl-2′-α-fluorocytidine 5′-[phenyl(methoxy-L-alaninyl)]phosphate described above. ³¹P NMR (CD₃OD, two isomers): δ 3.09, 3.08 (1:1). ¹H NMR (CD₃OD, two isomers): δ 7.57-7.48 (1H, two d); 7.32-7.26 (2H, m); 7.19-7.11 (3H, m); 6.08-6.03 (1H, m); 5.68-5.63 (1H, two d); 4.25-4.15 (1H, m); 3.98-3.86 (1H, m); 3.82-3.80 (1H, m); 3.60-3.58 (3H, two s), 1.54-1.38 (3H, two d), 1.30-1.20 (3H, m). MS: m/z 518.4 (M−1).

### Example 56

### Preparation of 2′-deoxy-2′-C-β-5′(R/S)—C-dimethyl-3′-O-(4-methoxytrityl)uridine 5′-[phenylmethoxy-L-alaninyl)]phosphate (D7)

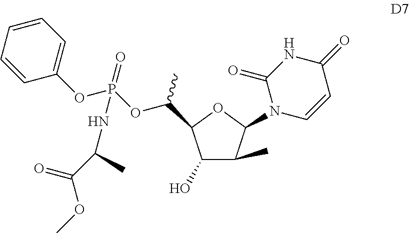

### Step 1. Preparation of 5′-O-(t-butyldimethylsilyl)-2′-deoxy-3′-O-(4-methoxytrity)-2′-C-β-methyluridine (D8)

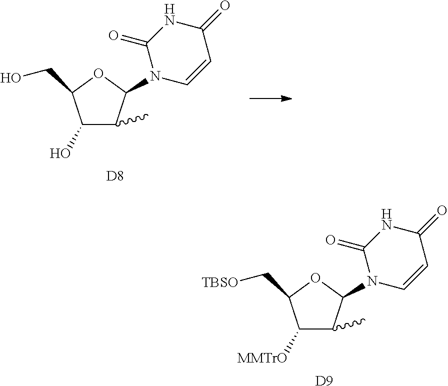

TBSCl (1.39 g, 8.84 mmol) was added to a solution of 2′-deoxy-2′-(β/α˜9:1)—C-methyluridine (D8) (prepared according to a published procedure: Journal of Organic Chemistry, 2003, 68, 6799) (1.78 g, 7.37 mmol) in anhydrous pyridine (30 mL) at 0° C. under N₂. The reaction mixture was stirred at RT overnight, and the progress of the reaction was monitored by TLC. The solvent was evaporated under reduced pressure. The residue was diluted with EA, washed with water and brine, dried over anhydrous Na₂SO₄ and concentrated in vacuo. The residue was purified by silica gel (DCM/MeOH; 95:5) to give 5′-O-(t-butyldimethylsilyl)-2′-deoxy-2′-C-(β/α˜9:1)-methyluridine (1.6 g, 60%) as a white solid.

MMTrCl (407 mg, 1.32 mmol) was added to a solution of (314 mg, 0.88 mmol) 5′-O-(t-butyldimethylsilyl)-2′-deoxy-2′(β/α)-C-methyluridine in anhydrous DCM (4 mL). AgNO₃ (225.0 mg, 1.32 mmol) and collidine (0.21 ml, 1.76 mmol) were added. The reaction mixture was stirred at RT overnight under N₂. TLC showed the reaction was complete. The reaction mixture was filtered and washed with saturated NaHCO₃ solution and brine. The organic layer was dried over Na₂SO₄ and concentrated in vacuo. The residue was purified by silica gel DCM/MeOH; 95:5) to give 5′-O-(t-butyldimethylsilyl)-2′-deoxy-3′-O-(4-methoxytrity)-2′-C-β/α(9:1)-methyluridine (D9) (542 mg, 98%).

### Step 2. Preparation of 2′-deoxy-3′-O-4-methoxytrity-2′-C-(β)-methyluridine (D10)

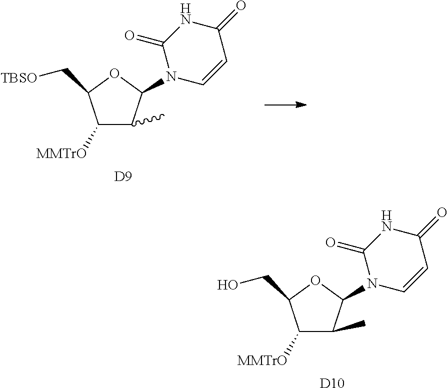

TEA-3HF (0.28 ml, 1.72 mmol)/TEA (0.25 ml, 1.72 mmol) was added dropwise to a solution of 5′-O-(t-butyldimethylsilyl)-2′-deoxy-3′-O-(4-methoxytrity)-2′-C-β/α-methyluridine (D9) (542 mg, 0.86 mmol) in anhydrous THF (13 mL). The reaction mixture was stirred at RT overnight. The reaction was monitored by TLC. The reaction was showed to be incomplete by TLF. TEA.3HF (0.54 ml, 3.3 mmol) and TEA (0.6 ml, 4.15 mmol) were added until the reaction was showed to be complete by TLC. The solvent was removed in vacuo at RT. DCM was added. The residue and washed with water and brine, dried over anhydrous Na₂SO₄ and concentrated in vacuo. The residue was purified by silica gel (Hexanes/EA=1:9) to give 2′-deoxy-3′-O-(4-methoxytrity)-2′-C-β-methyluridine (D10) (347 mg, 78%).

### Step 3. Preparation of 2′-deoxy-5-C,5′-O-didehydro-3′-O-(4-methoxytrityl)-2′-C-β-methyl-uridine (D11)

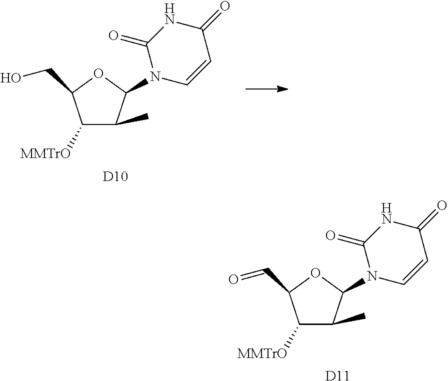

Pyridine (0.68 mL, 8.55 mmol) and Dess-Martin (324 mg, 0.76 mmol) were added to a solution of 2′-deoxy-3′-O-4-(methoxytrity)-2′-C-β-methyluridine (D10) (295 mg, 0.57 mmol) in anhydrous CH₂Cl₂ (7 mL) at 0° C. under N₂. The reaction mixture was stirred at RT for 4 h. The reaction was monitored by TLC. The reaction mixture was diluted with EA. The organic layer was washed with 10% Na₂S₂O₃ twice, followed by water and brine, dried over anhydrous Na₂SO₄ and concentrated in vacuo. The residue was purified by silica gel (DCM/EA=1/1) to give 2′-deoxy-5-C,5′-O-didehydro-3′-O-(4-methoxytrityl)-2′-C-β-methyl-uridine (D11) (273 mg, 94%).

### Step 4. Preparation of 2′-deoxy-2′-C-β-5′(R/S)—C-dimethyl-3′-O-(4-methoxytrityl)uridine (C11)

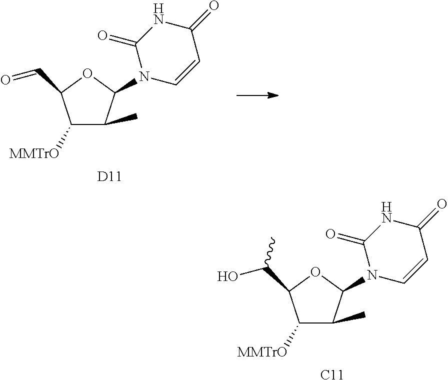

MeMgBr (1.52 mL, 2.13 mmol) was added dropwise to a solution of 2′-deoxy-5-C,5′-O-didehydro-3′-O-(4-methoxytrityl)-2′-C-β-methyl-uridine (D11) (273 mg, 0.53 mmol) in anhydrous THF (10 mL). The reaction mixture was cooled by an ice-EtOH bath under N₂. The reaction mixture was stirred at RT for 6 h. The reaction was monitored by TLC. The reaction mixture was quenched with saturated NH₄Cl. EA was added, and the organic layer was washed with water and brine, dried over anhydrous Na₂SO₄ and concentrated in vacuo. The residue was purified by silica gel (hexanes/EA=1/1 to 1/1) to give 2′-deoxy-2′-C-β-5′ (R/S)—C-dimethyl-3′-O-(4-methoxytrityl)uridine (C11) (116 mg, 41%).

### Step 5. Preparation of 2′-deoxy-2′-C-β-5′(R/S)—C-dimethyl-3′-O-(4-methoxytrityl)uridine 5′-[phenyl(methoxy-L-alaninyl)]phosphate (D 7)

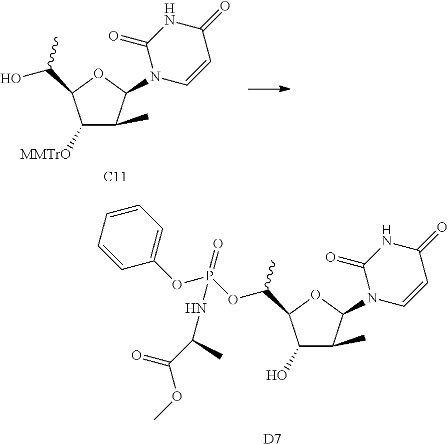

According to the procedure described for Example 41, 22.1 mg of 2′-deoxy-2′-C-β-5′(R/S)—C-dimethyl-3′-O-(4-methoxytrityl)uridine 5′-[phenyl(methoxy-L-alaninyl)]phosphate (D7) was prepared from 60.0 mg (0.11 mmol) of 2′-deoxy-3′-O-(4-methoxytrityl)-2′-C-(β)-methyl-5′(R/S)—C-methyluridine (C11). ¹H NMR (DMSO-d₆) 0.75, 0.82 (2d, each J=7.2 Hz, 3H), 1.19, 1.24 (2d, each J=7.2 Hz, 3H), 1.35, 1.39, 1.44 (3d, J=6.4, 6.8, 6.4 Hz, 3H), 2.43-2.47 (m, 1H), 3.54, 3.56 (2s, 3H), 3.59 (m, 1H), 3.75-3.81 (m, 1H), 3.86-3.90 (m, 1H), 4.67-4.72 (m, 1H), 5.44 (d, J=5.6 Hz, 1H), 5.48, 5.52 (2d, J=8.4, 8.0 Hz, 1H), 5.86 (t, J=12.0 Hz, 1H), 6.11 (d, J=7.6 Hz, 1H), 7.16-7.23 (m, 1H), 7.34-7.40 (m, 1H), 7.62 (d, J=8.0 Hz, 1H), 11.34 (s, 1H); ³¹P NMR (DMSO-d₆, four isomers) δ 3.33 (s), 3.59 (s), 3.64 (s), 3.70 (s); MS m/z 496.4 (M−H⁺).

### Example 57

### Preparation of 2′O,5′(R)—C-dimethyluridine-5′-[1-naphthyl(isopropoxy-L-alaninyl)]phosphate (D12)

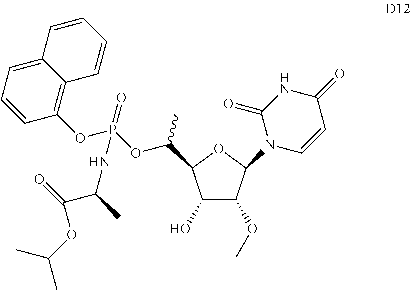

### Step 1. Preparation of 5′-O-(t-butyldimethylsilyl)-3′-O-(4-methoxytrityl)-2′-O-methyluridine (D15)

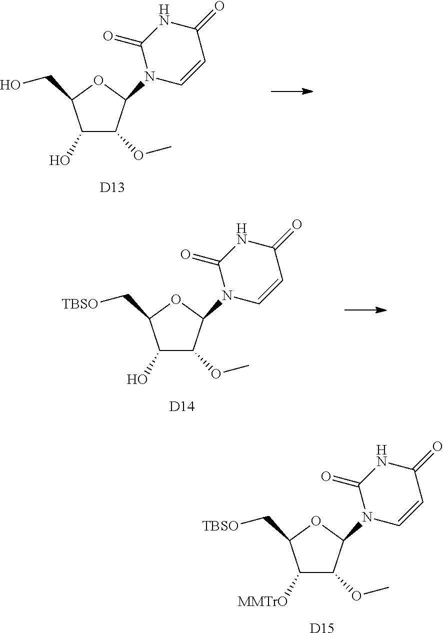

TBSCl (7.0 g, 46.5 mmol), and DMAP (0.95 g, 7.76 mmol) were added to a solution of commercially available 2′-O-methyl uridine (D13) (10.0 g, 38.8 mmol) in anhydrous pyridine (100 mL) at 0° C. under N₂. The reaction mixture was stirred at RT overnight. TLC was used to monitor the reaction. The solvent was evaporated under reduced pressure. The residue was diluted with EA, washed with water and brine, dried over anhydrous Na₂SO₄ and concentrated in vacuo. The desired product, 5′-O-(t-butyldimethylsilyl)-2′-O-methyluridine (D14) (12.6 g), was obtained as white solid, which was used in next step without further purification.

MMTrCl (7.5 g, 24.5 mmol) was added to a solution of 5′-O-(t-butyldimethylsilyl)-2′-O-methyluridine (D14) (7.0 g, 18.8 mmol) in anhydrous DCM (50 mL). AgNO₃ (4.2 g, 24.5 mmol) and collidine (3.4 ml, 37.6 mmol) was added. The reaction mixture was stirred at RT overnight under N₂. The reaction was monitored by TLC. The reaction mixture was filtered and washed with saturated NaHCO₃ solution and brine. The organic layer was dried over Na₂SO₄ and concentrated in vacuo. The residue was purified by silica gel DCM/MeOH; 97:3) to give 5′-O-(t-butyldimethylsilyl)-3′-O-(4-methoxytrityl)-2′-O-methyl uridine (D15) (9.5 g, 78%).

### Step 2. Preparation of 3′-O-(4-methoxytrityl)-2′O-methyluridine (D16)

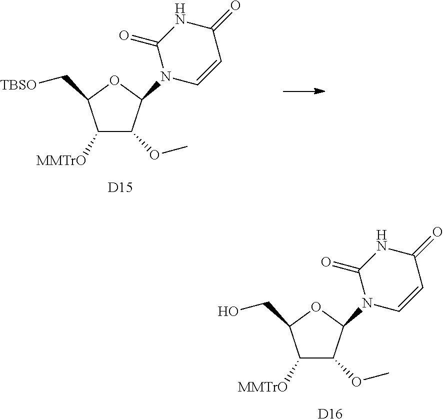

TEA-3HF (7.1 ml, 44.3 mmol) and TEA (10.6 ml, 73.8 mmol) was added dropwise to a solution of 5′-O-(t-butyldimethylsilyl)-3′-O-(4-methoxytrityl)-2′-O-methyluridine (D15) (9.5 g, 14.8 mmol) in anhydrous THF (90 mL). The reaction mixture was stirred at RT overnight. TLC showed the reaction was incomplete. Additional TEA-3HF (0.54 ml, 3.3 mmol) and TEA (0.6 ml, 4.15 mmol), were added. TLC showed the reaction went to completion. The solvent was removed in vacuo at RT. EA was added. The mixture were washed with water and brine, dried over anhydrous Na₂SO₄ and concentrated in vacuo. The residue was purified by silica gel DCM/MeOH; 95:5) to give 3′-O-(4-methoxytrityl)-2′O-methyluridine (D16) as white solid (7.01 g, 90%).

### Step 3. Preparation of 3′-O-(4-methoxytrityl)-5-C,5′-O-didehydro-2′-O-methyl uridine (D17)

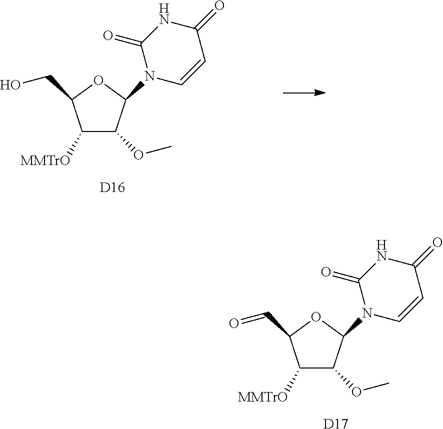

Pyridine (15.4 mL) and Dess-Martin (6.7 g, 15.8 mmol) were added to a solution of 3′-O-(4-methoxytrityl)-2′-O-methyl uridine (D16) (7.01 g, 13.2 mmol) in anhydrous CH₂Cl₂ (100 mL) at 0° C. under N₂. The reaction mixture was stirred at RT for 4 h. TLC showed the reaction went to completion. The reaction mixture was diluted with EA. The organic layer was washed with 10% Na₂S₂O₃ twice, followed by water and brine, dried over anhydrous Na₂SO₄ and concentrated in vacuo. The residue was purified by silica gel (DCM/EA=1/1) to give 3′-O-(4-methoxytrityl)-5-C,5′-O-didehydro-2′-O-methyl uridine (D17) (7.6 g).

### Step 4. Preparation of 2′-O-methyl-3′-O-(4-methoxytrityl)-5′-(S)—C-methyluridine (D18), and 2′-O-methyl-3′-O-(4-methoxytrityl)-5′-(R)—C-methyluridine (D19)

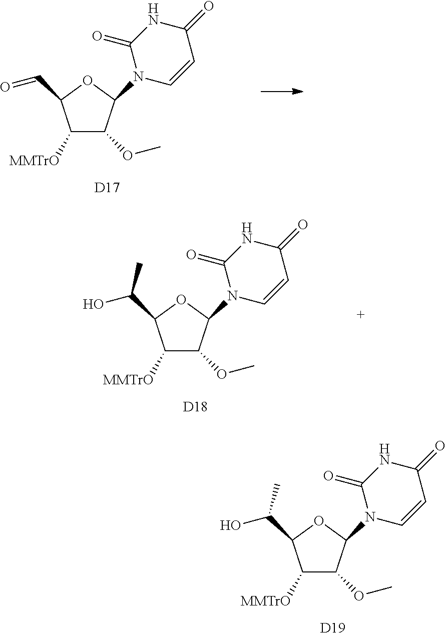

MeMgBr (31 mL, 43.2 mmol; 1.4M solution in hexanes) was added dropwise to a solution of 3′-O-(4-methoxytrityl)-5-C,5′-O-didehydro-2′-O-methyl uridine (D17) (7.6 g, 14.4 mmol) in anhydrous THF (120 mL) which was cooled by an ice-EtOH bath under N₂. The reaction mixture was stirred at RT for 4 h. TLC showed the reaction went to completion. The reaction mixture was quenched with saturated NH₄Cl. EA was added. The organic layer was washed with water and brine, dried over anhydrous Na₂SO₄ and concentrated in vacuo. The residue was purified by silica gel (DCM/EA=1:1) to give 2′-O-methyl-3′-O-(4-methoxytrityl)-5-(S)—C-methyluridine (D18) (3.04 g, 39%) and 2′O-methyl-3′-O-(4-methoxytrityl)-5′(R/S)—C-methyluridine (1.54 g, 20%) (D18+D19). Further purification on silica gel column (DCM/EA=1:1) gave 2′O-methyl-3′-O-(4-methoxytrityl)-5′(R)—C-methyluridine (D19) (140 mg, 2%) as white solid.

### Step 5. Preparation of 2′O-methoxy-5′-(R)—C-methyluridine-5′-[1-naphthyl(isopropoxy-L alaninyl)]phosphate (D12)

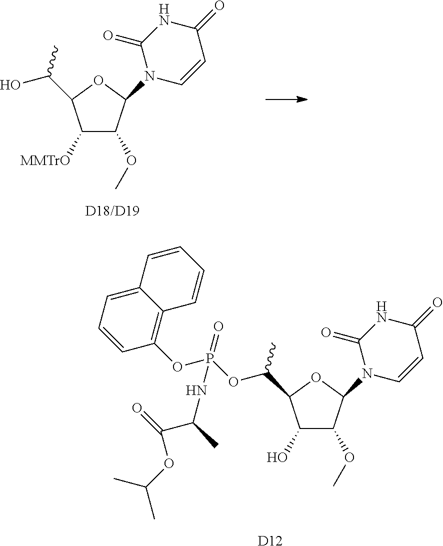

According to the procedure described for Example 41, 25.1 mg of 2′-β-methoxy-5′-(R)—C-methyluridine-5′-[1-naphthyl(isopropoxy-L-alaninyl)]phosphate was prepared from 100 mg (0.18 mmol) of 2′-O-methyl-3′-O-(4-methoxytrityl)-5(R/S)—C-methyluridine. ¹H NMR (CD₃OD, two isomers) δ 1.14-1.32 (m, 9H), 1.44 (2d, J=6.4, 1.55 Hz, 3H), 3.47, 3.49 (2s, 3H), 3.76-3.78 (m, 2H), 3.90-3.98 (m, 2H), 4.17, 4.26 (each t, J=5.2, 6.0 Hz, 1H), 4.8-5.01 (m, 1H), 5.39, 5.51 (2d, J=8.0 Hz, 1H), 5.85, 5.89 (2d, J=4.8, 3.6 Hz, 1H), 7.39-7.44 (m, 1H), 7.45 (d, J=8.4 Hz, 1H), 7.53-7.55 (m, 3H), 7.64 (d, J=8.0 Hz, 1H), 7.71 (t, 1H), 7.87-7.91 (m, 1H), 8.16-8.19 (m, 1H); ³¹P NMR (CD₃OD, two isomer) δ 3.39 (s), 3.74 (s). MS m/z 721.2 (MH⁺+diisopropylethylamine).

### Example 58

### Preparation of 5′(R)—C-methyluridine-5′-[phenyl(methoxy-L-alaninyl)]phosphate

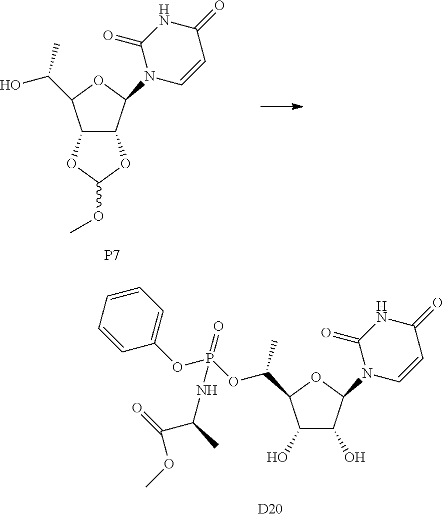

According to the procedure described for Example 54, 16.7 mg of 5′ (R)—C-methyluridine 5′-[phenyl(methoxy-L-alaninyl)]phosphate was prepared from 60 mg (0.2 mmol) of 2′,3′-O-methoxymethylidene-5′(R)-methyluridine (P7). ¹H NMR (CD₃OD, major isomers) δ ¹H NMR (CD₃OD, major isomer) δ 1.31, (d, J=7.2, Hz, 3H), 1.44 (d, J=6.4, Hz, 3H), 3.64 (s, 3H), 3.90-3.91 (m, 2H), 4.03 (t, J=4.0, Hz, 1H), 4.23 (t, J=4.0, Hz, 1H), 4.75 (m, 1H), 4.24 (dd, J=5.2, 4.0 Hz, 1H), 4.33 (t, J=5.2 Hz, 1H), 4.70-4.78 (m, 1H), 5.55 (d, J=8.4 Hz, 1H), 5.85 (d, J=6.0 Hz, 1H), 7.18-7.24 (m, 3H), 7.33-7.37 (m, 2H), 7.69 (d, J=8.0 Hz, 1H); ³¹P NMR (CD₃OD, major isomer) δ 2.88 (s, major), 2.96 (s, minor). MS m/z 498.0 (M−H⁺); ³¹P NMR (CD₃OD, major isomer) δ 2.38 (s), 2.65 (s). MS m/z 497.3 (M−H).

### Example 59

### Preparation of 2′-deoxy-2′-β-C-methyl-5′(S)—C-methyl-2′-α-fluorouridine-[phenyl(methoxy-L-alaninyl)]phosphate

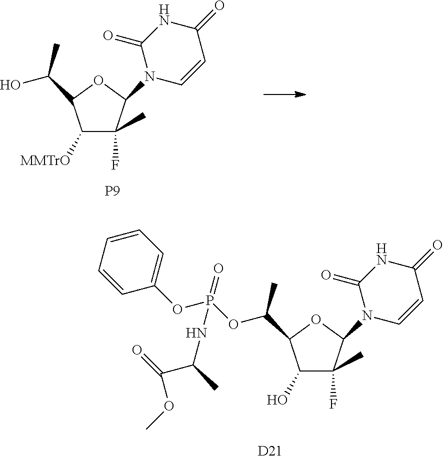

According to the procedure described for Example 41, 6.0 mg of 2′-deoxy-2′-β-C-methyl-5′(S)—C-methyl-2′-α-fluorouridine-5′-[phenyl(methoxy-L-alaninyl)]phosphate (D21) was prepared from 60 mg (0.11 mmol) of 2′-deoxy-3′-O,N⁴-di(4′-methoxytrityl)-2′-β-C-methyl-5′(S)—C-methyl-2′-α-fluorouridine (P9). ¹H NMR (CD₃OD, major isomer) δ 1.32-1.38 (m, 6H), 1.47 (d, J=6.8 Hz, 3H), 3.63 (s, 3H), 3.90-3.40 (m, 2H), 4.80 (m, 1H), 5.69 (d, J=8.0 Hz, 1H), 6.01 (brs, 1H), 7.16-7.38 (m, 5H), 7.64 (d, J=8.0 Hz, 1H); ³¹P NMR (CD₃OD, major isomer) δ 2.93 (s). MS m/z 514.0 (M−H).

### Example 60

### Preparation of 1-(2,6-diaminopurin-9-yl)-5(S)—C-methyl-β-D-ribofuranose 5-[phenyl(isopropoxy-L-alaninyl)]phosphate (E1)

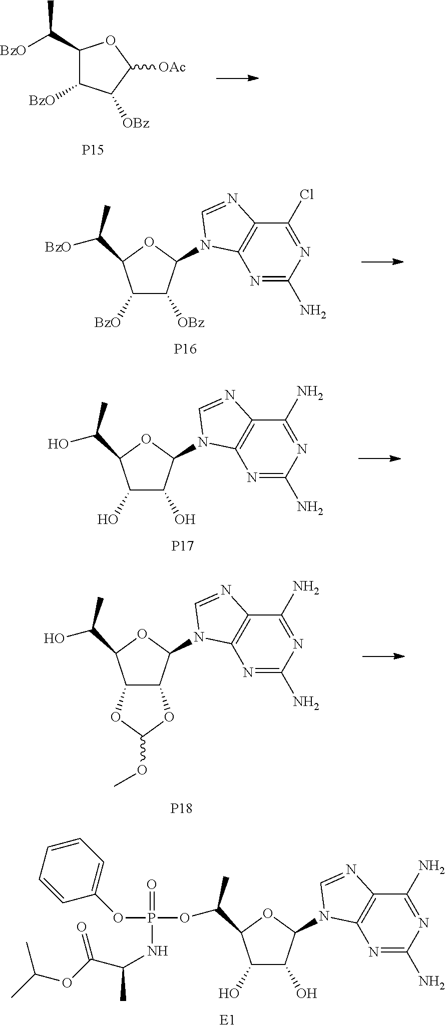

Step 1. Preparation of P16—To a stirred suspension of P15 (4.5 g, 7.99 mmol) and 6-chloroguanine (1.35 g, 7.99 mmol) in anhydrous MeCN (50 mL) was added DBU (3.84 g, 24 mmol) at 0° C. The mixture was stirred at 0° C. for 5 minutes and then TMSOTf (7.1 mL, 32 mmol) was added dropwise at 0° C. The mixture was stirred at 0° C. for 20 minutes and then was stirred at 70° C. for 3 hours. The reaction was cooled to RT and diluted with EA. The solution was washed with saturated NaHCO₃ and brine in sequence. The organic layer was dried over Na₂SO₄ and then concentrated. The residue was purified on a silica gel column (PE: EA=4:1 to 3:1) to give P16 (4.6 g, 86%) as light yellow foam.

Step 2. Preparation of P17—Compound P16 (7.74 g, 11.2 mmol) was dissolved in a minimum of 1,4-dioxane and then saturated aqueous ammonia was added (100 mL). The mixture was stirred at 100° C. in a sealed vessel for 10 hours. The mixture was cooled to RT and diluted with MeOH. The solvent was removed under reduced pressure and the residue was purified on a silica gel column (MeOH: DCM=1:20 to 1:8) to give P17 (2.47 g, 79%) as a white solid. ¹H NMR (DMSO-d6, 400 MHz) δ 8.13 (s, 1H), 6.78 (brs, 2H), 5.78 (d, J=7.2 Hz, 1H), 4.70-4.73 (m, 1H), 4.22-4.24 (m, 1H), 3.91-3.97 (m, 1H), 3.99 (t, J=2.0 Hz, 1H), 1.24 (d, J=6.4 Hz, 3H).

Step 3. Preparation of P18—To a suspension of P17 (600 mg, 2.0 mmol) in 10 mL of anhydrous THF was added trimethyl orthoformate (1.06 g, 10.0 mmol) and TsOH.H₂O (510 mg, 3.0 mmol). The mixture was stirred at RT for 16 hours. The reaction was quenched by NaHCO₃ and concentrated. The residue was purified by on a silica gel column (MeOH: DCM=1:20 to 1:10) to give P18 (410 mg, 60.6%) as white foam.

Step 4. Preparation of E1—Compound P18 (310 mg, 0.92 mmol) was dissolved in DMF-dimethylacetamide (10 mL) and the mixture was refluxed for 16 hours. The solvent was removed to give the crude fully blocked nucleoside (410 mg, 100%). To the solution of the crude nucleoside (410 mg, 0.92 mmol) in THF (3 mL) was added a solution of t-BuMgCl in THF (2.75 mL, 2.75 mmol) at 0° C. followed by a solution of phenyl(isopropoxy-L-alaninyl) phosphorochloridate (564 mg, 1.84 mmol in 2 mL THF). The mixture was stirred at RT for 16 hours and then quenched with water. The solvent was removed in vacuum. The residue was purified on a silica gel column (5% MeOH in DCM) to give the crude product (crude 280 mg) which was treated with 60% aqueous HCOOH solution at RT for 16 hours. The solvent was removed and the residue was purified by RP HPLC (MeCN and 0.1% HCOOH in water) to give compound E1 (single stereomer, 9.08 mg, 1.6%) as white solid. ¹H NMR (DMSO-d₆, 400 MHz) δ 7.86 (s, 1H), 7.42 (t, J=8.0 Hz, 2H), 7.20-7.35 (m, 3H), 6.88 (bs, 1H), 6.01-6.07 (m, 1H), 5.97 (bs, 1H), 5.85 (d, J=6.4 Hz, 1H), 5.54 (d, J=6.0 Hz, 1H), 5.31 (d, J=5.2 Hz, 1H), 4.94-4.97 (m, 1H), 4.73-4.80 (m, 1H), 4.37-4.44 (m, 1H), 4.25-4.30 (m, 1H), 3.96-3.98 (m, 1H), 3.83-3.91 (m, 1H), 1.46 (d, J=6.4 Hz, 3H), 1.24-1.29 (m, 9H); ³¹P NMR (DMSO-d₆, 162 MHz) δ 3.44; ESI-LCMS: m/z 566 [M+H]⁺.

### Example 61

### Preparation of 5′(S)—C-ethyladenosine 5′-[phenyl(isopropoxy-L-alaninyl)]phosphate (A24)

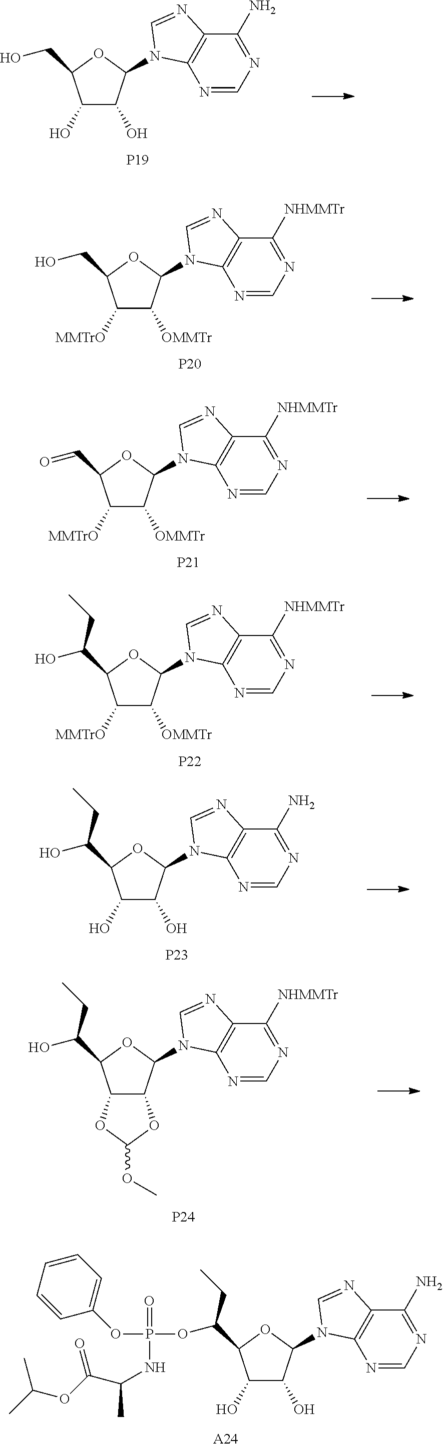

Step 1. Preparation of P20—To a suspension of P19 (50.0 g, 187 mmol) in anhydrous pyridine (500 mL) was added TBSCl (30.0 g, 200 mmol) at 0° C. The mixture was stirred at RT for 5 hours and then concentrated to dryness. The residue was dissolved in anhydrous DCM (500 mL). A mixture of sym-collidine (24.2 g, 200 mmol) and AgNO₃ (30.4 g, 200 mmol) was added followed by MMTrCl (283.0 g, 935 mmol). The mixture was stirred at RT for 24 hours, quenched by MeOH, filtered and the filtrate was concentrated. The residues was purified on a silica gel column (20% EA in PE) to give the crude product, which was dissolved in 1M TBAF in THF (200 mL) and stirred at RT for 2 hours. The solvent was removed and the residue was purified on a silica gel column (40% EA in PE) to give P20 (155.0 g, 77%) as a light yellow solid.

Step 2. Preparation of P21—To a suspension of P20 (2.0 g, 1.8 mmol) in anhydrous DCM (50 mL) was added DMP (1.27 g, 3.0 mmol) under N₂. The mixture was stirred at RT for 2 hours before quenched by saturated aqueous Na₂SO₃ and NaHCO₃. The mixture was extracted with DCM. The organic layer was dried and concentrated to give the crude product P2-3 (1.8 g, 90%) used for the next step without further purification.

Step 3. Preparation of P22—To an ice-EtOH cold solution of P21 (1.8 g, 1.65 mmol) in anhydrous THF (10 mL) was added with EtMgBr (1.0 M solution in THF, 10 mL, 10 mmol) dropwise under N₂. The reaction mixture was stirred at RT overnight. The mixture was cooled to 0° C. and quenched by saturated NH₄Cl. The solution was extracted with EA. The organic layer was dried over anhydrous Na₂SO₄ and concentrated. The residue was purified on a silica gel column (PE/EA=3/1 to 1/1) to give compound P22 (1.18 g, 44%) as a single stereomer.

Step 4. Preparation of P23—Compound P22 (200 mg, 0.18 mmol) was dissolved in 15 mL AcOH/H₂O (v/v=4:1). The mixture was stirred at 50° C. overnight. The solvent was removed under vacuum and the residue was purified on a silica gel column (DCM: MeOH=100:1 to 8:1) to give P23 (13 mg, 25%). ¹H NMR (DMSO-d₆,400 Hz) 8.31 (s, 1H), 8.08 (s, 1H), 7.34 (s, 2H), 5.82 (d, J=6.4 Hz, 1H), 5.44 (d, J=4.0 Hz, 1H), 5.37 (d, J=6.8 Hz, 1H), 5.08 (d, J=4.0 Hz, 1H), 4.51-4.53 (m, 1H), 4.07-4.10 (m, 1H), 3.87-3.88 (m, 1H), 3.41-3.47 (m, 1H), 1.37-1.44 (m, 2H), 0.85 (t, J=7.2 Hz, 3H).

Step 5. Preparation of P24—To a suspension of P23 (200 mg, 0.68 mmol) in 10 mL of anhydrous THF was added trimethyl orthoformate (1.06 g, 10.0 mmol) and TsOH.H₂O (171 mg, 1.0 mmol). The mixture was stirred at RT for 16 hours. The reaction was quenched by NaHCO₃ and then concentrated. The residue was purified on a column on silica gel (eluting with MeOH:DCM=1:20 to 1:10) to give the intermediate (180 mg) as white foam. The intermediate (180 mg, 0.53 mmol) was dissolved in anhydrous pyridine (10 mL) and cooled to 0° C. TMSCl (215 mg, 2.0 mmol) was added in dropwise. The mixture was stirred at RT for 3 hours before MMTrCl (400 mg, 1.3 mmol) was added. The mixture was stirred at 50° C. for 16 hours. The reaction was quenched by NH₄OH, the mixture was concentrated and purified by column on silica gel (1% MeOH in DCM) to give P24 (220 mg, 53%) as white foam.

Step 6. Preparation of A24—To a stirred solution of P24 (220 mg, 0.36 mmol) in anhydrous THF (4 mL) was added a solution of t-BuMgCl (0.72 mL, 1M in THF) dropwise at 40° C. The mixture was then stirred at 40° C. for 40 minutes. A solution of phenyl (isopropoxy-L-alaninyl) phosphorochloridate (219 mg, 0.72 mmol) in THF (1 mL) was added dropwise. After addition, the mixture was stirred at 40° C. for 16 hours. Then the reaction was quenched with H₂O and extracted with EA. The organic layer was dried over Na₂SO₄ and concentrated. The residue was purified on a column on silica gel (PE: EA=2:1 to 1:1) to give protected form of the prodrug (52 mg) as white solid. The product was dissolved in 60% HCOOH aqueous solution and the mixture was stirred at 25° C. for 16 hours. The solvent was removed and the residue was purified on a silica gel column (CH₃OH:DCM=1:100 to 1:20) to give the crude product which was further purified by RP HPLC (MeCN and 0.1% HCOOH in water) to give compound A24 (9.24 mg, 9.5%) as a white solid. ¹H NMR (DMSO-d₆, 400 MHz) δ 8.35, 8.29 (2s, 1H), 8.22, 8.20 (2s, 1H), 7.15-7.36 (m, 5H), 6.04 (s, 1H), 4.51-4.77 (m, 3H), 4.40 (s, 1H), 4.23, 4.65 (2d, J=4.0 Hz, 1H), 3.82-3.87 (m, 1H), 1.91-1.95 (m, 1H), 1.82-1.85 (m, 1H), 1.21 (s, 6H), 1.05-1.09 (m, 3H), 0.99-1.03 (m, 3.2H); ³¹P NMR (DMSO-d₆, 162 MHz) δ 1.71, 1.43; ESI-LCMS: m/z 565 [M+H]⁺.

### Example 62

### Preparation of 5′(R)—C-ethyladenosine 5′-[phenyl(isopropoxy-L-alaninyl)]phosphate (A25)

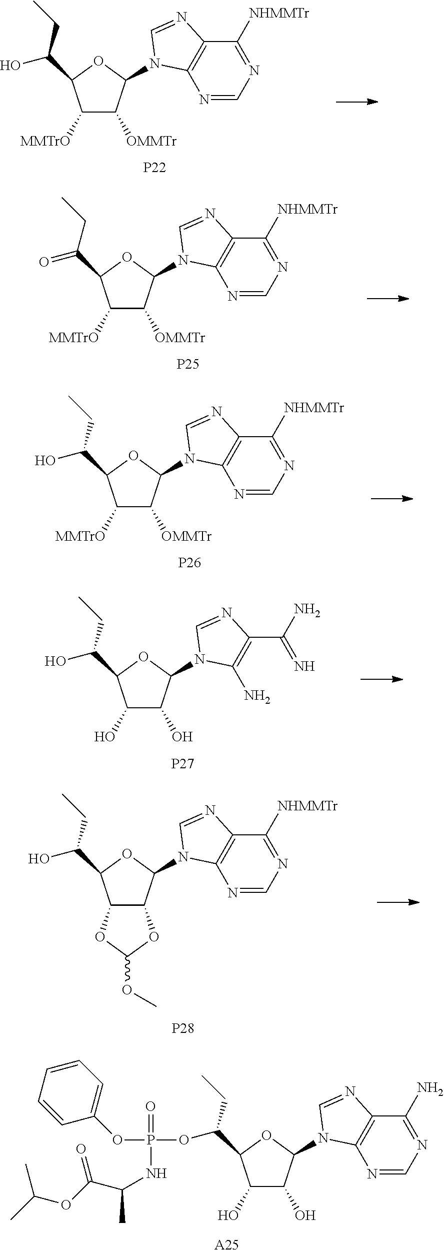

Step 1. Preparation of P25—To an ice-cold suspension of CrO₃ (135 mg, 1.35 mmol) in anhydrous DCM (5 mL) was added anhydrous pyridine (0.25 mL, 2.7 mmol) and Ac₂O (0.13 mL, 1.13 mmol) under N₂. The mixture was stirred at RT for about 10 min until the mixture became homogeneous. The mixture was cooled to 0° C. and a solution of P22 (500 mg, 0.45 mmol) in anhydrous DCM (5 mL) was added. The resultant mixture was stirred at RT for 1 h. The mixture was diluted with DCM (50 mL) and washed with aqueous NaHCO₃ and brine. The organic layer was dried over anhydrous Na₂SO₄ and filtered. The filtrate was concentrated in vacuum to give P25 (406 mg, 81%).

Step 2. Preparation of P26—To an ice-cold solution of P25 (400 mg, 0.36 mmol) in 95% EtOH (10 mL) was added NaBH₄ (126 mg, 3.6 mmol) under N₂. The reaction was stirred at RT overnight. The solvent was evaporated. The residue was diluted with EA (30 mL), washed with saturated NaHCO₃ aq. and brine. The organic layer was dried over Na₂SO₄ and concentrated. The residue was purified by prep-TLC to give P26 (398 mg, 98%) as a yellow solid.

Step 3. Preparation of P27—Compound P26 (220 mg, 0.2 mmol) was dissolved in 15 mL AcOH/H2O (v/v=4:1). The mixture was stirred at 50° C. overnight. The solvent was removed under vacuum and the residue was purified by silica gel column (DCM:MeOH=100:1 to 8:1) to give P27 (35 mg, 59%). ¹H NMR (DMSO-d6, 400 MHz) δ 8.31 (s, 1H), 8.11 (s, 1H), 7.38 (s, 2H), 5.82 (d, J=8.0 Hz, 1H), 5.72 (d, J=4.0 Hz, 1H), 5.36 (d, J=6.8 Hz, 1H), 5.14 (d, J=4.0 Hz, 1H), 4.64-4.67 (m, 1H), 4.12-4.13 (m, 1H), 3.82-3.83 (m, 1H), 3.56-3.59 (m, 1H), 1.31-1.36 (m, 2H), 0.91 (t, J=7.2 Hz, 3H).

Step 4. Preparation of P28—To a suspension of P27 (400 mg, 1.1 mmol) in 10 mL of anhydrous THF was added trimethyl orthoformate (636 mg, 6.0 mmol) and TsOH.H₂O (200 mg, 1.2 mmol). The mixture was stirred at RT for 16 hours. The reaction was quenched by NaHCO₃ and concentrated. The residue was purified on a silica gel column (MeOH:DCM=1:20 to 1:10) to give the intermediate (340 mg, 73%) as white foam. The product (340 mg, 0.91 mmol) was dissolved in anhydrous pyridine (10 mL) and cooled to 0° C. TMSCl (260 mg, 2.4 mmol) was added in dropwise and the mixture was stirred at RT for 3 hours before MMTrCl (480 mg, 1.6 mmol) was added. The mixture was stirred at 50° C. for 16 hours. The reaction was quenched by NH₄OH and concentrated. The residue was purified on silica gel column (1% MeOH in DCM) to give P28 (410 mg, 53%) as white foam.

Step 5. Preparation of A25—To a stirred solution of P28 (190 mg, 0.29 mmol) in anhydrous THF (5 mL) was added a solution of t-BuMgCl (0.9 mL, 1M in THF) dropwise at 40° C. The mixture was then stirred at 40° C. for 40 minutes. A solution of phenyl (isopropoxy-L-alaninyl) phosphorochloridate (270 mg, 0.885 mmol) in THF (1 mL) was added dropwise. After addition, the mixture was stirred at 40° C. for 16 hours. Then the reaction was quenched with H₂O and extracted with EA. The organic layer was dried over Na₂SO₄ and concentrated. The residue was purified on a silica gel column (PE:EA=2:1 to 1:1) to give crude protected prodrug (170 mg) which was treated with 60% HCOOH aqueous solution for 16 hours. The solvent was removed and the residue was purified by column on silica gel (MeOH:DCM=1:100 to 1:20) to give the crude product which was purified by RP HPLC separation (MeCN and 0.1% HCOOH in water) to give compound A25 (18.73 mg, 12.5%) as a white solid. ¹H NMR (DMSO-d6, 400 MHz) δ 8.30, 8.27 (2s, 1H), 8.12-8.18 (m, 1H), 7.28 (t, J=8.4 Hz, 2H), 7.10-7.15 (m, 3H), 6.04, 5.95 (2d, J=4.8 Hz, 1H), 4.44-4.95 (m, 1H), 4.71-4.74 (m, 2H), 4.45-4.49 (m, 1H), 4.11-4.15 (m, 1H), 3.84-3.86 (m, 1H), 1.84-1.86 (m, 2H), 1.26 (d, J=7.2 Hz, 3H), 1.17-1.23 (m, 7H), 0.96-1.09 (m, 3H); ³¹P NMR (DMSO-d6, 162 MHz) δ 3.19, 2.82; ESI-LCMS: m/z 565 [M+H]⁺.

### Example 63

### Preparation of 5′(S)—C-trifluoromethyladenosine 5′-[phenyl(isopropoxy-L-alaninyl)]phosphate (A26)

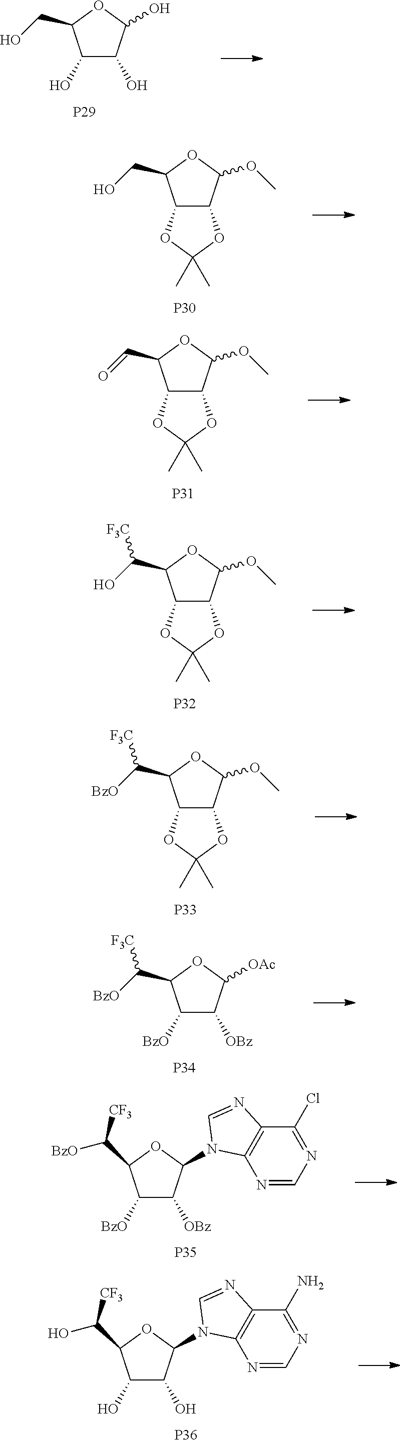

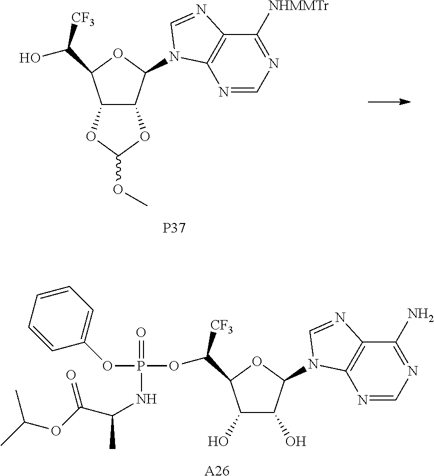

Step 1. Preparation of P30—To a solution of D-ribose (30.0 g, 1.33 mol) in acetone (285 mL) and MeOH (15 mL) was added concentrated H₂SO₄ (1.2 mL). The solution was refluxed for 24 hours. The reaction was cooled and neutralized with aqueous ammonia. The mixture was poured into H₂O (500 mL) and extracted with EA. The combined organic layers were dried with MgSO₄. The solvent was and the residue was purified on a silica gel column (PE:EA=4:1 to 2:1) to give P30 as colorless oil (25.5 g, 62.5%).

Step 2. Preparation of P31—To a solution of P30 (25.5 g, 125 mmol) in anhydrous DCM (800 mL) was added Dess-Martin preiodinane (78.2 g, 0.18 mol) at 0° C. under N₂. The resultant mixture was stirred at 15° C. overnight. The mixture was washed with saturated aqueous Na₂SO₃ and NaHCO₃ solution. The organic layer was separated, dried over anhydrous MgSO₄ and filtered. The filtrate was concentrated in vacuum to give compound P31 as a syrup which was used for the next step without further purification (16.5 g, 66%).

Step 3. Preparation of P32—To a solution of P31 (4.6 g, 22.8 mmol) and tetrabutylammonium acetate (TBAA) (345 mg, 1.15 mmol) in anhydrous THF (150 mL) was added a solution of TMSCF₃ (65.2 g, 459 mmol) at −50° C. under N₂. After the addition, the reaction mixture was warmed to 0° C. and stirred for 4 hours. The mixture was quenched with water and extracted with DCM. The combined organic layer was dried over anhydrous MgSO₄ and filtered. The filtrate was concentrated in vacuum to give a residue (4.7 g). The residue was dissolved in 150 mL THF and then was added TBAF (3.99 g, 13.7 mmol). The reaction mixture was stirred for 2 hours and then quenched with water, extracted with EtOAc, dried over anhydrous MgSO₄, filtered and concentrated to give syrup which was used for the next step without further purification

Step 4. Preparation of P33—To an ice-cooled solution of crude P32 in anhydrous pyridine (70 mL) was added BzCl (5.8 g, 38 mmol) dropwise under N₂. The reaction mixture was stirred at RT overnight. EA (300 mL) was added to the mixture and then washed with water (200 mL) and saturated aqueous NaHCO₃ (200 mL). The organic layer was separated, dried over anhydrous Na₂SO₄ and filtered. The filtrate was concentrated in vacuum to give a residue which was purified by on a silica gel column (PE/EA=20/1) to give P33 as syrup (3.6 g, 9.6 mmol).

Step 5. Preparation of P34—To a solution of Compound P33 (3.6 g, 9.6 mmol) in MeOH (200 mL) was added with concentrated aqueous HCl (2 mL). The resultant mixture was refluxed for 16 hours. The solvent was removed under vacuum. The residue was dissolved in DCM (200 mL) and washed with saturated aqueous NaHCO₃. The organic layer was separated, dried over anhydrous Na₂SO₄ and filtered. The filtrate was concentrated in vacuum to give syrup which was purified on a silica gel column (PE/EA=3/1) to give crude compound as syrup (2.73 g). The crude (2.73 g, 8.12 mmol) was dissolved in anhydrous pyridine (80 mL) and BzCl (6.8 g, 44.9 mmol) was added dropwise. The reaction mixture was stirred at RT overnight. EA (200 mL) was added to the mixture and then washed with water (100 mL) and saturated aqueous NaHCO₃ (100 mL). The organic layer was separated, dried over anhydrous Na₂SO₄ and filtered. The filtrate was concentrated in vacuum to give the residue (4.3 g). The residue was dissolved in HOAc (30 mL) and Ac₂O (3.3 mL) and the solution was cooled to 10° C. Concentrated H₂SO₄ was added dropwise The resultant mixture was stirred at RT for 5 h and then poured onto ice-water. The precipitate was collected by filtration. The collected solid was dissolved in EA (60 mL) and washed with saturated aqueous NaHCO₃ (50 mL). The organic layer was separated, dried over anhydrous Na₂SO₄ and filtered. The filtrate was concentrated in vacuum and the residue was purified on a silica gel column (PE/EA=20/1 to 20/1) to give P34 as foam (3.8 g, 68%).

Step 6. Preparation of P35—To an ice-cooled solution of P34 (1.14 g, 2.0 mmol) and 6-chloro-9H-purine (508 mg, 3.0 mmol) in anhydrous MeCN (20 mL) was added DBU (912 mg, 6 mmol). The mixture was stirred at for 30 minutes before TMSOTf (1.44 mL, 8.0 mmol) was added dropwise under N₂. The mixture was stirred at 70° C. overnight and then cooled to RT. The solution was diluted with EA and washed with aqueous NaHCO₃ and brine. The organic layer was dried over anhydrous Na₂SO₄ and filtered. The filtrate was concentrated in vacuum to give a residue which was purified on a silica gel column (PE/EA=4/1 to 3/1) to give the mixture of two isomers (1.1 g, 80.6%). Further purification by prep-TLC gave pure P35 (660 mg, 60%).

Step 7. Preparation of P36—Compound P35 (1.1 g, 1.6 mmol.) in 1,4-dioxane (10 mL) and NH₃.MeOH (30 mL) was added to a sealed heavy-wall pressure tube and the mixture was stirred at 100° C. overnight. Then concentrated and purified by silica gel column to give compound P36 (503 mg, 94%). ¹H NMR (CD₃OD, 400 MHz) δ 8.31 (s, 1H), 8.17 (s, 1H), 5.99 (d, J=6.4 Hz, 1H), 4.69 (t, J=4.8 Hz, 1H), 4.31-4.36 (m, 2H), 4.22-4.24 (m, 1H).

Step 8. Preparation of P37—To a suspension of P36 (670 mg, 2.0 mmol) in 20 mL of anhydrous THF was added trimethyl orthoformate (1.27 g, 12.0 mmol) and TsOH monohydrate (400 mg, 2.4 mmol). The mixture was stirred at RT for 16 hours. The reaction was quenched by NaHCO₃ and concentrated. The residue was purified on a silica gel column (MeOH:DCM=1:20 to 1:10) to give the crude (540 mg) as white foam. The crude was dissolved in anhydrous pyridine (10 mL) and cooled to 0° C. TMSCl (520 mg, 4.8 mmol) was added in dropwise. The mixture was stirred at RT for 3 hours, before MMTrCl (960 mg, 3.2 mmol) was added. The mixture was stirred at 50° C. for 16 hours. The reaction was quenched by NH₄OH, the mixture was concentrated and purified on a silica gel column (1% MeOH in DCM) to give P37 (610 mg, 48%) as white foam.

Step 9. Preparation of A26—To a mixture of compound P37 (323 mg, 0.5 mmol), N,N-Diisopropylethylamine (2 mL) and CH₃CN (20 mL) was added a solution of phenyl(isopropoxy-L-alaninyl) phosphorochloridate (610 mg, 2.0 mmol in THF). After addition, the mixture was refluxed for 16 hours. Then the solvent was removed in vacuum. The residue was purified on a silica gel column (PE:EA=2:1 to 1:1) to give the protected prodrug (220 mg, 48%) which was treated with 60% HCOOH for 16 hours at RT. The solvent was removed and the residue was purified on a silica gel column (MeOH:DCM=1:100 to 1:20) to give the crude product which was purified by RP HPLC (MeCN and 0.1% HCOOH in water) to give compound A26 (17.44 mg, 6.2%) as a white solid. ¹H NMR (DMSO-d₆, 400 MHz) δ 8.23 (s, 1H), 8.19 (s, 1H), 7.28 (t, J=8.0 Hz, 2H), 7.08-7.15 (m, 3H), 6.06 (d, J=3.2 Hz, 1H), 5.29 (d, J=7.2 Hz, 1H), 4.95 (brs, 1H), 4.49-4.53 (m, 2H), 4.41 (d, J=4.4 Hz, 1H), 3.81-3.88 (m, 1H), 1.26 (d, J=7.2 Hz, 3H), 1.15-1.19 (m, 6H); ³¹P NMR (DMSO-d₆, 162 MHz) δ 2.32; ESI-LCMS: m/z 605 [M+H]⁺.

### Example 64

### Preparation of 5′(R)—C-trifluoromethyladenosine 5′-[phenyl(isopropoxy-L-alaninyl)]phosphate (A27)

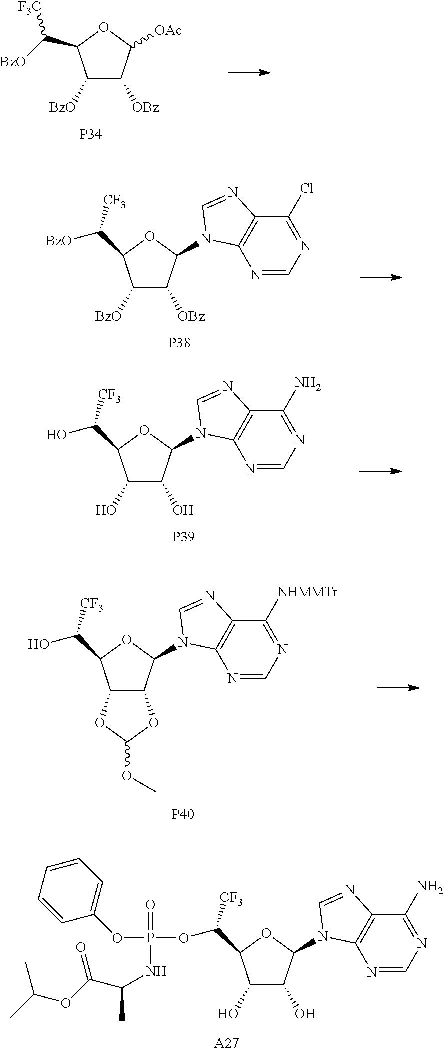

Step 1. Preparation of P38—To an ice-cooled solution of P34 (1.14 g, 2.0 mmol) and 6-chloro-9H-purine (508 mg, 3.0 mmol) in anhydrous MeCN (20 mL) was added DBU (912 mg, 6 mmol). The mixture was stirred at for 30 minutes before TMSOTf (1.44 mL, 8.0 mmol) was added dropwise under N₂. The mixture was stirred at 70° C. overnight and then cooled to RT. The solution was diluted with EA and washed with aqueous NaHCO₃ and brine. The organic layer was dried over anhydrous Na₂SO₄ and filtered. The filtrate was concentrated in vacuum to give a residue which was purified on a silica gel column (PE/EA=4/1 to 3/1) to give the mixture of two isomers (1.1 g, 80.6%). Further purification by prep-TLC gave pure P38 (230 mg, 21%).

Step 2. Preparation of P39—A suspension of P38 (230 mg, 0.35 mmol.) in 1,4-dioxane (2 mL) and conc. aqueous ammonia (10 mL, 28%) was stirred at 100° C. in a sealed vessel overnight. The solvent was removed and the residue was purified by on a silica gel column to give compound P39 (41.5 mg, 35.4%). ¹H NMR (CD₃OD, 400 MHz) δ 8.24 (s, 1H), 8.18 (s, 1H), 5.95 (d, J=8.0 Hz, 1H), 4.79-4.82 (m, 1H), 4.43-4.47 (d, J=5.6 Hz, 1H), 4.27-4.30 (m, 2H).

Step 3. Preparation of P40—To a suspension of P39 (90 mg, 0.27 mmol) in 10 mL of anhydrous THF was added trimethyl orthoformate (320 mg, 3.0 mmol) and TsOH.H₂O (51 mg, 0.3 mmol). The mixture was stirred at RT for 16 hours. The reaction was quenched by NaHCO₃ (to pH>7), then concentrated and purified by column on silica gel (eluting with MeOH:DCM=1:20 to 1:10) to give the crude (99 mg, 97%) as white foam. The crude product was dissolved in anhydrous pyridine (5 mL) and cooled to 0° C. TMSCl (52 mg, 0.48 mmol) was added. The mixture was stirred at RT for 3 hours before MMTrCl (200 mg, 0.67 mmol) was added. The mixture was stirred at 50° C. for 16 hours. The reaction was quenched by NH₄OH and the mixture was concentrated and purified by column on silica gel (1% MeOH in DCM) to give P40 (110 mg, 65%) as white foam.

Step 4. Preparation of A27—To a solution of P40 (110 mg, 0.17 mmol) in N,N-Diisopropylethylamine (2 mL) and CH₃CN (10 mL) was added a solution of phenyl (isopropoxy-L-alaninyl) phosphorochloridate (122 mg, 0.4 mmol in THF). The mixture was refluxed for 16 hours. Then the solvent was removed under vacuum. The residue was purified by column on silica gel (PE: EA=2:1 to 1:1) to give the protected compound (100 mg, 64%) as white foam. The protected precursor (100 mg, 0.11 mmol) was dissolved in 60% HCOOH aqueous solution and the mixture was stirred at RT for 16 hours. The solvent was removed and the residue was purified by column chromatography on silica gel (CH₃OH:CH₂Cl₂=1:100 to 1:20) to give the crude product, which was purified by RP HPLC (MeCN and 0.1% HCOOH in water) to give compound A27 (17.5 mg, 26%) as a white solid. ¹H NMR (DMSO-d₆, 400 MHz) δ 8.35 (s, 1H), 8.23 (s, 1H), 7.32 (t, J=8.0 Hz, 2H), 7.15-7.18 (m, 3H), 5.97 (d, J=7.2 Hz, 1H), 5.34-5.38 (m, 1H), 4.89-4.93 (m, 1H), 4.55-4.60 (m, 1H), 4.31-4.35 (m, 1H), 3.82-3.88 (m, 1H), 1.28 (d, J=6.8 Hz, 3H), 1.19 (br, 6H); ³¹P NMR (DMSO-d₆, 162 MHz) δ 3.53; ESI-LCMS: m/z 605 [M+H]⁺.

### Example 65

### Preparation of 5′(S)—C-methyladenosine 5′(S)-[phenyl(cyclohexoxy-L-alaninyl)]phosphate (A10a) and 5′(S)—C-methyladenosine 5′(R)-[phenyl(cyclohexoxy-L-alaninyl)]phosphate (A10b)

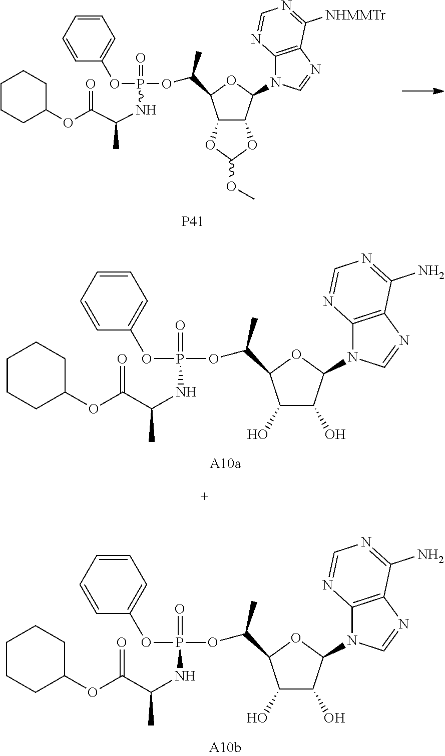

Compound P41 (1.2 g, 2.0 mmol) was dissolved in 80% HCOOH aqueous solution and the mixture was stirred at RT for 16 hours. The solvent was removed and the residue was purified by RP HPLC (column type: 150*21.5 mm, with organic phase gradient (28˜58% acetonitrile solution in neutral system)) to give A10a (22.7 mg, 1.9%) and A10b (189 mg, 16%).

¹H NMR for compound A10a (CD₃OD, 400 MHz) δ 8.34 (s, 1H), 8.24 (s, 1H), 7.20-7.38 (m, 5H), 6.06 (d, J=4.4 Hz, 1H), 4.79-4.84 (m, 1H), 4.62-4.66 (m, 1H), 4.56 (t, J=5.2 Hz, 1H), 4.49 (t, J=5.2 Hz, 1H), 4.04-4.07 (m, 1H), 3.85-3.93 (m, 1H), 1.26-1.76 (m, 16H); ³¹P NMR (CD₃OD, 162 MHz) δ3.13; ESI-LCMS: m/z 591 [M+H]⁺.

¹H NMR for compound A10b (CD₃OD, 400 MHz) δ 8.26 (s, 1H), 8.19 (s, 1H), 7.10-7.30 (m, 5H), 6.03 (d, J=5.2 Hz, 1H), 4.80-4.84 (m, 1H), 4.67-4.73 (m, 1H), 4.50 (t, J=5.2 Hz, 1H), 4.33 (t, J=4.8 Hz, 1H), 4.04-4.06 (m, 1H), 3.85-3.93 (s, 1H), 1.24-1.78 (m, 16H); ³¹P NMR (CD₃OD, 162 MHz) δ3.14; ESI-LCMS: m/z 591 [M+H]⁺.

### Example 66

### Preparation of 5′(S)—C-methyladenosine 5′-[2-chlorophenyl(cyclohexoxy-L-alaninyl)]phosphate (A28)

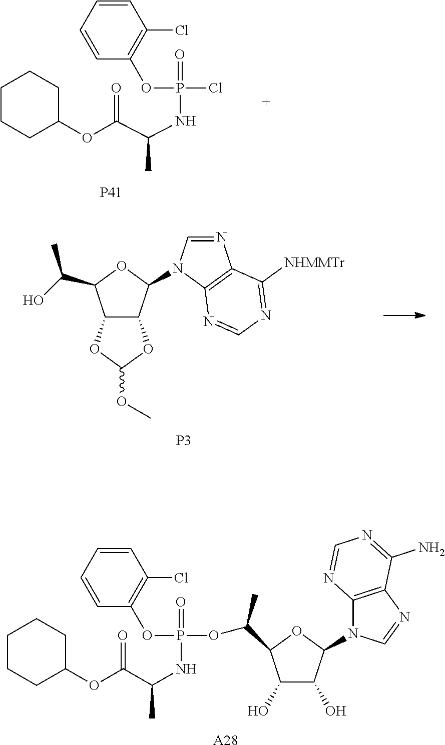

Step 1: Preparation of P41—To a stirred solution of phosphoryl trichloride (3.06 g, 20 mmol) and 2-chlorophenol (2.56 g, 20 mmol) in anhydrous DCM (100 mL) was added a solution of TEA (2.04 mL, 20 mmol) in DCM (20 mL) dropwise at −78° C. After addition, the mixture was warmed to RT gradually and stirred for 2 hours. Then the solution was re-cooled to −78° C. and (S)-cyclohexyl 2-aminopropanoate hydrochloride (3.73 g, 18 mmol) was added followed by TEA (3.67 g, 36 mmol) dropwise at −78° C. The mixture was warmed to RT gradually and stirred for 2 hours. Then the solvent was removed and the residue was dissolved in methyl-butyl ether. The precipitate was filtered off and the filtrate was concentrated. The residue was purified on a silica gel column (pure DCM) to give the phosphorylchlorodate as colorless oil (3.1 g, 40.9%).

Step 2: Preparation of A28—To a stirred solution of P3 (595.6 mg, 1 mmol) in anhydrous THF (10 mL) was added a solution of t-BuMgCl (3 mL, 1M in THF) dropwise at −78° C. The mixture was then stirred at RT for 30 minutes and re-cooled to −78° C. A solution of P41 (3 mL, 1M in THF) was added dropwise and then the mixture was stirred at RT overnight. The reaction was quenched with H₂O and extracted with EA. The organic layer was dried over Na₂SO₄ and concentrated. The residue was purified on a silica gel column (PE:EA=1:1 to 1:3) to give protected prodrug (670 mg), which was treated with 65% HCOOH aqueous solution at RT overnight. The solvent was removed under reduced pressure. The residue was purified by column chromatography on silica gel first and then by RP HPLC (0.1% HCOOH in water and MeCN) to give A28 as a white solid (191.8 mg, single stereomer, 30.7%). ¹H NMR (CD₃OD, 400 MHz) δ 8.28 (s, 1H), 8.19 (s, 1H), 7.39-7.46 (m, 2H), 7.07-7.20 (m, 2H), 6.03 (d, J=5.2 Hz, 1H), 4.90-4.95 (m, 1H), 4.67-4.73 (m, 1H), 4.51 (dd, J₁=J₂=5.6 Hz, 1H), 4.38 (dd, J₁=4.0 Hz, J₂=5.6 Hz, 1H), 4.05-4.08 (m, 1H), 3.91-3.99 (m, 1H), 1.66-1.82 (m, 4H), 1.54 (d, J=6.4 Hz, 3H), 1.28-1.51 (m, 9H); ³¹P NMR (CD₃OD, 162 MHz) δ: 2.91; ESI-LCMS: m/z 625 [M+H]⁺.

### Example 67

### Preparation of 5′-C—(S)-methyl adenosine 5′-phosphoramidates

By a similar procedure as described in Example 66, a number of 5′-C—(S)-methyladenosine 5′-phosphoramidates were prepared with the appropriate chlorophosphorylamino propanoate used in place of P41. The structures of the 5′-C—(S)-methyladenosine 5′-phosphoramidates, and corresponding characterization data, are listed in Table 6.

TABLE 6
Various 5′-C-(S)-methyladenosine 5′-phosphoramidates compounds

| Compound | | ³¹P NMR | ESI-LCMS |
|---|---|---|---|
| No. | Structure | ppm | m/z |
| A29 | 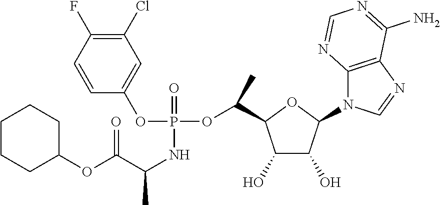 | 3.12 | 643.1 (M + 1)⁺ |
| A30 | 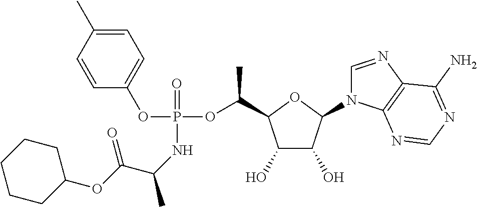 | 3.27 | 605.1 (M + 1)⁺ |
| A31 | 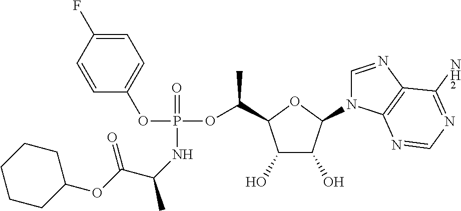 | 6.04 6.03 | 609.3 (M + 1)⁺ |
| A32 | 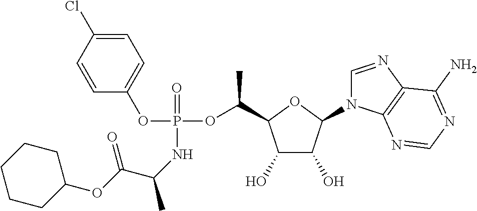 | 3.18 3.00 | 625.4 (M + 1)⁺ |
| A33 | 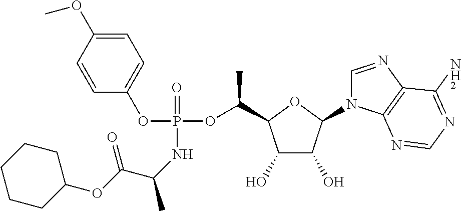 | 3.56 | 621.3 (M + 1)⁺ |
| A34 | 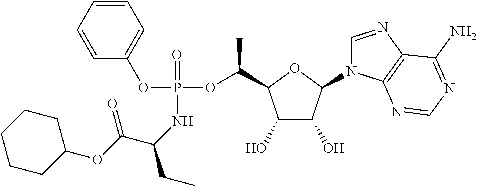 | 3.38 | 605.3 (M + 1)⁺ |
| A35 | 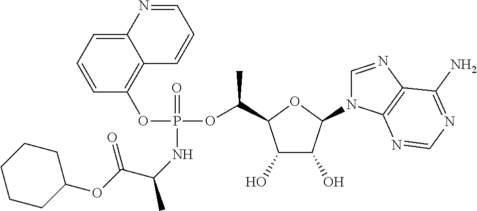 | 3.59 3.12 | 642.0 (M + 1)⁺ |
| A36 | 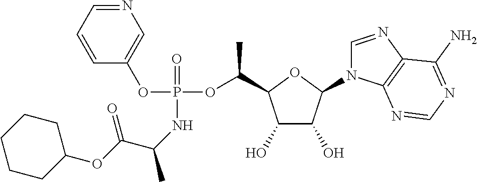 | 3.64 3.37 | 592.1 (M + 1)⁺ |
| A37 | 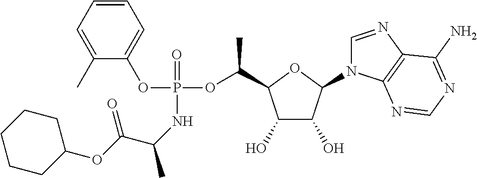 | 3.23 | 605.2 (M + 1)⁺ |
| A38 | 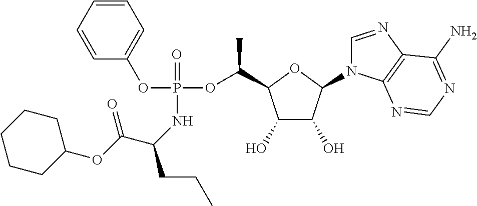 | 3.32 3.29 | 619.3 (M + 1)⁺ |
| A39 | 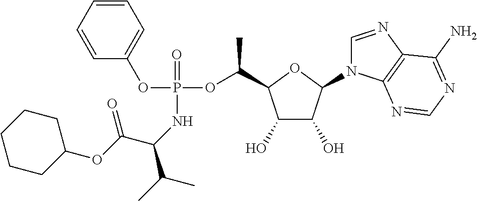 | 3.85 3.78 | 619.3 (M + 1)⁺ |
| A40 | 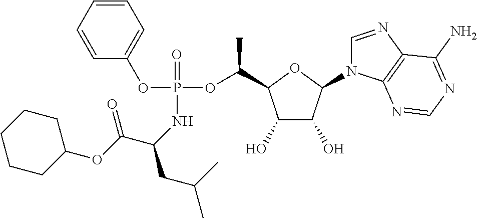 | 3.32 3.16 | 633.1 (M + 1)⁺ |
| A41 | 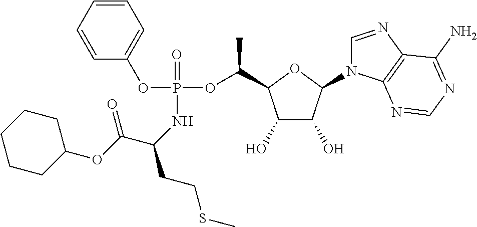 | 3.22 3.18 | 651.1 (M + 1)⁺ |
| A42 | 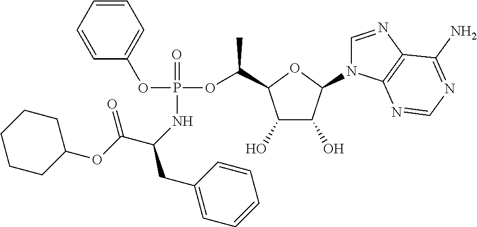 | 2.92 2.58 | 667.1 (M + 1)⁺ |
| A43 | 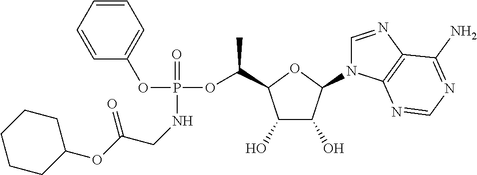 | 4.52 3.98 | 577.3 (M + 1)⁺ |

TABLE 7
Additional nucleosides/nucleotides/nucleotide
derivatives/compounds
Structure

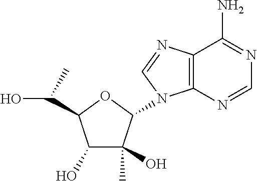

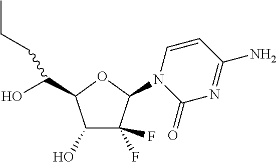

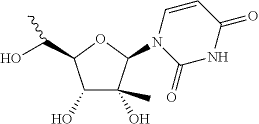

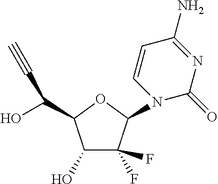

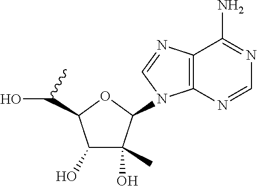

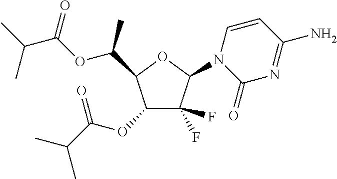

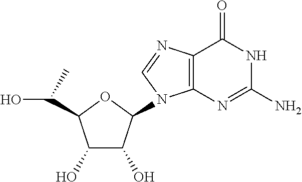

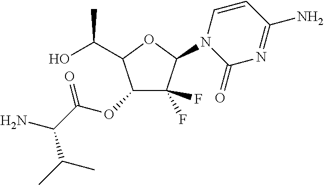

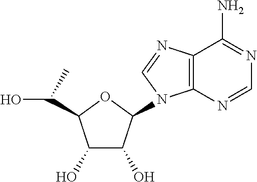

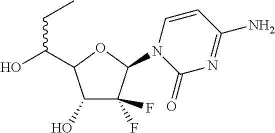

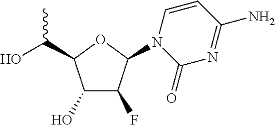

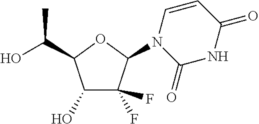

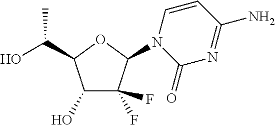

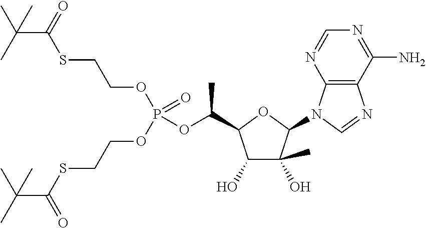

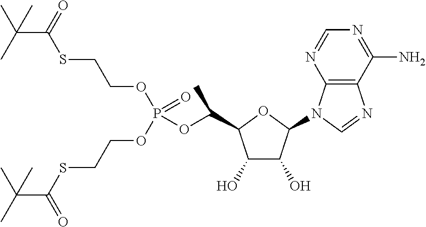

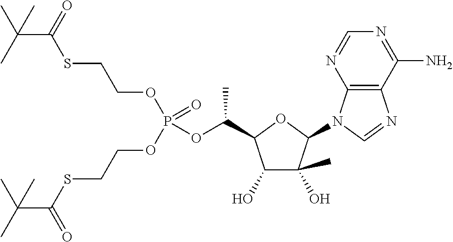

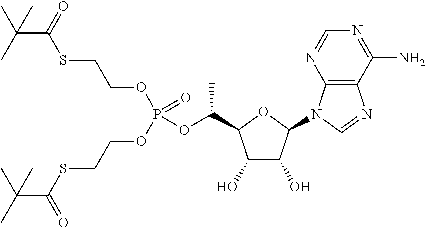

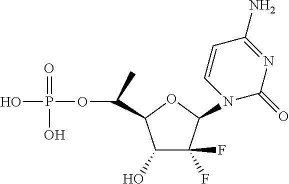

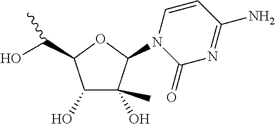

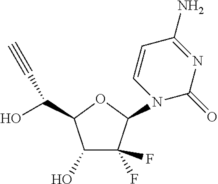

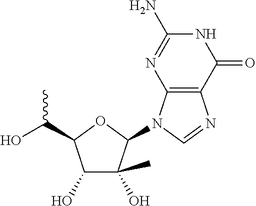

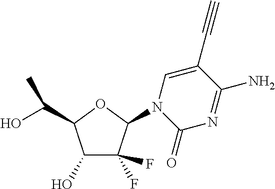

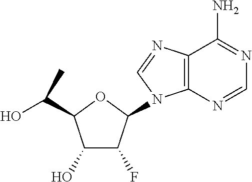

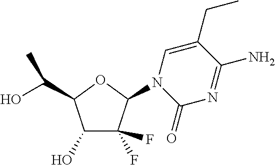

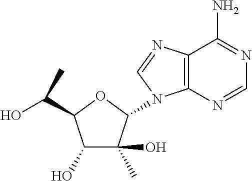

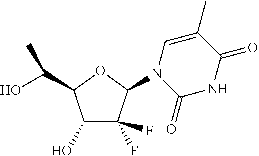

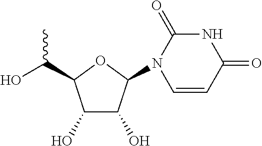

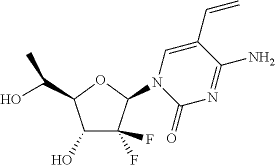

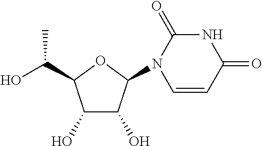

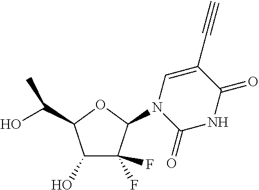

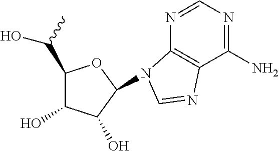

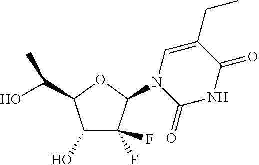

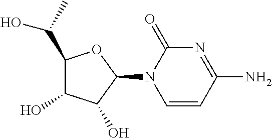

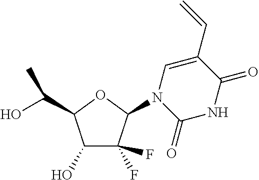

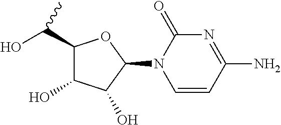

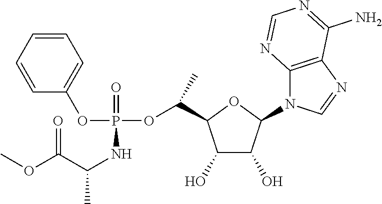

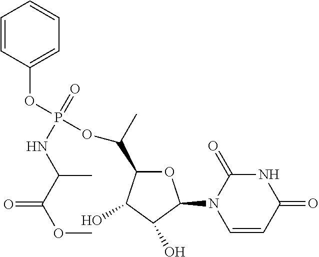

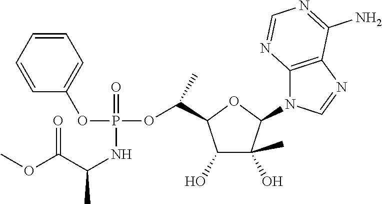

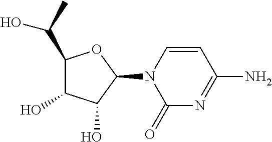

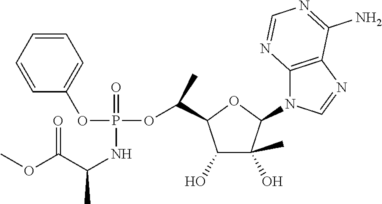

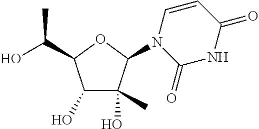

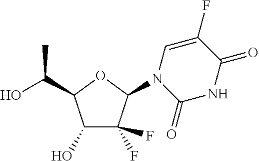

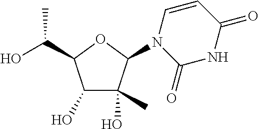

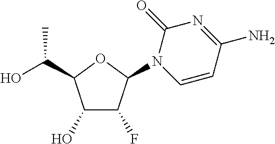

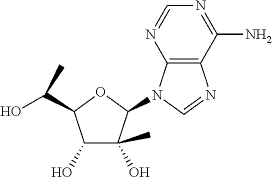

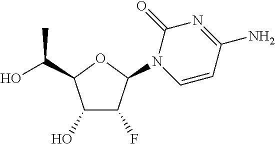

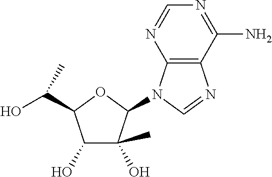

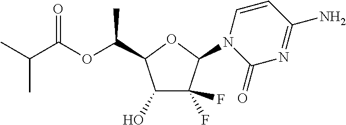

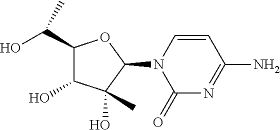

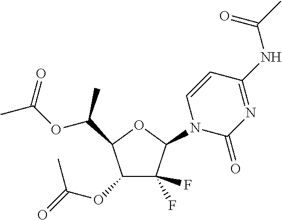

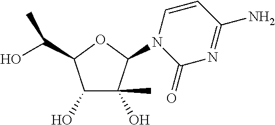

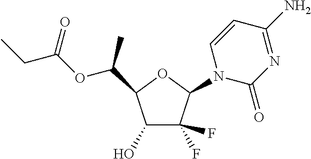

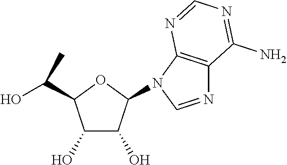

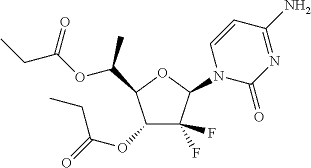

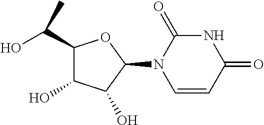

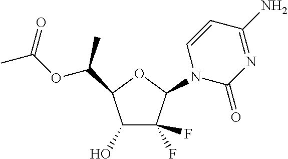

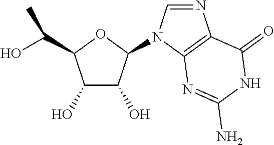

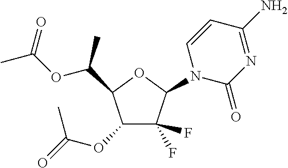

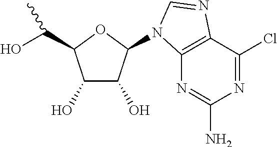

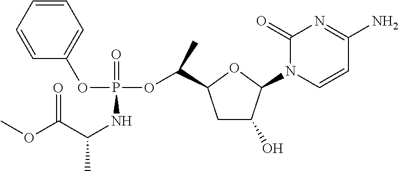

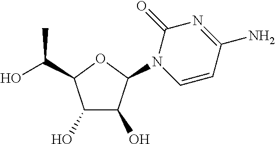

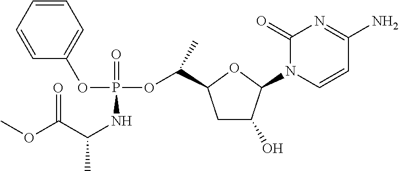

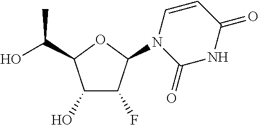

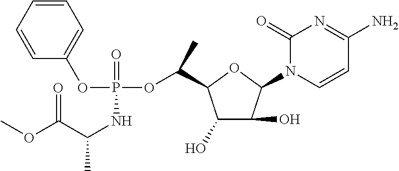

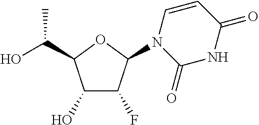

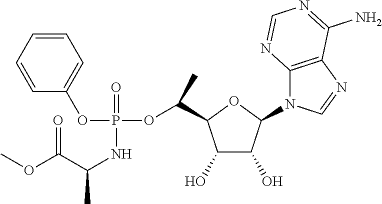

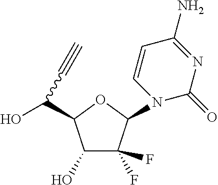

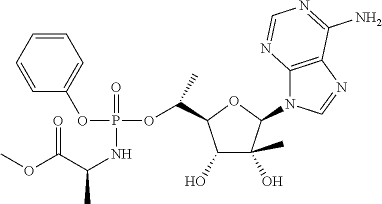

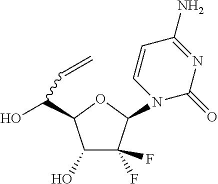

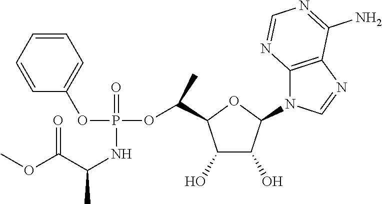

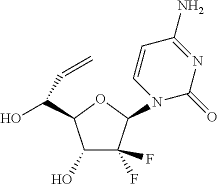

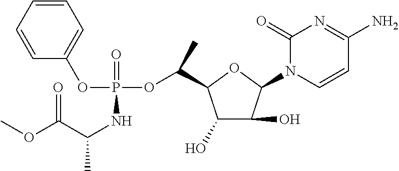

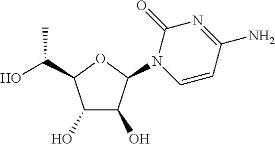

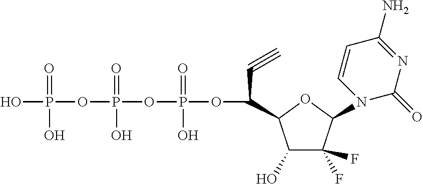

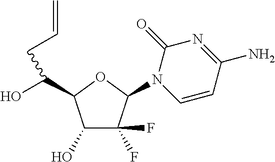

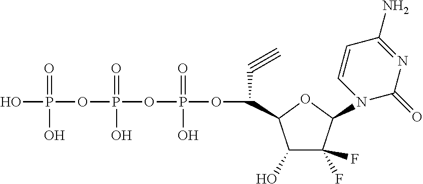

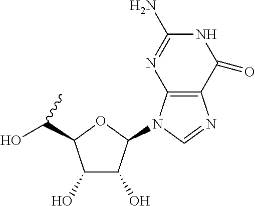

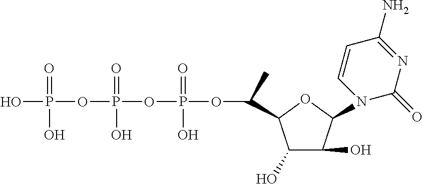

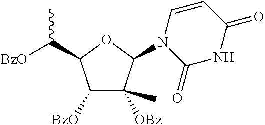

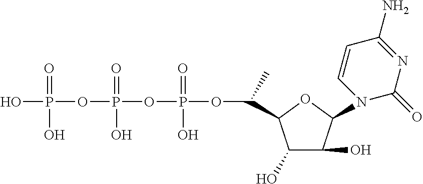

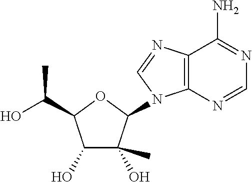

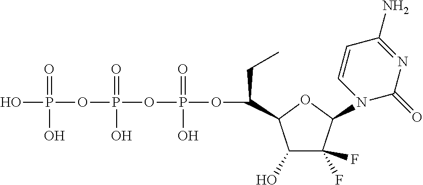

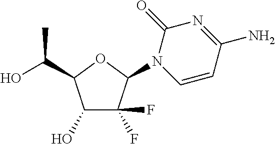

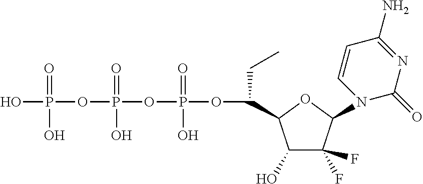

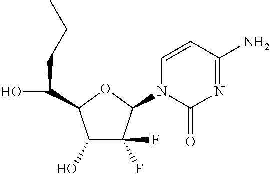

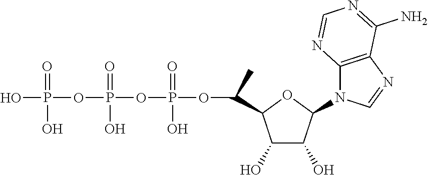

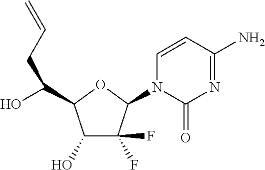

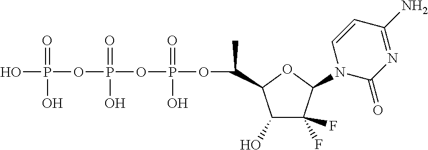

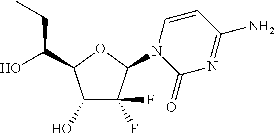

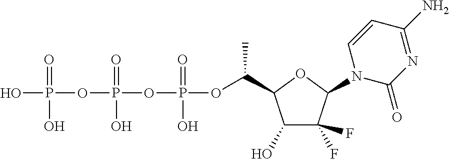

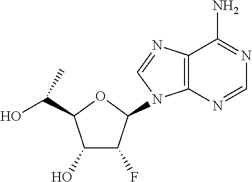

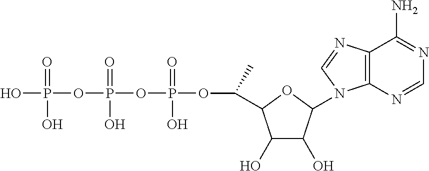

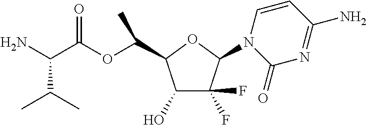

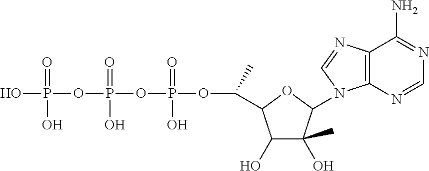

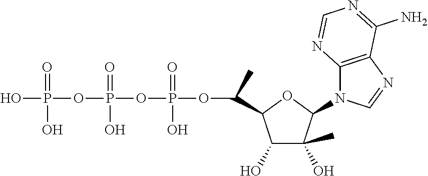

### Example 68

### 5′-alkylated nucleoside 5′-triphosphates

The following 5′-alkylated nucleoside 5′-triphosphates were prepared according to the procedure described in U.S. Publication No. 2010-0249068, which is hereby incorporated by reference:

| | |
|---|---|
| 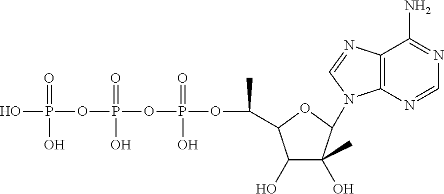 | MS: 534.1 (M − 1) ³¹P NMR (D₂O): −8.75 (d, 1P); −11.45 (d, 1P), −22.48 (t, 1P) |
| 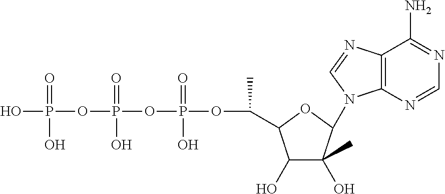 | MS: 534.4 (M − 1) ³¹P NMR (D₂O): −8.58 (bs, 1P); −11.09 (d, 1P), −22.15 (t, 1P) |
| 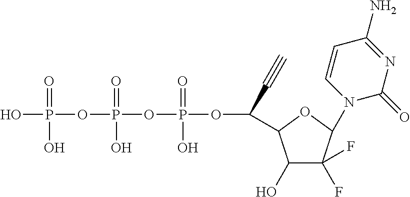 | MS: 526.2 (M − 1) ³¹P NMR (D₂O): −9.58 (bs, 1P); −11.65 (d, 1P), −21.92 (bs, 1P) |
| 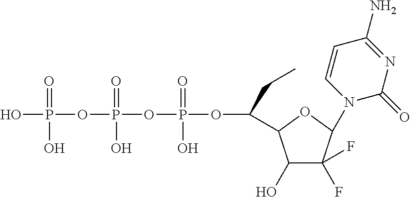 | MS: 529.9 (M − 1) ³¹P NMR (D₂O): −10.15 (d, 1P); −11.20 (d, 1P), −22.45 (t, 1P) |
| 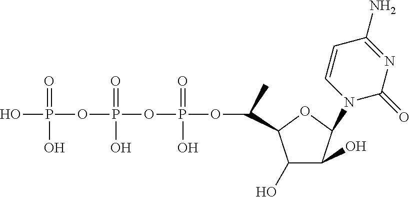 | MS: 496.0 (M − 1) ³¹P NMR (D₂O): −10.15 (d, 1P); −11.30 (d, 1P), −22.54 (t, 1P) |
| 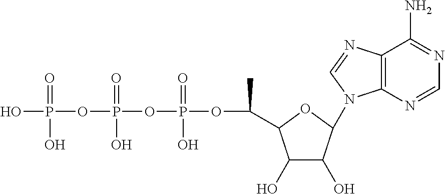 | MS: 520.1 (M − 1) ³¹P NMR (D₂O): −9.75 (d, 1P); −11.41 (d, 1P), −22.54 (t, 1P) |
| 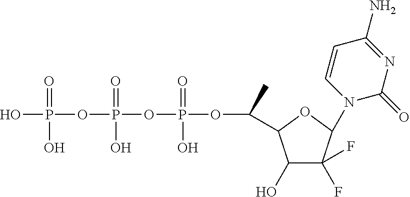 | MS: 516.0 (M − 1) ³¹P NMR (D₂O): −9.50 (bs, 1P); −11.30 (d, 1P), −22.33 (t, 1P) |

### Example 69

### HCV Replicon Assay

### Cells

Huh-7 cells containing the self-replicating, subgenomic HCV replicon with a stable luciferase (LUC) reporter were cultured in Dulbecco's modified Eagle's medium (DMEM) containing 2 mM L-glutamine and supplemented with 10% heat-inactivated fetal bovine serum (FBS), 1% penicillin-streptomyocin, 1% nonessential amino acids, and 0.5 mg/ml G418.

### Determination of Anti-HCV Activity

Determination of 50% inhibitory concentration (EC₅₀) of compounds in HCV replicon cells were performed by the following procedure. On the first day, 5,000 HCV replicon cells were plated per well in a 96-well plate. On the following day, test compounds were solubilized in 100% DMSO to 100× the desired final testing concentration. Each compound was then serially diluted (1:3) up to 9 different concentrations. Compounds in 100% DMSO are reduced to 10% DMSO by diluting 1:10 in cell culture media. The compounds were diluted to 10% DMSO with cell culture media, which were used to dose the HCV replicon cells in 96-well format. The final DMSO concentration was 1%. The HCV replicon cells were incubated at 37° C. for 72 hours. At 72 hours, cells were processed when the cells are still subconfluent. Compounds that reduce the LUC signal are determined by Bright-Glo Luciferase Assay (Promega, Madison, Wis.). % Inhibition was determined for each compound concentration in relation to the control cells (untreated HCV replicon) to calculate the EC₅₀.

The antiviral activity of exemplary compounds is shown in Table 8, wherein ‘A’ represents an EC₅₀ of <1 μM, ‘B’ represents an EC₅₀ of <30 μM, ‘C’ represents an EC₅₀ of <100 μM and ‘D’ represents an EC₅₀ of <1000 μM. In Table 9 ‘A’ represents an EC₅₀ of <5 “B” represents an EC₅₀ of <30 μM, and ‘C’ represents an EC₅₀ of <200 μM.

TABLE 8

| | | HCV |
|---|---|---|
| | | Replicon |
| Structure | Compound No. | Activity |
| 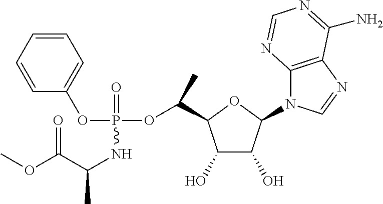 | A15 | B |
| 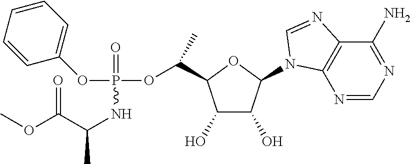 | | B |
| 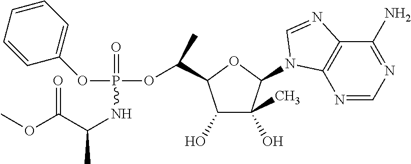 | | D |
| 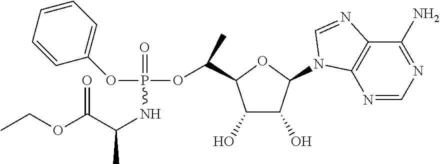 | | B |
| 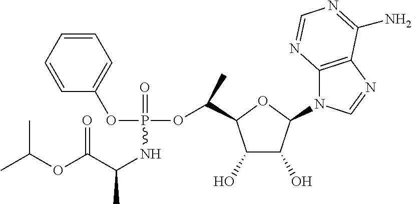 | A8 | A |
| 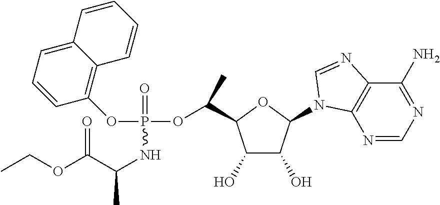 | A19 | A |
| 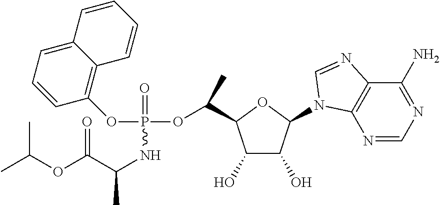 | A20 | A |
| 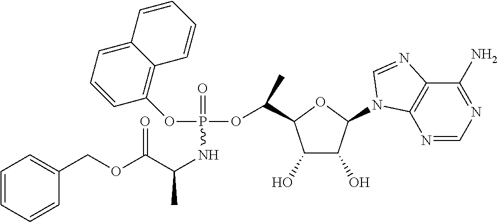 | A21 | A |
| 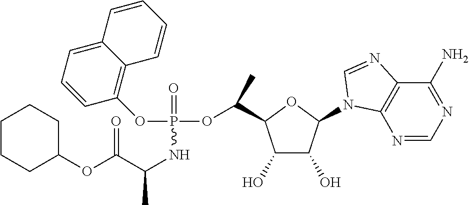 | A1 | A |
| 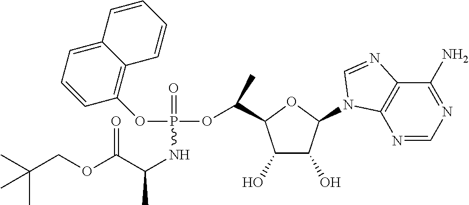 | A2 | A |
| 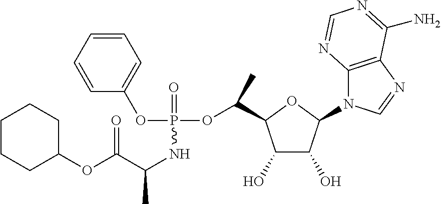 | A10 | A |
| 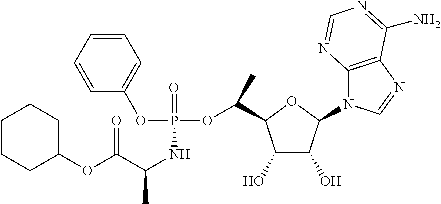 | A10a | A |
| 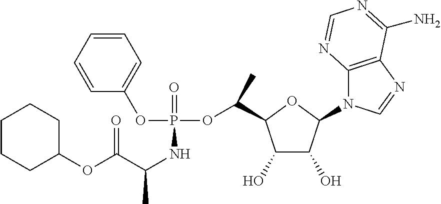 | A10b | A |
| 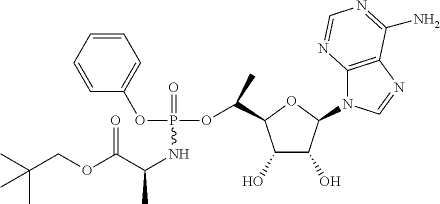 | A11 | A |
| 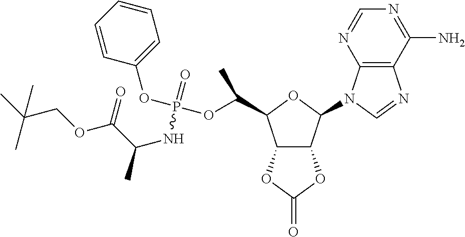 | A12 | A |
| 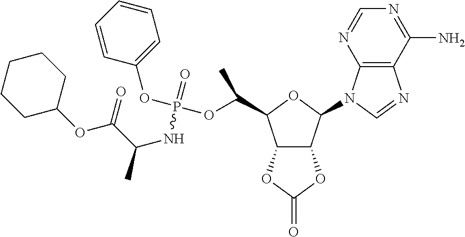 | A13 | A |
| 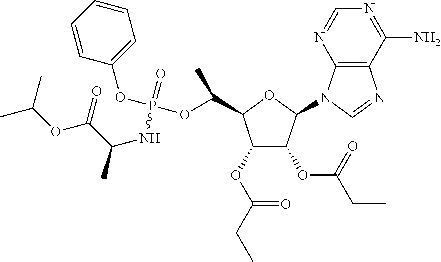 | A14 | A |
| 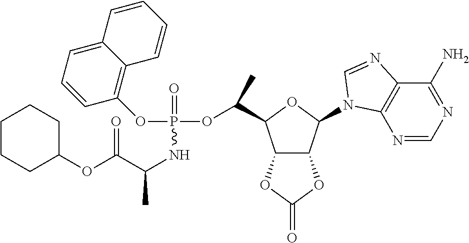 | A3 | A |
| 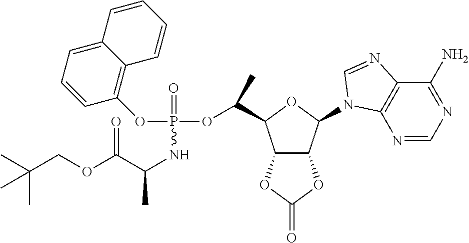 | A4 | A |
| 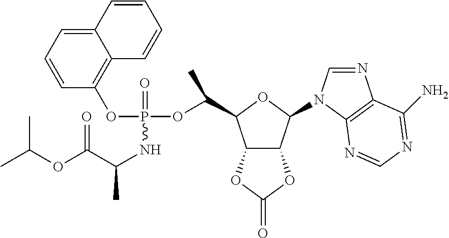 | A23 | A |
| 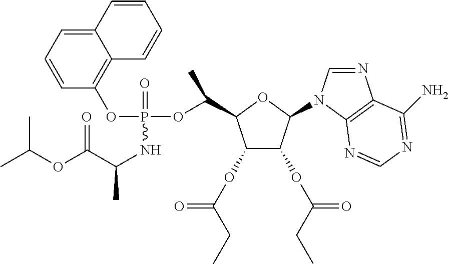 | A22 | A |
| 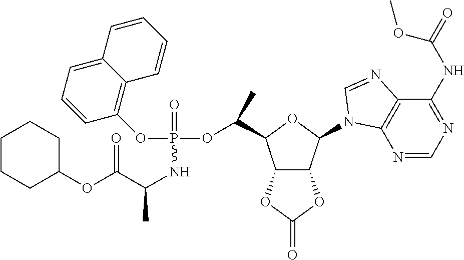 | A5 | B |
| 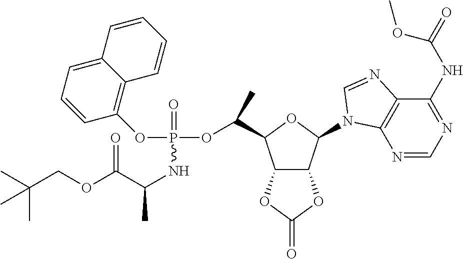 | A7 | B |
| 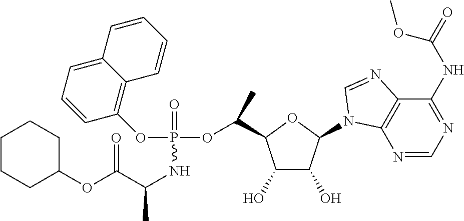 | A6 | B |
| 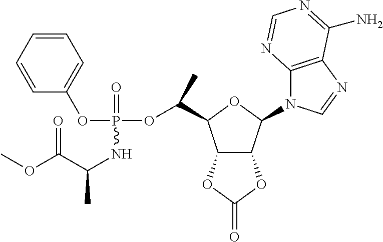 | A16 | B |
| 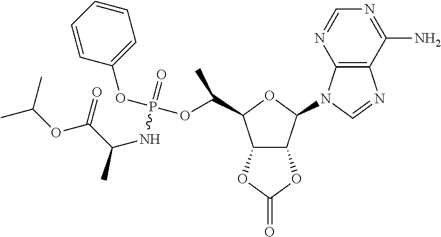 | A9 | A |
| 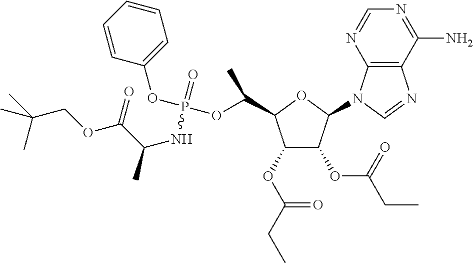 | A17 | A |
| 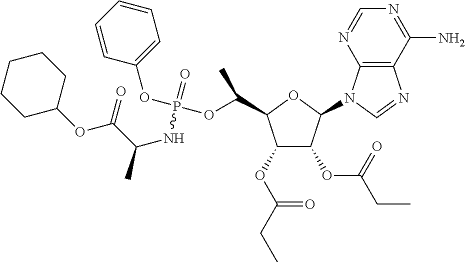 | A18 | A |
| 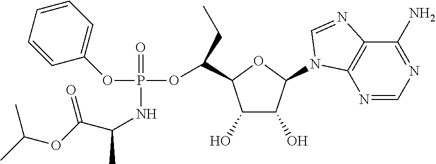 | A24 | C |
| 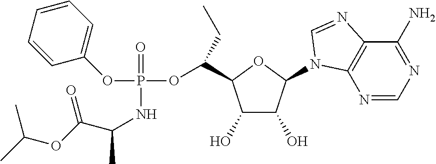 | A25 | D |
| 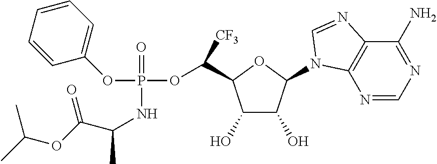 | A26 | C |
| 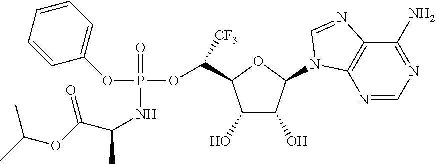 | A27 | B |
| 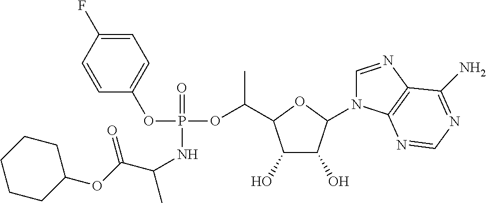 | A31 | A |
| 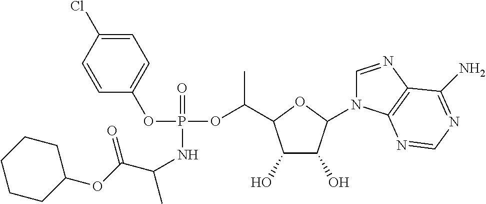 | A32 | A |
| 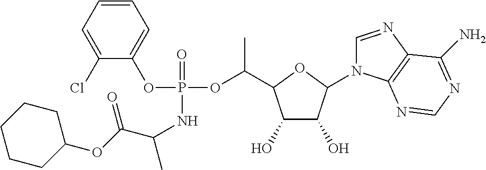 | A28 | A |
| 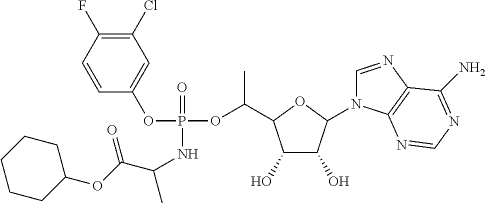 | A29 | A |
| 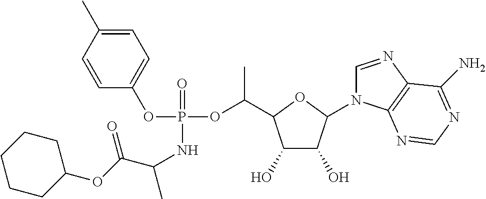 | A30 | A |
| 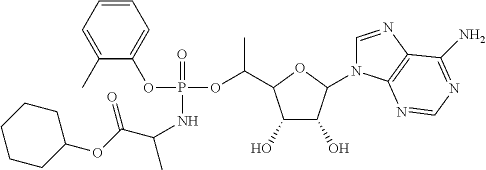 | A37 | A |
| 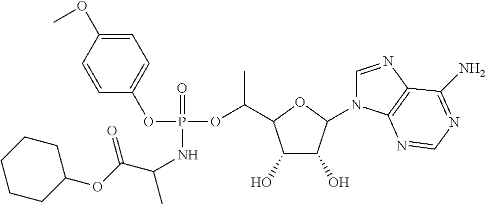 | A33 | A |
| 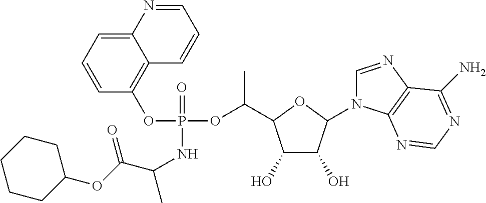 | A35 | B |
| 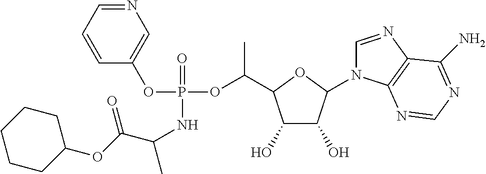 | A36 | B |
| 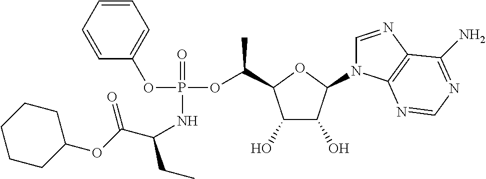 | A34 | A |
| 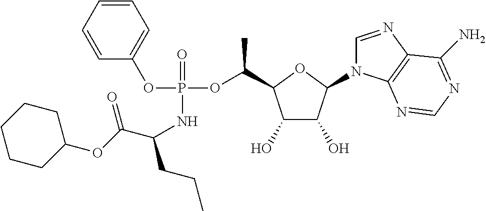 | A38 | A |
| 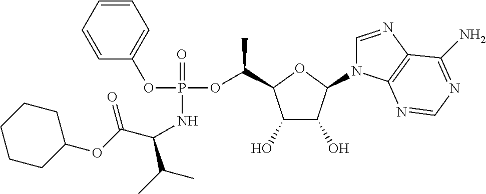 | A39 | A |
| 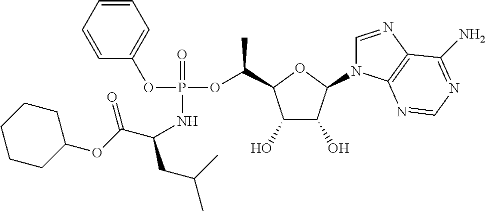 | A40 | A |
| 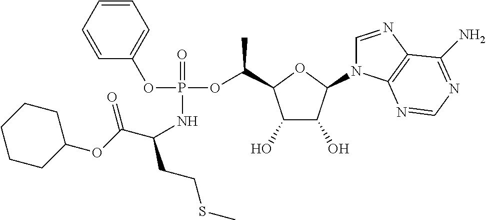 | A41 | A |
| 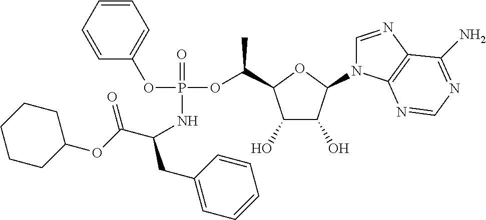 | A42 | B |
| 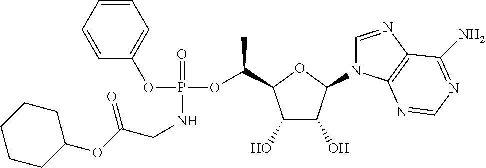 | A43 | A |
| 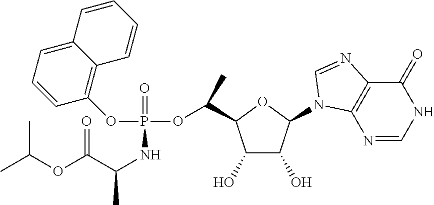 | B5 | C |
| 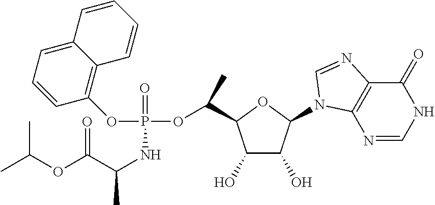 | B5 | C |
| 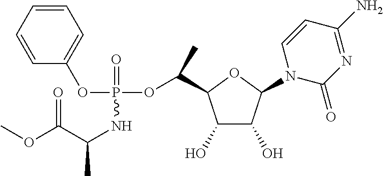 | C6 | C |
| 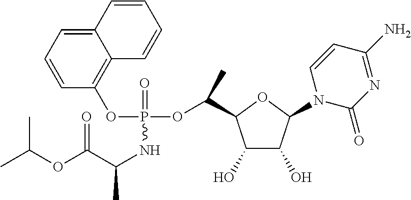 | C2 | C |
| 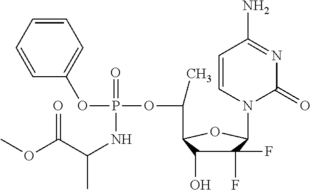 | C5 | A |
| 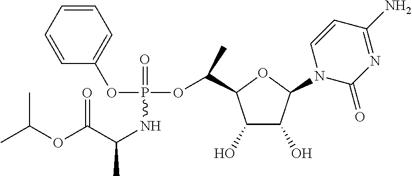 | C8 | C |
| 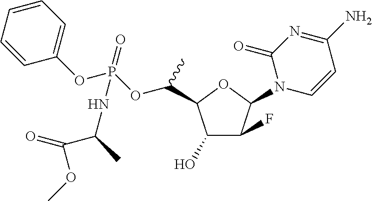 | C9 | C |
| 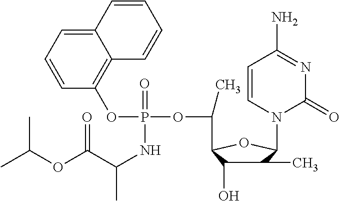 | C10 | B |
| 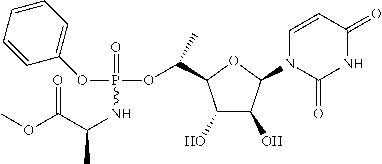 | D1 | D |
| 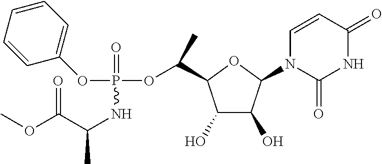 | D2 | D |
| 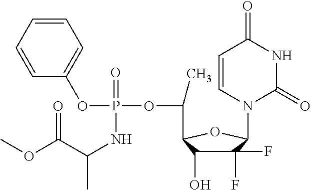 | D4 | B |
| 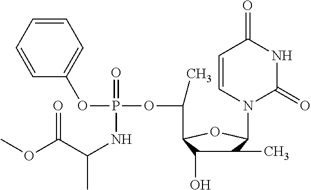 | D7 | B |
| 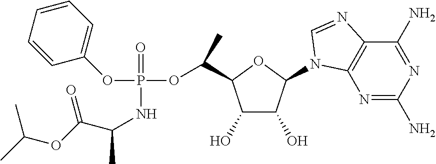 | E1 | B |

TABLE 9
Activity of Exemplary Compounds (C <200 μM, B <30 μM, A <5 μM)

| | HCV |
|---|---|
| | Repli- |
| | con |
| | Activ- |
| Structure | ity |
| 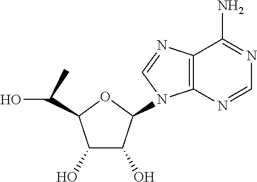 | B |
| 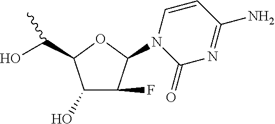 | B |
| 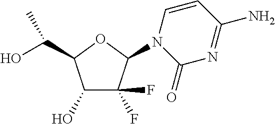 | A |
| 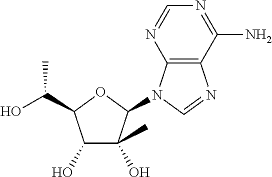 | B |
| 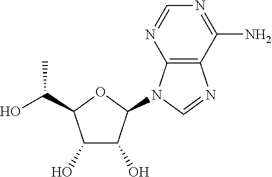 | C |
| 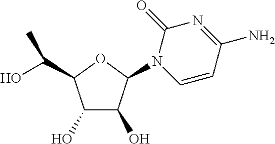 | C |
| 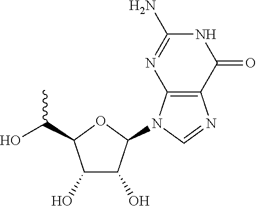 | C |
| 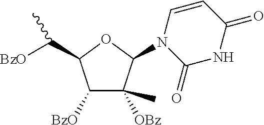 | B |
| 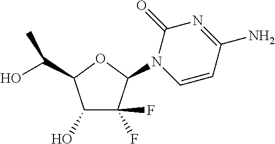 | A |
| 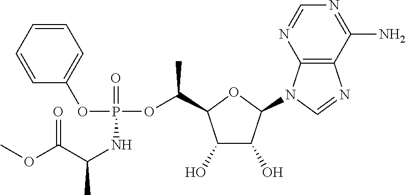 | A |
| 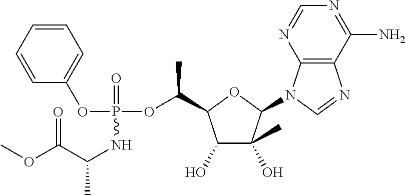 | C |
| 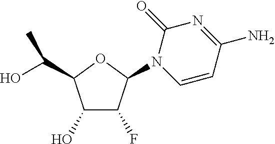 | B |
| 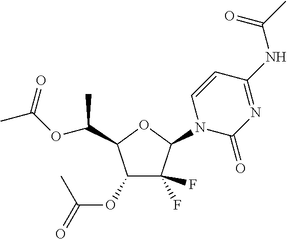 | A |
| 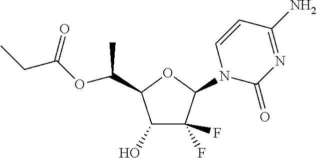 | A |
| 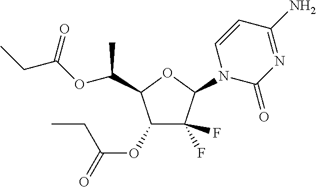 | A |
| 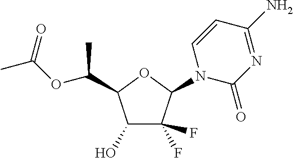 | A |
| 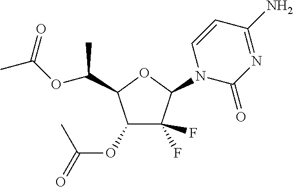 | A |
| 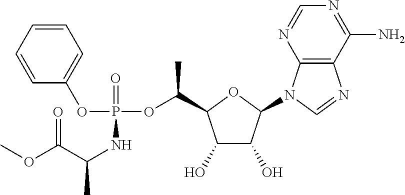 | A |
| 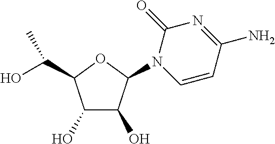 | A |
| 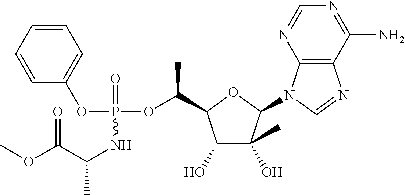 | C |

### Example 70

### Combination of Compounds

### Combination Testing

Two or more test compounds were tested in combination with each other using an HCV genotype 1b HCV replicon harbored in Huh7 cells with a stable luciferase (LUC) reporter. Cells were cultured under standard conditions in Dulbecco's modified Eagle's medium (DMEM; Mediatech Inc, Herndon, Va.) containing 10% heat-inactivated fetal bovine serum (FBS; Mediatech Inc, Herndon, Va.) 2 mM L-glutamine, and nonessential amino acids (JRH Biosciences). HCV replicon cells were plated in a 96-well plate at a density of 10⁴ cells per well in DMEM with 10% FBS. On the following day, the culture medium was replaced with DMEM containing either no compound as a control, the test compounds serially diluted in the presence of 2% FBS and 0.5% DMSO, or a combination of compound A10 with one or more test compounds serially diluted in the presence of 2% FBS and 0.5% DMSO. The cells were incubated with no compound as a control, with the test compounds, or the combination of compounds for 72 h. The direct effects of the combination of the test compounds were examined using a luciferase (LUC) based reporter as determined by the Bright-Glo Luciferase Assay (Promega, Madison, Wis.). Dose-response curves were determined for individual compounds and fixed ratio combinations of two or more test compounds.

The effects of test compound combinations were evaluated by two separate methods. In the Loewe additivity model, the experimental replicon data was analyzed by using CalcuSyn (Biosoft, Ferguson, Mo.), a computer program based on the method of Chou and Talalay. The program uses the experimental data to calculate a combination index (CI) value for each experimental combination tested. A CI value of <1 indicates a synergistic effect, a CI value of 1 indicates an additive effect, and a CI value of >1 indicates an antagonistic effect.

The second method utilized for evaluating combination effects used a program called MacSynergy II. MacSynergy II software was kindly provided by Dr. M. Prichard (University of Michigan). The Prichard Model allows for a three-dimensional examination of drug interactions and a calculation of the synergy volume (units: μM²%) generated from running the replicon assay using a checkerboard combination of two or more inhibitors. The volumes of synergy (positive volumes) or antagonism (negative volumes) represent the relative quantity of synergism or antagonism per change in the concentrations of the two drugs. Synergy and antagonism volumes are defined based on the Bliss independence model. In this model, synergy volumes of less than −25 indicate antagonistic interactions, volumes in the −25-25 range indicate additive behavior, volumes in the 25-100 range indicate synergistic behavior and volumes>100 indicate strong synergistic behavior. Determination of in vitro additive, synergistic and strongly synergistic behavior for combinations of compounds can be of utility in predicting therapeutic benefits for administering the combinations of compounds in vivo to infected patients.

The CI and synergy volume results for the combinations are provided in Table 10.

TABLE 10

| Combination | | Synergy Volume |
|---|---|---|
| Compound | CI at EC₅₀ | (μM² %) |
| INX-189 | 0.67 | 31 |
| PSI-938 | 1 | 36 |
| PSI-6130 | 1 | 21 |
| PSI-7851 | 1.2 | 14 |
| GS-9190 | 0.45 | 112 |
| Filibuvir | 0.46 | 38 |
| ANA-598 | 0.67 | 32 |
| VX-222 | 1.1 | 52 |
| VX-950 | 0.56 | 34 |
| ITMN-191 | 0.84 | 39 |
| TMC-435 | 0.85 | 104 |
| BMS-790052 | 0.48 | 25 |
| 6002 | 0.01 | 127 |
| Ribavirin | 0.88 | 0 |
| Pegylated | 1 | 13 |
| Interferon | | |
| Consensus | 1.1 | 18 |
| Interferon | | |
| Cyclosporin A | 0.75 | 67 |
| BILN-2061 | 0.86 | 12 |
| HCV-796 | 0.39 | 35 |
| IFN-Lambda 1 | 0.64 | 43 |
| IFN-Lambda 2 | 0.56 | 31 |
| IFN-Lambda 3 | 0.87 | 33 |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A compound of Formula (I) or a pharmaceutically acceptable salt thereof: 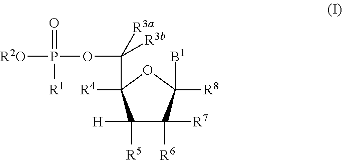
wherein:
B¹ is an optionally substituted heterocyclic base or an optionally substituted heterocyclic base with one or more protected amino groups;
R¹ is an optionally substituted N-linked amino acid or an optionally substituted N-linked amino acid ester derivative;
R² is selected from the group consisting of an optionally substituted aryl, an optionally substituted heteroaryl and an optionally substituted heterocyclyl;
R^{3a} and R^{3b} are independently selected from the group consisting of hydrogen, an optionally substituted C₁₋₆ alkyl, an optionally substituted C₂₋₆ alkenyl, an optionally substituted C₂₋₆ alkynyl, an optionally substituted C₁₋₆ haloalkyl and aryl(C₁₋₆ alkyl), provided that at least one of R^{3a} and R^{3b} is not hydrogen; or R^{3a} and R^{3b} are taken together to form a group selected from the group consisting of an optionally substituted C₃₋₆ cycloalkyl, an optionally substituted C₃₋₆ cycloalkenyl, an optionally substituted C₃₋₆ aryl and an optionally substituted C₃₋₆ heteroaryl;
R⁴ is hydrogen;
R⁵ is selected from the group consisting of hydrogen, —OR⁹ and —OC(═O)R¹⁰;
R⁶ is selected from the group consisting of hydrogen, halogen, —OR¹¹ and —OC(═O)R¹²;
or R⁵ and R⁶ are both oxygen atoms and linked together by a carbonyl group;
R⁷ is selected from the group consisting of hydrogen, halogen, an optionally substituted C₁₋₆ alkyl, —OR¹³ and —OC(═O)R¹⁴;
R⁸ is hydrogen or an optionally substituted C₁₋₆ alkyl;
R⁹, R¹¹ and R¹³ are independently selected from the group consisting of hydrogen and an optionally substituted C₁₋₆ alkyl; and
R¹⁰, R¹² and R¹⁴ are independently selected from the group consisting of an optionally substituted C₁₋₆ alkyl and an optionally substituted C₃₋₆ cycloalkyl;
provided a compound of Formula (I) cannot have a structure selected from the group consisting of:

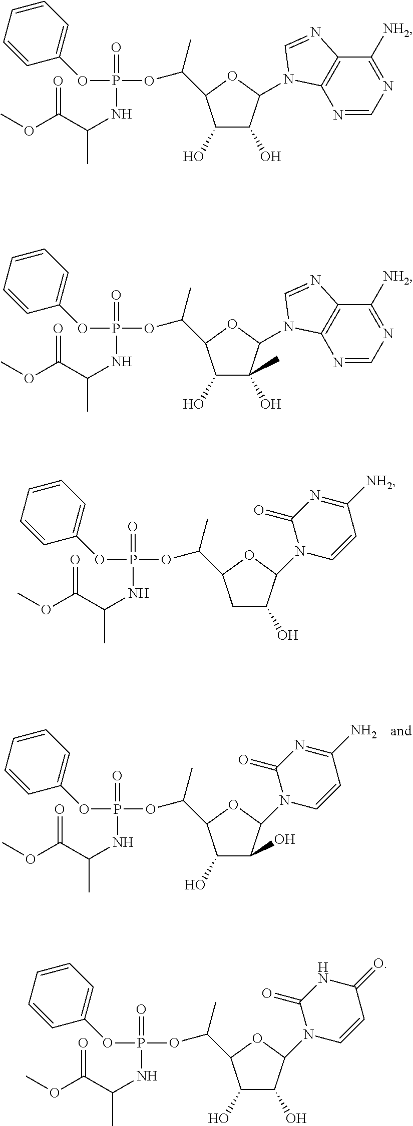

2. The compound of claim 1, wherein at least one of R^{3a} and R^{3b} is an optionally substituted C₁₋₆-alkyl; and the other of R^{3a} and R^{3b} is hydrogen.

3. The compound of claim 2, wherein the optionally substituted C₁₋₆-alkyl is methyl.

4. The compound of claim 1, wherein at least one of R^{3a} and R^{3b} is an optionally substituted C₂₋₆-alkyl; and the other of R^{3a} and R^{3b} is hydrogen.

5. The compound of claim 1, wherein R² is an optionally substituted aryl.

6. The compound of claim 5, wherein the optionally substituted aryl is an optionally substituted phenyl or an optionally substituted naphthyl.

7. The compound of claim 1, wherein R² is an optionally substituted heteroaryl.

8. The compound of claim 1, wherein R¹ is an optionally substituted N-linked α-amino acid.

9. The compound of claim 1, wherein R¹ is an optionally substituted N-linked α-amino acid ester derivative.

10. The compound of claim 1, wherein R¹ is selected from the group consisting of alanine, asparagine, aspartate, cysteine, glutamate, glutamine, glycine, proline, serine, tyrosine, arginine, histidine, isoleucine, leucine, lysine, methionine, phenylalanine, threonine, tryptophan, valine and ester derivatives thereof.

11. The compound of claim 10, wherein R¹ is selected from the group consisting of alanine isopropyl ester, alanine cyclohexyl ester, alanine neopentyl ester, valine isopropyl ester, and leucine isopropyl ester.

12. The compound of claim 11, wherein R¹ is alanine cyclohexyl ester.

13. The compound of claim 1, wherein R¹ has the structure 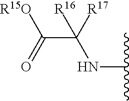
wherein R¹⁵ is selected from the group consisting of hydrogen, an optionally substituted C₁₋₆-alkyl, an optionally substituted C₃₋₆ cycloalkyl, an optionally substituted aryl, an optionally substituted aryl(C₁₋₆ alkyl) and an optionally substituted C₁₋₆ haloalkyl; and R¹⁶ is selected from the group consisting of hydrogen, an optionally substituted C₁₋₆ alkyl, an optionally substituted C₁₋₆ haloalkyl, an optionally substituted C₃₋₆ cycloalkyl, an optionally substituted C₆ aryl, an optionally substituted C₁₀ aryl and an optionally substituted aryl(C₁₋₆ alkyl); and R¹⁷ is hydrogen or an optionally substituted C₁₋₄-alkyl; or R¹⁶ and R¹⁷ are taken together to form an optionally substituted C₃₋₆ cycloalkyl.

14. The compound of claim 13, wherein R¹⁶ is an optionally substituted C₁₋₆-alkyl.

15. The compound of claim 14, wherein the optionally substituted C₁₋₆-alkyl is methyl.

16. The compound of claim 13, wherein R¹⁷ is hydrogen.

17. The compound of claim 13, wherein R¹⁵ is an optionally substituted C₁₋₆ alkyl or an optionally substituted C₃₋₆ cycloalkyl.

18. The compound claim 13, wherein

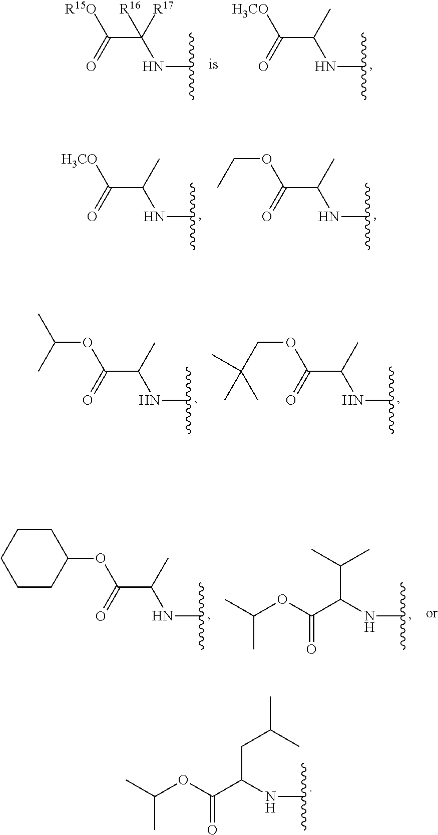

19. The compound of claim 18, wherein

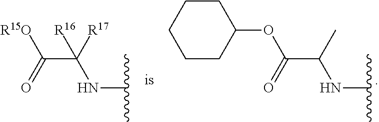

20. The compound of claim 1, wherein R⁷ is selected from the group consisting of hydrogen, halogen, an optionally substituted C₁₋₆ alkyl, and —OH.

21. The compound of claim 1, wherein R⁵ is hydrogen.

22. The compound of claim 1, wherein R⁵ is —OR⁹.

23. The compound of claim 22, wherein R⁹ is hydrogen.

24. The compound of claim 22, wherein R⁹ is an optionally substituted C₁₋₆ alkyl.

25. The compound of claim 1, wherein R⁵ is —OC(═O)R¹⁰.

26. The compound of claim 1, wherein R⁶ is —OR¹¹ or —OC(═O)R¹².

27. The compound of claim 1, wherein R⁵ and R⁶ are both oxygen atoms and linked together by a carbonyl group; or R⁵ and R⁶ are both hydrogen; or R⁵ is —OH, and R⁶ is —OH; or R⁵ is —OC(═O)R¹⁰ and R⁶ is —OC(═O)R¹².

28. The compound of claim 1, wherein R⁶ and R⁷ are both halogen; or R⁶ is hydrogen, and R⁷ is selected from halogen, an optionally substituted C₁₋₆ alkyl and —OR¹³; or R⁶ is halogen, and R⁷ is an optionally substituted C₁₋₆ alkyl.

29. The compound of claim 1, wherein R⁸ is hydrogen.

30. The compound of claim 1, wherein R⁸ is an optionally substituted C₁₋₆ alkyl.

31. The compound of claim 1, wherein B¹ is selected from the group consisting of: 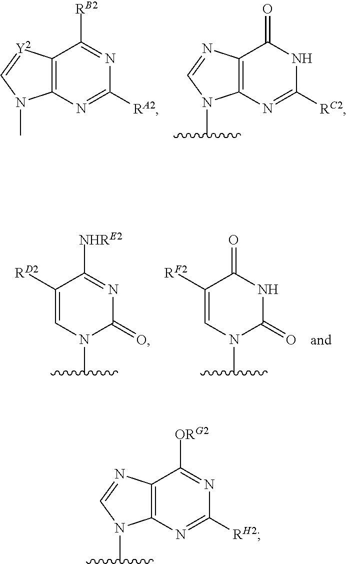
wherein:
R^{A2} is selected from the group consisting of hydrogen, halogen and NHR^{J2}, wherein R^{J2} is selected from the group consisting of hydrogen, —C(═O)R^{K2} and —C(═O)OR^{L2};
R^{B2} is halogen or NHR^{W2}, wherein R^{W2} is selected from the group consisting of hydrogen, an optionally substituted C₁₋₆ alkyl, an optionally substituted C₂₋₆ alkenyl, an optionally substituted C₃₋₈ cycloalkyl, —C(═O)R^{M2} and —C(═O)OR^{N2};
R^{C2} is hydrogen or NHR^{O2}, wherein R^{O2} is selected from the group consisting of hydrogen, —C(═O)R^{P2} and —C(═O)OR^{Q2};
R^{D2} is selected from the group consisting of hydrogen, halogen, an optionally substituted C₁₋₆ alkyl, an optionally substituted C₂₋₆ alkenyl and an optionally substituted C₂₋₆ alkynyl;
R^{E2} is selected from the group consisting of hydrogen, an optionally substituted C₁₋₆ alkyl, an optionally substituted C₃₋₈ cycloalkyl, —C(═O)R^{R2} and —C(═O)OR^{S2};
R^{F2} is selected from the group consisting of hydrogen, halogen, an optionally substituted C₁₋₆alkyl, an optionally substituted C₂₋₆ alkenyl and an optionally substituted C₂₋₆ alkynyl;
Y² is N or CR^{I2}, wherein R^{I2} is selected from the group consisting of hydrogen, halogen, an optionally substituted C₁₋₆-alkyl, an optionally substituted C₂₋₆-alkenyl and an optionally substituted C₂₋₆-alkynyl;
R^{G2} is an optionally substituted C₁₋₆ alkyl;
R^{H2} is hydrogen or NHR^{T2}, wherein R^{T2} is independently selected from the group consisting of hydrogen, —C(═O)R^{U2} and —C(═O)OR^{V2}, and
R^{K2}, R^{L2}, R^{M2}, R^{N2}, R^{P2}, R^{Q2} R^{R2}, R^{S2}, R^{U2} and R^{V2} are independently selected from the group consisting of C₁₋₆ alkyl, C₂₋₆ alkenyl, C₂₋₆ alkynyl, C₃₋₆ cycloalkyl, C₃₋₆ cycloalkenyl, C₃₋₆ cycloalkynyl, C₆₋₁₀ aryl, heteroaryl, heteroalicyclyl, aryl(C₁₋₆ alkyl), heteroaryl(C₁₋₆ alkyl) and heteroalicyclyl(C₁₋₆ alkyl).

32. The compound of claim 31, wherein B¹ is selected from the group consisting 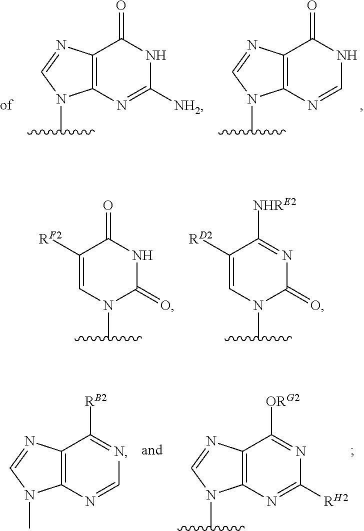
wherein R^{D2} is hydrogen, and R^{B2} is NH₂.

33. The compound of claim 1, wherein when R² is phenyl then R¹ cannot be

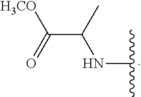

34. The compound of claim 1, wherein the compound of Formula (I) is selected from the group consisting of: 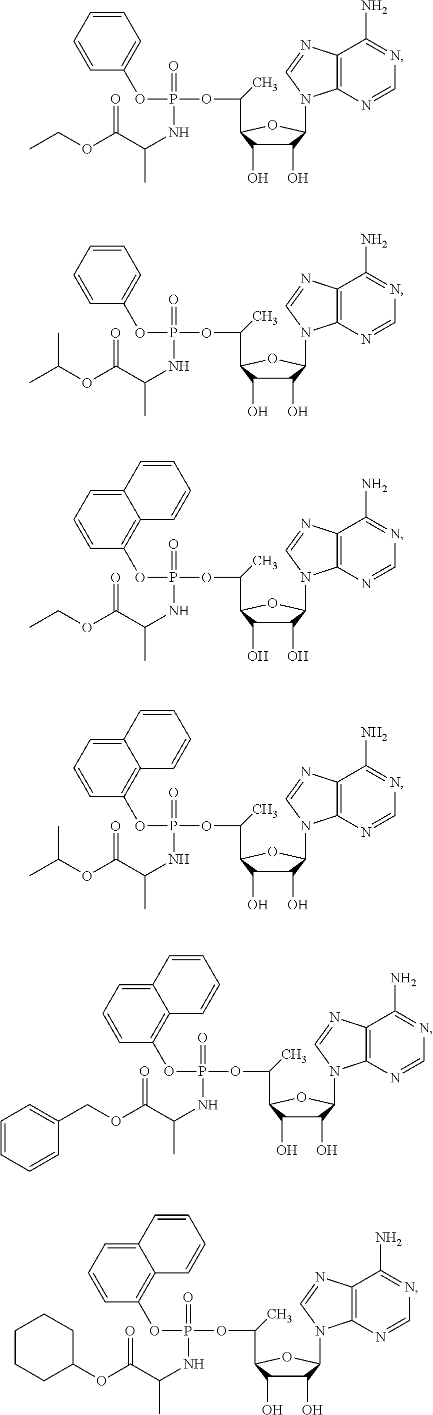 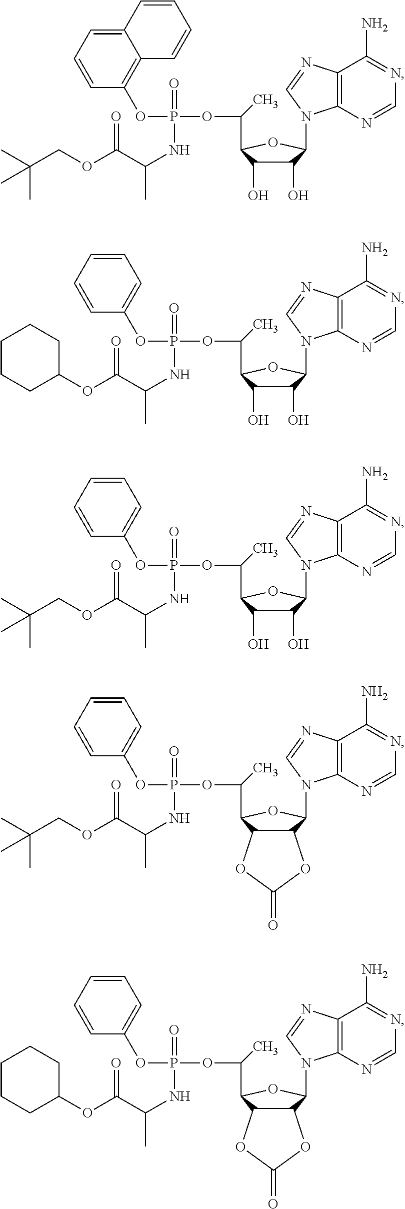 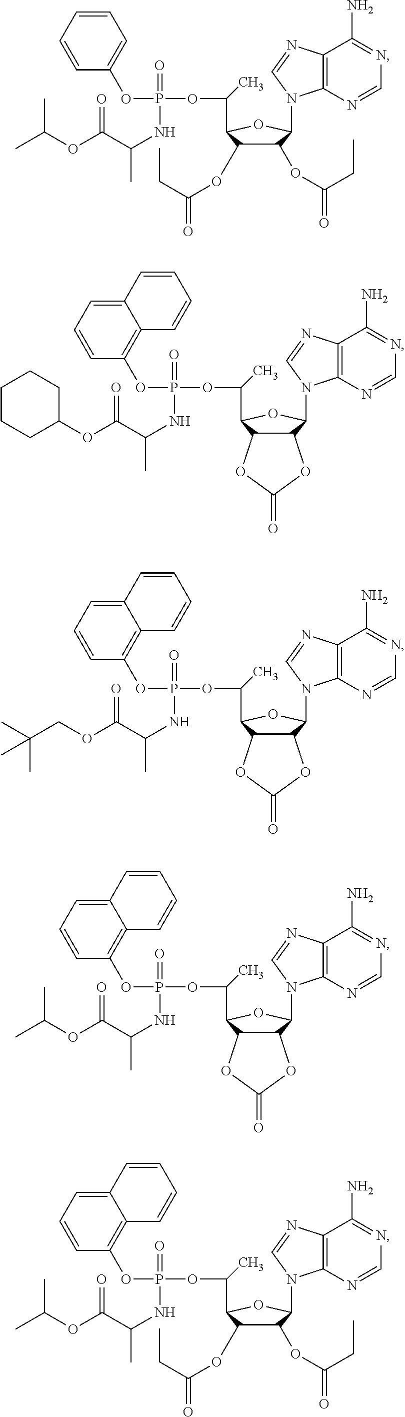 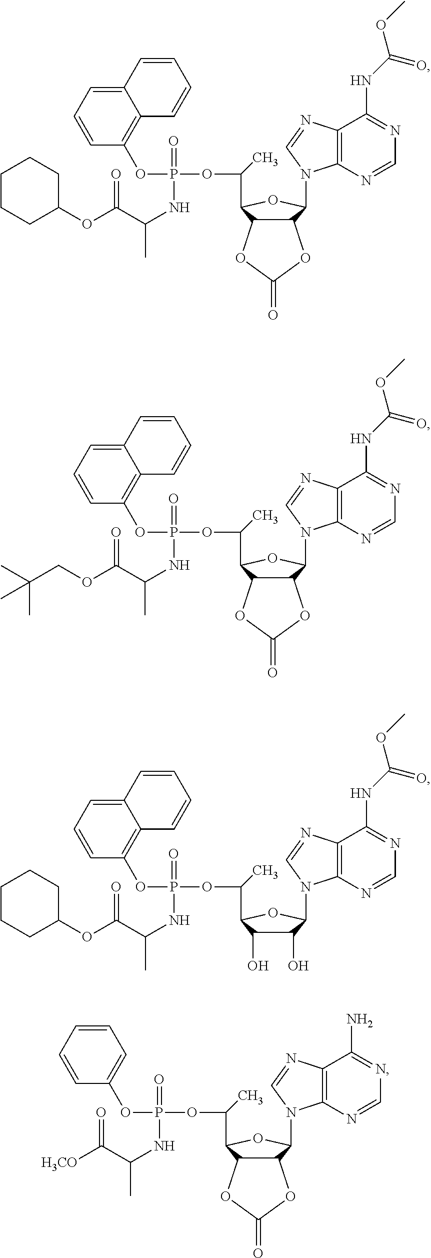 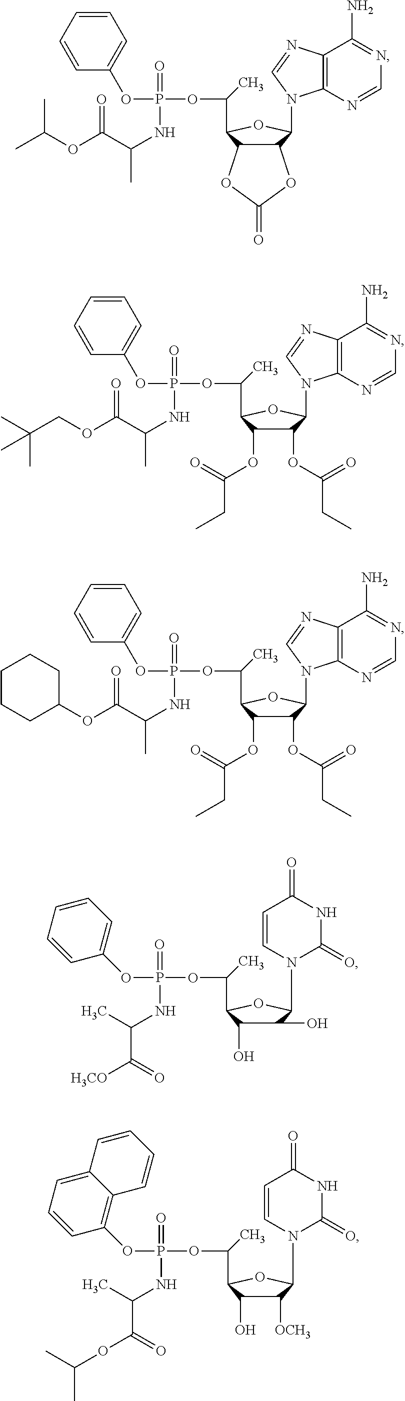 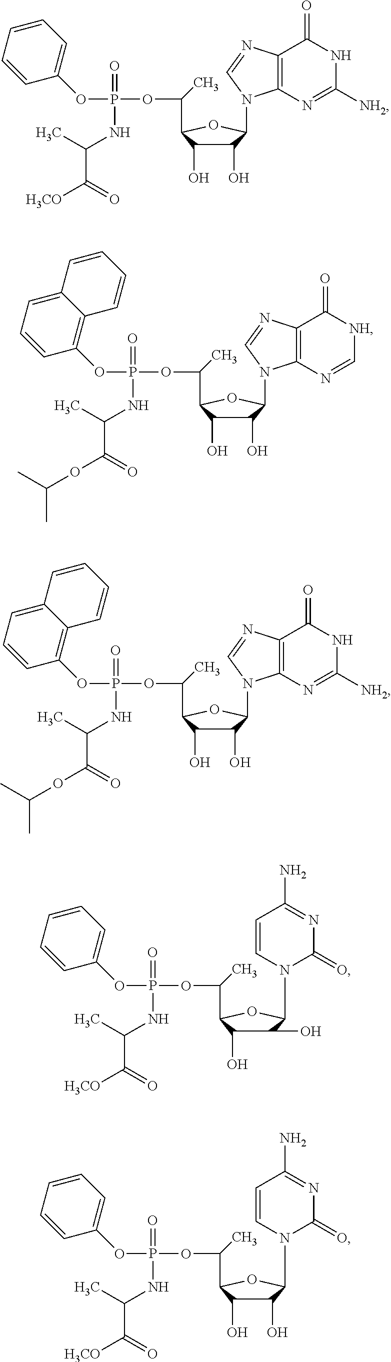 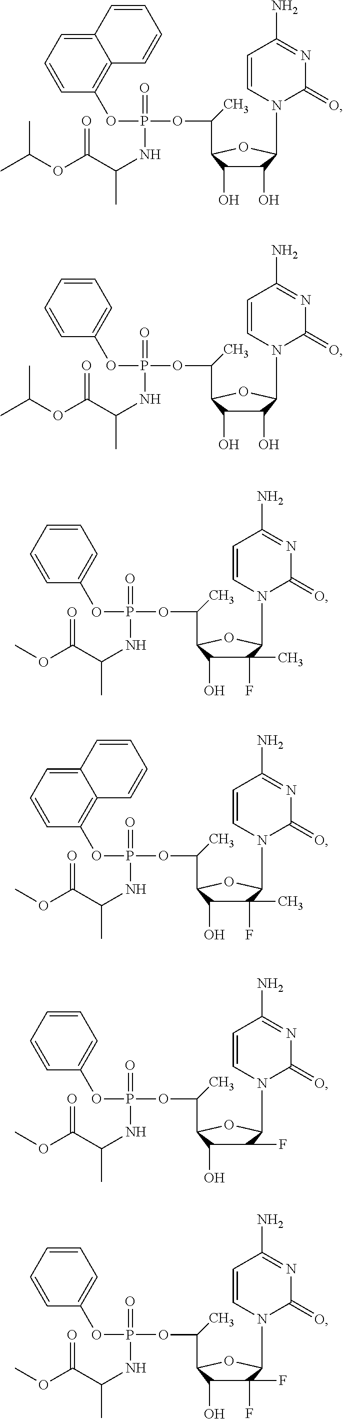 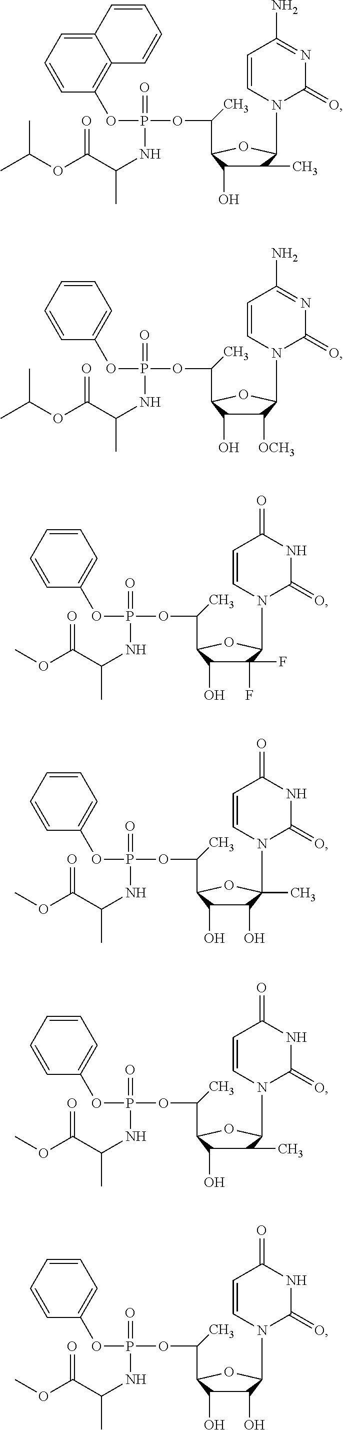 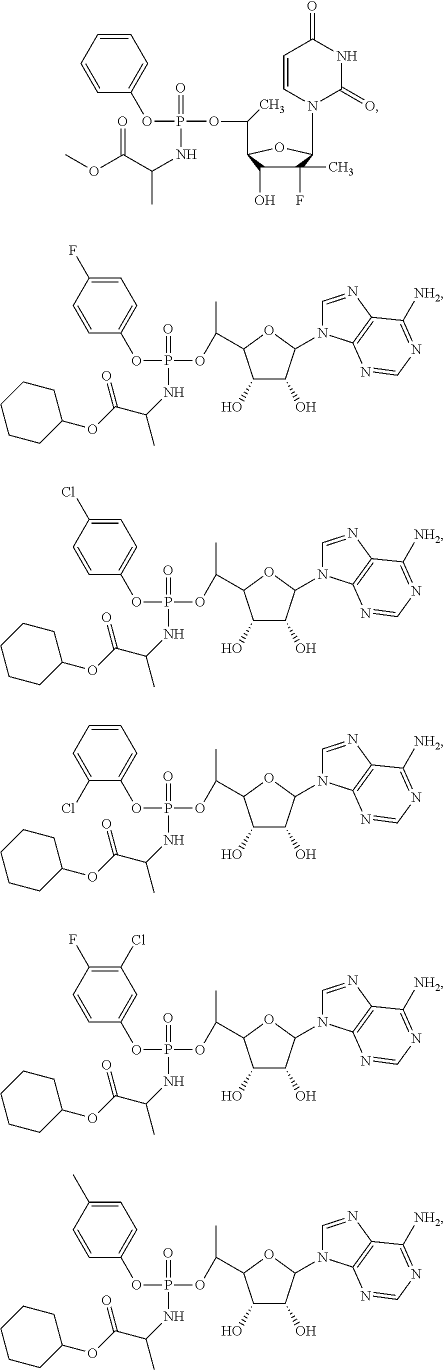 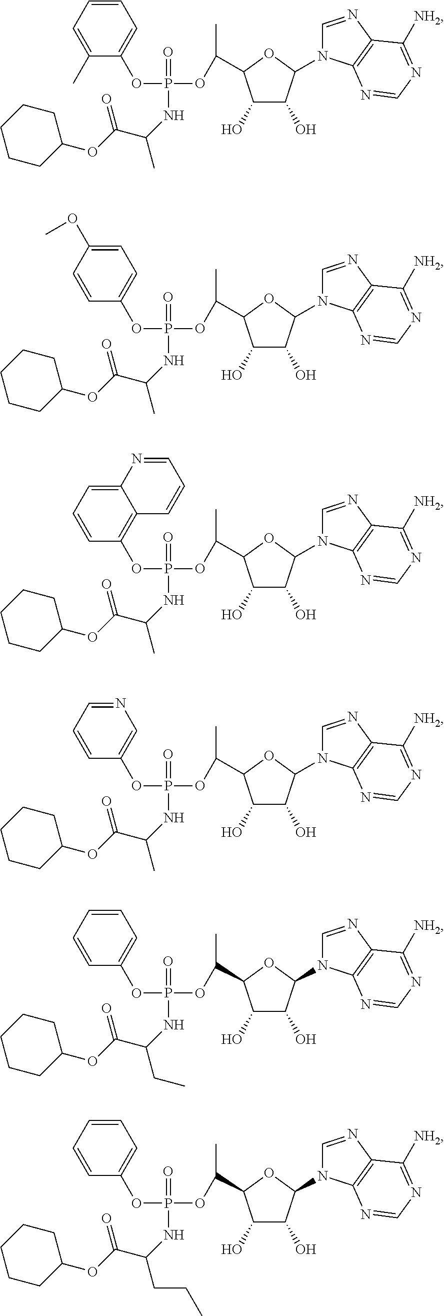 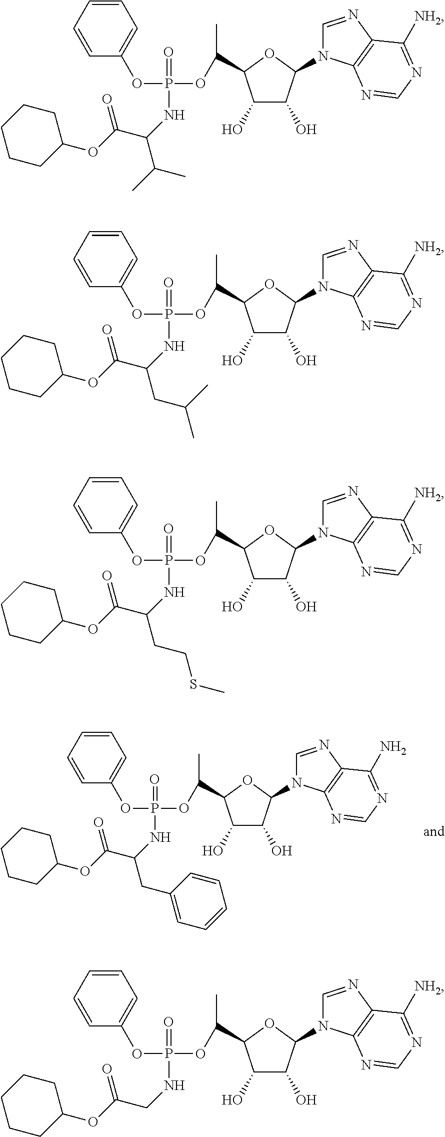
or a pharmaceutically acceptable salt of the foregoing.

35. The compound of claim 1, wherein the compound of Formula (I) is 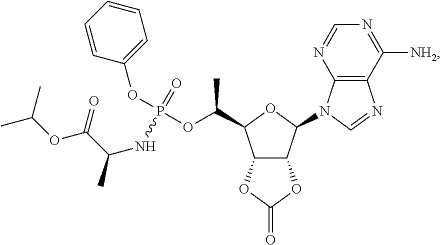
or a pharmaceutically acceptable salt thereof.

36. The compound of claim 1, wherein the compound of Formula (I) is 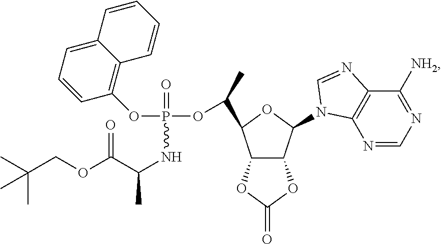
or a pharmaceutically acceptable salt thereof.

37. The compound of claim 1, wherein the compound of Formula (I) is 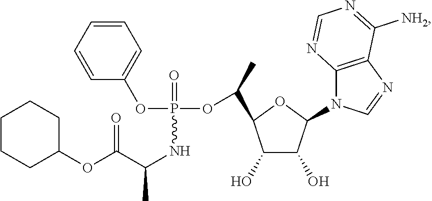
or a pharmaceutically acceptable salt thereof.

38. The compound of claim 1, wherein the compound of Formula (I) is 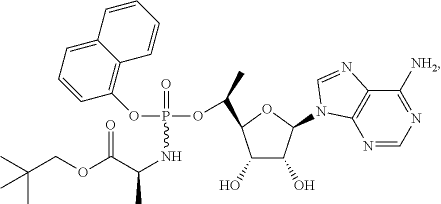
or a pharmaceutically acceptable salt thereof.

39. A pharmaceutical composition comprising a therapeutically effective amount of a compound of claim 1, or a pharmaceutically acceptable salt thereof, and a pharmaceutically acceptable carrier, diluent, excipient or combination thereof.

40. A method of ameliorating or treating a viral infection comprising administering to a subject suffering from the viral infection a therapeutically effective amount of a compound of claim 1, or a pharmaceutically acceptable salt thereof.

41. The method of claim 40, wherein the viral infection is caused by a virus selected from the group consisting of an adenovirus, an Alphaviridae, an Arbovirus, an Astrovirus, a Bunyaviridae, a Coronaviridae, a Filoviridae, a Flaviviridae, a Hepadnaviridae, a Herpesviridae, an Alphaherpesvirinae, a Betaherpesvirinae, a Gammaherpesvirinae, a Norwalk Virus, an Astroviridae, a Caliciviridae, an Orthomyxoviridae, a Paramyxoviridae, a Paramyxoviruses, a Rubulavirus, a Morbillivirus, a Papovaviridae, a Parvoviridae, a Picornaviridae, an Aphthoviridae, a Cardioviridae, an Enteroviridae, a Coxsackie virus, a Polio Virus, a Rhinoviridae, a Phycodnaviridae, a Poxyiridae, a Reoviridae, a Rotavirus, a Retroviridae, an A-Type Retrovirus, an Immunodeficiency Virus, a Leukemia Viruses, an Avian Sarcoma Viruses, a Rhabdoviruses, a Rubiviridae and a Togaviridae.

42. A method for ameliorating or treating an HCV infection comprising administering to a subject suffering from an HCV infection a therapeutically effective amount of a compound of claim 1, or a pharmaceutically acceptable salt thereof.

43. A method for inhibiting NS5B polymerase activity comprising contacting a cell with an effective amount of a compound of claim 1, or a pharmaceutically acceptable salt thereof.

44. A method for inhibiting replication of a virus comprising contacting a cell infected with the virus with a compound of claim 1, or a pharmaceutically acceptable salt thereof.

45. The method of claim 44, wherein the virus is HCV.

46. A method for ameliorating or treating a viral infection comprising contacting a cell infected with the virus with a compound of claim 1, or a pharmaceutically acceptable salt thereof.

47. The method of claim 46, wherein the viral infection is a HCV viral infection.

48. A method of ameliorating or treating a viral infection comprising contacting a cell infected with the viral infection with a therapeutically effective amount of a compound selected from a compound of claim 1, compound 7072, compound 7073, compound 7074, compound 7075, compound 7076, compound 7077, a monophosphate of any of the foregoing, a diphosphate of any of the foregoing, or a pharmaceutically acceptable salt the foregoing, in combination with one or more agents selected from the group consisting of an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

49. The method of claim 48, wherein the one or more agents are selected from the group consisting of Compounds 1001-1014, 2001-2010, 3001-3008, 4001-4005, 5001-5002, 6000-6078, 8000-8012 and 9000, or a pharmaceutically acceptable salt of any of the aforementioned compounds.

50. The method of claim 48, wherein the viral infection is a HCV viral infection.

51. A method of ameliorating or treating a viral infection comprising administering to a subject suffering from the viral infection a therapeutically effective amount of a compound selected from a compound of claim 1, compound 7072, compound 7073, compound 7074, compound 7075, compound 7076, and compound 7077, a monophosphate of any of the foregoing, a diphosphate of any of the foregoing, or a pharmaceutically acceptable salt the foregoing, in combination with one or more agents selected from the group consisting of an interferon, ribavirin, a HCV protease inhibitor, a HCV polymerase inhibitor, a NS5A inhibitor, an antiviral compound, a compound of Formula (BB), a compound of Formula (CC) and a compound of Formula (DD), or a pharmaceutically acceptable salt of any of the aforementioned compounds.

52. The method of claim 51, wherein the one or more agents are selected from the group consisting of Compounds 1001-1014, 2001-2010, 3001-3008, 4001-4005, 5001-5002, 6000-6078, 8000-8012 and 9000, or a pharmaceutically acceptable salt of any of the aforementioned compounds.

53. The method of claim 51, wherein the viral infection is a HCV viral infection.
